SEQUENCE LISTING (45 sequences)

SEQ ID NO: 1 - protein, 534 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNELINQSLAFINQSDELLHNVNAGKSTTNIMITTIIIVIIVILLSLIAVGLLLYCKARSTPVTLSKDQLSGINNIAFSN

SEQ ID NO: 2 - protein, 539 aa, synthetic construct
MSWKVVIIFSLLITPQHGLKESYLEESCSTITEGYLSVLRTGWYTVVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLAFAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHN

SEQ ID NO: 3 - protein, 539 aa, synthetic construct
MLISILSIITTMIMASHCQIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVCSSVGNCIVAIKSVQDYVNKEIVPSIARLGCEAAGLQLGIALTQHYSELTNCFGDNIGSLQEKGIKLQCIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKVKSDLEESKEWYRRSNQKLDSIGSWHQSSTTIIIILIMMIILFIINITIITIAIKYYRIQKRNRVDQNDKPYVLTNK

SEQ ID NO: 4 - RNA, 1605 nt, synthetic construct
  source 1..1605: mol_type = other RNA
  mRNA 1..1605: note = mRNA
atggaactgctgatcctcaaagccaacgcaatcaccaccattctcaccgctgtgaccttctgcttcgcatcggggcagaacatcactgaagagttttaccagagcacttgcagcgcggtgtcaaagggttacctttccgcactgcggaccggatggtacacttccgtgatcaccattgagctcagcaacatcaaggaaaacaagtgcaatggcaccgacgccaaggtcaagctgatcaaacaagaactggacaagtacaagaacgccgtgacagaattgcagctcctgatgggatccggaaacgtcggtctgggcggagccatcgcgagtggagtggctgtgtccaaggtcttgcacctcgagggagaagtgaacaagatcaagtccgcgctgctgtcaacgaacaaggccgtggtgtccctgtctaacggcgtcagcgtgctgacgttcaaggtcctggacctgaagaattacattgacaagcagctgctgcccatcctcaacaagcaatcctgctccatctccaaccccgaaaccgtgatcgagttccagcagaagaacaaccgcctgctggaaattactcgcgagttctctgtgaatgccggcgtgaccacccctgtgtccacctacatgctgaccaactccgagcttctctcccttatcaatgacatgcctatcacgaacgaccagaagaagctgatgtcgaacaacgtgcagattgtgcggcagcagtcatacagcatcatgtcgatcatcaaggaagaagtgctggcgtacgtggtgcaactcccgctgtacggcgtcatcgataccccgtgctggaagctgcacacctcgcctttgtgtaccaccaacaccaagaacggatccaacatctgcttaacccggactgatcggggttggtactgcgacaacgccgggaatgtttcgttcttcccacaagccgagacttgtaaagtgcagtcaaacagagtgttctgtgacaccatgaactcgagaaccctgcccagcgaagtgaacctgtgtaacgtcgacatctttaacccaaaatacgattgcaagattatgaccagcaaaaccgacgtgtcctcctccgtgataacaagcctgggggcgattgtgtcatgctacggaaagactaagtgcaccgcctcgaacaagaaccgcggcatcattaagactttctcgaatggttgcgactatgtgtccaacaagggcgtggatactgtgtcagtcgggaatactctttactacgtgaacaagcaggaggggaaaagcctctacgtgaagggagagcctattatcaacttttacgatccgctggtgttcccgtccgacgaattcgacgccagcatcagccaagtcaacgagctgattaaccagtccctcgccttcatcaaccaatccgacgagctcctgcataacgtgaacgccggaaagtccaccaccaacatcatgatcactactattatcatcgtgatcatcgtcatcctgctgagcctgattgctgtgggcctgttgctgtattgcaaagccaggtccaccccggtcaccctgtcgaaggatcagctgtccggaatcaacaacattgccttctccaactaa

SEQ ID NO: 5 - RNA, 1620 nt, synthetic construct
  source 1..1620: mol_type = other RNA
  mRNA 1..1620: note = mRNA
atgagctggaaggttgtgattattttctctctgctgattactccacagcacggcctgaaggagtcctacctggaggagtcctgttctactatcactgaggggtatctctctgtgctgcggacagggtggtatacagtggtgttcaccctggaggttggcgatgtggagaatctgacttgcagcgatggcccttctctgatcaagaccgagctggatctgacaaaaagcgccctcagagaactgaaaaccgtgtccgccgatcagctggcaagggaggagcagatcgagaacccacggcagagcaggtttgtgctgggcgctatcgctctgggcgtggccactgcagctgctgtcactgcaggggtcgcaatcgctaagactatcagactggaatccgaggtgaccgccattaagaatgccctgaagactaccaacgaggctgtgtccactctgggaaacggagtgagggtcctggccttcgcagtgagggagctgaaggattttgtgtcaaagaaccttacacgggccatcaacaagaataagtgcgatatcgatgacctgaagatggccgtgtccttctcccagttcaaccggcgctttctgaatgtggtgcgccagttttccgacaacgctggaatcacccctgctatcagcctggacctcatgaccgacgccgaactcgcaagggccgtttctaacatgcctacatccgctggacagattaagctgatgctggagaatagagcaatggtgaggagaaagggattcggcatcctgattggcgtgtacggatctagcgtgatctacatggtgcagctgccgatcttcggcgtgatcgatactccttgttggatcgtcaaggccgccccttcctgctccgagaagaagggcaattacgcttgtctgctgcgggaggaccagggctggtattgccagaacgccgggtctacagtgtactatcctaacgagaaggattgcgagaccagaggcgaccacgttttctgtgatacagccgccggaatcaatgtcgcagagcagtctaaggagtgcaacatcaatatctctacaaccaattacccatgtaaggtgagcactggacggcaccctatcagtatggtggctctgagcccactgggggcactggtggcttgctacaagggggtgagctgcagtatcggcagtaacagagtgggcattatcaagcagctgaacaaagggtgctcttatattacaaaccaggatgcagatactgtgaccatcgacaacactgtgtaccagctgtccaaggtggagggggagcagcatgtgatcaaagggagacccgtctcttcttctttcgatcccatcaagttccctgaagaccagttcaatgttgccctggaccaggttttcgagaacatcgaaaatagccaggccttggtcgatcaatccaacaggatcctgagcagcgcagagaaagggaacactggcttcatcatcgtgatcattctgatcgccgtgctggggagcagtatgattctggtgtccattttcatcatcatcaagaagaccaagaagcctacaggagcaccccctgagctgagcggagtgaccaacaacggctttatccctcacaactaa

SEQ ID NO: 6 - RNA, 1620 nt, synthetic construct
  source 1..1620: mol_type = other RNA
  mRNA 1..1620: note = mRNA
atgctgattagcattctgtccatcatcacaaccatgattatggcaagtcactgtcagatcgatatcaccaagctccagcacgtgggcgtgctggtgaattccccaaaggggatgaagatttctcagaactttgagactcggtacctgatcctgtccctgattcctaagattgaggatagcaactcctgtggcgaccagcagatcaaacagtacaaacgcctgctggacagactgatcatcccactgtacgacggcctgaagctgcagaaagacgtcattgttacaaaccaggaaagcaacgagaataccgaccctaggactgagcggttcttcgggggcgtcatcggaacaatcgcactgggcgttgccactagcgcacagatcaccgccgccgttgcactggtggaggctaagcaggcaaaatctgacatcgagaagctgaaagaagctatccgcgataccaacaaggccgtgcagtcagtgtgctccagcgtcggcaactgcatcgttgctattaagtccgtgcaggactacgtgaacaaggagatcgtgcccagtatcgcaaggctgggatgtgaggccgctggactgcagttggggatcgctctcacccagcactactccgagctgacaaattgcttcggagataacatcggatctctgcaggagaagggcatcaaactgcagtgtattgcctccctgtacagaacaaatatcacagagattttcacaaccagcactgtcgacaaatatgatatctatgacctgctgttcaccgagtccattaaggtgcgggtcatcgacgttgacctgaacgattattccatcaccctccaggtgagactgcctctgctgacacggctgctgaacactcagatctacaaggtcgactccatcagctacaacatccagaaccgcgagtggtacatcccactgcctagccacatcatgacaaagggcgcttttctgggaggagctgatgtgaaggaatgcattgaggccttcagcagctacatctgcccttccgatcccggcttcgtcctgaaccacgagatggagagctgtctgagcgggaacattagccagtgccccaggacaaccgtcacctctgacattgtgccaaggtatgccttcgtgaacggcggagtggttgccaattgcatcaccactacatgcacttgcaatgggattggcaacaggatcaaccagccaccagaccagggggtgaaaatcattacacacaaagagtgcaacaccatcggcatcaacggaatgctgttcaataccaacaaggagggcaccctggccttctacacacccgatgacatcaccctgaacaatagcgtggctctggatcccattgatatcagcattgagctgaataaggtcaagtctgatctggaggagagcaaggagtggtatagacggtctaatcagaagctcgactctatcggatcctggcaccagtcctccacaacaatcatcattatcctgatcatgatgatcatcctgttcatcatcaacatcacaattatcaccattgccatcaagtattaccggattcagaagcggaatcgcgttgaccagaacgacaaaccttatgttctgaccaacaaataa

SEQ ID NO: 7 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 8 - RNA, 100 nt, synthetic construct
  source 1..100: mol_type = other RNA
cgggtggcatccctgtgacccctccccagtgcctctcctggccctggaagttgccactccagtgcccaccagccttgtcctaataaaattaagttgcatc

SEQ ID NO: 9 - protein, 574 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 10 - protein, 539 aa, Human metapneumovirus
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHN

SEQ ID NO: 11 - protein, 539 aa, synthetic construct
MLISILSIITTMIMASHCQIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGSWHQSSTTIIIILIMMIILFIINITIITIAIKYYRIQKRNRVDQNDKPYVLTNK

SEQ ID NO: 12 - DNA, 1605 bp, synthetic construct
  source 1..1605: mol_type = other DNA
atggaactgctgatcctcaaagccaacgcaatcaccaccattctcaccgctgtgaccttctgcttcgcatcggggcagaacatcactgaagagttttaccagagcacttgcagcgcggtgtcaaagggttacctttccgcactgcggaccggatggtacacttccgtgatcaccattgagctcagcaacatcaaggaaaacaagtgcaatggcaccgacgccaaggtcaagctgatcaaacaagaactggacaagtacaagaacgccgtgacagaattgcagctcctgatgggatccggaaacgtcggtctgggcggagccatcgcgagtggagtggctgtgtccaaggtcttgcacctcgagggagaagtgaacaagatcaagtccgcgctgctgtcaacgaacaaggccgtggtgtccctgtctaacggcgtcagcgtgctgacgttcaaggtcctggacctgaagaattacattgacaagcagctgctgcccatcctcaacaagcaatcctgctccatctccaaccccgaaaccgtgatcgagttccagcagaagaacaaccgcctgctggaaattactcgcgagttctctgtgaatgccggcgtgaccacccctgtgtccacctacatgctgaccaactccgagcttctctcccttatcaatgacatgcctatcacgaacgaccagaagaagctgatgtcgaacaacgtgcagattgtgcggcagcagtcatacagcatcatgtcgatcatcaaggaagaagtgctggcgtacgtggtgcaactcccgctgtacggcgtcatcgataccccgtgctggaagctgcacacctcgcctttgtgtaccaccaacaccaagaacggatccaacatctgcttaacccggactgatcggggttggtactgcgacaacgccgggaatgtttcgttcttcccacaagccgagacttgtaaagtgcagtcaaacagagtgttctgtgacaccatgaactcgagaaccctgcccagcgaagtgaacctgtgtaacgtcgacatctttaacccaaaatacgattgcaagattatgaccagcaaaaccgacgtgtcctcctccgtgataacaagcctgggggcgattgtgtcatgctacggaaagactaagtgcaccgcctcgaacaagaaccgcggcatcattaagactttctcgaatggttgcgactatgtgtccaacaagggcgtggatactgtgtcagtcgggaatactctttactacgtgaacaagcaggaggggaaaagcctctacgtgaagggagagcctattatcaacttttacgatccgctggtgttcccgtccgacgaattcgacgccagcatcagccaagtcaacgagctgattaaccagtccctcgccttcatcaaccaatccgacgagctcctgcataacgtgaacgccggaaagtccaccaccaacatcatgatcactactattatcatcgtgatcatcgtcatcctgctgagcctgattgctgtgggcctgttgctgtattgcaaagccaggtccaccccggtcaccctgtcgaaggatcagctgtccggaatcaacaacattgccttctccaactaa

SEQ ID NO: 13 - DNA, 1620 bp, synthetic construct
  source 1..1620: mol_type = other DNA
atgagctggaaggttgtgattattttctctctgctgattactccacagcacggcctgaaggagtcctacctggaggagtcctgttctactatcactgaggggtatctctctgtgctgcggacagggtggtatacagtggtgttcaccctggaggttggcgatgtggagaatctgacttgcagcgatggcccttctctgatcaagaccgagctggatctgacaaaaagcgccctcagagaactgaaaaccgtgtccgccgatcagctggcaagggaggagcagatcgagaacccacggcagagcaggtttgtgctgggcgctatcgctctgggcgtggccactgcagctgctgtcactgcaggggtcgcaatcgctaagactatcagactggaatccgaggtgaccgccattaagaatgccctgaagactaccaacgaggctgtgtccactctgggaaacggagtgagggtcctggccttcgcagtgagggagctgaaggattttgtgtcaaagaaccttacacgggccatcaacaagaataagtgcgatatcgatgacctgaagatggccgtgtccttctcccagttcaaccggcgctttctgaatgtggtgcgccagttttccgacaacgctggaatcacccctgctatcagcctggacctcatgaccgacgccgaactcgcaagggccgtttctaacatgcctacatccgctggacagattaagctgatgctggagaatagagcaatggtgaggagaaagggattcggcatcctgattggcgtgtacggatctagcgtgatctacatggtgcagctgccgatcttcggcgtgatcgatactccttgttggatcgtcaaggccgccccttcctgctccgagaagaagggcaattacgcttgtctgctgcgggaggaccagggctggtattgccagaacgccgggtctacagtgtactatcctaacgagaaggattgcgagaccagaggcgaccacgttttctgtgatacagccgccggaatcaatgtcgcagagcagtctaaggagtgcaacatcaatatctctacaaccaattacccatgtaaggtgagcactggacggcaccctatcagtatggtggctctgagcccactgggggcactggtggcttgctacaagggggtgagctgcagtatcggcagtaacagagtgggcattatcaagcagctgaacaaagggtgctcttatattacaaaccaggatgcagatactgtgaccatcgacaacactgtgtaccagctgtccaaggtggagggggagcagcatgtgatcaaagggagacccgtctcttcttctttcgatcccatcaagttccctgaagaccagttcaatgttgccctggaccaggttttcgagaacatcgaaaatagccaggccttggtcgatcaatccaacaggatcctgagcagcgcagagaaagggaacactggcttcatcatcgtgatcattctgatcgccgtgctggggagcagtatgattctggtgtccattttcatcatcatcaagaagaccaagaagcctacaggagcaccccctgagctgagcggagtgaccaacaacggctttatccctcacaactaa

SEQ ID NO: 14 - DNA, 1620 bp, synthetic construct
  source 1..1620: mol_type = other DNA
atgctgattagcattctgtccatcatcacaaccatgattatggcaagtcactgtcagatcgatatcaccaagctccagcacgtgggcgtgctggtgaattccccaaaggggatgaagatttctcagaactttgagactcggtacctgatcctgtccctgattcctaagattgaggatagcaactcctgtggcgaccagcagatcaaacagtacaaacgcctgctggacagactgatcatcccactgtacgacggcctgaagctgcagaaagacgtcattgttacaaaccaggaaagcaacgagaataccgaccctaggactgagcggttcttcgggggcgtcatcggaacaatcgcactgggcgttgccactagcgcacagatcaccgccgccgttgcactggtggaggctaagcaggcaaaatctgacatcgagaagctgaaagaagctatccgcgataccaacaaggccgtgcagtcagtgtgctccagcgtcggcaactgcatcgttgctattaagtccgtgcaggactacgtgaacaaggagatcgtgcccagtatcgcaaggctgggatgtgaggccgctggactgcagttggggatcgctctcacccagcactactccgagctgacaaattgcttcggagataacatcggatctctgcaggagaagggcatcaaactgcagtgtattgcctccctgtacagaacaaatatcacagagattttcacaaccagcactgtcgacaaatatgatatctatgacctgctgttcaccgagtccattaaggtgcgggtcatcgacgttgacctgaacgattattccatcaccctccaggtgagactgcctctgctgacacggctgctgaacactcagatctacaaggtcgactccatcagctacaacatccagaaccgcgagtggtacatcccactgcctagccacatcatgacaaagggcgcttttctgggaggagctgatgtgaaggaatgcattgaggccttcagcagctacatctgcccttccgatcccggcttcgtcctgaaccacgagatggagagctgtctgagcgggaacattagccagtgccccaggacaaccgtcacctctgacattgtgccaaggtatgccttcgtgaacggcggagtggttgccaattgcatcaccactacatgcacttgcaatgggattggcaacaggatcaaccagccaccagaccagggggtgaaaatcattacacacaaagagtgcaacaccatcggcatcaacggaatgctgttcaataccaacaaggagggcaccctggccttctacacacccgatgacatcaccctgaacaatagcgtggctctggatcccattgatatcagcattgagctgaataaggtcaagtctgatctggaggagagcaaggagtggtatagacggtctaatcagaagctcgactctatcggatcctggcaccagtcctccacaacaatcatcattatcctgatcatgatgatcatcctgttcatcatcaacatcacaattatcaccattgccatcaagtattaccggattcagaagcggaatcgcgttgaccagaacgacaaaccttatgttctgaccaacaaataa

SEQ ID NO: 15 - RNA, 1845 nt, synthetic construct
  source 1..1845: mol_type = other RNA
  mRNA 1..1845: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggaactgctgatcctcaaagccaacgcaatcaccaccattctcaccgctgtgaccttctgcttcgcatcggggcagaacatcactgaagagttttaccagagcacttgcagcgcggtgtcaaagggttacctttccgcactgcggaccggatggtacacttccgtgatcaccattgagctcagcaacatcaaggaaaacaagtgcaatggcaccgacgccaaggtcaagctgatcaaacaagaactggacaagtacaagaacgccgtgacagaattgcagctcctgatgggatccggaaacgtcggtctgggcggagccatcgcgagtggagtggctgtgtccaaggtcttgcacctcgagggagaagtgaacaagatcaagtccgcgctgctgtcaacgaacaaggccgtggtgtccctgtctaacggcgtcagcgtgctgacgttcaaggtcctggacctgaagaattacattgacaagcagctgctgcccatcctcaacaagcaatcctgctccatctccaaccccgaaaccgtgatcgagttccagcagaagaacaaccgcctgctggaaattactcgcgagttctctgtgaatgccggcgtgaccacccctgtgtccacctacatgctgaccaactccgagcttctctcccttatcaatgacatgcctatcacgaacgaccagaagaagctgatgtcgaacaacgtgcagattgtgcggcagcagtcatacagcatcatgtcgatcatcaaggaagaagtgctggcgtacgtggtgcaactcccgctgtacggcgtcatcgataccccgtgctggaagctgcacacctcgcctttgtgtaccaccaacaccaagaacggatccaacatctgcttaacccggactgatcggggttggtactgcgacaacgccgggaatgtttcgttcttcccacaagccgagacttgtaaagtgcagtcaaacagagtgttctgtgacaccatgaactcgagaaccctgcccagcgaagtgaacctgtgtaacgtcgacatctttaacccaaaatacgattgcaagattatgaccagcaaaaccgacgtgtcctcctccgtgataacaagcctgggggcgattgtgtcatgctacggaaagactaagtgcaccgcctcgaacaagaaccgcggcatcattaagactttctcgaatggttgcgactatgtgtccaacaagggcgtggatactgtgtcagtcgggaatactctttactacgtgaacaagcaggaggggaaaagcctctacgtgaagggagagcctattatcaacttttacgatccgctggtgttcccgtccgacgaattcgacgccagcatcagccaagtcaacgagctgattaaccagtccctcgccttcatcaaccaatccgacgagctcctgcataacgtgaacgccggaaagtccaccaccaacatcatgatcactactattatcatcgtgatcatcgtcatcctgctgagcctgattgctgtgggcctgttgctgtattgcaaagccaggtccaccccggtcaccctgtcgaaggatcagctgtccggaatcaacaacattgccttctccaactaacgggtggcatccctgtgacccctccccagtgcctctcctggccctggaagttgccactccagtgcccaccagccttgtcctaataaaattaagttgcatc

SEQ ID NO: 16 - RNA, 1860 nt, synthetic construct
  source 1..1860: mol_type = other RNA
  mRNA 1..1860: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgagctggaaggttgtgattattttctctctgctgattactccacagcacggcctgaaggagtcctacctggaggagtcctgttctactatcactgaggggtatctctctgtgctgcggacagggtggtatacagtggtgttcaccctggaggttggcgatgtggagaatctgacttgcagcgatggcccttctctgatcaagaccgagctggatctgacaaaaagcgccctcagagaactgaaaaccgtgtccgccgatcagctggcaagggaggagcagatcgagaacccacggcagagcaggtttgtgctgggcgctatcgctctgggcgtggccactgcagctgctgtcactgcaggggtcgcaatcgctaagactatcagactggaatccgaggtgaccgccattaagaatgccctgaagactaccaacgaggctgtgtccactctgggaaacggagtgagggtcctggccttcgcagtgagggagctgaaggattttgtgtcaaagaaccttacacgggccatcaacaagaataagtgcgatatcgatgacctgaagatggccgtgtccttctcccagttcaaccggcgctttctgaatgtggtgcgccagttttccgacaacgctggaatcacccctgctatcagcctggacctcatgaccgacgccgaactcgcaagggccgtttctaacatgcctacatccgctggacagattaagctgatgctggagaatagagcaatggtgaggagaaagggattcggcatcctgattggcgtgtacggatctagcgtgatctacatggtgcagctgccgatcttcggcgtgatcgatactccttgttggatcgtcaaggccgccccttcctgctccgagaagaagggcaattacgcttgtctgctgcgggaggaccagggctggtattgccagaacgccgggtctacagtgtactatcctaacgagaaggattgcgagaccagaggcgaccacgttttctgtgatacagccgccggaatcaatgtcgcagagcagtctaaggagtgcaacatcaatatctctacaaccaattacccatgtaaggtgagcactggacggcaccctatcagtatggtggctctgagcccactgggggcactggtggcttgctacaagggggtgagctgcagtatcggcagtaacagagtgggcattatcaagcagctgaacaaagggtgctcttatattacaaaccaggatgcagatactgtgaccatcgacaacactgtgtaccagctgtccaaggtggagggggagcagcatgtgatcaaagggagacccgtctcttcttctttcgatcccatcaagttccctgaagaccagttcaatgttgccctggaccaggttttcgagaacatcgaaaatagccaggccttggtcgatcaatccaacaggatcctgagcagcgcagagaaagggaacactggcttcatcatcgtgatcattctgatcgccgtgctggggagcagtatgattctggtgtccattttcatcatcatcaagaagaccaagaagcctacaggagcaccccctgagctgagcggagtgaccaacaacggctttatccctcacaactaacgggtggcatccctgtgacccctccccagtgcctctcctggccctggaagttgccactccagtgcccaccagccttgtcctaataaaattaagttgcatc

SEQ ID NO: 17 - RNA, 1860 nt, synthetic construct
  source 1..1860: mol_type = other RNA
  mRNA 1..1860: note = mRNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgctgattagcattctgtccatcatcacaaccatgattatggcaagtcactgtcagatcgatatcaccaagctccagcacgtgggcgtgctggtgaattccccaaaggggatgaagatttctcagaactttgagactcggtacctgatcctgtccctgattcctaagattgaggatagcaactcctgtggcgaccagcagatcaaacagtacaaacgcctgctggacagactgatcatcccactgtacgacggcctgaagctgcagaaagacgtcattgttacaaaccaggaaagcaacgagaataccgaccctaggactgagcggttcttcgggggcgtcatcggaacaatcgcactgggcgttgccactagcgcacagatcaccgccgccgttgcactggtggaggctaagcaggcaaaatctgacatcgagaagctgaaagaagctatccgcgataccaacaaggccgtgcagtcagtgtgctccagcgtcggcaactgcatcgttgctattaagtccgtgcaggactacgtgaacaaggagatcgtgcccagtatcgcaaggctgggatgtgaggccgctggactgcagttggggatcgctctcacccagcactactccgagctgacaaattgcttcggagataacatcggatctctgcaggagaagggcatcaaactgcagtgtattgcctccctgtacagaacaaatatcacagagattttcacaaccagcactgtcgacaaatatgatatctatgacctgctgttcaccgagtccattaaggtgcgggtcatcgacgttgacctgaacgattattccatcaccctccaggtgagactgcctctgctgacacggctgctgaacactcagatctacaaggtcgactccatcagctacaacatccagaaccgcgagtggtacatcccactgcctagccacatcatgacaaagggcgcttttctgggaggagctgatgtgaaggaatgcattgaggccttcagcagctacatctgcccttccgatcccggcttcgtcctgaaccacgagatggagagctgtctgagcgggaacattagccagtgccccaggacaaccgtcacctctgacattgtgccaaggtatgccttcgtgaacggcggagtggttgccaattgcatcaccactacatgcacttgcaatgggattggcaacaggatcaaccagccaccagaccagggggtgaaaatcattacacacaaagagtgcaacaccatcggcatcaacggaatgctgttcaataccaacaaggagggcaccctggccttctacacacccgatgacatcaccctgaacaatagcgtggctctggatcccattgatatcagcattgagctgaataaggtcaagtctgatctggaggagagcaaggagtggtatagacggtctaatcagaagctcgactctatcggatcctggcaccagtcctccacaacaatcatcattatcctgatcatgatgatcatcctgttcatcatcaacatcacaattatcaccattgccatcaagtattaccggattcagaagcggaatcgcgttgaccagaacgacaaaccttatgttctgaccaacaaataacgggtggcatccctgtgacccctccccagtgcctctcctggccctggaagttgccactccagtgcccaccagccttgtcctaataaaattaagttgcatc

SEQ ID NO: 18 - RNA, 11 nt, synthetic construct
  source 1..11: mol_type = other RNA
gggatcctacc

SEQ ID NO: 19 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 20 - protein, 7 aa, synthetic construct
GSGNVGL

SEQ ID NO: 21 - protein, 9 aa, synthetic construct
GSGNVGLGG

SEQ ID NO: 22 - protein, 544 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 23 - protein, 50 aa, synthetic construct
IMITTIIIVIIVILLSLIAVGLLLYCKARSTPVTLSKDQLSGINNIAFSN

SEQ ID NO: 24 - RNA, 1725 nt, synthetic construct
  source 1..1725: mol_type = other RNA
atggaattgctgatcctcaaagcgaacgcaatcaccactatcctcactgcggtcaccttctgctttgcgagcggacagaacatcaccgaagaattctaccaatctacttgctccgccgtgtccaagggttacctgtccgccctgaggaccggatggtacacttccgtgattaccattgagttgtcgaatatcaagaagaacaagtgcaacggaaccgatgctaaggtcaagctgatcaagcaggagctggacaagtacaagaatgctgtgaccgagctgcagctgctgatgcagtccactcaagccaccaacaatcgcgcccggcgggaactcccaaggttcatgaactacaccttgaacaacgccaagaaaacgaacgtgaccctgtccaagaagcgcaagcgcagattccttggcttccttctgggcgtcggtagcgccatcgcctccggcgtggccgtcagcaaggtcctgcacctcgagggagaagtcaacaagattaagagcgccctgctgtccaccaacaaggccgtggtgtcgctatcaaacggcgtcagcgtactgaccagcaaagtgctggatctcaagaactacattgataagcaactcctccctatcgtgaataagcagagctgttcgatttccaacatcgagactgtgattgaattccagcagaagaacaaccggctgctggaaattaccagagaattcagcgtgaatgccggagtcactacccccgtgtccacctacatgctgacaaactccgagctgctgagcctgatcaacgatatgccgattaccaacgaccagaagaagctgatgtcgaacaacgtgcagatcgtgcgccagcagtcctactcaatcatgtcgatcatcaaggaagaggtcctggcctacgtggtgcagcttcctctgtacggcgtgattgacactccgtgttggaaactgcacactagtcccctgtgcactactaacaccaaggagggcagcaatatctgcctgactcggaccgatagaggctggtactgtgataacgccgggtccgtgtccttcttcccgcaagccgagacttgcaaagtgcagagcaaccgggtgttctgtgacactatgaactcactgaccttgccgagcgaagtcaacctttgcaacgtggacatctttaaccctaaatacgactgcaagatcatgacctccaagaccgacgtgtcgagctcagtgattacttcgctgggagccattgtgtcctgctacgggaaaaccaagtgcacggcctcaaacaagaaccggggtatcattaagaccttctccaacggctgcgactatgtgtccaacaagggggtggacactgtgtccgtgggaaacaccttgtattacgtgaacaagcaggagggaaagtccctctacgtgaagggcgaacccatcatcaatttctacgacccgctcgtgttcccctccgatgaattcgacgcatccatctcacaagtcaacgaaaagattaaccagtccctggctttcattcgcaagtccgacgaactgctccataacgtcaacgctggaaagtccaccaccaacatcatgatcaccacgatcattattgtgatcatcgtcatcctgctgtcactgatagcagtgggactgctcctctactgcaaagcgcggtcgaccccagtgacactctcgaaggaccagctgtccgggatcaacaacatcgcgttttcgaactga

SEQ ID NO: 25 - RNA, 1635 nt, synthetic construct
  source 1..1635: mol_type = other RNA
atggaactcctgatcctgaaggccaatgctatcactaccatcctgactgccgtcaccttctgcttcgcctccggacaaaatatcactgaagaattttaccaaagcacctgtagcgcggtgtccaagggatacctgagcgctctgaggaccggatggtacaccagcgtgattaccatcgagctgagtaacatcaagaagaacaagtgcaacgggaccgatgctaaggtcaagttgatcaaacaagagctcgacaagtacaagaacgccgtgactgagctgcagctgctgatgcagtcaactcaggccaccaacaaccgggccagacgggaactgccgagattcatgaactacaccctgaacaacgccaaaaagaccaacgtgaccctgtccaagaagagaaagcgccggttcctgggtttcctgcttggcgtgggatcagcaatcgcgtccggagtggcagtgtccaaggtcttgcacctcgagggcgaagtgaacaagatcaagtccgcgcttctgtcgaccaacaaggccgtcgtttccctgtcgaacggagtgtccgtgctcacgagcaaagtgctcgacctgaagaactacatcgacaaacagctgctgcccatcgtcaacaagcagagctgcagcatctcaaacattgaaaccgtgatcgagttccagcagaagaacaaccgcctgctcgagattaccagagagttttccgtgaacgccggcgtgaccaccccggtgtcgacctacatgctcacaaattcggaacttctctccctgattaatgacatgcccattactaacgatcagaaaaagctgatgtcgaacaatgtgcagattgtgcgccagcagtcctactccatcatgtccatcattaaggaagaggtcctggcctacgtggtgcagttgccgctgtacggtgtcatcgataccccctgctggaagctccatacttcgcccctgtgtactaccaacaccaaggaaggctccaacatctgcctgacccggacggatcgcggctggtactgtgacaatgccggatccgtgtcgttcttcccgcaagcggagacttgcaaagtgcagtccaaccgggtgttctgtgacactatgaactccctgactctgccgtccgaagtcaacctctgcaacgtggacattttcaatccaaaatacgactgcaagataatgacctccaagactgacgtgtcatcgtccgtgatcacatctctgggagccattgtctcctgctacggaaagactaagtgcaccgcgtcgaacaagaacaggggcattatcaagaccttcagcaacggttgcgactatgtgtccaacaagggcgtggataccgtgtccgtgggcaacaccttgtactacgtgaacaagcaggaggggaagtccctttatgtgaagggggagccaatcattaacttttacgaccccctggtgttccctagcgacgagttcgacgcctcaatctctcaagtcaacgaaaagatcaaccagagcctcgccttcatccgcaagtccgatgaactgctgtcagccattgggggttacatccctgaggcccctcgggacggacaggcatacgtccgcaaggacggcgaatgggtgctgcttagcaccttcctctaa

SEQ ID NO: 26 - RNA, 1605 nt, synthetic construct; identical to SEQ ID NO: 4
  source 1..1605: mol_type = other RNA

SEQ ID NO: 27 - RNA, 1965 nt, synthetic construct
  source 1..1965: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggaattgctgatcctcaaagcgaacgcaatcaccactatcctcactgcggtcaccttctgctttgcgagcggacagaacatcaccgaagaattctaccaatctacttgctccgccgtgtccaagggttacctgtccgccctgaggaccggatggtacacttccgtgattaccattgagttgtcgaatatcaagaagaacaagtgcaacggaaccgatgctaaggtcaagctgatcaagcaggagctggacaagtacaagaatgctgtgaccgagctgcagctgctgatgcagtccactcaagccaccaacaatcgcgcccggcgggaactcccaaggttcatgaactacaccttgaacaacgccaagaaaacgaacgtgaccctgtccaagaagcgcaagcgcagattccttggcttccttctgggcgtcggtagcgccatcgcctccggcgtggccgtcagcaaggtcctgcacctcgagggagaagtcaacaagattaagagcgccctgctgtccaccaacaaggccgtggtgtcgctatcaaacggcgtcagcgtactgaccagcaaagtgctggatctcaagaactacattgataagcaactcctccctatcgtgaataagcagagctgttcgatttccaacatcgagactgtgattgaattccagcagaagaacaaccggctgctggaaattaccagagaattcagcgtgaatgccggagtcactacccccgtgtccacctacatgctgacaaactccgagctgctgagcctgatcaacgatatgccgattaccaacgaccagaagaagctgatgtcgaacaacgtgcagatcgtgcgccagcagtcctactcaatcatgtcgatcatcaaggaagaggtcctggcctacgtggtgcagcttcctctgtacggcgtgattgacactccgtgttggaaactgcacactagtcccctgtgcactactaacaccaaggagggcagcaatatctgcctgactcggaccgatagaggctggtactgtgataacgccgggtccgtgtccttcttcccgcaagccgagacttgcaaagtgcagagcaaccgggtgttctgtgacactatgaactcactgaccttgccgagcgaagtcaacctttgcaacgtggacatctttaaccctaaatacgactgcaagatcatgacctccaagaccgacgtgtcgagctcagtgattacttcgctgggagccattgtgtcctgctacgggaaaaccaagtgcacggcctcaaacaagaaccggggtatcattaagaccttctccaacggctgcgactatgtgtccaacaagggggtggacactgtgtccgtgggaaacaccttgtattacgtgaacaagcaggagggaaagtccctctacgtgaagggcgaacccatcatcaatttctacgacccgctcgtgttcccctccgatgaattcgacgcatccatctcacaagtcaacgaaaagattaaccagtccctggctttcattcgcaagtccgacgaactgctccataacgtcaacgctggaaagtccaccaccaacatcatgatcaccacgatcattattgtgatcatcgtcatcctgctgtcactgatagcagtgggactgctcctctactgcaaagcgcggtcgaccccagtgacactctcgaaggaccagctgtccgggatcaacaacatcgcgttttcgaactgacgggtggcatccctgtgacccctccccagtgcctctcctggccctggaagttgccactccagtgcccaccagccttgtcctaataaaattaagttgcatc

SEQ ID NO: 28 - RNA, 1875 nt, synthetic construct
  source 1..1875: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggaactcctgatcctgaaggccaatgctatcactaccatcctgactgccgtcaccttctgcttcgcctccggacaaaatatcactgaagaattttaccaaagcacctgtagcgcggtgtccaagggatacctgagcgctctgaggaccggatggtacaccagcgtgattaccatcgagctgagtaacatcaagaagaacaagtgcaacgggaccgatgctaaggtcaagttgatcaaacaagagctcgacaagtacaagaacgccgtgactgagctgcagctgctgatgcagtcaactcaggccaccaacaaccgggccagacgggaactgccgagattcatgaactacaccctgaacaacgccaaaaagaccaacgtgaccctgtccaagaagagaaagcgccggttcctgggtttcctgcttggcgtgggatcagcaatcgcgtccggagtggcagtgtccaaggtcttgcacctcgagggcgaagtgaacaagatcaagtccgcgcttctgtcgaccaacaaggccgtcgtttccctgtcgaacggagtgtccgtgctcacgagcaaagtgctcgacctgaagaactacatcgacaaacagctgctgcccatcgtcaacaagcagagctgcagcatctcaaacattgaaaccgtgatcgagttccagcagaagaacaaccgcctgctcgagattaccagagagttttccgtgaacgccggcgtgaccaccccggtgtcgacctacatgctcacaaattcggaacttctctccctgattaatgacatgcccattactaacgatcagaaaaagctgatgtcgaacaatgtgcagattgtgcgccagcagtcctactccatcatgtccatcattaaggaagaggtcctggcctacgtggtgcagttgccgctgtacggtgtcatcgataccccctgctggaagctccatacttcgcccctgtgtactaccaacaccaaggaaggctccaacatctgcctgacccggacggatcgcggctggtactgtgacaatgccggatccgtgtcgttcttcccgcaagcggagacttgcaaagtgcagtccaaccgggtgttctgtgacactatgaactccctgactctgccgtccgaagtcaacctctgcaacgtggacattttcaatccaaaatacgactgcaagataatgacctccaagactgacgtgtcatcgtccgtgatcacatctctgggagccattgtctcctgctacggaaagactaagtgcaccgcgtcgaacaagaacaggggcattatcaagaccttcagcaacggttgcgactatgtgtccaacaagggcgtggataccgtgtccgtgggcaacaccttgtactacgtgaacaagcaggaggggaagtccctttatgtgaagggggagccaatcattaacttttacgaccccctggtgttccctagcgacgagttcgacgcctcaatctctcaagtcaacgaaaagatcaaccagagcctcgccttcatccgcaagtccgatgaactgctgtcagccattgggggttacatccctgaggcccctcgggacggacaggcatacgtccgcaaggacggcgaatgggtgctgcttagcaccttcctctaacgggtggcatccctgtgacccctccccagtgcctctcctggccctggaagttgccactccagtgcccaccagccttgtcctaataaaattaagttgcatc

SEQ ID NO: 29 - RNA, 1845 nt, synthetic construct; identical to SEQ ID NO: 15
  source 1..1845: mol_type = other RNA

SEQ ID NO: 30 - DNA, 1620 bp, synthetic construct
  source 1..1620: mol_type = other DNA
atgtcttggaaagtggtgatcattttttcattgctaataacacctcaacacggtcttaaagagagctacctagaagaatcatgtagcactataactgagggatatcttagtgttctgaggacaggttggtataccaacgtttttacattagaggtgggtgatgtagaaaaccttacatgttctgatggacctagcctaataaaaacagaattagatctgaccaaaagtgcactaagagagctcaaaacagtctctgctgaccaattggcaagagaggaacaaattgagaatcccagacaatctaggtttgttctaggagcaatagcactcggtgttgcaacagcagctgcagtcacagcaggtgttgcaattgccaaaaccatccggcttgagagtgaagtcacagcaattaagaatgccctcaaaacgaccaatgaagcagtatctacattggggaatggagttcgagtgttggcaactgcagtgagagagctgaaagactttgtgagcaagaatttaactcgtgcaatcaacaaaaacaagtgcgacattgatgacctaaaaatggccgttagcttcagtcaattcaacagaaggtttctaaatgttgtgcggcaattttcagacaatgctggaataacaccagcaatatctttggacttaatgacagatgctgaactagccagggccgtttctaacatgccgacatctgcaggacaaataaaattgatgttggagaaccgtgcgatggtgcgaagaaaggggttcggaatcctgataggggtctacgggagctccgtaatttacatggtgcagctgccaatctttggcgttatagacacgccttgctggatagtaaaagcagccccttcttgttccgaaaaaaagggaaactatgcttgcctcttaagagaagaccaagggtggtattgtcagaatgcagggtcaactgtttactacccaaatgagaaagactgtgaaacaagaggagaccatgtcttttgcgacacagcagcgggaattaatgttgctgagcaatcaaaggagtgcaacatcaacatatccactacaaattacccatgcaaagtcagcacaggaagacatcctatcagtatggttgcactgtctcctcttggggctctggttgcttgctacaaaggagtaagctgttccattggcagcaacagagtagggatcatcaagcagctgaacaagggttgctcctatataaccaaccaagatgcagacacagtgacaatagacaacactgtatatcagctaagcaaagttgagggtgaacagcatgttataaaaggcagaccagtgtcaagcagctttgatccaatcaagtttcctgaagatcaattcaatgttgcacttgaccaagtttttgagaacattgaaaacagccaggccttggtagatcaatcaaacagaatcctaagcagtgcagagaaagggaatactggcttcatcattgtaataattctaattgctgtccttggctctagcatgatcctagtgagcatcttcattataatcaagaaaacaaagaaaccaacgggagcacctccagagctgagtggtgtcacaaacaatggcttcataccacataattag

SEQ ID NO: 31 - protein, 551 aa, synthetic construct
MSWKVVIIFSLLITPQHGLKESYLEESCSTITEGYLSVLRTGWYTNVFTLEVGDVENLTCSDGPSLIKTELDLTKSALRELKTVSADQLAREEQIENPRRRRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGGGGSGYIPEAPRDGQAYVRKDGEWVLLSTFLGRSLEVLFQGPGHHHHHHHHSAWSHPQFEK

SEQ ID NO: 32 - DNA, 1656 bp, synthetic construct
  source 1..1656: mol_type = other DNA
atgagttggaaggtggtgattatcttctccctgctgattacaccacaacatggactgaaagagtcctacttggaggagtcctgtagcaccatcacagagggctacctgtctgtgctgaggacaggctggtacaccaatgtgttcaccttggaggtgggagatgtggagaacctgacttgttctgatggaccatccctgattaagacagaactggacctgaccaagtctgccctgagggaactgaaaacagtgtctgctgaccaacttgccagggaggaacagattgagaacccaaggaggaggaggtttgtgctgggagccattgccctgggagtggctacagcagcagcagtgacagcaggagtggctattgccaagaccatcagattggagtctgaggtgacagccatcaagaatgccctgaaaaccaccaatgaggctgtgagcaccctgggcaatggagtgagggtgctggctacagcagtgagggaactgaaagactttgtgagcaagaacctgaccagggctatcaacaagaacaagtgtgacatcgatgacctgaaaatggctgtgtccttcagccagttcaacaggaggttcctgaatgtggtgagacagttctctgacaatgctggcatcacacctgccatctccctggacctgatgacagatgctgaactggcaagggctgtgagcaatatgccaacctctgctggacaaatcaaactgatgttggagaacagggctatggtgaggaggaagggctttggcatcctgattggagtctatggctcctctgtgatttatatggtccaacttccaatctttggagtgattgacacaccatgttggattgtgaaggctgccccatcctgttctgagaagaagggcaactatgcctgtctgctgagggaggaccagggctggtattgtcagaatgctggcagcacagtctactacccaaatgagaaggactgtgagaccaggggagaccatgtgttctgtgacacagcagcaggcatcaatgtggctgaacagagcaaggagtgtaacatcaacatcagcaccaccaactacccatgtaaggtgagcacaggcagacacccaatcagtatggtggctctgagcccactgggagccctggtggcttgttacaagggagtgtcctgtagcattggcagcaacagggtgggcatcatcaagcaacttaacaagggctgttcctacatcaccaaccaggatgctgacacagtgaccattgacaacacagtctaccaacttagcaaggtggagggagaacagcatgtgattaagggcagacctgtgtcctcctcctttgacccaatcaagtttcctgaggaccagttcaatgtggctctggaccaggtgtttgagaacattgagaacagccaggctctggtggaccagagcaacaggattctgtcctctgctgagaagggcaacacaggaggaggaggctctggctacatccctgaggctccaagggatggacaagcctatgtgaggaaggatggagagtgggtgctgctgagcaccttcctgggcaggtccttggaggtgctgttccagggacctggacaccaccaccaccaccaccaccactctgcctggagccacccacagtttgagaagtaa

SEQ ID NO: 33 - protein, 551 aa, synthetic construct
MSWKVVIIFSLLITPQHGLKESYLEESCSTITEGYLSVLRTGWYTNVFTLEVGDVENLTCSDGPSLIKTELDLTKSALRELKTVSADQLAREEQIENPRRRRFVLGAIALGVATAAAVTAGVAIAKTIRLESEVTAIKNALKTTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGGGGSGYIPEAPRDGQAYVRKDGEWVLLSTFLGRSLEVLFQGPGHHHHHHHHSAWSHPQFEK

SEQ ID NO: 34 - DNA, 1656 bp, synthetic construct
  source 1..1656: mol_type = other DNA
atgagttggaaggtggtgattatcttctccctgctgattacaccacaacatggactgaaagagtcctacttggaggagtcctgtagcaccatcacagagggctacctgtctgtgctgaggacaggctggtacaccaatgtgttcaccttggaggtgggagatgtggagaacctgacttgttctgatggaccatccctgattaagacagaactggacctgaccaagtctgccctgagggaactgaaaacagtgtctgctgaccaacttgccagggaggaacagattgagaacccaaggaggaggaggtttgtgctgggagccattgccctgggagtggctacagcagcagcagtgacagcaggagtggctattgccaagaccatcagattggagtctgaggtgacagccatcaagaatgccctgaaaaccaccaatgaggctgtgagcaccctgggcaatggagtgagggtgctggctacagcagtgagggaactgaaagactttgtgagcaagaacctgaccagggctatcaacaagaacaagtgtgacatccctgacctgaaaatggctgtgtccttcagccagttcaacaggaggttcctgaatgtggtgagacagttctctgacaatgctggcatcacacctgccatctccctggacctgatgacagatgctgaactggcaagggctgtgagcaatatgccaacctctgctggacaaatcaaactgatgttggagaacagggctatggtgaggaggaagggctttggcatcctgattggagtctatggctcctctgtgatttatatggtccaacttccaatctttggagtgattgacacaccatgttggattgtgaaggctgccccatcctgttctgagaagaagggcaactatgcctgtctgctgagggaggaccagggctggtattgtcagaatgctggcagcacagtctactacccaaatgagaaggactgtgagaccaggggagaccatgtgttctgtgacacagcagcaggcatcaatgtggctgaacagagcaaggagtgtaacatcaacatcagcaccaccaactacccatgtaaggtgagcacaggcagacacccaatcagtatggtggctctgagcccactgggagccctggtggcttgttacaagggagtgtcctgtagcattggcagcaacagggtgggcatcatcaagcaacttaacaagggctgttcctacatcaccaaccaggatgctgacacagtgaccattgacaacacagtctaccaacttagcaaggtggagggagaacagcatgtgattaagggcagacctgtgtcctcctcctttgacccaatcaagtttcctgaggaccagttcaatgtggctctggaccaggtgtttgagaacattgagaacagccaggctctggtggaccagagcaacaggattctgtcctctgctgagaagggcaacacaggaggaggaggctctggctacatccctgaggctccaagggatggacaagcctatgtgaggaaggatggagagtgggtgctgctgagcaccttcctgggcaggtccttggaggtgctgttccagggacctggacaccaccaccaccaccaccaccactctgcctggagccacccacagtttgagaagtaa

SEQ ID NO: 35 - protein, 551 aa, synthetic construct
MSWKVVIIFSLLITPQHGLKESYLEESCSTITEGYLSVLRTGWYTVVFTLEVGDVENLTCSDGPSLIKTELDLTKSALRELKTVSADQLAREEQIENPRRRRFVLGAIALGVATAAAVTAGVAIAKTIRLESEVTAIKNALKTTNEAVSTLGNGVRVLAFAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGGGGSGYIPEAPRDGQAYVRKDGEWVLLSTFLGRSLEVLFQGPGHHHHHHHHSAWSHPQFEK

SEQ ID NO: 36 - RNA, 1620 nt, synthetic construct
  source 1..1620: mol_type = other RNA
  mRNA 1..1620: note = mRNA
atgtcttggaaagtcgtcatcatcttctctctgctgatcaccccacaacacggcctgaaggaatcttatctggaagagtcctgctccacaatcacagagggctacctgagcgtgctgagaaccggctggtacaccaacgtgttcactctggaggtgggcgacgtggagaacctgacttgtagtgacggcccctccctgatcaagactgagctggacctgacaaagagtgcactgagagaactcaagactgtgtccgcagaccagctggcccgcgaggagcagatcgaaaatcctagacagtcaaggttcgtcctgggagccattgctctgggagttgctacagctgccgctgtgaccgcaggggtggctattgctaaaaccatcaggctggagtccgaagtgacagcaatcaagaatgccctgaagaccaccaacgaggcagtctccacactgggcaatggagtgagggtgctggcaaccgccgtgagggagctgaaggacttcgtgtccaagaacctgaccagggctatcaacaaaaacaagtgcgacatccccgatctgaagatggcagttagcttttcccagtttaaccggagattcctgaatgtggttagacagttcagcgacaacgccgggatcaccccagctatttccctggacctgatgactgatgccgagctggcacgggctgtgtccaatatgcccaccagcgctgggcagattaagctgatgctggagaatcgggcaatggtgagaaggaaggggtttggcatcctgatcggcgtgtacgggtcctccgtgatctacatggtgcagctgcctatttttggagtgattgatacaccctgctggatcgttaaagcagcacccagctgctccgagaagaagggcaattacgcctgtctgctgcgggaggaccaggggtggtactgccagaacgccggctccacagtgtattaccccaatgaaaaggactgcgagacaaggggagaccacgtgttctgcgacactgccgctgggattaatgtggccgagcagagcaaggagtgcaacatcaacatttccaccacaaactacccctgcaaggtgagcaccggcaggcaccctatctccatggtggccctgtctcccctgggagctctggtggcttgctacaagggagtgagctgtagcatcgggtccaatagagtcgggattatcaagcagctgaataagggctgcagctatattaccaaccaggatgccgatactgtgactattgacaacacagtgtatcagctgtcaaaggtggaaggcgaacagcatgtgatcaaaggacggcccgtcagcagctcctttgaccctatcaaattccccgaagaccagtttaacgtggcactggaccaggttttcgaaaatattgagaattctcaggccctggtggaccagtctaaccggatcctctcctccgccgagaagggaaatacaggctttattatcgtgatcatcctgatcgcagtgctgggatccagtatgatcctggtctccattttcatcatcattaagaagaccaagaaacccactggcgcaccacctgaactgagcggcgtgactaacaatggctttatccctcacaattga

SEQ ID NO: 37 - RNA, 1656 nt, synthetic construct
  source 1..1656: mol_type = other RNA
  mRNA 1..1656: note = mRNA
atgagttggaaggtggtgattatcttctccctgctgattacaccacaacatggactgaaagagtcctacttggaggagtcctgtagcaccatcacagagggctacctgtctgtgctgaggacaggctggtacacagtggtgttcaccttggaggtgggagatgtggagaacctgacttgttctgatggaccatccctgattaagacagaactggacctgaccaagtctgccctgagggaactgaaaacagtgtctgctgaccaacttgccagggaggaacagattgagaacccaaggaggaggaggtttgtgctgggagccattgccctgggagtggctacagcagcagcagtgacagcaggagtggctattgccaagaccatcagattggagtctgaggtgacagccatcaagaatgccctgaaaaccaccaatgaggctgtgagcaccctgggcaatggagtgagggtgctggcttttgctgtgagggaactgaaagactttgtgagcaagaacctgaccagggctatcaacaagaacaagtgtgacattgatgacctgaaaatggctgtgtccttcagccagttcaacaggaggttcctgaatgtggtgagacagttctctgacaatgctggcatcacacctgccatctccctggacctgatgacagatgctgaactggcaagggctgtgagcaatatgccaacctctgctggacaaatcaaactgatgttggagaacagggctatggtgaggaggaagggctttggcatcctgattggagtctatggctcctctgtgatttatatggtccaacttccaatctttggagtgattgacacaccatgttggattgtgaaggctgccccatcctgttctgagaagaagggcaactatgcctgtctgctgagggaggaccagggctggtattgtcagaatgctggcagcacagtctactacccaaatgagaaggactgtgagaccaggggagaccatgtgttctgtgacacagcagcaggcatcaatgtggctgaacagagcaaggagtgtaacatcaacatcagcaccaccaactacccatgtaaggtgagcacaggcagacacccaatcagtatggtggctctgagcccactgggagccctggtggcttgttacaagggagtgtcctgtagcattggcagcaacagggtgggcatcatcaagcaacttaacaagggctgttcctacatcaccaaccaggatgctgacacagtgaccattgacaacacagtctaccaacttagcaaggtggagggagaacagcatgtgattaagggcagacctgtgtcctcctcctttgacccaatcaagtttcctgaggaccagttcaatgtggctctggaccaggtgtttgagaacattgagaacagccaggctctggtggaccagagcaacaggattctgtcctctgctgagaagggcaacacaggaggaggaggctctggctacatccctgaggctccaagggatggacaagcctatgtgaggaaggatggagagtgggtgctgctgagcaccttcctgggcaggtccttggaggtgctgttccagggacctggacaccaccaccaccaccaccaccactctgcctggagccacccacagtttgagaagtaa

SEQ ID NO: 38 - RNA, 1620 nt, synthetic construct
  source 1..1620: mol_type = other RNA
  mRNA 1..1620: note = mRNA
atgagctggaaggttgtgattattttctctctgctgattactccacagcacggcctgaaggagtcctacctggaggagtcctgttctactatcactgaggggtatctctctgtgctgcggacagggtggtatacagtggtgttcaccctggaggttggcgatgtggagaatctgacttgcagcgatggcccttctctgatcaagaccgagctggatctgacaaaaagcgccctcagagaactgaaaaccgtgtccgccgatcagctggcaagggaggagcagatcgagaacccacggcagagcaggtttgtgctgggcgctatcgctctgggcgtggccactgcagctgctgtcactgcaggggtcgcaatcgctaagactatcagactggaatccgaggtgaccgccattaagaatgccctgaagactaccaacgaggctgtgtccactctgggaaacggagtgagggtcctggccttcgcagtgagggagctgaaggattttgtgtcaaagaaccttacacgggccatcaacaagaataagtgcgatatcgatgacctgaagatggccgtgtccttctcccagttcaaccggcgctttctgaatgtggtgcgccagttttccgacaacgctggaatcacccctgctatcagcctggacctcatgaccgacgccgaactcgcaagggccgtttctaacatgcctacatccgctggacagattaagctgatgctggagaatagagcaatggtgaggagaaagggattcggcatcctgattggcgtgtacggatctagcgtgatctacatggtgcagctgccgatcttcggcgtgatcgatactccttgttggatcgtcaaggccgccccttcctgctccgagaagaagggcaattacgcttgtctgctgcgggaggaccagggctggtattgccagaacgccgggtctacagtgtactatcctaacgagaaggattgcgagaccagaggcgaccacgttttctgtgatacagccgccggaatcaatgtcgcagagcagtctaaggagtgcaacatcaatatctctacaaccaattacccatgtaaggtgagcactggacggcaccctatcagtatggtggctctgagcccactgggggcactggtggcttgctacaagggggtgagctgcagtatcggcagtaacagagtgggcattatcaagcagctgaacaaagggtgctcttatattacaaaccaggatgcagatactgtgaccatcgacaacactgtgtaccagctgtccaaggtggagggggagcagcatgtgatcaaagggagacccgtctcttcttctttcgatcccatcaagttccctgaagaccagttcaatgttgccctggaccaggttttcgagaacatcgaaaatagccaggccttggtcgatcaatccaacaggatcctgagcagcgcagagaaagggaacactggcttcatcatcgtgatcattctgatcgccgtgctggggagcagtatgattctggtgtccattttcatcatcatcaagaagaccaagaagcctacaggagcaccccctgagctgagcggagtgaccaacaacggctttatccctcacaactga

SEQ ID NO: 39 - RNA, 1620 nt, synthetic construct; identical to SEQ ID NO: 5
  source 1..1620: mol_type = other RNA
  mRNA 1..1620: note = mRNA

SEQ ID NO: 40 - protein, 539 aa, synthetic construct
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHN

SEQ ID NO: 41 - protein, 6 aa, synthetic construct
HHHHHH

SEQ ID NO: 42 - protein, 8 aa, synthetic construct
HHHHHHHH

SEQ ID NO: 43 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 44 - RNA, 10 nt, synthetic construct
  source 1..10: mol_type = other RNA
  variation 1: note = The Cytosine nucleotide can be repeated upto two hundred times
cccccccccc

SEQ ID NO: 45 - RNA, 10 nt, synthetic construct
  source 1..10: mol_type = other RNA
  variation 1: note = The Adenosine nucleotide can be repeated upto five hundred times
aaaaaaaaaa

(19) United States
(12) Patent Application Publication
(10) Pub. No.: US 2025/0009865 A1
(43) Pub. Date: Jan. 9, 2025

(54) COMBINATION RESPIRATORY MRNA VACCINES

(71) Applicant: SANOFI, Paris (FR)

(72) Inventors: Emilie Danve-Chery, Paris (FR); Nicholas Keith Clark, Cambridge, MA (US); Joshua DiNapoli, Cambridge, MA (US); Michael Kishko, Cambridge, MA (US); Bachra Rokbi, Paris (FR); Christopher Slade, Cambridge, MA (US); Timothy John Tibbitts, Cambridge, MA (US); William Warren, Cambridge, MA (US); Linong Zhang, Cambridge, MA (US)

(21) Appl. No.: 18/660,489

(22) Filed: May 10, 2024

Related U.S. Application Data
(60) Provisional application No. 63/465,389, filed on May 10, 2023.

(30) Foreign Application Priority Data
May 9, 2024 (EP) 24305723.9
May 9, 2024 (EP) 24315228.7

(51) Int. Cl. A61K 39/12; A61K 9/50; A61K 39/00; A61P 37/04

(52) U.S. Cl. CPC A61K 39/12; A61K 9/5015; A61K 9/5031; A61P 37/04; A61K 2039/53; A61K 2039/70

(57) ABSTRACT

The present disclosure provides compositions comprising at least two messenger RNAs (mRNAs) consisting of a human respiratory syncytial virus (hRSV or RSV) F protein antigen, a human metapneumovirus (hMPV) F protein antigen, and/or a human parainfluenza virus 3 (hPIV3 or PIV3) F protein antigen, and methods of eliciting an immune response by administering said compositions.

### RELATED APPLICATIONS

This application claims priority to U.S. Provisional Patent Application Ser. No. 63/465,389, filed on May 10, 2023, European Provisional Patent Application Serial Nos. 24315228.7, and 24305723.9, both filed on May 9, 2024, the disclosures of which are hereby incorporated by reference in its entirety.

### SEQUENCE LISTING

The instant application contains a Sequence Listing which has been submitted electronically in XML format and is hereby incorporated by reference in its entirety. Said XML file, created on Sep. 18, 2024, is named 754298_SA9-355_ST26.xml and is 88,205 bytes in size.

### BACKGROUND OF THE DISCLOSURE

Human respiratory syncytial virus (hRSV or RSV), human metapneumovirus (hMPV), and human parainfluenza virus type 3 (hPIV3 or PIV3) are respiratory pathogens associated with high morbidity and mortality in infants, children, and older adults with the combined disease burden outranking influenza. With no vaccines or specific therapies currently available, there exists an unmet need for a safe, effective, and convenient mRNA combination vaccine to protect individuals against these pathogens.

### BRIEF SUMMARY OF THE DISCLOSURE

In one aspect, the disclosure provides composition comprising at least two messenger RNAs (mRNAs), wherein the at least two mRNAs comprise an open reading frame (ORF) encoding a recombinant F protein antigenic polypeptide selected from the group consisting of: (i) a first mRNA encoding a human respiratory syncytial virus (hRSV) F protein antigen comprising an amino acid sequence of SEQ ID NO: 1; (ii) a second mRNA encoding a human metapneumovirus (hMPV) F protein antigen comprising an amino acid sequence of SEQ ID NO: 2; and (iii) a third mRNA encoding a human parainfluenza virus 3 (hPIV3) F protein antigen comprising an amino acid sequence of SEQ ID NO: 3.

In certain embodiments, the composition comprises the first mRNA encoding the amino acid sequence of SEQ ID NO: 1; and the second mRNA encoding the amino acid sequence of SEQ ID NO: 2.

In certain embodiments, the composition comprises the first mRNA encoding the amino acid sequence of SEQ ID NO: 1; and the third mRNA encoding the amino acid sequence of SEQ ID NO: 3.

In certain embodiments, the composition comprises the second mRNA encoding the amino acid sequence of SEQ ID NO: 2; and the third mRNA encoding the amino acid sequence of SEQ ID NO: 3.

In certain embodiments, the composition comprises at least three mRNAs, including: the first mRNA encoding the amino acid sequence of SEQ ID NO: 1; the second mRNA encoding the amino acid sequence of SEQ ID NO: 2; and the third mRNA encoding the amino acid sequence of SEQ ID NO: 3.

In certain embodiments, the composition comprises three mRNAs, including: the first mRNA encoding the amino acid sequence of SEQ ID NO: 1; the second mRNA encoding the amino acid sequence of SEQ ID NO: 2; and the third mRNA encoding the amino acid sequence of SEQ ID NO: 3.

In certain embodiments, the hRSV F protein comprises an amino acid sequence having at least 90% identity to SEQ ID NO: 1.

In certain embodiments, the hMPV F protein comprises an amino acid sequence having at least 90% identity to SEQ ID NO: 2.

In certain embodiments, the hPIV3 F protein comprises an amino acid sequence having at least 90% identity to SEQ ID NO: 3.

In certain embodiments, the first mRNA comprises a nucleic acid sequence having at least 80% identity to SEQ ID NO: 4.

In certain embodiments, the second mRNA comprises a nucleic acid sequence having at least 80% identity to SEQ ID NO: 5.

In certain embodiments, the third mRNA comprises a nucleic acid sequence having at least 80% identity to SEQ ID NO: 6.

In certain embodiments, at least one of the recombinant F protein antigenic polypeptides is a pre-fusion protein.

In certain embodiments, at least one of the mRNAs comprises a codon-optimized ORF.

In certain at least one of the mRNAs comprises at least one 5′ untranslated region (5′ UTR), at least one 3′ untranslated region (3′ UTR), and at least one polyadenylation (poly(A)) sequence.

In certain embodiments, at least one of the mRNAs comprises a 5′ UTR comprising a nucleic acid sequence with at least 80% identity to SEQ ID NO: 7.

In certain embodiments, at least one of the mRNAs comprises a 3′ UTR comprising a nucleic acid sequence with at least 80% identity to SEQ ID NO: 8.

In certain embodiments, at least one of the mRNAs comprises at least one chemical modification.

In certain embodiments, at least 20%, at least 30%, at least 40%, at least 50%, at least 60%, at least 70%, at least 80%, at least 85%, at least 90%, at least 95%, or 100% of the uracil nucleotides in at least one of the mRNAs are chemically modified.

In certain embodiments, at least 20%, at least 30%, at least 40%, at least 50%, at least 60%, at least 70%, at least 80%, at least 85%, at least 90%, at least 95%, or 100% of the uracil nucleotides in at least one of the ORFs are chemically modified.

In certain embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In certain embodiments, the chemical modification is selected from the group consisting of pseudouridine, N1-methylpseudouridine, 5-methylcytosine, 5-methoxyuridine, and a combination thereof.

In certain embodiments, the chemical modification is N1-methylpseudouridine.

In certain embodiments, the first mRNA, the second mRNA, and the third mRNA are present in a ratio (w/w) of about 1:1:1.

In certain embodiments, the first mRNA, the second mRNA, and the third mRNA are present in a ratio (w/w) of about 1:2:1.

In certain embodiments, the first mRNA, the second mRNA, and the third mRNA are present in a ratio (w/w) of about 1:3:1.

In certain embodiments, the first mRNA, the second mRNA, and the third mRNA are present in a ratio (w/w) of about 1:5:1.

In certain embodiments, the first mRNA, the second mRNA, and the third mRNA are present in a ratio (w/w) of about 1:1:1 to about 1:10:1.

In certain embodiments, the first mRNA, the second mRNA, and the third mRNA are present in a ratio (w/w) of about 1:1:0.1 to about 1:1:1.

In certain embodiments, the ratio is expressed in micrograms (pg).

In certain embodiments, the first mRNA, the second mRNA, and the third mRNA are present in a ratio (w/w) of about one microgram of the first mRNA to about one microgram of the second mRNA to about one microgram of the third mRNA.

In certain embodiments, the first mRNA, the second mRNA, and the third mRNA are present in a ratio (w/w) of about one microgram of the first mRNA to about two micrograms of the second mRNA to about one microgram of the third mRNA.

In certain embodiments, the first mRNA, the second mRNA, and the third mRNA are present in a ratio (w/w) of about one microgram of the first mRNA to about three micrograms of the second mRNA to about one microgram of the third mRNA.

In certain embodiments, the first mRNA, the second mRNA, and the third mRNA are present in a ratio (w/w) of about one microgram of the first mRNA to about five micrograms of the second mRNA to about one microgram of the third mRNA.

In certain embodiments, the first mRNA, the second mRNA, and the third mRNA are present in a ratio (w/w) of about 0.5 microgram of the first mRNA to about 0.5 microgram of the second mRNA to about 0.5 microgram of the third mRNA.

In certain embodiments, the first mRNA, the second mRNA, and the third mRNA are present in a ratio (w/w) of about 0.5 microgram of the first mRNA to about 1.5 microgram of the second mRNA to about 0.5 microgram of the third mRNA.

In certain embodiments, the first mRNA, the second mRNA, and the third mRNA are present in a ratio (w/w) of about 0.5 microgram of the first mRNA to about 0.5 microgram of the second mRNA to about 0.1 microgram of the third mRNA.

In certain embodiments, the first mRNA, the second mRNA, and the third mRNA are present in a ratio (w/w) of about 0.5 microgram of the first mRNA to about 1 microgram of the second mRNA to about 0.5 microgram of the third mRNA.

In certain embodiments, the first mRNA, the second mRNA, and the third mRNA are present in a ratio (w/w) of about 0.5 microgram of the first mRNA to about 2.5 micrograms of the second mRNA to about 0.5 microgram of the third mRNA.

In certain embodiments, the first mRNA, the second mRNA, and the third mRNA are not covalently linked to one another.

In certain embodiments, one or more of the first mRNA, the second mRNA, and the third mRNA are covalently linked to one another.

In certain embodiments, the first mRNA, the second mRNA, and the third mRNA are each formulated into a separate nanoparticle (LNP).

In certain embodiments, the first mRNA, the second mRNA, and the third mRNA are formulated into the same LNP.

In certain embodiments, the LNP comprises at least one cationic lipid.

In certain embodiments, the cationic lipid is biodegradable.

In certain embodiments, the cationic lipid is not biodegradable.

In certain embodiments, the cationic lipid is cleavable.

In certain embodiments, the cationic lipid is not cleavable.

In certain embodiments, the cationic lipid is selected from the group consisting of OF-02, cKK-E10, GL-HEPES-E3-E10-DS-3-E18-1, GL-HEPES-E3-E12-DS-4-E10, GL-HEPES-E3-E12-DS-3-E14, and IM-001.

In certain embodiments, the cationic lipid is cKK-E10.

In certain embodiments, the cationic lipid is GL-HEPES-E3-E12-DS-4-E10.

In certain embodiments, the cationic lipid is IM-001.

In certain embodiments, the LNP further comprises a polyethylene glycol (PEG) conjugated (PEGylated) lipid, a cholesterol-based lipid, and a helper lipid.

In certain embodiments, the LNP comprises: a cationic lipid at a molar ratio of 35% to 55%; a polyethylene glycol (PEG) conjugated (PEGylated) lipid at a molar ratio of 0.25% to 2.75%; a cholesterol-based lipid at a molar ratio of 20% to 45%; and a helper lipid at a molar ratio of 5% to 35%, wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the PEGylated lipid is dimyristoyl-PEG2000 (DMG-PEG2000) or 2-[(polyethylene glycol)-2000]-N,N-ditetradecylacetamide (ALC-0159).

In certain embodiments, the cholesterol-based lipid is cholesterol.

In certain embodiments, the helper lipid is 1,2-dioleoyl-SN-glycero-3-phosphoethanolamine (DOPE) or 1,2-distearoyl-sn-glycero-3-phosphocholine (DSPC).

In certain embodiments, the LNP comprises: GL-HEPES-E3-E12-DS-4-E10 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: IM-001 at a molar ratio of 40%; DMG-PEG2000 at a molar ratio of 1.5%; cholesterol at a molar ratio of 28.5%; and DOPE at a molar ratio of 30%.

In certain embodiments, the LNP has an average diameter of 30 nm to 200 nm.

In certain embodiments, the LNP has an average diameter of 80 nm to 150 nm.

In certain embodiments, at least one of the mRNAs comprises the following structural elements:
(i) a 5′ cap with the following structure:

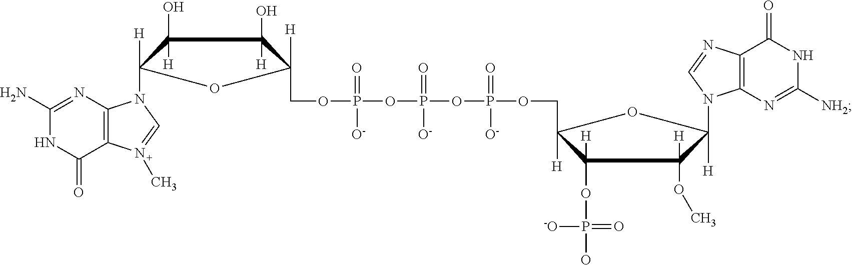

(ii) a 5′ untranslated region (5′ UTR) having the nucleic acid sequence of SEQ ID NO: 7;
(iii) a protein coding region having the nucleic acid sequence of SEQ ID NO: 4, 5, or 6;
(iv) a 3′ untranslated region (3′ UTR) having the nucleic acid sequence of SEQ ID NO: 8; and
(v) a poly(A) tail.

In one aspect, the disclosure provides a composition comprising at least two mRNAs, wherein each of the at least two mRNAs comprises an ORF encoding a recombinant F protein antigenic polypeptide, wherein at least one of the mRNAs comprises the following structural elements:
(i) a 5′ cap with the following structure:

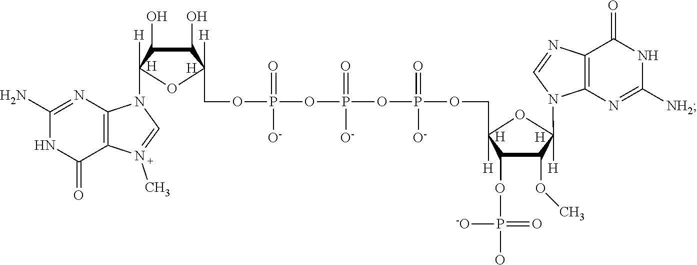

(ii) a 5′ untranslated region (5′ UTR) having the nucleic acid sequence of SEQ ID NO: 7;
(iii) a protein coding region having the nucleic acid sequence of SEQ ID NO: 4, 5, or 6;
(iv) a 3′ untranslated region (3′ UTR) having the nucleic acid sequence of SEQ ID NO: 8; and
(v) a poly(A) tail;
wherein the mRNA is formulated in a lipid nanoparticle (LNP) comprising:
GL-HEPES-E3-E12-DS-4-E10 at a molar ratio of 40%;
DMG-PEG2000 at a molar ratio of 1.5%;
cholesterol at a molar ratio of 28.5%; and
DOPE at a molar ratio of 30%.

In one aspect, the disclosure provides a composition comprising at least two mRNAs, wherein each of the at least two mRNAs comprises an ORF encoding a recombinant F protein antigenic polypeptide, wherein at least one of the mRNAs comprises the following structural elements:
(i) a 5′ cap with the following structure:

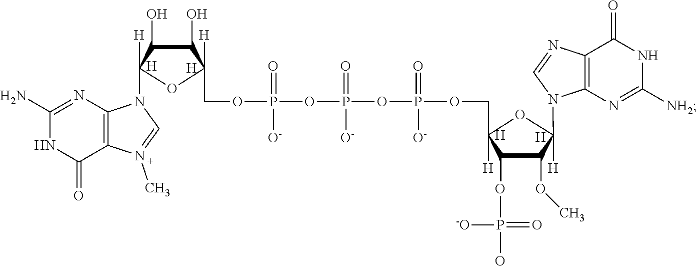

(ii) a 5′ untranslated region (5′ UTR) having the nucleic acid sequence of SEQ ID NO: 7;
(iii) a protein coding region having the nucleic acid sequence of SEQ ID NO: 4, 5, or 6;
(iv) a 3′ untranslated region (3′ UTR) having the nucleic acid sequence of SEQ ID NO: 8; and
(v) a poly(A) tail;
wherein the mRNA is formulated in a lipid nanoparticle (LNP) comprising:
cKK-E10 at a molar ratio of 40%;
DMG-PEG2000 at a molar ratio of 1.5%;
cholesterol at a molar ratio of 28.5%; and
DOPE at a molar ratio of 30%.

In one aspect, the disclosure provides a composition comprising at least two mRNAs, wherein each of the at least two mRNAs comprises an ORF encoding a recombinant F protein antigenic polypeptide, wherein at least one of the mRNAs comprises the following structural elements:
(i) a 5′ cap with the following structure:

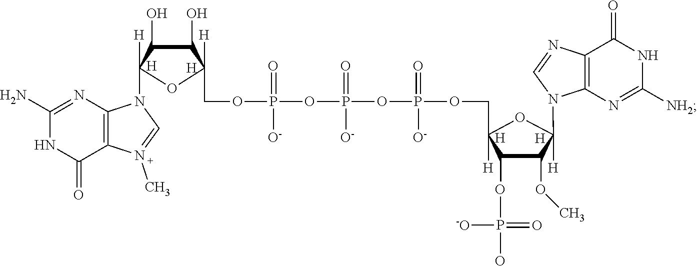

(ii) a 5′ untranslated region (5′ UTR) having the nucleic acid sequence of SEQ ID NO: 7;
(iii) a protein coding region having the nucleic acid sequence of SEQ ID NO: 4, 5, or 6;
(iv) a 3′ untranslated region (3′ UTR) having the nucleic acid sequence of SEQ ID NO: 8; and
(v) a poly(A) tail;
wherein the mRNA is formulated in a lipid nanoparticle (LNP) comprising:
IM-001 at a molar ratio of 40%;
DMG-PEG2000 at a molar ratio of 1.5%;
cholesterol at a molar ratio of 28.5%; and
DOPE at a molar ratio of 30%.

In one aspect, the disclosure provides a composition comprising at least two mRNAs, wherein each of the at least two mRNAs comprises an ORF encoding a recombinant F protein antigenic polypeptide, wherein at least one of the mRNAs comprises the following structural elements:
(i) a 5′ cap with the following structure:

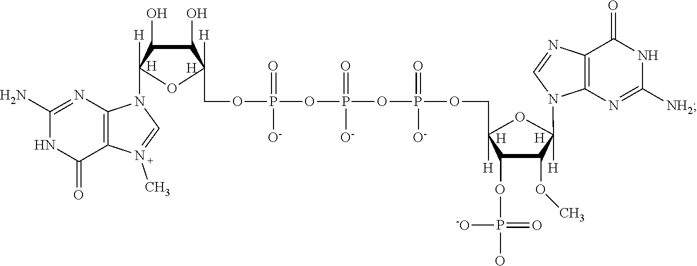

(ii) a 5′ untranslated region (5′ UTR) having the nucleic acid sequence of SEQ ID NO: 7;
(iii) a protein coding region having the nucleic acid sequence of SEQ ID NO: 4, 5, or 6;
(iv) a 3′ untranslated region (3′ UTR) having the nucleic acid sequence of SEQ ID NO: 8; and
(v) a poly(A) tail;
wherein the mRNA is formulated in a lipid nanoparticle (LNP) comprising: GL-HEPES-E3-E12-DS-4-E10, IM-001, OF-02, or cKK-E10 at a molar ratio of 35% to 55%.

In another aspect, the disclosure provides a method of eliciting an immune response to hRSV or protecting a subject against hRSV infection, comprising administering the compositions described above to a subject.

In another aspect, the disclosure provides a method of eliciting an immune response to hMPV or protecting a subject against hMPV infection, comprising administering the compositions described above to a subject.

In another aspect, the disclosure provides a method of eliciting an immune response to hPIV3 or protecting a subject against hPIV3 infection, comprising administering the compositions described above to a subject.

In certain embodiments, the subject has about the same or higher serum concentration of neutralizing antibodies against hRSV after administration of the composition, relative to a subject that is administered a single antigenic composition comprising an mRNA ORF encoding an hRSV F protein antigen of SEQ ID NO: 1.

In certain embodiments, the subject has about the same or higher serum concentration of neutralizing antibodies against hMPV after administration of the composition, relative to a subject that is administered a single antigenic composition comprising an mRNA ORF encoding an hMPV F protein antigen of SEQ ID NO: 2.

In certain embodiments, the subject has about the same or higher serum concentration of neutralizing antibodies against hPIV3 after administration of the composition, relative to a subject that is administered a single antigenic composition comprising an mRNA ORF encoding an hPIV3 F protein antigen of SEQ ID NO: 3.

In certain embodiments, the subject has a comparable serum concentration of neutralizing antibodies against hRSV after administration of the composition, relative to a subject that is administered a protein hRSV vaccine.

In certain embodiments, the subject has a comparable serum concentration of neutralizing antibodies against hMPV after administration of the composition, relative to a subject that is administered a protein hMPV vaccine.

In certain embodiments, the subject has a comparable serum concentration of neutralizing antibodies against hPIV3 after administration of the composition, relative to a subject that is administered a protein hPIV3 vaccine.

In certain embodiments, the composition increases the serum concentration of neutralizing antibodies in a subject with pre-existing hRSV immunity.

In certain embodiments, the composition increases the serum concentration of neutralizing antibodies in a subject with pre-existing hMPV immunity.

In certain embodiments, the composition increases the serum concentration of neutralizing antibodies in a subject with pre-existing hPIV3 immunity.

In another aspect, the disclosure provides a composition for use in eliciting an immune response to hRSV or protecting a subject against hRSV infection, comprising administering the compositions described above to a subject.

In another aspect, the disclosure provides a composition for use in eliciting an immune response to hMPV or protecting a subject against hMPV infection, comprising administering the compositions described above to a subject.

In another aspect, the disclosure provides a composition for use in eliciting an immune response to hPIV3 or protecting a subject against hPIV3 infection, comprising administering the compositions described above to a subject.

In another aspect, the disclosure provides use of the composition in the manufacture of a medicament for eliciting an immune response to hRSV or protecting a subject against hRSV infection.

In another aspect, the disclosure provides use of the composition in the manufacture of a medicament for eliciting an immune response to hMPV or protecting a subject against hMPV infection.

In another aspect, the disclosure provides use of the composition in the manufacture of a medicament for eliciting an immune response to hPIV3 or protecting a subject against hPIV3 infection.

In one aspect, the disclosure provides a composition comprising three mRNAs, wherein: (i) a first mRNA encodes an hRSV F protein antigen comprising an amino acid sequence of SEQ ID NO: 1; (ii) a second mRNA encodes a human metapneumovirus (hMPV) F protein antigen comprising an amino acid sequence of SEQ ID NO: 2; and (iii) a third mRNA encodes a human parainfluenza virus 3 (hPIV3) F protein antigen comprising an amino acid sequence of SEQ ID NO: 3; wherein the first mRNA, the second mRNA, and the third mRNA are formulated into the same LNP comprising: OF-02, cKK-E10, GL-HEPES-E3-E10-DS-3-E18-1, GL-HEPES-E3-E12-DS-4-E10, or GL-HEPES-E3-E12-DS-3-E14 at a molar ratio of 40%; DMG-PEG2000 at a molar ratio of 1.5%; cholesterol at a molar ratio of 28.5%; and DOPE at a molar ratio of 30%.

In one aspect, the disclosure provides a composition comprising three mRNAs, wherein: (i) a first mRNA encodes a human respiratory syncytial virus (hRSV) F protein antigen comprising an amino acid sequence of SEQ ID NO: 1; (ii) a second mRNA encodes a human metapneumovirus (hMPV) F protein antigen comprising an amino acid sequence of SEQ ID NO: 2; and (iii) a third mRNA encodes a human parainfluenza virus 3 (hPIV3) F protein antigen comprising an amino acid sequence of SEQ ID NO: 3.

In one aspect, the disclosure provides a composition comprising a first messenger RNA (mRNA) and a second mRNA, wherein: (i) the first mRNA encodes a human respiratory syncytial virus (hRSV) F protein antigen comprising an amino acid sequence of SEQ ID NO: 1; and (ii) the second mRNA encodes a human metapneumovirus (hMPV) F protein antigen comprising an amino acid sequence of SEQ ID NO: 2.

In certain embodiments, the first mRNA and the second mRNA are present in a ratio (w/w) of about 1:1.

In certain embodiments, the first mRNA and the second mRNA are present in a ratio (w/w) of about 1:2.

In certain embodiments, the first mRNA and the second mRNA are present in a ratio (w/w) of about 1:3.

In certain embodiments, the first mRNA and the second mRNA are present in a ratio (w/w) of about 1:5.

In certain embodiments, the first mRNA and the second mRNA are present in a ratio (w/w) of about 1:1 to about 1:10.

In certain embodiments, the ratio of the first mRNA and the second mRNA is expressed in micrograms (pg).

In certain embodiments, the first mRNA and the second mRNA are present in a ratio (w/w) of about 0.5 microgram of the first mRNA to about 0.5 microgram of the second mRNA.

In certain embodiments, the first mRNA and the second mRNA are present in a ratio (w/w) of about 0.5 microgram of the first mRNA to about 1.5 microgram of the second mRNA.

In certain embodiments, the first mRNA and the second mRNA are present in a ratio (w/w) of about 0.5 microgram of the first mRNA to about 1 microgram of the second mRNA.

In certain embodiments, the first mRNA and the second mRNA are present in a ratio (w/w) of about 0.5 microgram of the first mRNA to about 2.5 microgram of the second mRNA.

In certain embodiments, the first mRNA and the second mRNA are present in a ratio (w/w) of about 1 microgram of the first mRNA to about 1 microgram of the second mRNA.

In certain embodiments, the first mRNA and the second mRNA are present in a ratio (w/w) of about 1 microgram of the first mRNA to about 2 micrograms of the second mRNA.

In certain embodiments, the first mRNA and the second mRNA are present in a ratio (w/w) of about 1 microgram of the first mRNA to about 3 micrograms of the second mRNA.

In certain embodiments, the first mRNA and the second mRNA are present in a ratio (w/w) of about 1 microgram of the first mRNA to about 5 micrograms of the second mRNA.

In certain embodiments, the first mRNA and the second mRNA are each formulated into a separate LNP.

In certain embodiments, the first mRNA and the second mRNA are formulated into the same LNP.

In certain embodiments, the LNP comprises at least one cationic lipid.

In certain embodiments, the cationic lipid is biodegradable.

In certain embodiments, the cationic lipid is not biodegradable.

In certain embodiments, the cationic lipid is cleavable.

In certain embodiments, the cationic lipid is not cleavable.

In certain embodiments, the cationic lipid is selected from the group consisting of OF-02, cKK-E10, GL-HEPES-E3-E10-DS-3-E18-1, GL-HEPES-E3-E12-DS-4-E10, GL-HEPES-E3-E12-DS-3-E14 and IM-001.

In certain embodiments, the cationic lipid is cKK-E10.

In certain embodiments, the cationic lipid is GL-HEPES-E3-E12-DS-4-E10.

In certain embodiments, the cationic lipid is IM-001.

In certain embodiments, the LNP further comprises a polyethylene glycol (PEG) conjugated (PEGylated) lipid, a cholesterol-based lipid, and a helper lipid.

In certain embodiments, the LNP comprises: a cationic lipid at a molar ratio of 35% to 55%; a polyethylene glycol (PEG) conjugated (PEGylated) lipid at a molar ratio of 0.25% to 2.75%; a cholesterol-based lipid at a molar ratio of 20% to 45%; and a helper lipid at a molar ratio of 5% to 35%, wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the PEGylated lipid is dimyristoyl-PEG2000 (DMG-PEG2000) or 2-[(polyethylene glycol)-2000]-N,N-ditetradecylacetamide (ALC-0159).

In certain embodiments, the cholesterol-based lipid is cholesterol.

In certain embodiments, the helper lipid is 1,2-dioleoyl-SN-glycero-3-phosphoethanolamine (DOPE) or 1,2-distearoyl-sn-glycero-3-phosphocholine (DSPC).

In certain embodiments, the LNP comprises: GL-HEPES-E3-E12-DS-4-E10 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%.

In certain embodiments, the LNP comprises: IM-001 at a molar ratio of 40%; DMG-PEG2000 at a molar ratio of 1.5%; cholesterol at a molar ratio of 28.5%; and DOPE at a molar ratio of 30%.

In certain embodiments, the LNP has an average diameter of 30 nm to 200 nm.

In certain embodiments, the LNP has an average diameter of 80 nm to 150 nm.

In another aspect, the disclosure provides a method of eliciting an immune response to hRSV or protecting a subject against hRSV infection, comprising administering the compositions described above to a subject.

In another aspect, the disclosure provides a method of eliciting an immune response to hMPV or protecting a subject against hMPV infection, comprising administering the compositions described above to a subject.

In certain embodiments, the subject has about the same or higher serum concentration of neutralizing antibodies against hRSV after administration of the composition, relative to a subject that is administered a single antigenic composition comprising an mRNA ORF encoding an hRSV F protein antigen of SEQ ID NO: 1.

In certain embodiments, the subject has about the same or higher serum concentration of neutralizing antibodies against hMPV after administration of the composition, relative to a subject that is administered a single antigenic composition comprising an mRNA ORF encoding an hMPV F protein antigen of SEQ ID NO: 2.

In certain embodiments, the subject has a comparable serum concentration of neutralizing antibodies against hRSV after administration of the composition, relative to a subject that is administered a protein hRSV vaccine.

In certain embodiments, the subject has a comparable serum concentration of neutralizing antibodies against hMPV after administration of the composition, relative to a subject that is administered a protein hMPV vaccine.

In certain embodiments, the composition increases the serum concentration of neutralizing antibodies in a subject with pre-existing hRSV immunity.

In certain embodiments, the composition increases the serum concentration of neutralizing antibodies in a subject with pre-existing hMPV immunity.

In another aspect, the disclosure provides a composition for use in eliciting an immune response to hRSV or protecting a subject against hRSV infection, comprising administering the compositions described above to a subject.

In another aspect, the disclosure provides a composition for use in eliciting an immune response to hMPV or protecting a subject against hMPV infection, comprising administering the compositions described above to a subject.

In another aspect, the disclosure provides use of the composition in the manufacture of a medicament for eliciting an immune response to hRSV or protecting a subject against hRSV infection.

In another aspect, the disclosure provides use of the composition in the manufacture of a medicament for eliciting an immune response to hMPV or protecting a subject against hMPV infection.

### BRIEF DESCRIPTION OF THE DRAWINGS/FIGURES

The foregoing and other features and advantages of the present disclosure will be more fully understood from the following detailed description of illustrative embodiments taken in conjunction with the accompanying drawings.

FIG. 1A-FIG. 1F depict protein expression in human skeletal muscle (hSKM) cells transfected with monovalent mRNA-LNP formulations of hRSV, hPIV3, or hMPV (with doses ranging from 0.088 μg to 3 μg) and trivalent mRNA-LNP co-formulations of hRSV, hPIV3, and hMPV (with doses ranging from 0.088 μg to 3 μg in a 1:1:1 ratio). mRNA constructs were formulated with LNP OF-02. 24 hours post-transfection, the hSKM cells were harvested and stained with pre-fusion binding monoclonal antibodies (FIGS. 1A, 1C, and 1E) or common region binding monoclonal antibodies (FIGS. 1B, 1D, and 1F) and a fluorescently labeled secondary antibody. Florescence was quantified by flow cytometry and is displayed as integrated Mean Fluorescent Intensity (iMFI).

FIG. 2A-FIG. 2F depict immunogenicity in mice following vaccination with either the trivalent mRNA-LNP co-formulation (containing 0.5 μg of pre-fusion hRSV mRNA, 0.5 μg of pre-fusion hPIV3 mRNA, and 0.5 μg of pre-fusion hMPV mRNA per dose), an admix of the three monovalent mRNA-LNP formulations (containing 0.5 μg/dose of pre-fusion hRSV mRNA, 0.5 μg/dose of pre-fusion hPIV3 mRNA, and 0.5 μg/dose of pre-fusion hMPV mRNA), or a monovalent mRNA-LNP formulation (containing 0.5 μg/dose of either pre-fusion hRSV mRNA, pre-fusion hPIV3 mRNA, or pre-fusion hMPV mRNA). mRNA constructs were formulated with LNP OF-02. All formulations were diluted in trehalose. The mice were boosted with the same regimens 21 days post-prime, with blood collected before the first immunization (Day 0) and 2 weeks after the second immunization (Day 35). Binding antibody titers were determined by ELISA using plates coated with pre-fusion hRSV (FIG. 2A), pre-fusion hPIV3 (FIG. 2C), or pre-fusion hMPV (FIG. 2E). Neutralizing antibody titers were determined by microneutralization against hRSV (FIG. 2B), hPIV3 (FIG. 2D), or hMPV (FIG. 2F) expressing Green Fluorescent Protein (GFP) reporters. Day 0 samples were tested as group pools in the microneutralization assay. Dotted lines represent lower limits of quantification. Error bars represent 1 geometric standard deviation from the geometric mean. Statistical analysis was performed using mixed model two-way ANOVA (*p<0.05, **p<0.01, ***p<0.001).

FIG. 3A-FIG. 3F depict immunogenicity in mice following vaccination with an admix of hRSV, hPIV3, and hMPV pre-fusion recombinant proteins, or a monovalent pre-fusion recombinant protein of hRSV, hPIV3, or hMPV. Groups of mice were intramuscularly vaccinated with either an admix (containing 0.5 μg each of hRSV, hPIV3, and hMPV pre-fusion recombinant proteins) adjuvanted with Alhydrogel® 2% (aluminum hydroxide gel), or a monovalent pre-fusion recombinant protein (containing a 0.5 μg/dose of either pre-fusion hRSV protein, pre-fusion hPIV3 protein, or pre-fusion hMPV protein) adjuvanted with Alhydrogel®. All formulations were diluted in trehalose. The mice were boosted with the same regimens 21 days post-prime, with blood collected before the first immunization (Day 0) and 2 weeks after the second immunization (Day 35). Binding antibody titers were determined by ELISA using plates coated with pre-fusion hRSV (FIG. 3A), hPIV3 (FIG. 3B), or hMPV (FIG. 3C). Neutralizing antibody titers were determined by microneutralization against hRSV (FIG. 3D), hPIV3 (FIG. 3E), or hMPV (FIG. 3F) expressing a GFP reporter. Day 0 samples were run in the microneutralization assay as group pools. Dotted lines represent lower limits of quantification. Error bars represent 1 geometric standard deviation from the geometric mean. Statistical analyses were performed using mixed model two-way ANOVA (*p<0.05, **p<0.01, ***p<0.001).

FIG. 4A-FIG. 4F depict immunogenicity in mice after increasing hMPV doses in an admix of the hRSV, hMPV, and hPIV3 monovalent mRNA-LNP formulations in LNP OF-02. Groups of mice were intramuscularly immunized with a 1:1:1, 1:2:1, or 1:5:1 admix of pre-fusion hRSV:hMPV:hPIV3 mRNA, or a 0.5 μg/dose of monovalent hMPV mRNA. All formulations were diluted in trehalose. The mice were boosted with the same regimens 21 days post-prime, with blood collected before the first immunization (Day 0) and 2 weeks after the second immunization (Day 35). Binding antibody titers were determined by ELISA using plates coated with pre-fusion hRSV (FIG. 4A), hPIV3 (FIG. 4B), or hMPV (FIG. 4C). Neutralizing antibody titers were determined by microneutralization against hRSV (FIG. 4D), hPIV3 (FIG. 4E), or hMPV (FIG. 4F) expressing a GFP reporter. Day 0 samples were run in the microneutralization assay as group pools. Dotted lines represent lower limits of quantification. Error bars represent 1 geometric standard deviation from the geometric mean. Statistical analyses were performed using mixed model two-way ANOVA (*p<0.05, **p<0.01, ***p<0.001).

FIG. 5A-FIG. 5F depict immunogenicity in mice after decreasing hPIV3 doses in an admix of the hRSV, hMPV, and hPIV3 monovalent mRNA-LNP formulations in LNP OF-02. Groups of mice were intramuscularly immunized with a 1:1:1 admix of pre-fusion hRSV:hMPV:hPIV3 mRNA (i.e., 0.5 μg of pre-fusion hRSV mRNA, 0.5 μg of pre-fusion hMPV, and 0.5 μg of hPIV3 mRNA), a 1:1:0.2 admix of pre-fusion hRSV:hMPV:hPIV3 mRNA (i.e., 0.5 μg of pre-fusion hRSV mRNA, 0.5 μg of pre-fusion hMPV, and 0.1 μg of hPIV3 mRNA), or a 0.1 μg/dose of monovalent hPIV3 mRNA. All formulations were diluted in trehalose. The mice were boosted with the same regimens 21 days post-prime, with blood collected before the first immunization (Day 0) and 2 weeks after the second immunization (Day 35). Binding antibody titers were determined by ELISA using plates coated with pre-fusion hRSV (FIG. 5A), hPIV3 (FIG. 5B), or hMPV (FIG. 5C). Neutralizing antibody titers were determined by microneutralization against hRSV (FIG. 5D), hPIV3 (FIG. 5E), or hMPV (FIG. 5F) expressing a GFP reporter. Day 0 samples were run in the microneutralization assay as group pools. Dotted lines represent lower limits of quantification. Error bars represent 1 geometric standard deviation from the geometric mean. Statistical analyses were performed using mixed model two-way ANOVA (*p<0.05, **p<0.01, ***p<0.001).

FIG. 6A-FIG. 6F depict immunogenicity in mice after increasing hMPV doses in trivalent hRSV:hMPV:hPIV3 co-formulations tested in LNP OF-02. Groups of mice were intramuscularly immunized with a 1:1:1, 1:2:1, or 1:5:1 coformulation of pre-fusion hRSV:hMPV:hPIV3 mRNA, or a monovalent formulation (containing 0.5 μg/dose of either pre-fusion hRSV mRNA, pre-fusion hPIV3 mRNA, or pre-fusion hMPV mRNA). All formulations were diluted in trehalose. The mice were boosted with the same regimens 21 days post-prime, with blood collected before the first immunization (Day 0) and 2 weeks after the second immunization (Day 35). Binding antibody titers were determined by ELISA using plates coated with pre-fusion hRSV (FIG. 6A), hPIV3 (FIG. 6C), or hMPV (FIG. 6E). Neutralizing antibody titers were determined by microneutralization against hRSV (FIG. 6B), hPIV3 (FIG. 6D), or hMPV (FIG. 6F) expressing a GFP reporter. Day 0 samples were run in the microneutralization assay as group pools. Dotted lines represent lower limits of quantification. Error bars represent 1 geometric standard deviation from the geometric mean. Statistical analyses were performed using mixed model two-way ANOVA (*p<0.05, **p<0.01, ***p<0.001).

FIG. 7A-FIG. 7F depict immunogenicity in mice after increasing hMPV doses in trivalent hRSV:hMPV:hPIV3 co-formulations tested in LNP cKK-E10. Groups of mice were intramuscularly immunized with a 1:2:1 or 1:5:1 co-formulation of pre-fusion hRSV:hMPV:hPIV3 mRNA, or a monovalent formulation (containing 0.5 μg/dose of either pre-fusion hRSV mRNA, pre-fusion hPIV3 mRNA, or pre-fusion hMPV mRNA). All formulations were diluted in trehalose. The mice were boosted with the same regimens 21 days post-prime, with blood collected before the first immunization (Day 0) and 2 weeks after the second immunization (Day 35). Binding antibody titers were determined by ELISA using plates coated with pre-fusion hRSV (FIG. 7A), hPIV3 (FIG. 7C), or hMPV (FIG. 7E). Neutralizing antibody titers were determined by microneutralization against hRSV (FIG. 7B), hPIV3 (FIG. 7D), or hMPV (FIG. 7F) expressing a GFP reporter. Day 0 samples were run in the microneutralization assay as group pools. Dotted lines represent lower limits of quantification. Error bars represent 1 geometric standard deviation from the geometric mean. Statistical analyses were performed using mixed model two-way ANOVA (*p<0.05, **p<0.01, ***p<0.001).

FIG. 8A-FIG. 8C depict Western blots for hRSV (FIG. 8A), hMPV (FIG. 8B), and hPIV3 (FIG. 8C) protein expression in 1:1:1 co-formulations of hRSV:hMPV:hPIV3 mRNA, 1:3:1 co-formulations of hRSV:hMPV:hPIV3, and monovalent formulations of hRSV, hMPV, and hPIV3 tested with LNP OF-02, LNP cKK-E10, or LNP GL-HEPES-E3-E12-DS-4-E10. HeLa cells plated in 24-well plates at 0.075 million cells/well overnight were transfected the next day with 1pg/million cells of the indicated mRNA formulations. Twenty-four hours post-transfection, the cells were lysed and clarified lysates were run on an 8-16% gradient SDS-PAGE. Following transfer to a nitrocellulose membrane, hRSV, hMPV, and hPIV3 protein expression was detected with antibodies CR9501, SAN27K-37, or SAN52-36, respectively. All formulations containing hRSV, hMPV, or hPIV3 mRNA were positive for expression of the corresponding proteins.

FIG. 9A-FIG. 9F depict immunogenicity in mice following immunization with trivalent hRSV:hMPV:hPIV3 co-formulations tested in LNP OF-02, LNP cKK-E10, or LNP GL-HEPES-E3-E12-DS-4-E10 whose protein expression was confirmed in FIG. 8. Groups of mice were intramuscularly immunized with a 1:1:1 co-formulation of hRSV:hMPV:hPIV3 mRNA (containing 0.5 μg of pre-fusion hRSV mRNA, 0.5 μg of pre-fusion hMPV mRNA, and 0.5 μg of pre-fusion hPIV3 mRNA per dose) formulated with LNP OF-02, LNP cKK-E10, or LNP GL-HEPES-E3-E12-DS-4-E10; a 1:3:1 co-formulation of hRSV:hMPV:hPIV3 mRNA (containing 0.5 μg of pre-fusion hRSV mRNA, 1.5 μg of pre-fusion hMPV mRNA, and 0.5 μg of pre-fusion hPIV3 mRNA per dose) formulated with LNP OF-02, LNP cKK-E10, or LNP GL-HEPES-E3-E12-DS-4-E10; or a 0.5 μg/dose of monovalent RSV, hMPV, or hPIV3 mRNA formulated with LNP OF-02, LNP cKK-E10, or LNP GL-HEPES-E3-E12-DS-4-E10. The formulations were diluted to the indicated concentrations with phosphate-buffered saline (PBS). The mice were boosted with the same regimens 21 days post-prime, with blood collected before the first immunization (Day 0), before the second immunization (Day 21), and 2 weeks after the second immunization (Day 35). Binding antibody titers were determined by ELISA using plates coated with pre-fusion hMPV (FIG. 9B), hPIV3 (FIG. 9D), or hRSV (FIG. 9F). Neutralizing antibody titers were determined by microneutralization against hMPV (FIG. 9A), hPIV3 (FIG. 9C), or hRSV (FIG. 9E) expressing a GFP reporter. Dotted lines represent lower limits of quantification. All day 0 samples were below the lower limit of quantification in all assays. Samples from groups immunized with monovalent formulations were tested only against the corresponding targets. Error bars represent 1 geometric standard deviation from the geometric mean. Statistical analyses were performed using mixed model two-way ANOVA (*p<0.05, **p<0.01, ***p<0.001).

FIG. 10 depicts neutralizing antibody titers for hRSV, hMPV, and hPIV3 mRNA constructs diluted with PBS compared to neutralizing antibody titers for hRSV, hMPV, and hPIV3 mRNA constructs diluted with trehalose. Groups of mice were intramuscularly immunized with a 1:1:1 co-formulation of hRSV:hMPV:hPIV3 mRNA (containing 0.5 μg of pre-fusion hRSV mRNA, 0.5 μg of pre-fusion hMPV mRNA, and 0.5 μg of pre-fusion hPIV3 mRNA per dose) formulated with LNP OF-02 diluted to this concentration with either trehalose or PBS. The mice were boosted with the same regimen 21 days post-prime, with blood collected before the first immunization (Day 0), before the second immunization (Day 21), and 2 weeks after the second immunization (Day 35). Neutralizing antibody titers were determined by microneutralization against hMPV, hPIV3, or hRSV expressing a GFP reporter. Dotted lines represent lower limits of quantification. All day 0 samples were below the lower limit of quantification in all assays. Error bars represent 1 geometric standard deviation from the geometric mean. Statistical analyses were performed using mixed model two-way ANOVA (*p<0.05, **p<0.01, ***p<0.001).

FIG. 11A-FIG. 11D depict immunogenicity in mice following immunization with bivalent hRSV:hMPV co-formulations tested in LNP OF-02, LNP GL-HEPES-E3-E12-DS-4-E10, or LNP IM-001. Groups of mice were immunized with a 1:1 co-formulation of hRSV:hMPV mRNA (containing 0.5 μg of pre-fusion hRSV mRNA and 0.5 μg of pre-fusion hMPV mRNA per dose) formulated with LNP OF-02, LNP GL-HEPES-E3-E12-DS-4-E10, or LNP IM-001; or a 0.5 μg/dose of monovalent hRSV or hMPV mRNA formulated with LNP OF-02, LNP GL-HEPES-E3-E12-DS-4-E10, or LNP IM-001. All formulations were diluted in PBS. The mice were boosted with the same regimens 21 days post-prime, with blood collected before the first immunization (Day 0), before the second immunization (Day 21), and 2 weeks after the second immunization (Day 35). Binding antibody titers were determined by ELISA using plates coated with pre-fusion hRSV (FIG. 11B) or hMPV (FIG. 9D). Neutralizing antibody titers were determined by microneutralization against hRSV (FIG. 11A) or hMPV (FIG. 11C) expressing a GFP reporter. Dotted lines represent lower limits of quantification. All day 0 samples were below the lower limit of quantification in all assays. Samples from groups immunized with monovalent formulations were tested only against the corresponding targets. Error bars represent 1 geometric standard deviation from the geometric mean. Statistical analyses were performed using mixed model two-way ANOVA (*p<0.05, **p<0.01, ***p<0.001).

FIG. 12A-12B depict hRSV, hMPV, and hPIV3 immunogenicity in mice following immunization with a bivalent hRSV:hMPV co-formulation alone or administered together with an hPIV3 admix. Groups of mice were immunized with a bivalent 1:1 co-formulation of hRSV:hMPV mRNA (containing 0.5 μg of pre-fusion hRSV mRNA and 0.5 μg of pre-fusion hMPV mRNA per dose); a bivalent 1:1 co-formulation of hRSV:hMPV mRNA (containing 0.5 μg of pre-fusion hRSV mRNA and 0.5 μg of pre-fusion hMPV mRNA per dose) administered together with an hPIV3 admix (containing 0.5 μg/dose of pre-fusion hPIV3 mRNA); or monovalent hPIV3 (containing 0.5 μg/dose of pre-fusion hPIV3 mRNA) as the control. Each mRNA construct was formulated with LNP GL-HEPES-E3-E12-DS-4-E10 and diluted in PBS. The mice were boosted with the same regimens 21 days post-prime, with blood collected before the first immunization (Day 0), before the second immunization (Day 21), and 2 weeks after the second immunization (Day 35). Binding antibody titers were determined by ELISA (FIG. 12A) using plates coated with pre-fusion hMPV, hPIV3 or hRSV. Neutralizing antibody titers (FIG. 12B) were determined by microneutralization against hMPV, hPIV3 or hRSV expressing a GFP reporter. Dotted lines represent lower limits of quantification. All day 0 samples were below the lower limit of quantification in all assays. All samples were tested against all three targets. Error bars represent 1 geometric standard deviation from the geometric mean. Statistical analyses were performed using mixed model two-way ANOVA (*p<0.05, **p<0.01, ***p<0.001).

### DETAILED DESCRIPTION OF THE DISCLOSURE

The present disclosure is directed to, inter alia, novel RNA (e.g., mRNA) compositions encoding a combination of hRSV F proteins, hMPV proteins, and/or hPIV3 proteins, and methods of vaccination with the same. In particular, the disclosures relate to mRNA encoding these proteins formulated in a lipid nanoparticle (LNP).

### I. Definitions

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, protein and nucleic acid chemistry, and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, may provide one of skill with a general dictionary of many of the terms used in this disclosure.

Units, prefixes, and symbols are denoted in their International System of Units (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, 5%, 4%, 3%, 2%, 1%, 0.9%, 0.8%, 0.7%, ±0.6%, 0.5%, 0.4%, 0.3%, 0.2%, 0.1%, 0.05%, or 0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

As used herein, the term “messenger RNA” or “mRNA” refers to a polynucleotide that encodes at least one polypeptide. mRNA, as used herein, encompasses both modified and unmodified RNA. mRNA may contain one or more coding and non-coding regions. A coding region is alternatively referred to as an open reading frame (ORF). Non-coding regions in mRNA include the 5′ cap, 5′ untranslated region (UTR), 3′ UTR, and a poly(A) tail. mRNA can be purified from natural sources, produced using recombinant expression systems (e.g., in vitro transcription) and optionally purified or chemically synthesized.

As used herein, the term “antigenic site Ø” or “site Ø epitope” for RSV refers to a site located at the apex of the pre-fusion RSV F trimer comprising amino acid residues 62-69 and 196-209 of wild-type RSV F (SEQ ID NO: 9). The RSV F site Ø epitope is a binding site for antibodies that have specificity for pre-fusion RSV F, such as D25 and AM14, and binding of antibodies to the site Ø epitope blocks cell-surface attachment of RSV (see, e.g., McLellan et al., Science, 340(6136): 1113-1117, 2013). Recombinant human anti-RSV antibody D25 (Creative Biolabs®; CAT #: PABL-322) and recombinant human anti-RSV antibody AM14 (Creative Biolabs®; CAT #: PABL-321) are each commercially available.

As used herein, the term “antigenic site Ø” or “site Ø epitope” for hMPV refers to a site located in the pre-fusion form of the hMPV F trimer. The hMPV site Ø epitope is a binding site for antibodies that have specificity for pre-fusion hMPV F.

As used herein, the term “antigenic site Ø” or “site Ø epitope” for hPIV3 refers to a site located in the pre-fusion form of the hPIV3 trimer. The hPIV3 site Ø epitope is a binding site for antibodies that have specificity for pre-fusion hPIV3.

As used herein, the term “antigenic site V” or “site V epitope” refers to a site located in the pre-fusion form of the hMPV F trimer. The site V epitope is a binding site for antibodies that have specificity for pre-fusion hMPV F.

As used herein, the term “antigen stability” refers to stability of the antigen over time or in solution.

As used herein, the term “cavity filling substitutions” refers to engineered hydrophobic substitutions to fill cavities present in the pre-fusion RSV F trimer, the pre-fusion hMPV F trimer, or the pre-fusion hPIV3 trimer.

As used herein, the term “RSV F protein” or “F protein” with respect to RSV refers to the protein of RSV responsible for driving fusion of the viral envelope with host cell membrane during viral entry.

As used herein, the term “hMPV F protein” or “F protein” with respect to hMPV refers to the protein of hMPV responsible for mediating fusion of the viral envelope and the host cell membrane during viral entry.

As used herein, the term “hPIV3 F protein” or “F protein” with respect to hPIV3 refers to the protein of hPIV3 responsible for mediating fusion of the viral envelope and the host cell membrane during viral entry.

As used herein, the terms “RSV F polypeptide” refers to a polypeptide comprising at least one epitope of F protein.

As used herein, the terms “hMPV F polypeptide” refers to a polypeptide comprising at least one epitope of the hMPV F protein.

As used herein, the terms “hPIV3 F polypeptide” refers to a polypeptide comprising at least one epitope of the hPIV3 F protein.

As used herein, the term “polypeptide” refers to any chain of amino acids, regardless of length or port-translational modification (e.g., glycosylation or phosphorylation). “Polypeptide” applies to amino acid polymers including naturally occurring amino acid polymers and non-naturally occurring amino acid polymers as well as in which one or more amino acid residues is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein and is used herein to refer to a polymer of amino acid residues.

As used herein, the term “glycan addition” refers to the addition of mutations which introduce glycosylation sites not present in wild-type RSV F, which can be engineered to increase construct expression, increase construct stability, or block epitopes shared between the pre-fusion and post-fusion conformation. A modified protein comprising glycan additions would have more glycosylation and therefore a higher molecular weight. Glycan addition can reduce the extent to which an RSV F polypeptide elicits antibodies to the post-fusion conformation of RSV F.

As used herein, a “foldon domain” refers to a trimerization domain of T4 fibritin.

As used herein, a “signal peptide” or “signal sequence” refers to a peptide of approximately 16-30 amino acids in length present at the amino-terminus or the carboxy-terminus of a polypeptide that functions to translocate the polypeptide to the secretory pathway in the endoplasmic reticulum and the Golgi apparatus.

As used herein a “tag sequence” or “affinity tag” refers to a polypeptide sequence that may be used to purify a polypeptide or a protein comprising the tag sequence. Tag sequences include, for example, polyhistidine-tags (e.g., hexahistidine (6×His tag)(SEQ ID NO:41), octahistidine (8×His tag)(SEQ ID NO:42), etc.), glutathione S-transferase (GST), FLAG, streptavidin-binding peptide (SBP), strep II, maltose-binding protein (MBP), calmodulin-binding protein (CBP), chitin-binding domain (CBD), S protein of RNase A, hemagglutinin (HA), c-Myc, and the like.

As used herein, the term “intra-protomer stabilizing substitutions” refers to amino acid substitutions in RSV F, hMPV F, or hPIV3 F that stabilize the pre-fusion conformation by stabilizing the interaction within a protomer of the RSV F trimer, within a protomer of the hMPV F trimer, or within a protomer of the hPIV3 trimer, respectively.

As used herein, the term “inter-protomer stabilizing substitutions” refers to amino acid substitutions in RSV F, hMPV F, or hPIV3 F that stabilize the pre-fusion conformation by stabilizing the interaction of the protomers of the RSV F trimer with each other, the protomers of the hMPV F with each other, or the protomers of the hPIV3 F trimer with each other, respectively.

As used herein, the term “protease cleavage” refers to proteolysis (sometimes also referred to as “clipping”) of susceptible residues (e.g., lysine or arginine) in a polypeptide sequence. Protease cleavage sites include viral protease cleavage sites such as, e.g., an hMPV F0 protease cleavage site, an RSV F0 protease cleavage site, a human rhinovirus 3C (HRV-3C) protease cleavage site, and an hPIV3 F0 protease cleavage site.

As used herein, the term “post-fusion” with respect to RSV F, hMPV F, and hPIV3 F refers to a stable conformation of RSV F, hMPV F, and hPIV3 F that occurs after merging of the virus and cell membranes.

As used herein, the term “pre-fusion” with respect to RSV F, hMPV F, and hPIV3 F refers to a conformation of RSV F, hMPV F, and hPIV3 F that is adopted before virus-cell interaction.

As used herein, the term “protomer” refers to a structural unit of an oligomeric protein. In the case of RSV F, an individual unit of the RSV F trimer is a protomer. In the case of RSV F, an individual unit of the RSV F trimer is a protomer. In the case of hMPV F, an individual unit of the hMPV F trimer is a protomer. In the case of hPIV3 F, an individual unit of the hPIV3 F trimer is a protomer.

As used herein, the term “N-glycan” refers to a saccharide chain attached to a protein at the amide nitrogen of an N (asparagine) residue of the protein. As such, an N-glycan is formed by the process of N-glycosylation. This glycan may be a polysaccharide.

As used herein, the term “glycosylation” refers to the addition of a saccharide unit to a protein.

As used herein, the term “immune response” refers to a response of a cell of the immune system, such as a B cell, T cell, dendritic cell, macrophage, or polymorphonucleocyte to a stimulus such as an antigen or vaccine. An immune response can include any cell of the body involved in a host defense response, including, for example, an epithelial cell that secretes an interferon or a cytokine. An immune response includes, but is not limited to, an innate and/or adaptive immune response.

As used herein, a “protective immune response” refers to an immune response that protects a subject from infection (e.g., prevents infection or prevents the development of disease associated with infection). Methods of measuring immune responses include measuring, for example, proliferation and/or activity of lymphocytes (such as B or T cells), secretion of cytokines or chemokines, inflammation, antibody production, and the like.

As used herein, an “antibody response” is an immune response in which antibodies are produced.

As used herein, an “antigen” refers to an agent that elicits an immune response, and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. A particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity. Antigens include the RSV polypeptides, the hMPV polypeptides, and the hPIV3 polypeptides encoded by mRNA as described herein.

As used herein, an “adjuvant” refers to a substance or vehicle that enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes, killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-like receptor (TLR) agonists and costimulatory molecules.

As used herein, an “antigenic polypeptide” refers to a polypeptide comprising all or part of an RSV amino acid sequence of sufficient length that the molecule is antigenic with respect to RSV, all or part of an hMPV amino acid sequence of sufficient length that the molecule is antigenic with respect to hMPV, or all or part of an hPIV3 amino acid sequence of sufficient length that the molecule is antigenic with respect to hPIV3.

As used herein, a “subject” refers to any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically engineered animal, and/or a clone. In certain embodiments, the subject is an adult, an adolescent, or an infant. In some embodiments, the terms “individual” or “patient” are used and are intended to be interchangeable with “subject.” In certain exemplary embodiments, the subject is a preterm newborn infant (e.g., gestational age less than 37 weeks), a newborn (e.g., 0-27 days of age), an infant or toddler (e.g., 28 days to 23 months of age), a child (e.g., 2 to 11 years of age), an adolescent (e.g., 12 to 17 years of age), an adult (e.g., 18 to 50 years of age or 18 to 64 years of age), or an elderly person (e.g., 65 years of age or older). In exemplary embodiments, the subject is 18 to 50 years of age. In other exemplary embodiments, the subject is an older adult (e.g., an adult aged 60 years of age or older).

As used herein, the term “vaccination” or “vaccinate” refers to the administration of a composition intended to generate an immune response, for example, to a disease-causing agent. Vaccination can be administered before, during, and/or after exposure to a disease-causing agent, and/or to the development of one or more symptoms, and in some embodiments, before, during, and/or shortly after exposure to the disease-causing agent. In some embodiments, vaccination includes multiple administrations, appropriately spaced in time, of a vaccinating composition.

The disclosure describes nucleic acid sequences (e.g., DNA and RNA sequences) and amino acid sequences having a certain degree of identity to a given nucleic acid sequence or amino acid sequence, respectively (a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90%, or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments, in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. In some embodiments, a nucleic acid sequence or amino acid sequence having a particular degree of identity to a given nucleic acid sequence or amino acid sequence is functionally equivalent to said given sequence.

As used herein, the term “kit” refers to a packaged set of related components, such as one or more compounds or compositions and one or more related materials such as solvents, solutions, buffers, instructions, or desiccants.

### II. RNA

The vaccines of the present disclosure may comprise at least two ribonucleic acids (RNAs) each comprising an ORF encoding a recombinant F protein antigenic polypeptide, such as an RSV F protein antigen, an hMPV F protein antigen, or an hPIV3 protein antigen. In certain embodiments, the RNAs are messenger RNAs (mRNAs) each comprising an ORF encoding an RSV F protein antigen, an hMPV F protein antigen, or an hPIV3 protein antigen. In certain embodiments, the RNAs (e.g., mRNAs) further comprises at least one 5′ UTR, 3′ UTR, poly(A) tail, and/or 5′ cap.

### II. A. 5′ Cap

An mRNA 5′ cap can provide resistance to nucleases found in most eukaryotic cells and promote translation efficiency. Several types of 5′ caps are known. A 7-methylguanosine cap (also referred to as “m7G” or “Cap-0”) comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase.

Examples of cap structures include, but are not limited to, m7G(5′)ppp, (5′(A,G(5′)ppp(5′)A, and G(5′)ppp(5′)G. Additional cap structures are described in U.S. Publication No. US 2016/0032356 and U.S. Publication No. US 2018/0125989, which are incorporated herein by reference. [0205] 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, MA; TriLink Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

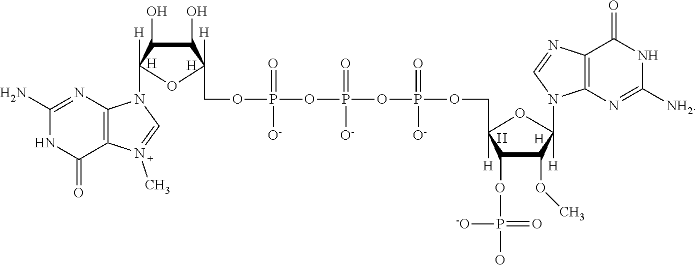

### II. B. Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 18) (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of a hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S.

Publication No. 2016/0166710, supra).

In some embodiments, an internal ribosome entry site (IRES) is used instead of a 5′ UTR.

In some embodiments, the 5′ UTR comprises a nucleic acid sequence of

(SEQ ID NO: 7)
GGACAGAUCGCCUGGAGACGCCAUCCACGCUGUUUUGACCUCCAUAGAA
GACACCGGGACCGAUCCAGCCUCCGCGGCCGGGAACGGUGCAUUGGAAC
GCGGAUUCCCCGUGCCAAGAGUGACUCACCGUCCUUGACACG.

In some embodiments, the 3′ UTR comprises a nucleic acid sequence of

(SEQ ID NO: 8)
CGGGUGGCAUCCCUGUGACCCCUCCCCAGUGCCUCUCCUGGCCCUGGAA
GUUGCCACUCCAGUGCCCACCAGCCUUGUCCUAAUAAAAUUAAGUUGCA
UC.

The 5′ UTR and 3′ UTR are described in further detail in International Pub. No. WO 2012/075040, incorporated herein by reference.

### II. C. Polyadenylated Tail

As used herein, the terms “poly(A) sequence,” “poly(A) tail,” and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides (SEQ ID NO:43) may have essentially a length of 100 nucleotides. In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, that are different from an adenosine nucleotide). In certain embodiments, the poly(A) tail comprises the sequence:

(SEQ ID NO: 19)
AAAAAAAAAAAAAAAAAAAAAAAAAAAAAAGCAUAUGACUAAAAAAAAAA
AAAAAAAAAAAAAAAAAAAAAAAAAAAAAAAAAAAAAAAAAAAAAAAAAA
AAAAAAAAAA

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides (SEQ ID NO:45), about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A) polymerases, e.g., using methods and means as described in International Pub. No. WO 2016/174271.

The nucleic acid may comprise a poly(A) tail obtained by enzymatic polyadenylation, wherein the majority of nucleic acid molecules comprise about 100 (+/−20) to about 500 (+/−50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in International Pub. No. WO 2016/091391.

In certain embodiments, the nucleic acid comprises at least one polyadenylation signal.

In various embodiments, the nucleic acid may comprise at least one poly(C) sequence.

The term “poly(C) sequence,” as used herein, is intended to be a sequence of cytosine nucleotides of up to about 200 cytosine nucleotides. In some embodiments, the poly(C) sequence (SEQ ID NO:44) comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In some embodiments, the poly(C) sequence comprises about 30 cytosine nucleotides.

### II. D. Chemical Modification

The mRNA disclosed herein may be modified or unmodified. In some embodiments, the mRNA may comprise at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, p-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

II. E. mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25(2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 RNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

In certain embodiments, at least one mRNA is comprised of the following structural elements:
(i) a 5′ cap with the following structure:

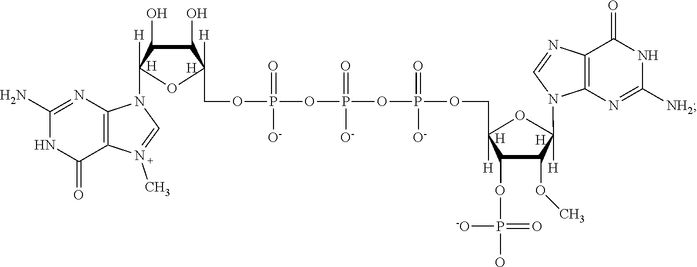

(ii) a 5′ untranslated region (5′ UTR) having the nucleic acid sequence of SEQ ID NO: 7;
(iii) a protein coding region having the nucleic acid sequence of SEQ ID NO: 4, 5, or 6;
(iv) a 3′ untranslated region (3′ UTR) having the nucleic acid sequence of SEQ ID NO: 8; and
(v) a poly(A) tail.

In certain embodiments, at least one mRNA is comprised of the following structural elements:
(i) a 5 cap with the following structure:

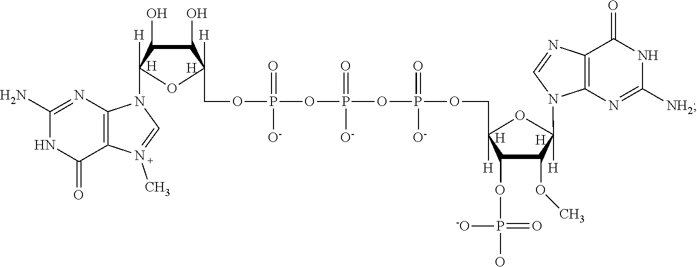

(ii) a 5′ untranslated region (5′ UTR) having the nucleic acid sequence of SEQ ID NO: 7;
(iii) a protein coding region having the nucleic acid sequence of SEQ ID NO: 4;
(iv) a 3′ untranslated region (3′ UTR) having the nucleic acid sequence of SEQ ID NO: 8; and
(v) a poly(A) tail.

In certain embodiments, at least one mRNA is comprised of the following structural elements:
(i) a 5′ cap with the following structure:

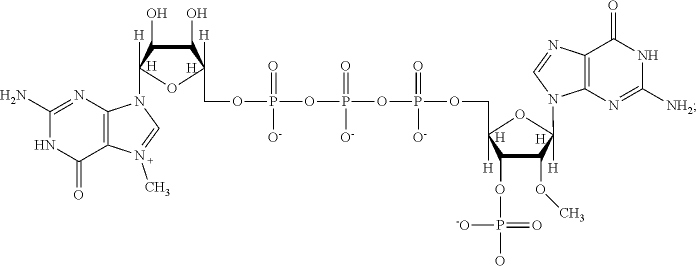

(ii) a 5′ untranslated region (5′ UTR) having the nucleic acid sequence of SEQ ID NO: 7;
(iii) a protein coding region having the nucleic acid sequence of SEQ ID NO: 5;
(iv) a 3′ untranslated region (3′ UTR) having the nucleic acid sequence of SEQ ID NO: 8; and
(v) a poly(A) tail.

In certain embodiments, at least one mRNA is comprised of the following structural elements:
(i) a 5′ cap with the following structure:

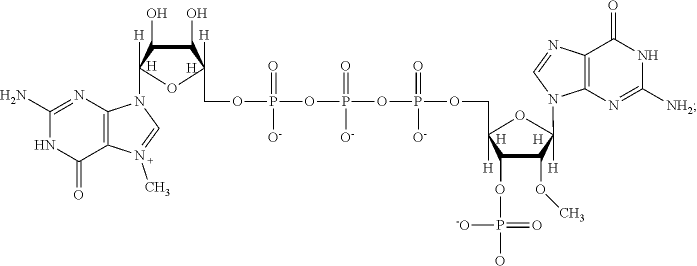

(ii) a 5′ untranslated region (5′ UTR) having the nucleic acid sequence of SEQ ID NO: 7;
(iii) a protein coding region having the nucleic acid sequence of SEQ ID NO: 6;
(iv) a 3′ untranslated region (3′ UTR) having the nucleic acid sequence of SEQ ID NO: 8; and
(v) a poly(A) tail.

In certain embodiments, the composition comprises at least two messenger mRNAs, wherein each of the at least two mRNAs comprises an open reading frame (ORF) encoding a recombinant F protein antigenic polypeptide, wherein at least one of the mRNAs comprises the following structural elements:
(i) a 5′ cap with the following structure:

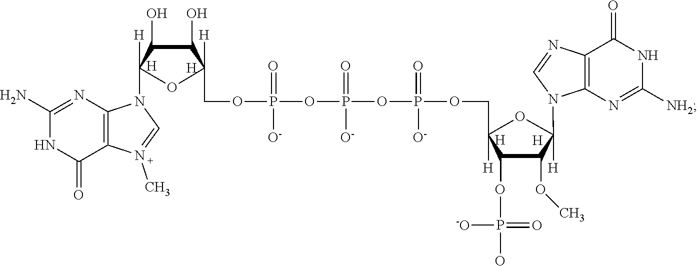

(ii) a 5′ untranslated region (5′ UTR) having the nucleic acid sequence of SEQ ID NO: 7;
(iii) a protein coding region having the nucleic acid sequence of SEQ ID NOs: 4, 5, or 6;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising:
GL-HEPES-E3-E12-DS-4-E10 at a molar ratio of 40%;
DMG-PEG2000 at a molar ratio of 1.5%;
cholesterol at a molar ratio of 28.5%; and
DOPE at a molar ratio of 30%.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

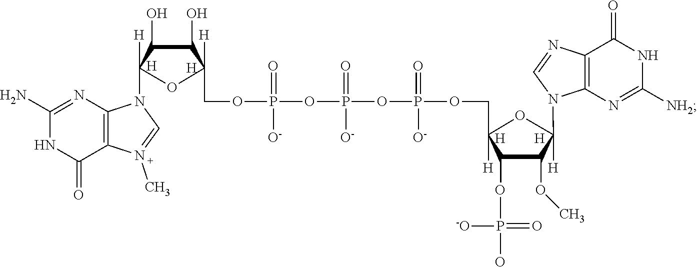

(ii) a 5′ untranslated region (5′ UTR) having the nucleic acid sequence of SEQ ID NO: 7;
(iii) a protein coding region having the nucleic acid sequence of SEQ ID NO: 4;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising:
GL-HEPES-E3-E12-DS-4-E10 at a molar ratio of 40%;
DMG-PEG2000 at a molar ratio of 1.5%;
cholesterol at a molar ratio of 28.5%; and
DOPE at a molar ratio of 30%.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

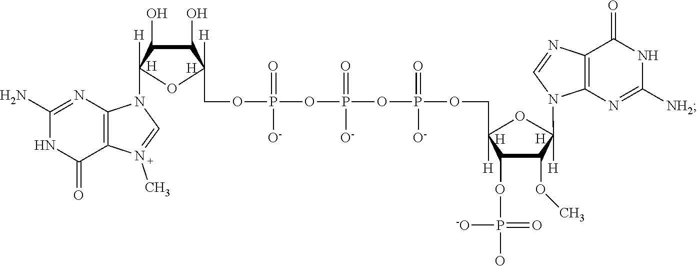

(ii) a 5′ untranslated region (5′ UTR) having the nucleic acid sequence of SEQ ID NO: 7;
(iii) a protein coding region having the nucleic acid sequence of SEQ ID NO: 5;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising:
GL-HEPES-E3-E12-DS-4-E10 at a molar ratio of 40%;
DMG-PEG2000 at a molar ratio of 1.5%;
cholesterol at a molar ratio of 28.5%; and
DOPE at a molar ratio of 30%.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

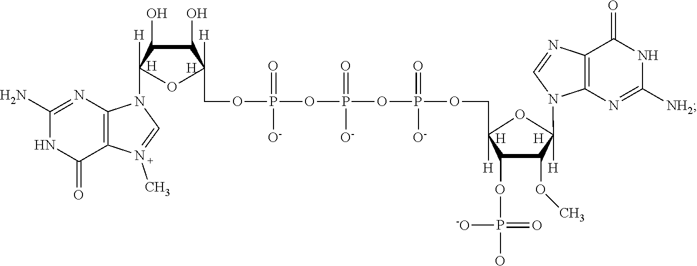

(ii) a 5′ untranslated region (5′ UTR) having the nucleic acid sequence of SEQ ID NO: 7;
(iii) a protein coding region having the nucleic acid sequence of SEQ ID NO: 6;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising:
GL-HEPES-E3-E12-DS-4-E10 at a molar ratio of 40%;
DMG-PEG2000 at a molar ratio of 1.5%;
cholesterol at a molar ratio of 28.5%; and
DOPE at a molar ratio of 30%.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

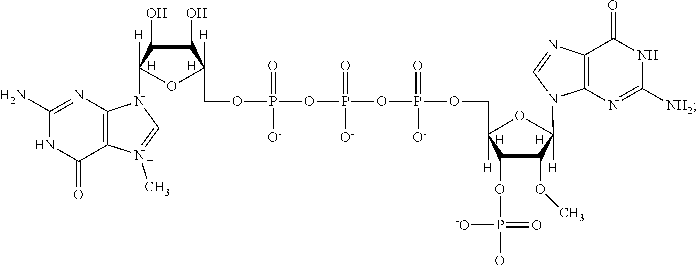

(ii) a 5′ untranslated region (5′ UTR) having the nucleic acid sequence of SEQ ID NO: 7;
(iii) a protein coding region having the nucleic acid sequence of SEQ ID NO: 4, 5, or 6;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising:
cKK-E10 at a molar ratio of 40%;
DMG-PEG2000 at a molar ratio of 1.5%;
cholesterol at a molar ratio of 28.5%; and
DOPE at a molar ratio of 30%.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

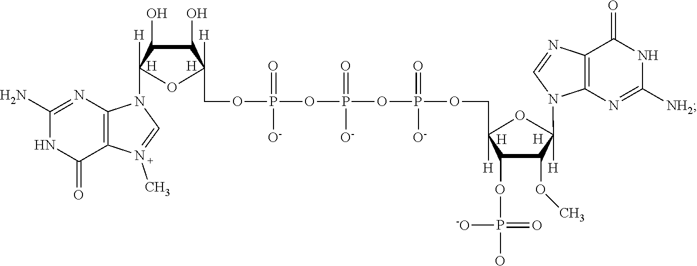

(ii) a 5′ untranslated region (5′ UTR) having the nucleic acid sequence of SEQ ID NO: 7;
(iii) a protein coding region having the nucleic acid sequence of SEQ ID NO: 4;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising:
cKK-E10 at a molar ratio of 40%;
DMG-PEG2000 at a molar ratio of 1.5%;
cholesterol at a molar ratio of 28.5%; and
DOPE at a molar ratio of 30%.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

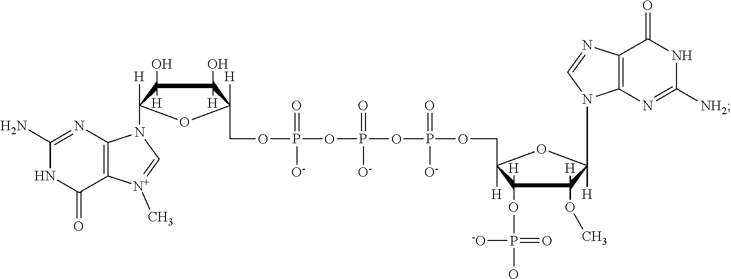

(ii) a 5′ untranslated region (5′ UTR) having the nucleic acid sequence of SEQ ID NO: 7;
(iii) a protein coding region having the nucleic acid sequence of SEQ ID NO: 5;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising:
cKK-E10 at a molar ratio of 40%;
DMG-PEG2000 at a molar ratio of 1.5%;
cholesterol at a molar ratio of 28.5%; and
DOPE at a molar ratio of 30%.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

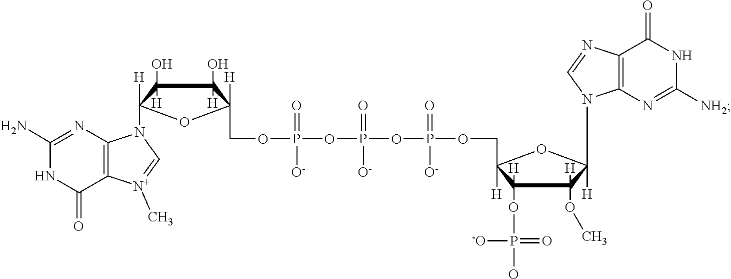

(ii) a 5′ untranslated region (5′ UTR) having the nucleic acid sequence of SEQ ID NO: 7;
(iii) a protein coding region having the nucleic acid sequence of SEQ ID NO: 6;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising:
cKK-E10 at a molar ratio of 40%;
DMG-PEG2000 at a molar ratio of 1.5%;
cholesterol at a molar ratio of 28.5%; and
DOPE at a molar ratio of 30%.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

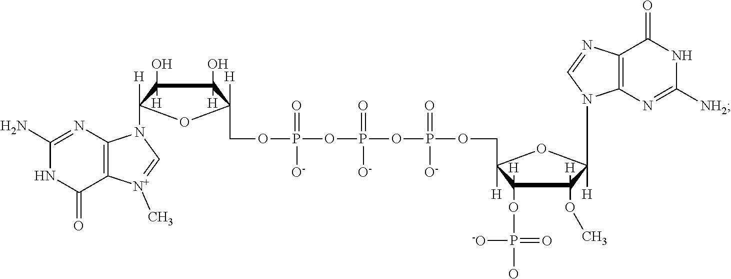

(ii) a 5′ untranslated region (5′ UTR) having the nucleic acid sequence of SEQ ID NO: 7;
(iii) a protein coding region having the nucleic acid sequence of SEQ ID NO: 4, 5, or 6;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising:
IM-001 at a molar ratio of 40%;
DMG-PEG2000 at a molar ratio of 1.5%;
cholesterol at a molar ratio of 28.5%; and
DOPE at a molar ratio of 30%.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

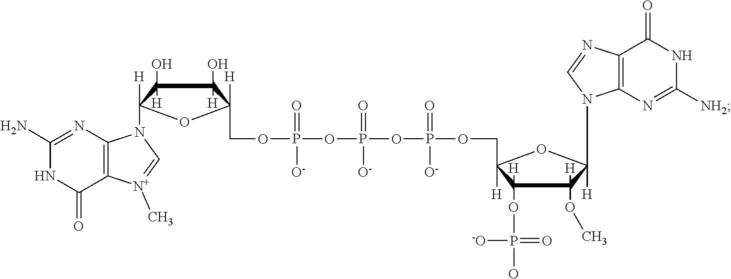

ii a untranslated region (5′ UTR) having the nucleic acid sequence of SEQ ID NO: 7;
(iii) a protein coding region having the nucleic acid sequence of SEQ ID NO: 4;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising:
IM-001 at a molar ratio of 40%;
DMG-PEG2000 at a molar ratio of 1.5%;
cholesterol at a molar ratio of 28.5%; and
DOPE at a molar ratio of 30%.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

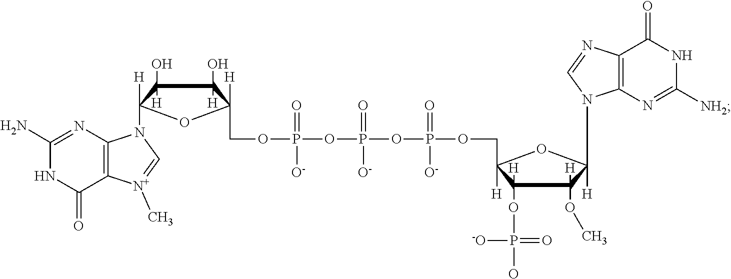

(ii) a 5′ untranslated region (5′ UTR) having the nucleic acid sequence of SEQ ID NO: 7;
(iii) a protein coding region having the nucleic acid sequence of SEQ ID NO: 5;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising:
IM-001 at a molar ratio of 40%;
DMG-PEG2000 at a molar ratio of 1.5%;
cholesterol at a molar ratio of 28.5%; and
DOPE at a molar ratio of 30%.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

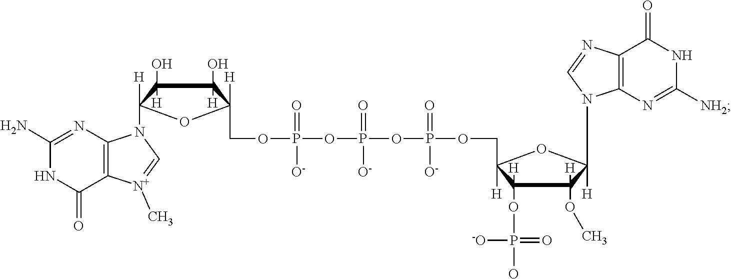

(ii) a 5′ untranslated region (5′ UTR) having the nucleic acid sequence of SEQ ID NO: 7;
(iii) a protein coding region having the nucleic acid sequence of SEQ ID NO: 6;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising:
IM-001 at a molar ratio of 40%;
DMG-PEG2000 at a molar ratio of 1.5%;
cholesterol at a molar ratio of 28.5%; and
DOPE at a molar ratio of 30%.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

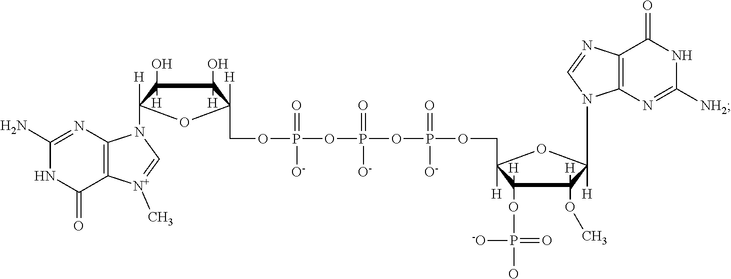

(ii) a 5′ untranslated region (5′ UTR) having the nucleic acid sequence of SEQ ID NO: 7;
(iii) a protein coding region having the nucleic acid sequence of SEQ ID NO: 4, 5, or 6;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising GL-HEPES-E3-E12-DS-4-E10, IM-001, OF-02, or cKK-E10.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

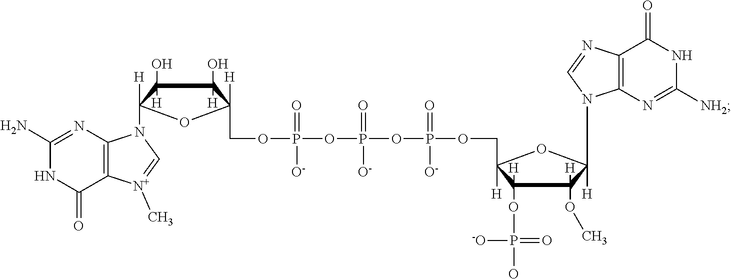

(ii) a 5′ untranslated region (5′ UTR) having the nucleic acid sequence of SEQ ID NO: 7;
(iii) a protein coding region having the nucleic acid sequence of SEQ ID NO: 4;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising GL-HEPES-E3-E12-DS-4-E10, IM-001, OF-02, or cKK-E10.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

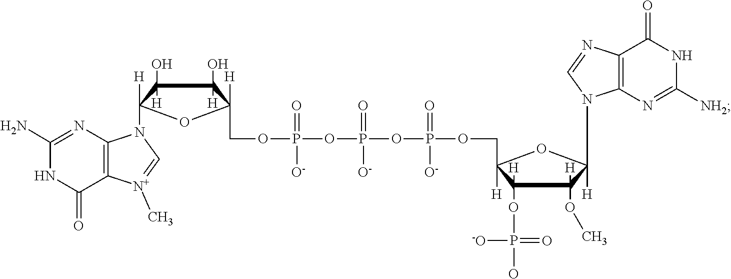

(ii) a 5′ untranslated region (5′ UTR) having the nucleic acid sequence of SEQ ID NO: 7;
(iii) a protein coding region having the nucleic acid sequence of SEQ ID NO: 5;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising GL-HEPES-E3-E12-DS-4-E10, IM-001, OF-02, or cKK-E10.

In certain embodiments, the composition comprises at least two messenger mRNAs, wherein each of the at least two mRNAs comprises an ORF encoding a recombinant F protein antigenic polypeptide, wherein at least one of the mRNAs comprises the following structural elements:
(i) a 5′ cap with the following structure:

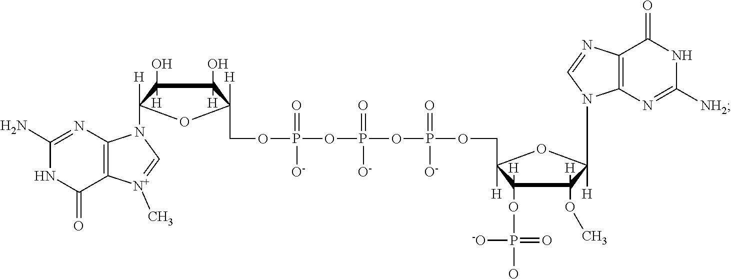

(ii) a 5′ untranslated region (5′ UTR) having the nucleic acid sequence of SEQ ID NO: 7;
(iii) a protein coding region having the nucleic acid sequence of SEQ ID NO: 6;
(iv) a 3′ untranslated region (3′ UTR) having the nucleic acid sequence of SEQ ID NO: 8; and
(v) a poly(A) tail;
   wherein the mRNA is formulated in a lipid nanoparticle (LNP) comprising GL-HEPES-E3-E12-DS-4-E10, IM-001, OF-02, or cKK-E10.

In certain embodiments, the poly(A) tail has a length of about 10 to about 500 adenosine nucleotides.

### III. RSV F Protein

RSV is a negative-sense, single-stranded RNA virus belonging to the Pneumoviridae family. RSV can cause infection of the respiratory tract. RSV is an enveloped virus with a glycoprotein (G protein), small hydrophobic protein (SH protein), and a fusion protein (F protein) on the surface. The RSV F protein is responsible for fusion of viral and host cell membranes and takes on at least three conformations (pre-fusion, intermediate, and post-fusion conformations). In the pre-fusion conformation (pre-fusion, Pre-F), the F protein exists in a trimeric form with the major antigenic site Ø exposed. Site Ø serves as a primary target of neutralizing antibodies produced by RSV-infected subjects (see, Coultas et al., Thorax. 74: 986-993. 2019; McLellan et al., Science. 340(6136): 1113-7. 2013). After binding to its target on the host cell surface, Pre-F undergoes a conformational change during which sites Ø and V are no longer exposed. Pre-F transitions into a transient intermediate conformation, enabling the F protein to insert into the host cell membrane, leading to fusion of the viral and host cell membranes. A final conformational shift results in a more stable and elongated form of the protein (post-fusion, Post-F). Site II and Site IV of the F protein are present in both the Pre-F and Post-F conformations. Site I is also present in both the Pre-F and Post-F conformations but antibodies against Site I bind tighter to the Post-F than to the Pre-F conformation.

Provided herein are RNAs (e.g., mRNAs) that encode for antigenic RSV F polypeptides.

In one aspect, the disclosure provides a respiratory syncytial virus (RSV) vaccine comprising a messenger RNA (mRNA) comprising an open reading frame (ORF) encoding an RSV F protein antigen, wherein the RSV F protein antigen comprises an amino acid sequence with at least 98% identity to SEQ ID NO: 1 or consists of an amino acid sequence of SEQ ID NO: 1.

In some embodiments, an epitope of the RSV F protein that is shared between Pre-F and Post-F is blocked. Blocking an epitope reduces or eliminates the generation of antibodies against the epitope when the RNA (e.g., mRNA) that encodes for the antigenic RSV F polypeptide is administered to a subject. This can increase the proportion of antibodies that target an epitope specific to a particular conformation of F, such as the pre-fusion conformation (e.g., antibodies that target site Ø). Because F has the pre-fusion conformation in viruses that have not yet entered cells, an increased proportion of antibodies that target Pre-F can provide a greater degree of neutralization (e.g., expressed as a neutralizing to binding ratio, as described herein). Blocking can be achieved by engineering a bulky moiety such as an N-glycan in the vicinity of the shared epitope. For example, an N-glycosylation site not present in wild-type F can be added, e.g., by mutating an appropriate residue to asparagine. In some embodiments, the blocked epitope is an epitope of antigenic site I of RSV F. In some embodiments, two or more epitopes shared between pre-F and post-F are blocked. In some embodiments, two or more epitopes of antigenic site I of RSV F are blocked. In some embodiments, one or more, or all, epitopes that topologically overlap with the blocked epitope are also blocked, optionally wherein the blocked epitope is an epitope of antigenic site I of RSV F.

In some embodiments, the RSV F polypeptide comprises an asparagine substitution at one or more positions corresponding to position 328, 348, or 507 of SEQ ID NO: 9 (i.e., E328N, S348N, or R507N). In some embodiments, the RSV F polypeptide comprises an asparagine substitution at two or more positions corresponding to position 328, 348, or 507 of SEQ ID NO: 9 (i.e., E328N, S348N, or R507N). In some embodiments, the RSV F polypeptide comprises an asparagine substitution at positions 328, 348, and 507 of SEQ ID NO: 9 (i.e., E328N, S348N, and R507N).

As shown previously, it has been found that such asparagines can function as glycosylation sites (see, International Pub. No. WO 2019/195291, incorporated herein by reference). Furthermore, without wishing to be bound by any particular theory, glycans at these sites may inhibit development of antibodies to nearby epitopes, which include epitopes common to pre- and post-fusion RSV F protein, when the RNA (e.g., mRNA) that encodes for the antigenic RSV F polypeptide is administered to a subject. In some embodiments, glycosylation of the asparagine corresponding to position 328, 348, or 507 of SEQ ID NO: 9 blocks at least one epitope shared between pre-fusion RSV F and post-fusion RSV F, such as an epitope of antigenic site 1. Inhibiting the development of antibodies to epitopes common to pre- and post-fusion RSV F protein can be beneficial because it can direct antibody development against epitopes specific to pre-fusion RSV F protein, such as the site 0 epitope, which may have more effective neutralizing activity than antibodies to other RSV F epitopes. The site Ø epitope involves amino acid residues 62-69 and 196-209 of SEQ ID NO: 9. Accordingly, in some embodiments, the RSV F polypeptide comprises amino acid residues 62-69 and 196-209 of SEQ ID NO: 9.

The RSV F polypeptides described herein may have deletions or substitutions of different length relative to wild-type RSV F. For example, in the RSV F polypeptide of SEQ ID NO: 9, positions 98-144 of the wild-type sequence (SEQ ID NO: 9) are replaced with GSGNVGL (SEQ ID NO: 20), resulting in a net removal of 40 amino acids, such that positions 328, 348, or 507 of SEQ ID NO: 9 correspond to positions 288, 308, and 467 of SEQ ID NO: 1. In the alternative, in the RSV F polypeptide of SEQ ID NO: 1, positions 98-146 of the wild-type sequence (SEQ ID NO: 9) are replaced with GSGNVGLGG (SEQ ID NO: 21, positions 98-106 of SEQ ID NO: 1), resulting in a net removal of 40 amino acids, such that positions 328, 348, or 507 of SEQ ID NO: 9 correspond to positions 290, 310, and 469 of SEQ ID NO: 1.

In general, positions in constructs described herein can be mapped onto the wild-type sequence of SEQ ID NO: 9 by pairwise alignment, e.g., using the Needleman-Wunsch algorithm with standard parameters (EBLOSUM62 matrix, Gap penalty 10, gap extension penalty 0.5). See also the discussion of structural alignment provided herein as an alternative approach for identifying corresponding positions.

In some embodiments, the RSV F polypeptide comprises mutations that add glycans to block epitopes on the pre-fusion antigen that are structurally similar to those on the surface of the post-fusion RSV F. In some embodiments, glycans are added to specifically block epitopes that may be present in the post-fusion conformation of RSV F. In some embodiments, glycans are added that block epitopes that may be present in the post-fusion conformation of RSV F but do not affect one or more epitopes present on the pre-fusion conformation of RSV F, such as the site Ø epitope.

In some embodiments, the RSV F polypeptide comprises a sequence having at least 85%, 90%, 95%, 97%, 98%, 99%, or 99.5% identity to the amino acid sequence set forth below:

(SEQ ID NO: 22)
MELLILKANAITTILTAVTFCFASGQNITEEFYQSTCSAVSKGYLSALRT
GWYTSVITIELSNIKKNKCNGTDAKVKLIKQELDKYKNAVTELQLLMQST
QATNNRARRELPRFMNYTLNNAKKTNVTLSKKRKRRFLGFLLGVGSAIAS
GVAVSKVLHLEGEVNKIKSALLSTNKAVVSLSNGVSVLTSKVLDLKNYID
KQLLPIVNKQSCSISNIETVIEFQQKNNRLLEITREFSVNAGVTTPVSTY
MLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVS
FFPQAETCKVQSNRVFCDTMNSLTLPSEVNLCNVDIFNPKYDCKIMTSKT
DVSSSVITSLGAIVSCYGKTKCTASNKNRGIIKTFSNGCDYVSNKGVDTV
SVGNTLYYVNKQEGKSLYVKGEPIINFYDPLVFPSDEFDASISQVNEKIN
QSLAFIRKSDELLSAIGGYIPEAPRDGQAYVRKDGEWVLLSTFL

In some embodiments, the RSV F polypeptide comprises a sequence having at least 85%, 90%, 95%, 97%, 98%, 99%, or 99.5% identity to the amino acid sequence set forth below:

(SEQ ID NO: 1)
MELLILKANAITTILTAVTFCFASGQNITEEFYQSTCSAVSKGYLSALRT
GWYTSVITIELSNIKENKCNGTDAKVKLIKQELDKYKNAVTELQLLMGSG
NVGLGGAIASGVAVSKVLHLEGEVNKIKSALLSTNKAVVSLSNGVSVLTF
KVLDLKNYIDKQLLPILNKQSCSISNPETVIEFQQKNNRLLEITREFSVN
AGVTTPVSTYMLTNSELLSLINDMPITNDQKKLMSNNVQIVRQQSYSIMS
IIKEEVLAYVVQLPLYGVIDTPCWKLHTSPLCTTNTKNGSNICLTRTDRG
WYCDNAGNVSFFPQAETCKVQSNRVFCDTMNSRTLPSEVNLCNVDIFNPK
YDCKIMTSKTDVSSSVITSLGAIVSCYGKTKCTASNKNRGIIKTFSNGCD
YVSNKGVDTVSVGNTLYYVNKQEGKSLYVKGEPIINFYDPLVFPSDEFDA
SISQVNELINQSLAFINQSDELLHNVNAGKSTTNIMITTIIIVIIVILLS
LIAVGLLLYCKARSTPVTLSKDQLSGINNIAFSN

In some embodiments, the RSV F polypeptide comprises the DS-CAV1 amino acid substitutions (as described, for example, in McLellan et al., Science, 342(6158): 592-598, 2013) in which further modifications are made including at least one, two, or three of the asparagines described above. The CAV1 mutations are S190F and V207L relative to SEQ ID NO: 9. The DS mutations are S155C and S290C relative to SEQ ID NO: 9.

In some embodiments, an amino acid substitution or pair of amino acid substitutions are inter-protomer stabilizing substitution(s). Exemplary substitutions that can be inter-protomer stabilizing are V207L; N228F; I217V and E218F; I221L and E222M; or Q224A and Q225L, using the position numbering of SEQ ID NO: 9.

In some embodiments, an amino acid substitution or pair of amino acid substitutions are intra-protomer stabilizing. Exemplary substitutions that can be intra-protomer stabilizing are V220I; and A74L and Q81 L, using the position numbering of SEQ ID NO: 9.

In some embodiments, an amino acid substitution is helix stabilizing, i.e., predicted to stabilize the helical domain of RSV F. Stabilization of the helical domain can contribute to the stability of the site Ø epitope and of the pre-fusion conformation of RSV F generally. Exemplary substitutions that can be helix stabilizing are N216P or I217P, using the position numbering of SEQ ID NO: 9. Position 217 in SEQ ID NO: 9 corresponds to position 177 in SEQ ID NO: 1.

In some embodiments, an amino acid substitution is helix capping. In some embodiments, an amino acid substitution is helix PRO capping. Helix capping is based on the biophysical observation that, while a proline residue mutation placed in an alpha helix may disrupt the helix formation, a proline at the N-terminus of a helical region may help induce helical formation by stabilizing the PHI/PSI bond angles. Exemplary substitutions that can be helix capping are N216P or I217P, using the position numbering of SEQ ID NO: 9.

In some embodiments, an amino acid substitution replaces a disulfide mutation of DS-CAV1. In some embodiments, the engineered disulfide of DS-CAV1 is reverted to wild-type (C69S and/or C212S mutations of DS-CAV1 using the position numbering of SEQ ID NO: 9). In some embodiments, one or more C residue of DS-CAV1 is replaced with a S residue to eliminate a disulfide bond. In some embodiments, C69S or C212S substitution using the position numbering of SEQ ID NO: 9 eliminates a disulfide bond. In some embodiments, an RSV F polypeptide comprises both C69S and C212S using the position numbering of SEQ ID NO: 9. In some embodiments, replacing such cysteines and thereby eliminating a disulfide bond blocks reduction (i.e., acceptance of electrons from a reducing agent) of the RSV F polypeptide. In some embodiments, an I217P substitution using the position numbering of SEQ ID NO: 9 is comprised in an antigen instead of substitution at C69 and/or C212.

In some embodiments, an amino acid substitution prevents proteolysis by trypsin or trypsin-like proteases. In some embodiments, the amino acid substitution that prevents such proteolysis is in the heptad repeat region B (HRB) region of RSV F.

Appearance of fragments consistent with proteolysis of an RSV F polypeptide that comprised a wild-type HRB region suggested a lysine or arginine in this region was being targeted for proteolysis. An amino acid substitution to remove a K or R residue may be termed a knockout (KO). In some embodiments, a K or R is substituted for L or Q. In some embodiments, a K is substituted for L or Q. In some embodiments, the RSV F polypeptide comprises K498L and/or K508Q, using the position numbering of SEQ ID NO: 9. The corresponding positions in SEQ ID NO: 1 are 458 and 468, respectively. In some embodiments, the RSV F polypeptide comprises both K498L and K508Q.

In some embodiments, an amino acid substitution adds glycans. In some embodiments, an amino acid substitution increases glycosylation by adding glycans to RSV F polypeptides. Substitutions to add glycans may also be referred to as engineered glycosylation, as compared to native glycosylation (without additional glycans).

In some embodiments, the amino acid substitution to add glycans is substitution with an N. In some embodiments, amino acid substitution with an N allows N-linked glycosylation. In some embodiments, substitution with an N is accompanied by substitution with a T or S at the second amino acid position C-terminal to the N, which forms an N×T/S glycosylation motif. In some embodiments, the N is surface-exposed.

Each of the above recited substitutions and mutations in the RSV F polypeptide are described in more detail in International Pub. No. WO 2019/195291, which is incorporated herein by reference.

In one aspect, the disclosure provides, an RSV vaccine, comprising a messenger RNA (mRNA) comprising an open reading frame (ORF) encoding an RSV F protein antigen, wherein the RSV F protein antigen comprises one or more of the following substitutions relative to an amino acid sequence set forth in SEQ ID NO: 9:
1) amino acid positions 98-146 of SEQ ID NO: 9 are replaced with an amino acid sequence of GSGNVGLGG (SEQ ID NO: 21);
2) amino acid substitutions S190F and V207L;
3) amino acid substitution I217P;
4) amino acid substitutions E328N, S348N, and R507N;
5) amino acid substitution L373R;
6) amino acid substitution K498L; and
7) amino acid substitution K508Q.

In another aspect, the disclosure provides an RSV vaccine comprising an mRNA comprising an ORF encoding an RSV F protein antigen, wherein the RSV F protein antigen comprises each of the following substitutions relative to an amino acid sequence set forth in SEQ ID NO: 9:
1) amino acid positions 98-146 of SEQ ID NO: 9 are replaced with an amino acid sequence of GSGNVGLGG (SEQ ID NO: 21);
2) amino acid substitutions S190F and V207L;
3) amino acid substitution I217P;
4) amino acid substitutions E328N, S348N, and R507N;
5) amino acid substitution L373R;
6) amino acid substitution K498L; and
7) amino acid substitution K508Q.

In certain embodiments, the RSV F protein antigen comprises a transmembrane domain and cytoplasmic tail amino acid sequence of

(SEQ ID NO: 23)
IMITTIIIVIIVILLSLIAVGLLLYCKARSTPVTLSKDQLSGINNIAFSN

In some embodiments, the mRNA comprises a nucleic acid sequence with at least 80%, at least 85%, at least 90%, at least 91%, at least 92%, at least 93%, at least 94%, at least 95, at least 96%, at least 97%, at least 98%, at least 99%, or 100% identity to a nucleic acid sequence set forth in any one of SEQ ID NOs: 24-26 below:

(SEQ ID NO: 24)
AUGGAAUUGCUGAUCCUCAAAGCGAACGCAAUCACCACUAUCCUCACUGCGGUCACCUUCU
GCUUUGCGAGCGGACAGAACAUCACCGAAGAAUUCUACCAAUCUACUUGCUCCGCCGUGUC
CAAGGGUUACCUGUCCGCCCUGAGGACCGGAUGGUACACUUCCGUGAUUACCAUUGAGUU
GUCGAAUAUCAAGAAGAACAAGUGCAACGGAACCGAUGCUAAGGUCAAGCUGAUCAAGCAG
GAGCUGGACAAGUACAAGAAUGCUGUGACCGAGCUGCAGCUGCUGAUGCAGUCCACUCAAG
CCACCAACAAUCGCGCCCGGCGGGAACUCCCAAGGUUCAUGAACUACACCUUGAACAACGC
CAAGAAAACGAACGUGACCCUGUCCAAGAAGCGCAAGCGCAGAUUCCUUGGCUUCCUUCUG
GGCGUCGGUAGCGCCAUCGCCUCCGGCGUGGCCGUCAGCAAGGUCCUGCACCUCGAGGGA
GAAGUCAACAAGAUUAAGAGCGCCCUGCUGUCCACCAACAAGGCCGUGGUGUCGCUAUCAA
ACGGCGUCAGCGUACUGACCAGCAAAGUGCUGGAUCUCAAGAACUACAUUGAUAAGCAACU
CCUCCCUAUCGUGAAUAAGCAGAGCUGUUCGAUUUCCAACAUCGAGACUGUGAUUGAAUUC
CAGCAGAAGAACAACCGGCUGCUGGAAAUUACCAGAGAAUUCAGCGUGAAUGCCGGAGUCA
CUACCCCCGUGUCCACCUACAUGCUGACAAACUCCGAGCUGCUGAGCCUGAUCAACGAUAU
GCCGAUUACCAACGACCAGAAGAAGCUGAUGUCGAACAACGUGCAGAUCGUGCGCCAGCAG
UCCUACUCAAUCAUGUCGAUCAUCAAGGAAGAGGUCCUGGCCUACGUGGUGCAGCUUCCUC
UGUACGGCGUGAUUGACACUCCGUGUUGGAAACUGCACACUAGUCCCCUGUGCACUACUAA
CACCAAGGAGGGCAGCAAUAUCUGCCUGACUCGGACCGAUAGAGGCUGGUACUGUGAUAAC
GCCGGGUCCGUGUCCUUCUUCCCGCAAGCCGAGACUUGCAAAGUGCAGAGCAACCGGGUG
UUCUGUGACACUAUGAACUCACUGACCUUGCCGAGCGAAGUCAACCUUUGCAACGUGGACA
UCUUUAACCCUAAAUACGACUGCAAGAUCAUGACCUCCAAGACCGACGUGUCGAGCUCAGU
GAUUACUUCGCUGGGAGCCAUUGUGUCCUGCUACGGGAAAACCAAGUGCACGGCCUCAAAC
AAGAACCGGGGUAUCAUUAAGACCUUCUCCAACGGCUGCGACUAUGUGUCCAACAAGGGGG
UGGACACUGUGUCCGUGGGAAACACCUUGUAUUACGUGAACAAGCAGGAGGGAAAGUCCCU
CUACGUGAAGGGCGAACCCAUCAUCAAUUUCUACGACCCGCUCGUGUUCCCCUCCGAUGAA
UUCGACGCAUCCAUCUCACAAGUCAACGAAAAGAUUAACCAGUCCCUGGCUUUCAUUCGCA
AGUCCGACGAACUGCUCCAUAACGUCAACGCUGGAAAGUCCACCACCAACAUCAUGAUCAC
CACGAUCAUUAUUGUGAUCAUCGUCAUCCUGCUGUCACUGAUAGCAGUGGGACUGCUCCUC
UACUGCAAAGCGCGGUCGACCCCAGUGACACUCUCGAAGGACCAGCUGUCCGGGAUCAACA
ACAUCGCGUUUUCGAACUGA
(SEQ ID NO: 25)
AUGGAACUCCUGAUCCUGAAGGCCAAUGCUAUCACUACCAUCCUGACUGCCGUCACCUUCU
GCUUCGCCUCCGGACAAAAUAUCACUGAAGAAUUUUACCAAAGCACCUGUAGCGCGGUGUC
CAAGGGAUACCUGAGCGCUCUGAGGACCGGAUGGUACACCAGCGUGAUUACCAUCGAGCU
GAGUAACAUCAAGAAGAACAAGUGCAACGGGACCGAUGCUAAGGUCAAGUUGAUCAAACAA
GAGCUCGACAAGUACAAGAACGCCGUGACUGAGCUGCAGCUGCUGAUGCAGUCAACUCAGG
CCACCAACAACCGGGCCAGACGGGAACUGCCGAGAUUCAUGAACUACACCCUGAACAACGC
CAAAAAGACCAACGUGACCCUGUCCAAGAAGAGAAAGCGCCGGUUCCUGGGUUUCCUGCUU
GGCGUGGGAUCAGCAAUCGCGUCCGGAGUGGCAGUGUCCAAGGUCUUGCACCUCGAGGGC
GAAGUGAACAAGAUCAAGUCCGCGCUUCUGUCGACCAACAAGGCCGUCGUUUCCCUGUCGA
ACGGAGUGUCCGUGCUCACGAGCAAAGUGCUCGACCUGAAGAACUACAUCGACAAACAGCU
GCUGCCCAUCGUCAACAAGCAGAGCUGCAGCAUCUCAAACAUUGAAACCGUGAUCGAGUUC
CAGCAGAAGAACAACCGCCUGCUCGAGAUUACCAGAGAGUUUUCCGUGAACGCCGGCGUGA
CCACCCCGGUGUCGACCUACAUGCUCACAAAUUCGGAACUUCUCUCCCUGAUUAAUGACAU
GCCCAUUACUAACGAUCAGAAAAAGCUGAUGUCGAACAAUGUGCAGAUUGUGCGCCAGCAG
UCCUACUCCAUCAUGUCCAUCAUUAAGGAAGAGGUCCUGGCCUACGUGGUGCAGUUGCCG
CUGUACGGUGUCAUCGAUACCCCCUGCUGGAAGCUCCAUACUUCGCCCCUGUGUACUACCA
ACACCAAGGAAGGCUCCAACAUCUGCCUGACCCGGACGGAUCGCGGCUGGUACUGUGACAA
UGCCGGAUCCGUGUCGUUCUUCCCGCAAGCGGAGACUUGCAAAGUGCAGUCCAACCGGGU
GUUCUGUGACACUAUGAACUCCCUGACUCUGCCGUCCGAAGUCAACCUCUGCAACGUGGAC
AUUUUCAAUCCAAAAUACGACUGCAAGAUAAUGACCUCCAAGACUGACGUGUCAUCGUCCG
UGAUCACAUCUCUGGGAGCCAUUGUCUCCUGCUACGGAAAGACUAAGUGCACCGCGUCGAA
CAAGAACAGGGGCAUUAUCAAGACCUUCAGCAACGGUUGCGACUAUGUGUCCAACAAGGGC
GUGGAUACCGUGUCCGUGGGCAACACCUUGUACUACGUGAACAAGCAGGAGGGGAAGUCC
CUUUAUGUGAAGGGGGAGCCAAUCAUUAACUUUUACGACCCCCUGGUGUUCCCUAGCGACG
AGUUCGACGCCUCAAUCUCUCAAGUCAACGAAAAGAUCAACCAGAGCCUCGCCUUCAUCCG
CAAGUCCGAUGAACUGCUGUCAGCCAUUGGGGGUUACAUCCCUGAGGCCCCUCGGGACGG
ACAGGCAUACGUCCGCAAGGACGGCGAAUGGGUGCUGCUUAGCACCUUCCUCUAA
(SEQ ID NO: 26)
AUGGAACUGCUGAUCCUCAAAGCCAACGCAAUCACCACCAUUCUCACCGCUGUGACCUUCU
GCUUCGCAUCGGGGCAGAACAUCACUGAAGAGUUUUACCAGAGCACUUGCAGCGCGGUGU
CAAAGGGUUACCUUUCCGCACUGCGGACCGGAUGGUACACUUCCGUGAUCACCAUUGAGCU
CAGCAACAUCAAGGAAAACAAGUGCAAUGGCACCGACGCCAAGGUCAAGCUGAUCAAACAAG
AACUGGACAAGUACAAGAACGCCGUGACAGAAUUGCAGCUCCUGAUGGGAUCCGGAAACGU
CGGUCUGGGCGGAGCCAUCGCGAGUGGAGUGGCUGUGUCCAAGGUCUUGCACCUCGAGG
GAGAAGUGAACAAGAUCAAGUCCGCGCUGCUGUCAACGAACAAGGCCGUGGUGUCCCUGUC
UAACGGCGUCAGCGUGCUGACGUUCAAGGUCCUGGACCUGAAGAAUUACAUUGACAAGCAG
CUGCUGCCCAUCCUCAACAAGCAAUCCUGCUCCAUCUCCAACCCCGAAACCGUGAUCGAGU
UCCAGCAGAAGAACAACCGCCUGCUGGAAAUUACUCGCGAGUUCUCUGUGAAUGCCGGCGU
GACCACCCCUGUGUCCACCUACAUGCUGACCAACUCCGAGCUUCUCUCCCUUAUCAAUGAC
AUGCCUAUCACGAACGACCAGAAGAAGCUGAUGUCGAACAACGUGCAGAUUGUGCGGCAGC
AGUCAUACAGCAUCAUGUCGAUCAUCAAGGAAGAAGUGCUGGCGUACGUGGUGCAACUCCC
GCUGUACGGCGUCAUCGAUACCCCGUGCUGGAAGCUGCACACCUCGCCUUUGUGUACCAC
CAACACCAAGAACGGAUCCAACAUCUGCUUAACCCGGACUGAUCGGGGUUGGUACUGCGAC
AACGCCGGGAAUGUUUCGUUCUUCCCACAAGCCGAGACUUGUAAAGUGCAGUCAAACAGAG
UGUUCUGUGACACCAUGAACUCGAGAACCCUGCCCAGCGAAGUGAACCUGUGUAACGUCGA
CAUCUUUAACCCAAAAUACGAUUGCAAGAUUAUGACCAGCAAAACCGACGUGUCCUCCUCCG
UGAUAACAAGCCUGGGGGCGAUUGUGUCAUGCUACGGAAAGACUAAGUGCACCGCCUCGAA
CAAGAACCGCGGCAUCAUUAAGACUUUCUCGAAUGGUUGCGACUAUGUGUCCAACAAGGGC
GUGGAUACUGUGUCAGUCGGGAAUACUCUUUACUACGUGAACAAGCAGGAGGGGAAAAGCC
UCUACGUGAAGGGAGAGCCUAUUAUCAACUUUUACGAUCCGCUGGUGUUCCCGUCCGACGA
AUUCGACGCCAGCAUCAGCCAAGUCAACGAGCUGAUUAACCAGUCCCUCGCCUUCAUCAAC
CAAUCCGACGAGCUCCUGCAUAACGUGAACGCCGGAAAGUCCACCACCAACAUCAUGAUCA
CUACUAUUAUCAUCGUGAUCAUCGUCAUCCUGCUGAGCCUGAUUGCUGUGGGCCUGUUGC
UGUAUUGCAAAGCCAGGUCCACCCCGGUCACCCUGUCGAAGGAUCAGCUGUCCGGAAUCAA
CAACAUUGCCUUCUCCAACUAA

In some embodiments, the mRNA comprises a nucleic acid sequence with at least 80%, at least 85%, at least 90%, at least 91%, at least 92%, at least 93%, at least 94%, at least 95, at least 96%, at least 97%, at least 98%, at least 99%, or 100% identity to a nucleic acid sequence set forth in any one of SEQ ID NOs: 27-29 below:

(SEQ ID NO: 27)
GGACAGAUCGCCUGGAGACGCCAUCCACGCUGUUUUGACCUCCAUAGAAGACACCGGGACC
GAUCCAGCCUCCGCGGCCGGGAACGGUGCAUUGGAACGCGGAUUCCCCGUGCCAAGAGUG
ACUCACCGUCCUUGACACGAUGGAAUUGCUGAUCCUCAAAGCGAACGCAAUCACCACUAUC
CUCACUGCGGUCACCUUCUGCUUUGCGAGCGGACAGAACAUCACCGAAGAAUUCUACCAAU
CUACUUGCUCCGCCGUGUCCAAGGGUUACCUGUCCGCCCUGAGGACCGGAUGGUACACUU
CCGUGAUUACCAUUGAGUUGUCGAAUAUCAAGAAGAACAAGUGCAACGGAACCGAUGCUAA
GGUCAAGCUGAUCAAGCAGGAGCUGGACAAGUACAAGAAUGCUGUGACCGAGCUGCAGCUG
CUGAUGCAGUCCACUCAAGCCACCAACAAUCGCGCCCGGCGGGAACUCCCAAGGUUCAUGA
ACUACACCUUGAACAACGCCAAGAAAACGAACGUGACCCUGUCCAAGAAGCGCAAGCGCAGA
UUCCUUGGCUUCCUUCUGGGCGUCGGUAGCGCCAUCGCCUCCGGCGUGGCCGUCAGCAAG
GUCCUGCACCUCGAGGGAGAAGUCAACAAGAUUAAGAGCGCCCUGCUGUCCACCAACAAGG
CCGUGGUGUCGCUAUCAAACGGCGUCAGCGUACUGACCAGCAAAGUGCUGGAUCUCAAGAA
CUACAUUGAUAAGCAACUCCUCCCUAUCGUGAAUAAGCAGAGCUGUUCGAUUUCCAACAUC
GAGACUGUGAUUGAAUUCCAGCAGAAGAACAACCGGCUGCUGGAAAUUACCAGAGAAUUCA
GCGUGAAUGCCGGAGUCACUACCCCCGUGUCCACCUACAUGCUGACAAACUCCGAGCUGCU
GAGCCUGAUCAACGAUAUGCCGAUUACCAACGACCAGAAGAAGCUGAUGUCGAACAACGUG
CAGAUCGUGCGCCAGCAGUCCUACUCAAUCAUGUCGAUCAUCAAGGAAGAGGUCCUGGCCU
ACGUGGUGCAGCUUCCUCUGUACGGCGUGAUUGACACUCCGUGUUGGAAACUGCACACUA
GUCCCCUGUGCACUACUAACACCAAGGAGGGCAGCAAUAUCUGCCUGACUCGGACCGAUAG
AGGCUGGUACUGUGAUAACGCCGGGUCCGUGUCCUUCUUCCCGCAAGCCGAGACUUGCAA
AGUGCAGAGCAACCGGGUGUUCUGUGACACUAUGAACUCACUGACCUUGCCGAGCGAAGUC
AACCUUUGCAACGUGGACAUCUUUAACCCUAAAUACGACUGCAAGAUCAUGACCUCCAAGAC
CGACGUGUCGAGCUCAGUGAUUACUUCGCUGGGAGCCAUUGUGUCCUGCUACGGGAAAAC
CAAGUGCACGGCCUCAAACAAGAACCGGGGUAUCAUUAAGACCUUCUCCAACGGCUGCGAC
UAUGUGUCCAACAAGGGGGUGGACACUGUGUCCGUGGGAAACACCUUGUAUUACGUGAACA
AGCAGGAGGGAAAGUCCCUCUACGUGAAGGGCGAACCCAUCAUCAAUUUCUACGACCCGCU
CGUGUUCCCCUCCGAUGAAUUCGACGCAUCCAUCUCACAAGUCAACGAAAAGAUUAACCAG
UCCCUGGCUUUCAUUCGCAAGUCCGACGAACUGCUCCAUAACGUCAACGCUGGAAAGUCCA
CCACCAACAUCAUGAUCACCACGAUCAUUAUUGUGAUCAUCGUCAUCCUGCUGUCACUGAU
AGCAGUGGGACUGCUCCUCUACUGCAAAGCGCGGUCGACCCCAGUGACACUCUCGAAGGA
CCAGCUGUCCGGGAUCAACAACAUCGCGUUUUCGAACUGACGGGUGGCAUCCCUGUGACC
CCUCCCCAGUGCCUCUCCUGGCCCUGGAAGUUGCCACUCCAGUGCCCACCAGCCUUGUCC
UAAUAAAAUUAAGUUGCAUC
(SEQ ID NO: 28)
GGACAGAUCGCCUGGAGACGCCAUCCACGCUGUUUUGACCUCCAUAGAAGACACCGGGACC
GAUCCAGCCUCCGCGGCCGGGAACGGUGCAUUGGAACGCGGAUUCCCCGUGCCAAGAGUG
ACUCACCGUCCUUGACACGAUGGAACUCCUGAUCCUGAAGGCCAAUGCUAUCACUACCAUC
CUGACUGCCGUCACCUUCUGCUUCGCCUCCGGACAAAAUAUCACUGAAGAAUUUUACCAAA
GCACCUGUAGCGCGGUGUCCAAGGGAUACCUGAGCGCUCUGAGGACCGGAUGGUACACCA
GCGUGAUUACCAUCGAGCUGAGUAACAUCAAGAAGAACAAGUGCAACGGGACCGAUGCUAA
GGUCAAGUUGAUCAAACAAGAGCUCGACAAGUACAAGAACGCCGUGACUGAGCUGCAGCUG
CUGAUGCAGUCAACUCAGGCCACCAACAACCGGGCCAGACGGGAACUGCCGAGAUUCAUGA
ACUACACCCUGAACAACGCCAAAAAGACCAACGUGACCCUGUCCAAGAAGAGAAAGCGCCGG
UUCCUGGGUUUCCUGCUUGGCGUGGGAUCAGCAAUCGCGUCCGGAGUGGCAGUGUCCAAG
GUCUUGCACCUCGAGGGCGAAGUGAACAAGAUCAAGUCCGCGCUUCUGUCGACCAACAAGG
CCGUCGUUUCCCUGUCGAACGGAGUGUCCGUGCUCACGAGCAAAGUGCUCGACCUGAAGA
ACUACAUCGACAAACAGCUGCUGCCCAUCGUCAACAAGCAGAGCUGCAGCAUCUCAAACAUU
GAAACCGUGAUCGAGUUCCAGCAGAAGAACAACCGCCUGCUCGAGAUUACCAGAGAGUUUU
CCGUGAACGCCGGCGUGACCACCCCGGUGUCGACCUACAUGCUCACAAAUUCGGAACUUCU
CUCCCUGAUUAAUGACAUGCCCAUUACUAACGAUCAGAAAAAGCUGAUGUCGAACAAUGUG
CAGAUUGUGCGCCAGCAGUCCUACUCCAUCAUGUCCAUCAUUAAGGAAGAGGUCCUGGCCU
ACGUGGUGCAGUUGCCGCUGUACGGUGUCAUCGAUACCCCCUGCUGGAAGCUCCAUACUU
CGCCCCUGUGUACUACCAACACCAAGGAAGGCUCCAACAUCUGCCUGACCCGGACGGAUCG
CGGCUGGUACUGUGACAAUGCCGGAUCCGUGUCGUUCUUCCCGCAAGCGGAGACUUGCAA
AGUGCAGUCCAACCGGGUGUUCUGUGACACUAUGAACUCCCUGACUCUGCCGUCCGAAGU
CAACCUCUGCAACGUGGACAUUUUCAAUCCAAAAUACGACUGCAAGAUAAUGACCUCCAAGA
CUGACGUGUCAUCGUCCGUGAUCACAUCUCUGGGAGCCAUUGUCUCCUGCUACGGAAAGA
CUAAGUGCACCGCGUCGAACAAGAACAGGGGCAUUAUCAAGACCUUCAGCAACGGUUGCGA
CUAUGUGUCCAACAAGGGCGUGGAUACCGUGUCCGUGGGCAACACCUUGUACUACGUGAA
CAAGCAGGAGGGGAAGUCCCUUUAUGUGAAGGGGGAGCCAAUCAUUAACUUUUACGACCCC
CUGGUGUUCCCUAGCGACGAGUUCGACGCCUCAAUCUCUCAAGUCAACGAAAAGAUCAACC
AGAGCCUCGCCUUCAUCCGCAAGUCCGAUGAACUGCUGUCAGCCAUUGGGGGUUACAUCC
CUGAGGCCCCUCGGGACGGACAGGCAUACGUCCGCAAGGACGGCGAAUGGGUGCUGCUUA
GCACCUUCCUCUAACGGGUGGCAUCCCUGUGACCCCUCCCCAGUGCCUCUCCUGGCCCUG
GAAGUUGCCACUCCAGUGCCCACCAGCCUUGUCCUAAUAAAAUUAAGUUGCAUC
(SEQ ID NO: 29)
GGACAGAUCGCCUGGAGACGCCAUCCACGCUGUUUUGACCUCCAUAGAAGACACCGGGACC
GAUCCAGCCUCCGCGGCCGGGAACGGUGCAUUGGAACGCGGAUUCCCCGUGCCAAGAGUG
ACUCACCGUCCUUGACACGAUGGAACUGCUGAUCCUCAAAGCCAACGCAAUCACCACCAUU
CUCACCGCUGUGACCUUCUGCUUCGCAUCGGGGCAGAACAUCACUGAAGAGUUUUACCAGA
GCACUUGCAGCGCGGUGUCAAAGGGUUACCUUUCCGCACUGCGGACCGGAUGGUACACUU
CCGUGAUCACCAUUGAGCUCAGCAACAUCAAGGAAAACAAGUGCAAUGGCACCGACGCCAA
GGUCAAGCUGAUCAAACAAGAACUGGACAAGUACAAGAACGCCGUGACAGAAUUGCAGCUC
CUGAUGGGAUCCGGAAACGUCGGUCUGGGCGGAGCCAUCGCGAGUGGAGUGGCUGUGUC
CAAGGUCUUGCACCUCGAGGGAGAAGUGAACAAGAUCAAGUCCGCGCUGCUGUCAACGAAC
AAGGCCGUGGUGUCCCUGUCUAACGGCGUCAGCGUGCUGACGUUCAAGGUCCUGGACCUG
AAGAAUUACAUUGACAAGCAGCUGCUGCCCAUCCUCAACAAGCAAUCCUGCUCCAUCUCCAA
CCCCGAAACCGUGAUCGAGUUCCAGCAGAAGAACAACCGCCUGCUGGAAAUUACUCGCGAG
UUCUCUGUGAAUGCCGGCGUGACCACCCCUGUGUCCACCUACAUGCUGACCAACUCCGAGC
UUCUCUCCCUUAUCAAUGACAUGCCUAUCACGAACGACCAGAAGAAGCUGAUGUCGAACAA
CGUGCAGAUUGUGCGGCAGCAGUCAUACAGCAUCAUGUCGAUCAUCAAGGAAGAAGUGCUG
GCGUACGUGGUGCAACUCCCGCUGUACGGCGUCAUCGAUACCCCGUGCUGGAAGCUGCAC
ACCUCGCCUUUGUGUACCACCAACACCAAGAACGGAUCCAACAUCUGCUUAACCCGGACUG
AUCGGGGUUGGUACUGCGACAACGCCGGGAAUGUUUCGUUCUUCCCACAAGCCGAGACUU
GUAAAGUGCAGUCAAACAGAGUGUUCUGUGACACCAUGAACUCGAGAACCCUGCCCAGCGA
AGUGAACCUGUGUAACGUCGACAUCUUUAACCCAAAAUACGAUUGCAAGAUUAUGACCAGCA
AAACCGACGUGUCCUCCUCCGUGAUAACAAGCCUGGGGGCGAUUGUGUCAUGCUACGGAAA
GACUAAGUGCACCGCCUCGAACAAGAACCGCGGCAUCAUUAAGACUUUCUCGAAUGGUUGC
GACUAUGUGUCCAACAAGGGCGUGGAUACUGUGUCAGUCGGGAAUACUCUUUACUACGUGA
ACAAGCAGGAGGGGAAAAGCCUCUACGUGAAGGGAGAGCCUAUUAUCAACUUUUACGAUCC
GCUGGUGUUCCCGUCCGACGAAUUCGACGCCAGCAUCAGCCAAGUCAACGAGCUGAUUAAC
CAGUCCCUCGCCUUCAUCAACCAAUCCGACGAGCUCCUGCAUAACGUGAACGCCGGAAAGU
CCACCACCAACAUCAUGAUCACUACUAUUAUCAUCGUGAUCAUCGUCAUCCUGCUGAGCCU
GAUUGCUGUGGGCCUGUUGCUGUAUUGCAAAGCCAGGUCCACCCCGGUCACCCUGUCGAA
GGAUCAGCUGUCCGGAAUCAACAACAUUGCCUUCUCCAACUAACGGGUGGCAUCCCUGUGA
CCCCUCCCCAGUGCCUCUCCUGGCCCUGGAAGUUGCCACUCCAGUGCCCACCAGCCUUGU
CCUAAUAAAAUUAAGUUGCAUC

In some embodiments, the ORF (e.g., the ORF of hRSV, hMPV, or hPIV3) is codon-optimized. As used herein, “codon-optimized” or “codon optimization” refers to the introduction of certain codons (in exchange for the respective wild-type codons encoding the same amino acid), which may be more favorable with respect to stability of RNA and/or with respect to codon usage in a subject.

In certain embodiments, the hRSV F protein antigen is encoded by a codon-optimized mRNA ORF set forth as:

(SEQ ID NO: 4)
AUGGAACUGCUGAUCCUCAAAGCCAACGCAAUCACCACCAUUCUCACCGCUGUGACCUUCU
GCUUCGCAUCGGGGCAGAACAUCACUGAAGAGUUUUACCAGAGCACUUGCAGCGCGGUGU
CAAAGGGUUACCUUUCCGCACUGCGGACCGGAUGGUACACUUCCGUGAUCACCAUUGAGCU
CAGCAACAUCAAGGAAAACAAGUGCAAUGGCACCGACGCCAAGGUCAAGCUGAUCAAACAAG
AACUGGACAAGUACAAGAACGCCGUGACAGAAUUGCAGCUCCUGAUGGGAUCCGGAAACGU
CGGUCUGGGCGGAGCCAUCGCGAGUGGAGUGGCUGUGUCCAAGGUCUUGCACCUCGAGG
GAGAAGUGAACAAGAUCAAGUCCGCGCUGCUGUCAACGAACAAGGCCGUGGUGUCCCUGUC
UAACGGCGUCAGCGUGCUGACGUUCAAGGUCCUGGACCUGAAGAAUUACAUUGACAAGCAG
CUGCUGCCCAUCCUCAACAAGCAAUCCUGCUCCAUCUCCAACCCCGAAACCGUGAUCGAGU
UCCAGCAGAAGAACAACCGCCUGCUGGAAAUUACUCGCGAGUUCUCUGUGAAUGCCGGCGU
GACCACCCCUGUGUCCACCUACAUGCUGACCAACUCCGAGCUUCUCUCCCUUAUCAAUGAC
AUGCCUAUCACGAACGACCAGAAGAAGCUGAUGUCGAACAACGUGCAGAUUGUGCGGCAGC
AGUCAUACAGCAUCAUGUCGAUCAUCAAGGAAGAAGUGCUGGCGUACGUGGUGCAACUCCC
GCUGUACGGCGUCAUCGAUACCCCGUGCUGGAAGCUGCACACCUCGCCUUUGUGUACCAC
CAACACCAAGAACGGAUCCAACAUCUGCUUAACCCGGACUGAUCGGGGUUGGUACUGCGAC
AACGCCGGGAAUGUUUCGUUCUUCCCACAAGCCGAGACUUGUAAAGUGCAGUCAAACAGAG
UGUUCUGUGACACCAUGAACUCGAGAACCCUGCCCAGCGAAGUGAACCUGUGUAACGUCGA
CAUCUUUAACCCAAAAUACGAUUGCAAGAUUAUGACCAGCAAAACCGACGUGUCCUCCUCCG
UGAUAACAAGCCUGGGGGCGAUUGUGUCAUGCUACGGAAAGACUAAGUGCACCGCCUCGAA
CAAGAACCGCGGCAUCAUUAAGACUUUCUCGAAUGGUUGCGACUAUGUGUCCAACAAGGGC
GUGGAUACUGUGUCAGUCGGGAAUACUCUUUACUACGUGAACAAGCAGGAGGGGAAAAGCC
UCUACGUGAAGGGAGAGCCUAUUAUCAACUUUUACGAUCCGCUGGUGUUCCCGUCCGACGA
AUUCGACGCCAGCAUCAGCCAAGUCAACGAGCUGAUUAACCAGUCCCUCGCCUUCAUCAAC
CAAUCCGACGAGCUCCUGCAUAACGUGAACGCCGGAAAGUCCACCACCAACAUCAUGAUCA
CUACUAUUAUCAUCGUGAUCAUCGUCAUCCUGCUGAGCCUGAUUGCUGUGGGCCUGUUGC
UGUAUUGCAAAGCCAGGUCCACCCCGGUCACCCUGUCGAAGGAUCAGCUGUCCGGAAUCAA
CAACAUUGCCUUCUCCAACUAA

### IV. hMPV F Protein

hMPV is a negative-sense, single-stranded RNA virus belonging to the pneumovirus subfamily within the paramyxovirus family. hMPV infects airway epithelial cells in the nose and lung and is the second most common cause, after respiratory syncytial virus (RSV), of lower respiratory infection in young children. hMPV is an enveloped virus with a glycoprotein (G protein), small hydrophobic protein (SH protein), and a fusion protein (F protein) on the virion surface.

As it is an enveloped virus, entry of hMPV into host cells requires the fusion of viral and cellular membranes. Paramyxovirus entry usually requires two viral glycoproteins, the fusion (F) and attachment (G, H, or HN) proteins, and membrane fusion promoted by all paramyxovirus glycoproteins that have been examined takes place at neutral pH, with one possible exception (i.e., the SER virus). In addition to virus-cell membrane fusion, paramyxovirus glycoproteins also promote cell-cell fusion. Multinucleated giant cells, termed syncytia, can be found in tissues that have been infected by a variety of paramyxoviruses. Cultured cells infected with hMPV form syncytia, but examination of primary human airway epithelial cells infected with hMPV suggests that syncytium formation by this virus may not be a common in vivo occurrence.

hMPV F is a class I fusion glycoprotein synthesized as an inactive precursor (F0) that needs to be cleaved to become fusion competent. Proteolytic cleavage generates two disulfide-linked subunits (F2 N-terminal to F1) that assemble into a homotrimer. Cleavage occurs at a monobasic cleavage site immediately upstream of the hydrophobic fusion peptide. Cleavage can be achieved in tissue culture by addition of exogenous trypsin to the medium or by addition of a furin-expression plasmid. However, in vivo, other serine proteases, such as TMPRSS2, are thought to be likely more relevant for cleavage. The F trimer is incorporated into the virus particle in a metastable, “pre-fusion” or “pre-F” conformation. To initiate membrane fusion, hMPV F is activated and undergoes a series of stepwise conformational changes in the F protein that drive membrane fusion and result in hMPV F adopting a highly stable “postfusion” or “post-F” conformation.

In certain exemplary embodiments, proteolytic cleavage of F0 is achieved by co-transfection of a plasmid encoding an hMPV F polypeptide and a plasmid encoding furin at a 4:1 ratio hMPV plasmid:furin plasmid.

Provided herein are antigenic hMPV polypeptides comprising an hMPV F polypeptide. The hMPV F polypeptide may comprise the whole sequence of hMPV F or a portion of hMPV F. In certain embodiments, the portion is the ectodomain.

In some embodiments, the hMPV F polypeptide comprises a sequence having at least 80%, 85%, 90%, 95%, 96%, 97%, 98%, 99%, or 99.5% identity to any one of SEQ ID NOs: 10, 31, 33, and 35.

In some embodiments, the hMPV F polypeptide comprises a modified hMPV F polypeptide having at least 80% identity to the polypeptides of any one of SEQ ID NOs: 10, 31, 33, and 35, wherein the hMPV F polypeptide is antigenic.

In some embodiments, the hMPV F polypeptide comprises only the ectodomain portion of the F protein.

The amino acid sequence of F0 for A2-CAN97-83 is:

(SEQ ID NO: 10)
MSWKVVIIFSLLITPQHGLKESYLEESCSTITEGYLSVLRTGWYTNVFTLEVGDVENLTCSDGPSLIK
TELDLTKSALRELKTVSADQLAREEQIENPRQSRFVLGAIALGVATAAAVTAGVAIAKTIRLESEVTA
IKNALKTTNEAVSTLGNGVRVLATAVRELKDFVSKNLTRAINKNKCDIDDLKMAVSFSQFNRRFLNVV
RQFSDNAGITPAISLDLMTDAELARAVSNMPTSAGQIKLMLENRAMVRRKGFGILIGVYGSSVIYMVQ
LPIFGVIDTPCWIVKAAPSCSEKKGNYACLLREDQGWYCQNAGSTVYYPNEKDCETRGDHVFCDTAAG
INVAEQSKECNINISTTNYPCKVSTGRHPISMVALSPLGALVACYKGVSCSIGSNRVGIIKQLNKGCS
YITNQDADTVTIDNTVYQLSKVEGEQHVIKGRPVSSSFDPIKFPEDQFNVALDQVFENIENSQALVDQ
SNRILSSAEKGNTGFIIVIILIAVLGSSMILVSIFIIIKKTKKPTGAPPELSGVTNNGFIPHN.
(Accession AAN52910; Version AAN52910.1; DB Source Accession
AY145296.1.)
The transmembrane domain is bolded and underlined, and the
cytoplasmic tail is bolded.

The nucleotide sequence of F0 for A2-CAN97-83 is:

(SEQ ID NO: 30)
ATGTCTTGGAAAGTGGTGATCATTTTTTCATTGCTAATAACACCTCAACACGGTCTTAAAGAGA
GCTACCTAGAAGAATCATGTAGCACTATAACTGAGGGATATCTTAGTGTTCTGAGGACAGGTTG
GTATACCAACGTTTTTACATTAGAGGTGGGTGATGTAGAAAACCTTACATGTTCTGATGGACCT
AGCCTAATAAAAACAGAATTAGATCTGACCAAAAGTGCACTAAGAGAGCTCAAAACAGTCTCTG
CTGACCAATTGGCAAGAGAGGAACAAATTGAGAATCCCAGACAATCTAGGTTTGTTCTAGGAG
CAATAGCACTCGGTGTTGCAACAGCAGCTGCAGTCACAGCAGGTGTTGCAATTGCCAAAACCA
TCCGGCTTGAGAGTGAAGTCACAGCAATTAAGAATGCCCTCAAAACGACCAATGAAGCAGTAT
CTACATTGGGGAATGGAGTTCGAGTGTTGGCAACTGCAGTGAGAGAGCTGAAAGACTTTGTGA
GCAAGAATTTAACTCGTGCAATCAACAAAAACAAGTGCGACATTGATGACCTAAAAATGGCCGT
TAGCTTCAGTCAATTCAACAGAAGGTTTCTAAATGTTGTGCGGCAATTTTCAGACAATGCTGGA
ATAACACCAGCAATATCTTTGGACTTAATGACAGATGCTGAACTAGCCAGGGCCGTTTCTAACA
TGCCGACATCTGCAGGACAAATAAAATTGATGTTGGAGAACCGTGCGATGGTGCGAAGAAAGG
GGTTCGGAATCCTGATAGGGGTCTACGGGAGCTCCGTAATTTACATGGTGCAGCTGCCAATCT
TTGGCGTTATAGACACGCCTTGCTGGATAGTAAAAGCAGCCCCTTCTTGTTCCGAAAAAAAGG
GAAACTATGCTTGCCTCTTAAGAGAAGACCAAGGGTGGTATTGTCAGAATGCAGGGTCAACTG
TTTACTACCCAAATGAGAAAGACTGTGAAACAAGAGGAGACCATGTCTTTTGCGACACAGCAG
CGGGAATTAATGTTGCTGAGCAATCAAAGGAGTGCAACATCAACATATCCACTACAAATTACCC
ATGCAAAGTCAGCACAGGAAGACATCCTATCAGTATGGTTGCACTGTCTCCTCTTGGGGCTCT
GGTTGCTTGCTACAAAGGAGTAAGCTGTTCCATTGGCAGCAACAGAGTAGGGATCATCAAGCA
GCTGAACAAGGGTTGCTCCTATATAACCAACCAAGATGCAGACACAGTGACAATAGACAACAC
TGTATATCAGCTAAGCAAAGTTGAGGGTGAACAGCATGTTATAAAAGGCAGACCAGTGTCAAG
CAGCTTTGATCCAATCAAGTTTCCTGAAGATCAATTCAATGTTGCACTTGACCAAGTTTTTGAGA
ACATTGAAAACAGCCAGGCCTTGGTAGATCAATCAAACAGAATCCTAAGCAGTGCAGAGAAAG
GGAATACTGGCTTCATCATTGTAATAATTCTAATTGCTGTCCTTGGCTCTAGCATGATCCTAGTG
AGCATCTTCATTATAATCAAGAAAACAAAGAAACCAACGGGAGCACCTCCAGAGCTGAGTGGT
GTCACAAACAATGGCTTCATACCACATAATTAG.

In some embodiments, an epitope of the hMPV F protein that is shared between pre-F and post-F is blocked. Blocking an epitope reduces or eliminates the generation of antibodies against the epitope when an RNA (e.g., mRNA) that encodes for the antigenic hMPV F polypeptide is administered to a subject or when an antigenic hMPV F polypeptide is administered to a subject. This can increase the proportion of antibodies that target an epitope specific to a particular conformation of F, such as the pre-fusion conformation (e.g., antibodies that target site Ø and/or site V). Because F has the pre-fusion conformation in viruses that have not yet entered cells, an increased proportion of antibodies that target pre-F can provide a greater degree of neutralization (e.g., expressed as a neutralizing to binding ratio, as described herein).

The hMPV F polypeptides described herein may have deletions or substitutions relative to the wild-type hMPV F protein (e.g., SEQ ID NO: 10).

For example, in certain embodiments, an hMPV polypeptide: (a) lacks a transmembrane domain and lacks a cytoplasmic tail, and comprises an human rhinovirus 3C (HRV-3C) protease cleavage site; (b) comprises a F₀ cleavage site mutation comprising amino acid substitutions Q100R and S101R relative to SEQ ID NO: 10, replacing glutamine at amino acid position 100 with arginine, and replacing serine at amino acid position 101 with arginine; (c) comprises a heterologous signal peptide; (d) comprises at least one tag sequence that is optionally a polyhistidine-tag (e.g., a 6×His tag(SEQ ID NO:41), 8×His tag(SEQ ID NO:42), etc.) and/or a Strep II tag; and/or (e) comprises a foldon domain.

In certain embodiments, an hMPV polypeptide lacks a transmembrane domain and lacks a cytoplasmic tail, and comprises: an F0 cleavage site mutation comprising amino acid substitutions Q100R and S101R relative to SEQ ID NO: 10; replacing glutamine at amino acid position 100 with arginine, and replacing serine at amino acid position 101 with arginine; a human rhinovirus 3C (HRV-3C) protease cleavage site; a heterologous signal peptide; a polyhistidine-tag (e.g., a 6×His tag(SEQ ID NO:41), 8×His tag(SEQ ID NO:42), etc.) and/or a Strep II tag; and a foldon domain.

In certain embodiments, an hMPV polypeptide includes a valine, alanine, glycine, isoleucine, leucine, or proline substitution at position 185 of SEQ ID NO: 10.

In certain embodiments, an hMPV polypeptide includes a phenylalanine, tryptophan, tyrosine, valine, alanine, isoleucine, or leucine substitution at position 160 of SEQ ID NO: 10, and/or a valine, alanine, isoleucine, leucine, phenylalanine, tyrosine, or proline substitution at position 46 of SEQ ID NO: 10.

In certain embodiments, an hMPV polypeptide includes a substitution at position 160 of SEQ ID NO: 10 and a substitution at position 46 of SEQ ID NO: 10 wherein the substitutions are “stabilizing substitutions” that stabilize the tertiary and/or quaternary structure of an hMPV polypeptide. Stabilizing substitutions include, but are not limited to, substitution of: hydrophobic amino acids (e.g., glycine, alanine, valine, leucine, isoleucine, phenylalanine, tyrosine, tryptophan, proline, and methionine); hydrophilic amino acids (e.g., cysteine, serine, threonine, asparagine, and glutamine; amino acids that forms a disulfide bond (e.g., cysteine); amino acids that form hydrogen bonds (e.g., tryptophan, histidine, tyrosine, and phenylalanine); charged amino acids (e.g., aspartic acid, glutamic acid, arginine, lysine, and histidine), and the like.

In certain embodiments, an hMPV polypeptide is from an A strain hMPV (e.g., an A1 subtype or an A2 subtype) or from a B strain hMPV (e.g., a 1 subtype or a B2 subtype).

In certain embodiments, an amino acid sequence comprising a “backbone” F0 polypeptide sequence is provided, set forth as:

(SEQ ID NO: 31)
MSWKVVIIFSLLITPQHGLKESYLEESCSTITEGYLSVLRTGWYTNVFTL
EVGDVENLTCSDGPSLIKTELDLTKSALRELKTVSADQLAREEQIENPRr
rRFVLGAIALGVATAAAVTAGVAIAKTIRLESEVTAIKNALKTTNEAVST
LGNGVRVLATAVRELKDFVSKNLTRAINKNKCDIDDLKMAVSFSQFNRRF
LNVVRQFSDNAGITPAISLDLMTDAELARAVSNMPTSAGQIKLMLENRAM
VRRKGFGILIGVYGSSVIYMVQLPIFGVIDTPCWIVKAAPSCSEKKGNYA
CLLREDQGWYCQNAGSTVYYPNEKDCETRGDHVFCDTAAGINVAEQSKEC
NINISTTNYPCKVSTGRHPISMVALSPLGALVACYKGVSCSIGSNRVGII
KQLNKGCSYITNQDADTVTIDNTVYQLSKVEGEQHVIKGRPVSSSFDPIK
FPEDQFNVALDQVFENIENSQALVDQSNRILSSAEKGNTggggsgyipea
prdgqayvrkdgewvllstflgrslevlfqgpghhhhhhhhsawshpqfe
k.
(Lower case amino acids denote a linker, foldon
motif, linker, HRV-3C cleavage site, linker, 8X-
His-tag(SEQ ID NO: 42), and strep-tag II region.)

In certain embodiments, a nucleotide sequence encoding a F0 polypeptide sequence is provided, set forth as:

(SEQ ID NO: 32)
ATGAGTTGGAAGGTGGTGATTATCTTCTCCCTGCTGATTACACCACAACATGGACTGAAAGAGT
CCTACTTGGAGGAGTCCTGTAGCACCATCACAGAGGGCTACCTGTCTGTGCTGAGGACAGGC
TGGTACACCAATGTGTTCACCTTGGAGGTGGGAGATGTGGAGAACCTGACTTGTTCTGATGGA
CCATCCCTGATTAAGACAGAACTGGACCTGACCAAGTCTGCCCTGAGGGAACTGAAAACAGTG
TCTGCTGACCAACTTGCCAGGGAGGAACAGATTGAGAACCCAAGGAGGAGGAGGTTTGTGCT
GGGAGCCATTGCCCTGGGAGTGGCTACAGCAGCAGCAGTGACAGCAGGAGTGGCTATTGCCA
AGACCATCAGATTGGAGTCTGAGGTGACAGCCATCAAGAATGCCCTGAAAACCACCAATGAGG
CTGTGAGCACCCTGGGCAATGGAGTGAGGGTGCTGGCTACAGCAGTGAGGGAACTGAAAGAC
TTTGTGAGCAAGAACCTGACCAGGGCTATCAACAAGAACAAGTGTGACATCGATGACCTGAAA
ATGGCTGTGTCCTTCAGCCAGTTCAACAGGAGGTTCCTGAATGTGGTGAGACAGTTCTCTGAC
AATGCTGGCATCACACCTGCCATCTCCCTGGACCTGATGACAGATGCTGAACTGGCAAGGGCT
GTGAGCAATATGCCAACCTCTGCTGGACAAATCAAACTGATGTTGGAGAACAGGGCTATGGTG
AGGAGGAAGGGCTTTGGCATCCTGATTGGAGTCTATGGCTCCTCTGTGATTTATATGGTCCAA
CTTCCAATCTTTGGAGTGATTGACACACCATGTTGGATTGTGAAGGCTGCCCCATCCTGTTCTG
AGAAGAAGGGCAACTATGCCTGTCTGCTGAGGGAGGACCAGGGCTGGTATTGTCAGAATGCT
GGCAGCACAGTCTACTACCCAAATGAGAAGGACTGTGAGACCAGGGGAGACCATGTGTTCTG
TGACACAGCAGCAGGCATCAATGTGGCTGAACAGAGCAAGGAGTGTAACATCAACATCAGCAC
CACCAACTACCCATGTAAGGTGAGCACAGGCAGACACCCAATCAGTATGGTGGCTCTGAGCC
CACTGGGAGCCCTGGTGGCTTGTTACAAGGGAGTGTCCTGTAGCATTGGCAGCAACAGGGTG
GGCATCATCAAGCAACTTAACAAGGGCTGTTCCTACATCACCAACCAGGATGCTGACACAGTG
ACCATTGACAACACAGTCTACCAACTTAGCAAGGTGGAGGGAGAACAGCATGTGATTAAGGGC
AGACCTGTGTCCTCCTCCTTTGACCCAATCAAGTTTCCTGAGGACCAGTTCAATGTGGCTCTG
GACCAGGTGTTTGAGAACATTGAGAACAGCCAGGCTCTGGTGGACCAGAGCAACAGGATTCT
GTCCTCTGCTGAGAAGGGCAACACAGGAGGAGGAGGCTCTGGCTACATCCCTGAGGCTCCAA
GGGATGGACAAGCCTATGTGAGGAAGGATGGAGAGTGGGTGCTGCTGAGCACCTTCCTGGGC
AGGTCCTTGGAGGTGCTGTTCCAGGGACCTGGACACCACCACCACCACCACCACCACTCTGC
CTGGAGCCACCCACAGTTTGAGAAGTAA

In certain embodiments, an hMPV polypeptide comprises a “backbone” hMPV sequence set forth as SEQ ID NO: 31 and may optionally contain one or more amino acid substitutions. For example, in certain embodiments, an hMPV polypeptide includes a valine, alanine, glycine, isoleucine, leucine, or proline substitution at position 185 of SEQ ID NO: 31. In certain embodiments, an hMPV polypeptide includes a phenylalanine, tryptophan, or tyrosine substitution at position 160 of SEQ ID NO: 31, and/or a valine, alanine, glycine, isoleucine, leucine, or proline substitution at position 46 of SEQ ID NO: 31. In certain embodiments, an hMPV polypeptide includes an arginine substitution at one or both of positions 100 and 101 of SEQ ID NO: 31.

In certain embodiments, an hMPV polypeptide has at least 80%, 85%, 90%, 91%, 92%, 93%, 94%, 95%, 96%, 97%, 98%, or 99% sequence identity to SEQ ID NO: 31.

In certain embodiments, an amino acid sequence comprising an hMPV polypeptide sequence is provided, set forth as:

(SEQ ID NO: 33)
MSWKVVIIFSLLITPQHGLKESYLEESCSTITEGYLSVLRTGWYTNVFTL
EVGDVENLTCSDGPSLIKTELDLTKSALRELKTVSADQLAREEQIENPRr
rRFVLGAIALGVATAAAVTAGVAIAKTIRLESEVTAIKNALKTTNEAVST
LGNGVRVLATAVRELKDFVSKNLTRAINKNKCDIpDLKMAVSFSQFNRRF
LNVVRQFSDNAGITPAISLDLMTDAELARAVSNMPTSAGQIKLMLENRAM
VRRKGFGILIGVYGSSVIYMVQLPIFGVIDTPCWIVKAAPSCSEKKGNYA
CLLREDQGWYCQNAGSTVYYPNEKDCETRGDHVFCDTAAGINVAEQSKEC
NINISTTNYPCKVSTGRHPISMVALSPLGALVACYKGVSCSIGSNRVGII
KQLNKGCSYITNQDADTVTIDNTVYQLSKVEGEQHVIKGRPVSSSFDPIK
FPEDQFNVALDQVFENIENSQALVDQSNRILSSAEKGNTggggsgyipea
prdgqayvrkdgewvllstflgrslevlfqgpghhhhhhhhsawshpqfe
k (D185P).
(Lower case amino acids denote a linker, foldon
motif, linker, HRV-3C cleavage site, linker, 8X-
His-tag(SEQ ID NO: 42), and strep-tag Il region.)

In certain embodiments, a nucleotide sequence encoding an hMPV polypeptide sequence is provided, set forth as:

(SEQ ID NO: 34)
ATGAGTTGGAAGGTGGTGATTATCTTCTCCCTGCTGATTACACCACAACATGGACTGAAAGAGT
CCTACTTGGAGGAGTCCTGTAGCACCATCACAGAGGGCTACCTGTCTGTGCTGAGGACAGGC
TGGTACACCAATGTGTTCACCTTGGAGGTGGGAGATGTGGAGAACCTGACTTGTTCTGATGGA
CCATCCCTGATTAAGACAGAACTGGACCTGACCAAGTCTGCCCTGAGGGAACTGAAAACAGTG
TCTGCTGACCAACTTGCCAGGGAGGAACAGATTGAGAACCCAAGGAGGAGGAGGTTTGTGCT
GGGAGCCATTGCCCTGGGAGTGGCTACAGCAGCAGCAGTGACAGCAGGAGTGGCTATTGCCA
AGACCATCAGATTGGAGTCTGAGGTGACAGCCATCAAGAATGCCCTGAAAACCACCAATGAGG
CTGTGAGCACCCTGGGCAATGGAGTGAGGGTGCTGGCTACAGCAGTGAGGGAACTGAAAGAC
TTTGTGAGCAAGAACCTGACCAGGGCTATCAACAAGAACAAGTGTGACATCCCTGACCTGAAA
ATGGCTGTGTCCTTCAGCCAGTTCAACAGGAGGTTCCTGAATGTGGTGAGACAGTTCTCTGAC
AATGCTGGCATCACACCTGCCATCTCCCTGGACCTGATGACAGATGCTGAACTGGCAAGGGCT
GTGAGCAATATGCCAACCTCTGCTGGACAAATCAAACTGATGTTGGAGAACAGGGCTATGGTG
AGGAGGAAGGGCTTTGGCATCCTGATTGGAGTCTATGGCTCCTCTGTGATTTATATGGTCCAA
CTTCCAATCTTTGGAGTGATTGACACACCATGTTGGATTGTGAAGGCTGCCCCATCCTGTTCTG
AGAAGAAGGGCAACTATGCCTGTCTGCTGAGGGAGGACCAGGGCTGGTATTGTCAGAATGCT
GGCAGCACAGTCTACTACCCAAATGAGAAGGACTGTGAGACCAGGGGAGACCATGTGTTCTG
TGACACAGCAGCAGGCATCAATGTGGCTGAACAGAGCAAGGAGTGTAACATCAACATCAGCAC
CACCAACTACCCATGTAAGGTGAGCACAGGCAGACACCCAATCAGTATGGTGGCTCTGAGCC
CACTGGGAGCCCTGGTGGCTTGTTACAAGGGAGTGTCCTGTAGCATTGGCAGCAACAGGGTG
GGCATCATCAAGCAACTTAACAAGGGCTGTTCCTACATCACCAACCAGGATGCTGACACAGTG
ACCATTGACAACACAGTCTACCAACTTAGCAAGGTGGAGGGAGAACAGCATGTGATTAAGGGC
AGACCTGTGTCCTCCTCCTTTGACCCAATCAAGTTTCCTGAGGACCAGTTCAATGTGGCTCTG
GACCAGGTGTTTGAGAACATTGAGAACAGCCAGGCTCTGGTGGACCAGAGCAACAGGATTCT
GTCCTCTGCTGAGAAGGGCAACACAGGAGGAGGAGGCTCTGGCTACATCCCTGAGGCTCCAA
GGGATGGACAAGCCTATGTGAGGAAGGATGGAGAGTGGGTGCTGCTGAGCACCTTCCTGGGC
AGGTCCTTGGAGGTGCTGTTCCAGGGACCTGGACACCACCACCACCACCACCACCACTCTGC
CTGGAGCCACCCACAGTTTGAGAAGTAA (D185P).

In certain embodiments, a nucleotide sequence encoding an hMPV polypeptide sequence is provided, set forth as:

(SEQ ID NO: 36)
ATGTCTTGGAAAGTCGTCATCATCTTCTCTCTGCTGATCACCCCACAACACGGCCTGAAGGAAT
CTTATCTGGAAGAGTCCTGCTCCACAATCACAGAGGGCTACCTGAGCGTGCTGAGAACCGGCT
GGTACACCAACGTGTTCACTCTGGAGGTGGGCGACGTGGAGAACCTGACTTGTAGTGACGGC
CCCTCCCTGATCAAGACTGAGCTGGACCTGACAAAGAGTGCACTGAGAGAACTCAAGACTGTG
TCCGCAGACCAGCTGGCCCGCGAGGAGCAGATCGAAAATCCTAGACAGTCAAGGTTCGTCCT
GGGAGCCATTGCTCTGGGAGTTGCTACAGCTGCCGCTGTGACCGCAGGGGTGGCTATTGCTA
AAACCATCAGGCTGGAGTCCGAAGTGACAGCAATCAAGAATGCCCTGAAGACCACCAACGAG
GCAGTCTCCACACTGGGCAATGGAGTGAGGGTGCTGGCAACCGCCGTGAGGGAGCTGAAGG
ACTTCGTGTCCAAGAACCTGACCAGGGCTATCAACAAAAACAAGTGCGACATCCCCGATCTGA
AGATGGCAGTTAGCTTTTCCCAGTTTAACCGGAGATTCCTGAATGTGGTTAGACAGTTCAGCGA
CAACGCCGGGATCACCCCAGCTATTTCCCTGGACCTGATGACTGATGCCGAGCTGGCACGGG
CTGTGTCCAATATGCCCACCAGCGCTGGGCAGATTAAGCTGATGCTGGAGAATCGGGCAATG
GTGAGAAGGAAGGGGTTTGGCATCCTGATCGGCGTGTACGGGTCCTCCGTGATCTACATGGT
GCAGCTGCCTATTTTTGGAGTGATTGATACACCCTGCTGGATCGTTAAAGCAGCACCCAGCTG
CTCCGAGAAGAAGGGCAATTACGCCTGTCTGCTGCGGGAGGACCAGGGGTGGTACTGCCAGA
ACGCCGGCTCCACAGTGTATTACCCCAATGAAAAGGACTGCGAGACAAGGGGAGACCACGTG
TTCTGCGACACTGCCGCTGGGATTAATGTGGCCGAGCAGAGCAAGGAGTGCAACATCAACATT
TCCACCACAAACTACCCCTGCAAGGTGAGCACCGGCAGGCACCCTATCTCCATGGTGGCCCT
GTCTCCCCTGGGAGCTCTGGTGGCTTGCTACAAGGGAGTGAGCTGTAGCATCGGGTCCAATA
GAGTCGGGATTATCAAGCAGCTGAATAAGGGCTGCAGCTATATTACCAACCAGGATGCCGATA
CTGTGACTATTGACAACACAGTGTATCAGCTGTCAAAGGTGGAAGGCGAACAGCATGTGATCA
AAGGACGGCCCGTCAGCAGCTCCTTTGACCCTATCAAATTCCCCGAAGACCAGTTTAACGTGG
CACTGGACCAGGTTTTCGAAAATATTGAGAATTCTCAGGCCCTGGTGGACCAGTCTAACCGGA
TCCTCTCCTCCGCCGAGAAGGGAAATACAGGCTTTATTATCGTGATCATCCTGATCGCAGTGC
TGGGATCCAGTATGATCCTGGTCTCCATTTTCATCATCATTAAGAAGACCAAGAAACCCACTGG
CGCACCACCTGAACTGAGCGGCGTGACTAACAATGGCTTTATCCCTCACAATTGA (D185P mRNA).

In certain embodiments, an hMPV polypeptide has at least 80%, 85%, 90%, 91%, 92%, 93%, 94%, 95%, 96%, 97%, 98%, or 99% sequence identity to SEQ ID NO: 33. In certain embodiments, an hMPV polypeptide comprises SEQ ID NO: 33. In certain embodiments, an hMPV polynucleotide has at least 80%, 85%, 90%, 91%, 92%, 93%, 94%, 95%, 96%, 97%, 98%, or 99% sequence identity to SEQ ID NO: 34. In certain embodiments, an hMPV polynucleotide comprises SEQ ID NO: 34. In certain embodiments, an hMPV polynucleotide has at least 80%, 85%, 90%, 91%, 92%, 93%, 94%, 95%, 96%, 97%, 98%, or 99% sequence identity to SEQ ID NO: 36. In certain embodiments, an hMPV polynucleotide comprises SEQ ID NO: 36.

In certain embodiments, an amino acid sequence comprising an hMPV polypeptide sequence is provided, set forth as:

(SEQ ID NO: 35)
MSWKVVIIFSLLITPQHGLKESYLEESCSTITEGYLSVLRTGWYTVVFTLEV
GDVENLTCSDGPSLIKTELDLTKSALRELKTVSADQLAREEQIENPRrrRFV
LGAIALGVATAAAVTAGVAIAKTIRLESEVTAIKNALKTTNEAVSTLGNGVR
VLAFAVRELKDFVSKNLTRAINKNKCDIDDLKMAVSFSQFNRRFLNVVRQFS
DNAGITPAISLDLMTDAELARAVSNMPTSAGQIKLMLENRAMVRRKGFGILI
GVYGSSVIYMVQLPIFGVIDTPCWIVKAAPSCSEKKGNYACLLREDQGWYCQ
NAGSTVYYPNEKDCETRGDHVFCDTAAGINVAEQSKECNINISTTNYPCKVS
TGRHPISMVALSPLGALVACYKGVSCSIGSNRVGIIKQLNKGCSYITNQDAD
TVTIDNTVYQLSKVEGEQHVIKGRPVSSSFDPIKFPEDQFNVALDQVFENIE
NSQALVDQSNRILSSAEKGNTggggsgyipeaprdgqayvrkdgewvllst
flgrslevlfqgpghhhhhhhhsawshpqfek (T160F_N46V).
(Lower case amino acids denote a linker, foldon
motif, linker, HRV-3C cleavage site, linker, 8X-
His-tag(SEQ ID NO: 42), and strep-tag II region.)

In certain embodiments, a nucleotide sequence encoding an hMPV polypeptide sequence is provided, set forth as:

(SEQ ID NO: 37)
ATGAGTTGGAAGGTGGTGATTATCTTCTCCCTGCTGATTACACCACAACATGGACTGAAAGAGT
CCTACTTGGAGGAGTCCTGTAGCACCATCACAGAGGGCTACCTGTCTGTGCTGAGGACAGGC
TGGTACACAGTGGTGTTCACCTTGGAGGTGGGAGATGTGGAGAACCTGACTTGTTCTGATGGA
CCATCCCTGATTAAGACAGAACTGGACCTGACCAAGTCTGCCCTGAGGGAACTGAAAACAGTG
TCTGCTGACCAACTTGCCAGGGAGGAACAGATTGAGAACCCAAGGAGGAGGAGGTTTGTGCT
GGGAGCCATTGCCCTGGGAGTGGCTACAGCAGCAGCAGTGACAGCAGGAGTGGCTATTGCCA
AGACCATCAGATTGGAGTCTGAGGTGACAGCCATCAAGAATGCCCTGAAAACCACCAATGAGG
CTGTGAGCACCCTGGGCAATGGAGTGAGGGTGCTGGCTTTTGCTGTGAGGGAACTGAAAGAC
TTTGTGAGCAAGAACCTGACCAGGGCTATCAACAAGAACAAGTGTGACATTGATGACCTGAAA
ATGGCTGTGTCCTTCAGCCAGTTCAACAGGAGGTTCCTGAATGTGGTGAGACAGTTCTCTGAC
AATGCTGGCATCACACCTGCCATCTCCCTGGACCTGATGACAGATGCTGAACTGGCAAGGGCT
GTGAGCAATATGCCAACCTCTGCTGGACAAATCAAACTGATGTTGGAGAACAGGGCTATGGTG
AGGAGGAAGGGCTTTGGCATCCTGATTGGAGTCTATGGCTCCTCTGTGATTTATATGGTCCAA
CTTCCAATCTTTGGAGTGATTGACACACCATGTTGGATTGTGAAGGCTGCCCCATCCTGTTCTG
AGAAGAAGGGCAACTATGCCTGTCTGCTGAGGGAGGACCAGGGCTGGTATTGTCAGAATGCT
GGCAGCACAGTCTACTACCCAAATGAGAAGGACTGTGAGACCAGGGGAGACCATGTGTTCTG
TGACACAGCAGCAGGCATCAATGTGGCTGAACAGAGCAAGGAGTGTAACATCAACATCAGCAC
CACCAACTACCCATGTAAGGTGAGCACAGGCAGACACCCAATCAGTATGGTGGCTCTGAGCC
CACTGGGAGCCCTGGTGGCTTGTTACAAGGGAGTGTCCTGTAGCATTGGCAGCAACAGGGTG
GGCATCATCAAGCAACTTAACAAGGGCTGTTCCTACATCACCAACCAGGATGCTGACACAGTG
ACCATTGACAACACAGTCTACCAACTTAGCAAGGTGGAGGGAGAACAGCATGTGATTAAGGGC
AGACCTGTGTCCTCCTCCTTTGACCCAATCAAGTTTCCTGAGGACCAGTTCAATGTGGCTCTG
GACCAGGTGTTTGAGAACATTGAGAACAGCCAGGCTCTGGTGGACCAGAGCAACAGGATTCT
GTCCTCTGCTGAGAAGGGCAACACAGGAGGAGGAGGCTCTGGCTACATCCCTGAGGCTCCAA
GGGATGGACAAGCCTATGTGAGGAAGGATGGAGAGTGGGTGCTGCTGAGCACCTTCCTGGGC
AGGTCCTTGGAGGTGCTGTTCCAGGGACCTGGACACCACCACCACCACCACCACCACTCTGC
CTGGAGCCACCCACAGTTTGAGAAGTAA (T160F_N46V).

In certain embodiments, a nucleotide sequence encoding an hMPV polypeptide sequence is provided, set forth as:

(SEQ ID NO: 38)
ATGAGCTGGAAGGTTGTGATTATTTTCTCTCTGCTGATTACTCCACAGCACGGCCTGAAGGAGT
CCTACCTGGAGGAGTCCTGTTCTACTATCACTGAGGGGTATCTCTCTGTGCTGCGGACAGGGT
GGTATACAGTGGTGTTCACCCTGGAGGTTGGCGATGTGGAGAATCTGACTTGCAGCGATGGC
CCTTCTCTGATCAAGACCGAGCTGGATCTGACAAAAAGCGCCCTCAGAGAACTGAAAACCGTG
TCCGCCGATCAGCTGGCAAGGGAGGAGCAGATCGAGAACCCACGGCAGAGCAGGTTTGTGCT
GGGCGCTATCGCTCTGGGCGTGGCCACTGCAGCTGCTGTCACTGCAGGGGTCGCAATCGCTA
AGACTATCAGACTGGAATCCGAGGTGACCGCCATTAAGAATGCCCTGAAGACTACCAACGAGG
CTGTGTCCACTCTGGGAAACGGAGTGAGGGTCCTGGCCTTCGCAGTGAGGGAGCTGAAGGAT
TTTGTGTCAAAGAACCTTACACGGGCCATCAACAAGAATAAGTGCGATATCGATGACCTGAAGA
TGGCCGTGTCCTTCTCCCAGTTCAACCGGCGCTTTCTGAATGTGGTGCGCCAGTTTTCCGACA
ACGCTGGAATCACCCCTGCTATCAGCCTGGACCTCATGACCGACGCCGAACTCGCAAGGGCC
GTTTCTAACATGCCTACATCCGCTGGACAGATTAAGCTGATGCTGGAGAATAGAGCAATGGTG
AGGAGAAAGGGATTCGGCATCCTGATTGGCGTGTACGGATCTAGCGTGATCTACATGGTGCA
GCTGCCGATCTTCGGCGTGATCGATACTCCTTGTTGGATCGTCAAGGCCGCCCCTTCCTGCTC
CGAGAAGAAGGGCAATTACGCTTGTCTGCTGCGGGAGGACCAGGGCTGGTATTGCCAGAACG
CCGGGTCTACAGTGTACTATCCTAACGAGAAGGATTGCGAGACCAGAGGCGACCACGTTTTCT
GTGATACAGCCGCCGGAATCAATGTCGCAGAGCAGTCTAAGGAGTGCAACATCAATATCTCTA
CAACCAATTACCCATGTAAGGTGAGCACTGGACGGCACCCTATCAGTATGGTGGCTCTGAGCC
CACTGGGGGCACTGGTGGCTTGCTACAAGGGGGTGAGCTGCAGTATCGGCAGTAACAGAGTG
GGCATTATCAAGCAGCTGAACAAAGGGTGCTCTTATATTACAAACCAGGATGCAGATACTGTGA
CCATCGACAACACTGTGTACCAGCTGTCCAAGGTGGAGGGGGAGCAGCATGTGATCAAAGGG
AGACCCGTCTCTTCTTCTTTCGATCCCATCAAGTTCCCTGAAGACCAGTTCAATGTTGCCCTGG
ACCAGGTTTTCGAGAACATCGAAAATAGCCAGGCCTTGGTCGATCAATCCAACAGGATCCTGA
GCAGCGCAGAGAAAGGGAACACTGGCTTCATCATCGTGATCATTCTGATCGCCGTGCTGGGG
AGCAGTATGATTCTGGTGTCCATTTTCATCATCATCAAGAAGACCAAGAAGCCTACAGGAGCAC
CCCCTGAGCTGAGCGGAGTGACCAACAACGGCTTTATCCCTCACAACTGA (T160F_N46V).

In certain embodiments, a nucleotide sequence encoding an hMPV polypeptide sequence is provided, set forth as:

(SEQ ID NO: 39)
ATGAGCTGGAAGGTTGTGATTATTTTCTCTCTGCTGATTACTCCACAGCACGGCCTGAAGGAGT
CCTACCTGGAGGAGTCCTGTTCTACTATCACTGAGGGGTATCTCTCTGTGCTGCGGACAGGGT
GGTATACAGTGGTGTTCACCCTGGAGGTTGGCGATGTGGAGAATCTGACTTGCAGCGATGGC
CCTTCTCTGATCAAGACCGAGCTGGATCTGACAAAAAGCGCCCTCAGAGAACTGAAAACCGTG
TCCGCCGATCAGCTGGCAAGGGAGGAGCAGATCGAGAACCCACGGCAGAGCAGGTTTGTGCT
GGGCGCTATCGCTCTGGGCGTGGCCACTGCAGCTGCTGTCACTGCAGGGGTCGCAATCGCTA
AGACTATCAGACTGGAATCCGAGGTGACCGCCATTAAGAATGCCCTGAAGACTACCAACGAGG
CTGTGTCCACTCTGGGAAACGGAGTGAGGGTCCTGGCCTTCGCAGTGAGGGAGCTGAAGGAT
TTTGTGTCAAAGAACCTTACACGGGCCATCAACAAGAATAAGTGCGATATCGATGACCTGAAGA
TGGCCGTGTCCTTCTCCCAGTTCAACCGGCGCTTTCTGAATGTGGTGCGCCAGTTTTCCGACA
ACGCTGGAATCACCCCTGCTATCAGCCTGGACCTCATGACCGACGCCGAACTCGCAAGGGCC
GTTTCTAACATGCCTACATCCGCTGGACAGATTAAGCTGATGCTGGAGAATAGAGCAATGGTG
AGGAGAAAGGGATTCGGCATCCTGATTGGCGTGTACGGATCTAGCGTGATCTACATGGTGCA
GCTGCCGATCTTCGGCGTGATCGATACTCCTTGTTGGATCGTCAAGGCCGCCCCTTCCTGCTC
CGAGAAGAAGGGCAATTACGCTTGTCTGCTGCGGGAGGACCAGGGCTGGTATTGCCAGAACG
CCGGGTCTACAGTGTACTATCCTAACGAGAAGGATTGCGAGACCAGAGGCGACCACGTTTTCT
GTGATACAGCCGCCGGAATCAATGTCGCAGAGCAGTCTAAGGAGTGCAACATCAATATCTCTA
CAACCAATTACCCATGTAAGGTGAGCACTGGACGGCACCCTATCAGTATGGTGGCTCTGAGCC
CACTGGGGGCACTGGTGGCTTGCTACAAGGGGGTGAGCTGCAGTATCGGCAGTAACAGAGTG
GGCATTATCAAGCAGCTGAACAAAGGGTGCTCTTATATTACAAACCAGGATGCAGATACTGTGA
CCATCGACAACACTGTGTACCAGCTGTCCAAGGTGGAGGGGGAGCAGCATGTGATCAAAGGG
AGACCCGTCTCTTCTTCTTTCGATCCCATCAAGTTCCCTGAAGACCAGTTCAATGTTGCCCTGG
ACCAGGTTTTCGAGAACATCGAAAATAGCCAGGCCTTGGTCGATCAATCCAACAGGATCCTGA
GCAGCGCAGAGAAAGGGAACACTGGCTTCATCATCGTGATCATTCTGATCGCCGTGCTGGGG
AGCAGTATGATTCTGGTGTCCATTTTCATCATCATCAAGAAGACCAAGAAGCCTACAGGAGCAC
CCCCTGAGCTGAGCGGAGTGACCAACAACGGCTTTATCCCTCACAACTAA (T160F_N46V).

In certain embodiments, an hMPV polypeptide has at least 80%, 85%, 90%, 91%, 92%, 93%, 94%, 95%, 96%, 97%, 98%, or 99% sequence identity to SEQ ID NO: 35. In certain embodiments, an hMPV polypeptide comprises SEQ ID NO: 35. In certain embodiments, an hMPV polynucleotide has at least 80%, 85%, 90%, 91%, 92%, 93%, 94%, 95%, 96%, 97%, 98%, or 99% sequence identity to SEQ ID NO: 37. In certain embodiments, an hMPV polynucleotide comprises SEQ ID NO: 37. In certain embodiments, an hMPV polynucleotide has at least 80%, 85%, 90%, 91%, 92%, 93%, 94%, 95%, 96%, 97%, 98%, or 99% sequence identity to SEQ ID NO: 38. In certain embodiments, an hMPV polynucleotide comprises SEQ ID NO: 38. In certain embodiments, an hMPV polynucleotide has at least 80%, 85%, 90%, 91%, 92%, 93%, 94%, 95%, 96%, 97%, 98%, or 99% sequence identity to SEQ ID NO: 37. In certain embodiments, an hMPV polynucleotide comprises SEQ ID NO: 37. In certain embodiments, an hMPV polynucleotide has at least 80%, 85%, 90%, 91%, 92%, 93%, 94%, 95%, 96%, 97%, 98%, or 99% sequence identity to SEQ ID NO: 39. In certain embodiments, an hMPV polynucleotide comprises SEQ ID NO: 39.

In general, positions in constructs described herein can be mapped onto a reference sequence, e.g., the wild-type sequence of SEQ ID NO: 10 or the backbone sequence of SEQ ID NO: 31, by pairwise alignment, e.g., using the Needleman-Wunsch algorithm with standard parameters (EBLOSUM62 matrix, Gap penalty 10, gap extension penalty 0.5).

In certain embodiments, vaccines of the present disclosure may comprise at least one hMPV F protein antigen. hMPV F protein antigens of the disclosure can be made by a variety of methods. In one embodiment, a host cell line that can be of eukaryotic or prokaryotic origin is used for expression of an hMPV F polypeptide. In one embodiment, a host cell line used for expression of an hMPV F polypeptide is of bacterial origin. In one embodiment, a host cell line used for expression of an hMPV F polypeptide is of mammalian origin. Particular host cell lines which are best suited for the desired gene product to be expressed therein can be determined. Exemplary host cell lines include, but are not limited to, DG44 and DUXB11 (Chinese hamster ovary lines, DHFR minus), HELA (human cervical carcinoma), CVI (monkey kidney line), COS (a derivative of CVI with SV40 T antigen), CHO (Chinese hamster ovary), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), and 293 (human kidney). Host cell lines are typically available from commercial services, the American Tissue Culture Collection (ATCC), or from published literature.

In certain embodiments, the hMPV F protein antigen is set forth as:

(SEQ ID NO: 40)
MSWKVVIIFSLLITPQHGLKESYLEESCSTITEGYLSVLRTGWYTNVFTL
EVGDVENLTCSDGPSLIKTELDLTKSALRELKTVSADQLAREEQIENPRQ
SRFVLGAIALGVATAAAVTAGVAIAKTIRLESEVTAIKNALKTTNEAVST
LGNGVRVLATAVRELKDFVSKNLTRAINKNKCDIpDLKMAVSFSQFNRRF
LNVVRQFSDNAGITPAISLDLMTDAELARAVSNMPTSAGQIKLMLENRAM
VRRKGFGILIGVYGSSVIYMVQLPIFGVIDTPCWIVKAAPSCSEKKGNYA
CLLREDQGWYCQNAGSTVYYPNEKDCETRGDHVFCDTAAGINVAEQSKEC
NINISTTNYPCKVSTGRHPISMVALSPLGALVACYKGVSCSIGSNRVGII
KQLNKGCSYITNQDADTVTIDNTVYQLSKVEGEQHVIKGRPVSSSFDPIK
FPEDQFNVALDQVFENIENSQALVDQSNRILSSAEKGNTGFIIVIILIAV
LGSSMILVSIFIIIKKTKKPTGAPPELSGVTNNGFIPHN
(A2-D185P).

In certain embodiments, the hMPV F protein antigen is encoded by an mRNA ORF set forth as (SEQ ID NO: 34) (A2-D185P mRNA ORF).

In certain embodiments, the hMPV F protein antigen is encoded by a codon-optimized mRNA ORF set forth as (SEQ ID NO: 36) (AD185P mRNA ORF).

In certain embodiments, the hMPV F protein antigen is set forth as:

(SEQ ID NO: 2)
MSWKVVIIFSLLITPQHGLKESYLEESCSTITEGYLSVLRTGWYTvVFTL
EVGDVENLTCSDGPSLIKTELDLTKSALRELKTVSADQLAREEQIENPRQ
SRFVLGAIALGVATAAAVTAGVAIAKTIRLESEVTAIKNALKTTNEAVST
LGNGVRVLAFAVRELKDFVSKNLTRAINKNKCDIDDLKMAVSFSQFNRRF
LNVVRQFSDNAGITPAISLDLMTDAELARAVSNMPTSAGQIKLMLENRAM
VRRKGFGILIGVYGSSVIYMVQLPIFGVIDTPCWIVKAAPSCSEKKGNYA
CLLREDQGWYCQNAGSTVYYPNEKDCETRGDHVFCDTAAGINVAEQSKEC
NINISTTNYPCKVSTGRHPISMVALSPLGALVACYKGVSCSIGSNRVGII
KQLNKGCSYITNQDADTVTIDNTVYQLSKVEGEQHVIKGRPVSSSFDPIK
FPEDQFNVALDQVFENIENSQALVDQSNRILSSAEKGNTGFIIVIILIAV
LGSSMILVSIFIIIKKTKKPTGAPPELSGVTNNGFIPHN
(A2-T160F_N46V).

In certain embodiments, the hMPV F protein antigen is encoded by a codon-optimized mRNA ORF set forth as

(SEQ ID NO: 5)
AUGAGCUGGAAGGUUGUGAUUAUUUUCUCUCUGCUGAUUACUCCACAGCACGGCCUGAAG
GAGUCCUACCUGGAGGAGUCCUGUUCUACUAUCACUGAGGGGUAUCUCUCUGUGCUGCGG
ACAGGGUGGUAUACAGUGGUGUUCACCCUGGAGGUUGGCGAUGUGGAGAAUCUGACUUGC
AGCGAUGGCCCUUCUCUGAUCAAGACCGAGCUGGAUCUGACAAAAAGCGCCCUCAGAGAAC
UGAAAACCGUGUCCGCCGAUCAGCUGGCAAGGGAGGAGCAGAUCGAGAACCCACGGCAGA
GCAGGUUUGUGCUGGGCGCUAUCGCUCUGGGCGUGGCCACUGCAGCUGCUGUCACUGCA
GGGGUCGCAAUCGCUAAGACUAUCAGACUGGAAUCCGAGGUGACCGCCAUUAAGAAUGCCC
UGAAGACUACCAACGAGGCUGUGUCCACUCUGGGAAACGGAGUGAGGGUCCUGGCCUUCG
CAGUGAGGGAGCUGAAGGAUUUUGUGUCAAAGAACCUUACACGGGCCAUCAACAAGAAUAA
GUGCGAUAUCGAUGACCUGAAGAUGGCCGUGUCCUUCUCCCAGUUCAACCGGCGCUUUCU
GAAUGUGGUGCGCCAGUUUUCCGACAACGCUGGAAUCACCCCUGCUAUCAGCCUGGACCU
CAUGACCGACGCCGAACUCGCAAGGGCCGUUUCUAACAUGCCUACAUCCGCUGGACAGAUU
AAGCUGAUGCUGGAGAAUAGAGCAAUGGUGAGGAGAAAGGGAUUCGGCAUCCUGAUUGGC
GUGUACGGAUCUAGCGUGAUCUACAUGGUGCAGCUGCCGAUCUUCGGCGUGAUCGAUACU
CCUUGUUGGAUCGUCAAGGCCGCCCCUUCCUGCUCCGAGAAGAAGGGCAAUUACGCUUGU
CUGCUGCGGGAGGACCAGGGCUGGUAUUGCCAGAACGCCGGGUCUACAGUGUACUAUCCU
AACGAGAAGGAUUGCGAGACCAGAGGCGACCACGUUUUCUGUGAUACAGCCGCCGGAAUCA
AUGUCGCAGAGCAGUCUAAGGAGUGCAACAUCAAUAUCUCUACAACCAAUUACCCAUGUAAG
GUGAGCACUGGACGGCACCCUAUCAGUAUGGUGGCUCUGAGCCCACUGGGGGCACUGGUG
GCUUGCUACAAGGGGGUGAGCUGCAGUAUCGGCAGUAACAGAGUGGGCAUUAUCAAGCAG
CUGAACAAAGGGUGCUCUUAUAUUACAAACCAGGAUGCAGAUACUGUGACCAUCGACAACA
CUGUGUACCAGCUGUCCAAGGUGGAGGGGGAGCAGCAUGUGAUCAAAGGGAGACCCGUCU
CUUCUUCUUUCGAUCCCAUCAAGUUCCCUGAAGACCAGUUCAAUGUUGCCCUGGACCAGGU
UUUCGAGAACAUCGAAAAUAGCCAGGCCUUGGUCGAUCAAUCCAACAGGAUCCUGAGCAGC
GCAGAGAAAGGGAACACUGGCUUCAUCAUCGUGAUCAUUCUGAUCGCCGUGCUGGGGAGC
AGUAUGAUUCUGGUGUCCAUUUUCAUCAUCAUCAAGAAGACCAAGAAGCCUACAGGAGCAC
CCCCUGAGCUGAGCGGAGUGACCAACAACGGCUUUAUCCCUCACAACUAA

In certain embodiments, the hMPV F protein antigen is encoded by an mRNA ORF set forth as (SEQ ID NO: 37) (A2-T160F_N46V mRNA ORF).

In certain embodiments, the hMPV F protein antigen is encoded by a codon-optimized mRNA ORF set forth as (SEQ ID NO: 38) (T160F_N46V mRNA ORF).

In certain embodiments, the hMPV F protein antigen is encoded by a codon-optimized mRNA ORF set forth as (SEQ ID NO: 39) (T160F_N46V mRNA ORF).

### V. hPIV3 F Protein

hPIV3 is an enveloped, non-segmented, negative-sense, single-stranded RNA virus belonging to the paramyxovirinae subfamily within the paramyxovirus family. It is a significant cause of childhood illness and hospitalization worldwide. The entry of hPIV3 requires the merger of viral and cellular membranes, which is catalyzed by the hPIV3 fusion (F) protein on the virion surface.

hPIV3 F is a class I fusion glycoprotein expressed as an inactive precursor, F0, that needs to be cleaved to become fusion competent. F0 undergoes proteolytic cleavage during transport through the Golgi apparatus, generating a disulfide-linked heterodimer of F2 and F1 polypeptides (F2 N-terminal to F1). Three protomers of the F2-F1 heterodimer assemble to form a mature F protein, which adopts a metastable “pre-fusion” or “pre-F” conformation. To initiate membrane fusion, hPIV3 F is activated and undergoes a series of stepwise conformational changes in the F protein that drive membrane fusion and result in hPIV3 F adopting a highly stable “post-fusion” or “post-F” conformation.

Provided herein are antigenic hPIV3 polypeptides comprising an hPIV3 F polypeptide. The hPIV3 F polypeptide may comprise the whole sequence of hPIV F or a portion of hPIV F. In certain embodiments, the portion is the ectodomain.

The hPIV3 F polypeptides described herein may have deletions or substitutions relative to the wild-type hPIV3 F protein set forth as:

(SEQ ID NO: 11)
MLISILSIITTMIMASHCQIDITKLQHVGVLVNSPKGMKISQNFETRYLI
LSLIPKIEDSNSCGDQQIKQYKRLLDRLIIPLYDGLKLQKDVIVTNQESN
ENTDPRTERFFGGVIGTIALGVATSAQITAAVALVEAKQAKSDIEKLKEA
IRDTNKAVQSVQSSVGNLIVAIKSVQDYVNKEIVPSIARLGCEAAGLQLG
IALTQHYSELTNIFGDNIGSLQEKGIKLQGIASLYRTNITEIFTTSTVDK
YDIYDLLFTESIKVRVIDVDLNDYSITLQVRLPLLTRLLNTQIYKVDSIS
YNIQNREWYIPLPSHIMTKGAFLGGADVKECIEAFSSYICPSDPGFVLNH
EMESCLSGNISQCPRTTVTSDIVPRYAFVNGGVVANCITTTCTCNGIGNR
INQPPDQGVKIITHKECNTIGINGMLFNTNKEGTLAFYTPDDITLNNSVA
LDPIDISIELNKAKSDLEESKEWIRRSNQKLDSIGSWHQSSTTIIIILIM
MIILFIINITIITIAIKYYRIQKRNRVDQNDKPYVLTNK

For example, in some embodiments, an hPIV3 F polypeptide comprises amino acid substitutions Q162C, L168C, I213C, G230C, A463V, and I474Y relative to SEQ ID NO: 11.

In some embodiments, the hPIV3 F ectodomain trimer comprises protomers comprising one or more amino acid substitutions or deletions that stabilize the hPIV3 ectodomain trimer in a prefusion conformation, wherein the one or more amino acid substitutions or deletions comprise one or more of the following sets of substitutions to form a disulfide bond to stabilize the hPIV3 ectodomain trimer in a prefusion conformation: 162C and 168C; 213C and 230C.

In some embodiments, the protomers of the hPIV3 F ectodomain trimer include hPIV3 F positions 19-481, an amino acid substitution (such as K108E) to remove the consensus furin cleavage site between F2 and F1 (if the consensus site is present in the native sequence), a non-native disulfide bond between 162C and 168C (such as Q162C and L168C) substitutions, and linkage to a C-terminal GCN4 trimerization domain (for example, via an SA peptide linker), that stabilize the hPIV3 F ectodomain trimer in a prefusion conformation. In some such embodiments, protomers further comprise 463V and 474Y (such as A463V and I474Y) cavity filling substitutions for stabilization in the prefusion conformation.

In some embodiments, the protomers of the hPIV3 F ectodomain trimer include hPIV3 F positions 19-481, an amino acid substitution (such as K108E) to remove the consensus furin cleavage site between F2 and F1 (if the consensus site is present in the native sequence), a non-native disulfide bond between 162C and 168C (such as Q162C and L168C) substitutions, a non-native disulfide bond between 213C and 230C (such as I213C and G230C) substitutions, and linkage to a C-terminal GCN4 trimerization domain (for example, via an SA peptide linker), that stabilize the hPIV3 F ectodomain trimer in a prefusion conformation. In some such embodiments, protomers further comprise 463V and 474Y (such as A463V and I474Y) cavity filling substitutions for stabilization in the prefusion conformation.

In some embodiments, the protomers of the hPIV3 F ectodomain trimer include a full-length hPIV3 F protein (minus the signal peptide) comprising an amino acid substitution (such as K108E) to remove the consensus furin cleavage site between F2 and F1 (if the consensus site is present in the native sequence), and a non-native disulfide bond between 162C and 168C (such as Q162C and L168C) substitutions that stabilize the hPIV3 F ectodomain trimer in a prefusion conformation. In some such embodiments, protomers further comprise 463V and 474Y (such as A463V and I474Y) cavity filling substitutions for stabilization in the prefusion conformation.

In some embodiments, the protomers of the hPIV3 F ectodomain trimer include a full-length hPIV3 F protein (minus the signal peptide) comprising an amino acid substitution (such as K108E) to remove the consensus furin cleavage site between F2 and F1 (if the consensus site is present in the native sequence), a non-native disulfide bond between 162C and 168C (such as Q162C and L168C) substitutions, and a non-native disulfide bond between 213C and 230C (such as I213C and G230C) substitutions that stabilize the hPIV3 F ectodomain trimer in a prefusion conformation. In some such embodiments, protomers further comprise 463V and 474Y (such as A463V and I474Y) cavity filling substitutions for stabilization in the prefusion conformation.

In some embodiments, an amino acid sequence comprising an hPIV3 F polypeptide sequence is provided, set forth as:

(SEQ ID NO: 3)
MLISILSIITTMIMASHCQIDITKLQHVGVLVNSPKGMKISQNFETRYLI
LSLIPKIEDSNSCGDQQIKQYKRLLDRLIIPLYDGLKLQKDVIVTNQESN
ENTDPRTERFFGGVIGTIALGVATSAQITAAVALVEAKQAKSDIEKLKEA
IRDTNKAVQSVCSSVGNCIVAIKSVQDYVNKEIVPSIARLGCEAAGLQLG
IALTQHYSELTNCFGDNIGSLQEKGIKLQCIASLYRTNITEIFTTSTVDK
YDIYDLLFTESIKVRVIDVDLNDYSITLQVRLPLLTRLLNTQIYKVDSIS
YNIQNREWYIPLPSHIMTKGAFLGGADVKECIEAFSSYICPSDPGFVLNH
EMESCLSGNISQCPRTTVTSDIVPRYAFVNGGVVANCITTTCTCNGIGNR
INQPPDQGVKIITHKECNTIGINGMLFNTNKEGTLAFYTPDDITLNNSVA
LDPIDISIELNKVKSDLEESKEWYRRSNQKLDSIGSWHQSSTTIIIILIM
MIILFIINITIITIAIKYYRIQKRNRVDQNDKPYVLTNK

In some embodiments, a nucleotide sequence encoding a codon-optimized hPIV3 F polypeptide sequence is provided, set forth as:

(SEQ ID NO: 14)
ATGCTGATTAGCATTCTGTCCATCATCACAACCATGATTATGGCAAGTCACTGTCAGATCGATA
TCACCAAGCTCCAGCACGTGGGCGTGCTGGTGAATTCCCCAAAGGGGATGAAGATTTCTCAGA
ACTTTGAGACTCGGTACCTGATCCTGTCCCTGATTCCTAAGATTGAGGATAGCAACTCCTGTGG
CGACCAGCAGATCAAACAGTACAAACGCCTGCTGGACAGACTGATCATCCCACTGTACGACGG
CCTGAAGCTGCAGAAAGACGTCATTGTTACAAACCAGGAAAGCAACGAGAATACCGACCCTAG
GACTGAGCGGTTCTTCGGGGGCGTCATCGGAACAATCGCACTGGGCGTTGCCACTAGCGCAC
AGATCACCGCCGCCGTTGCACTGGTGGAGGCTAAGCAGGCAAAATCTGACATCGAGAAGCTG
AAAGAAGCTATCCGCGATACCAACAAGGCCGTGCAGTCAGTGTGCTCCAGCGTCGGCAACTG
CATCGTTGCTATTAAGTCCGTGCAGGACTACGTGAACAAGGAGATCGTGCCCAGTATCGCAAG
GCTGGGATGTGAGGCCGCTGGACTGCAGTTGGGGATCGCTCTCACCCAGCACTACTCCGAGC
TGACAAATTGCTTCGGAGATAACATCGGATCTCTGCAGGAGAAGGGCATCAAACTGCAGTGTA
TTGCCTCCCTGTACAGAACAAATATCACAGAGATTTTCACAACCAGCACTGTCGACAAATATGA
TATCTATGACCTGCTGTTCACCGAGTCCATTAAGGTGCGGGTCATCGACGTTGACCTGAACGA
TTATTCCATCACCCTCCAGGTGAGACTGCCTCTGCTGACACGGCTGCTGAACACTCAGATCTA
CAAGGTCGACTCCATCAGCTACAACATCCAGAACCGCGAGTGGTACATCCCACTGCCTAGCCA
CATCATGACAAAGGGCGCTTTTCTGGGAGGAGCTGATGTGAAGGAATGCATTGAGGCCTTCAG
CAGCTACATCTGCCCTTCCGATCCCGGCTTCGTCCTGAACCACGAGATGGAGAGCTGTCTGAG
CGGGAACATTAGCCAGTGCCCCAGGACAACCGTCACCTCTGACATTGTGCCAAGGTATGCCTT
CGTGAACGGCGGAGTGGTTGCCAATTGCATCACCACTACATGCACTTGCAATGGGATTGGCAA
CAGGATCAACCAGCCACCAGACCAGGGGGTGAAAATCATTACACACAAAGAGTGCAACACCAT
CGGCATCAACGGAATGCTGTTCAATACCAACAAGGAGGGCACCCTGGCCTTCTACACACCCGA
TGACATCACCCTGAACAATAGCGTGGCTCTGGATCCCATTGATATCAGCATTGAGCTGAATAAG
GTCAAGTCTGATCTGGAGGAGAGCAAGGAGTGGTATAGACGGTCTAATCAGAAGCTCGACTCT
ATCGGATCCTGGCACCAGTCCTCCACAACAATCATCATTATCCTGATCATGATGATCATCCTGT
TCATCATCAACATCACAATTATCACCATTGCCATCAAGTATTACCGGATTCAGAAGCGGAATCG
CGTTGACCAGAACGACAAACCTTATGTTCTGACCAACAAATAA

In some embodiments, the hPIV3 F polypeptide comprises a sequence having at least 80%, 85%, 90%, 91%, 92%, 93%, 94%, 95%, 96%, 97%, 98%, or 99.5% identity to any one of SEQ ID NOs: 3 and 11.

In some embodiments, the hPIV3 F polypeptide comprises a modified hPIV3 F polypeptide having at least 80%, 85%, 90%, 91%, 92%, 93%, 94%, 95%, 96%, 97%, 98%, or 99.5% identity to the polypeptides of any one of SEQ ID NOs: 3 and 11, wherein the hPIV3 F polypeptide is antigenic.

In some embodiments, the hPIV3 F polypeptide comprises only the ectodomain portion of the F protein.

Provided herein are RNAs (e.g., mRNAs) that encode for antigenic hPIV3 F polypeptides.

In one aspect, the disclosure provides an hPIV3 vaccine comprising a mRNA comprising an open reading frame (ORF) encoding an hPIV3 F protein antigen, wherein the hPIV3 F protein antigen comprises an amino acid sequence with at least 80%, 85%, 90%, 91%, 92%, 93%, 94%, 95%, 96%, 97%, 98%, or 99.5% identity to SEQ ID NO: 3 or consists of an amino acid sequence of SEQ ID NO: 3.

In general, positions in constructs described herein can be mapped onto the wild-type sequence of SEQ ID NO: 11 by pairwise alignment, e.g., using the Needleman-Wunsch algorithm with standard parameters (EBLOSUM62 matrix, Gap penalty 10, gap extension penalty 0.5). See also the discussion of structural alignment provided herein as an alternative approach for identifying corresponding positions.

In some embodiments the hPIV3 F protein antigen is encoded by a codon-optimized mRNA ORF set forth as:

(SEQ ID NO: 6)
AUGCUGAUUAGCAUUCUGUCCAUCAUCACAACCAUGAUUAUGGCAAGUCACUGUCAGAUCG
AUAUCACCAAGCUCCAGCACGUGGGCGUGCUGGUGAAUUCCCCAAAGGGGAUGAAGAUUUC
UCAGAACUUUGAGACUCGGUACCUGAUCCUGUCCCUGAUUCCUAAGAUUGAGGAUAGCAAC
UCCUGUGGCGACCAGCAGAUCAAACAGUACAAACGCCUGCUGGACAGACUGAUCAUCCCAC
UGUACGACGGCCUGAAGCUGCAGAAAGACGUCAUUGUUACAAACCAGGAAAGCAACGAGAA
UACCGACCCUAGGACUGAGCGGUUCUUCGGGGGCGUCAUCGGAACAAUCGCACUGGGCGU
UGCCACUAGCGCACAGAUCACCGCCGCCGUUGCACUGGUGGAGGCUAAGCAGGCAAAAUCU
GACAUCGAGAAGCUGAAAGAAGCUAUCCGCGAUACCAACAAGGCCGUGCAGUCAGUGUGCU
CCAGCGUCGGCAACUGCAUCGUUGCUAUUAAGUCCGUGCAGGACUACGUGAACAAGGAGAU
CGUGCCCAGUAUCGCAAGGCUGGGAUGUGAGGCCGCUGGACUGCAGUUGGGGAUCGCUCU
CACCCAGCACUACUCCGAGCUGACAAAUUGCUUCGGAGAUAACAUCGGAUCUCUGCAGGAG
AAGGGCAUCAAACUGCAGUGUAUUGCCUCCCUGUACAGAACAAAUAUCACAGAGAUUUUCA
CAACCAGCACUGUCGACAAAUAUGAUAUCUAUGACCUGCUGUUCACCGAGUCCAUUAAGGU
GCGGGUCAUCGACGUUGACCUGAACGAUUAUUCCAUCACCCUCCAGGUGAGACUGCCUCU
GCUGACACGGCUGCUGAACACUCAGAUCUACAAGGUCGACUCCAUCAGCUACAACAUCCAG
AACCGCGAGUGGUACAUCCCACUGCCUAGCCACAUCAUGACAAAGGGCGCUUUUCUGGGAG
GAGCUGAUGUGAAGGAAUGCAUUGAGGCCUUCAGCAGCUACAUCUGCCCUUCCGAUCCCG
GCUUCGUCCUGAACCACGAGAUGGAGAGCUGUCUGAGCGGGAACAUUAGCCAGUGCCCCA
GGACAACCGUCACCUCUGACAUUGUGCCAAGGUAUGCCUUCGUGAACGGCGGAGUGGUUG
CCAAUUGCAUCACCACUACAUGCACUUGCAAUGGGAUUGGCAACAGGAUCAACCAGCCACC
AGACCAGGGGGUGAAAAUCAUUACACACAAAGAGUGCAACACCAUCGGCAUCAACGGAAUG
CUGUUCAAUACCAACAAGGAGGGCACCCUGGCCUUCUACACACCCGAUGACAUCACCCUGA
ACAAUAGCGUGGCUCUGGAUCCCAUUGAUAUCAGCAUUGAGCUGAAUAAGGUCAAGUCUGA
UCUGGAGGAGAGCAAGGAGUGGUAUAGACGGUCUAAUCAGAAGCUCGACUCUAUCGGAUCC
UGGCACCAGUCCUCCACAACAAUCAUCAUUAUCCUGAUCAUGAUGAUCAUCCUGUUCAUCA
UCAACAUCACAAUUAUCACCAUUGCCAUCAAGUAUUACCGGAUUCAGAAGCGGAAUCGCGU
UGACCAGAACGACAAACCUUAUGUUCUGACCAACAAAUAA

Recombinant hPIV3 F polypeptides are described in further detail in U.S. Pat. No. 11,078,239, which is incorporated herein by reference.

### VI. Combination Respiratory mRNA Vaccine Compositions

Provided herein are compositions that target two or more of the three most common respiratory pathogens associated with high morbidity and mortality in infants, children, and older adults: hRSV, hMPV, and hPIV3. These respiratory pathogens have a combined disease burden outranking influenza. With no vaccines or specific therapies currently available, there exists an unmet need for a safe, effective, and convenient mRNA combination vaccine to protect individuals against these pathogens.

In some embodiments, the composition comprises at least two mRNAs, wherein the at least two mRNAs comprise an open reading frame (ORF) encoding a recombinant F protein antigenic polypeptide selected from the group consisting of: (i) a first mRNA encoding an hRSV F protein antigen comprising an amino acid sequence of SEQ ID NO: 1; (ii) a second mRNA encoding an hMPV F protein antigen comprising an amino acid sequence of SEQ ID NO: 2; and (iii) a third mRNA encoding an hPIV3 F protein antigen comprising an amino acid sequence of SEQ ID NO: 3.

In some embodiments, the composition above comprises at least two mRNAs, wherein the at least two mRNAs comprise an ORF encoding a recombinant F protein antigenic polypeptide comprising: (i) a first mRNA encoding an hRSV F protein antigen comprising an amino acid sequence of SEQ ID NO: 1; and (ii) a second mRNA encoding an hMPV F protein antigen comprising an amino acid sequence of SEQ ID NO: 2.

In some embodiments, the composition comprises at least two mRNAs, wherein the at least two mRNAs comprise an ORF encoding a recombinant F protein antigenic polypeptide comprising: (i) a first mRNA encoding an hRSV F protein antigen comprising an amino acid sequence of SEQ ID NO: 1; and (ii) a second mRNA encoding an hPIV3 F protein antigen comprising an amino acid sequence of SEQ ID NO: 3.

In some embodiments, the composition comprises at least two mRNAs, wherein the at least two mRNAs comprise an ORF encoding a recombinant F protein antigenic polypeptide comprising: (i) a first mRNA encoding an hMPV F protein antigen comprising an amino acid sequence of SEQ ID NO: 2; and (ii) a second mRNA encoding an hPIV3 F protein antigen comprising an amino acid sequence of SEQ ID NO: 3.

In some embodiments, the composition comprises at least three mRNAs, wherein the at least three mRNAs comprise an ORF encoding a recombinant F protein antigenic polypeptide comprising: (i) a first mRNA encoding an hRSV F protein antigen comprising an amino acid sequence of SEQ ID NO: 1; and (ii) a second mRNA encoding an hMPV F protein antigen comprising an amino acid sequence of SEQ ID NO: 2; and (ii) a second mRNA encoding an hPIV3 F protein antigen comprising an amino acid sequence of SEQ ID NO: 3.

In some embodiments, the composition comprises three mRNAs, wherein the three mRNAs comprise an ORF encoding a recombinant F protein antigenic polypeptide including: (i) a first mRNA encoding an hRSV F protein antigen comprising an amino acid sequence of SEQ ID NO: 1; and (ii) a second mRNA encoding an hMPV F protein antigen comprising an amino acid sequence of SEQ ID NO: 2; and (ii) a second mRNA encoding an hPIV3 F protein antigen comprising an amino acid sequence of SEQ ID NO: 3.

In some embodiments, the composition comprises three mRNAs, wherein: (i) a first mRNA encodes an hRSV F protein antigen comprising an amino acid sequence of SEQ ID NO: 1; (ii) a second mRNA encodes an hMPV F protein antigen comprising an amino acid sequence of SEQ ID NO: 2; and (iii) a third mRNA encodes an hPIV3 F protein antigen comprising an amino acid sequence of SEQ ID NO: 3.

In some embodiments, the composition comprises three mRNAs, wherein: (i) a first mRNA encodes an hRSV F protein antigen comprising an amino acid sequence of SEQ ID NO: 1; (ii) a second mRNA encodes an hMPV F protein antigen comprising an amino acid sequence of SEQ ID NO: 2; and (iii) a third mRNA encodes an hPIV3 F protein antigen comprising an amino acid sequence of SEQ ID NO: 3, wherein the first mRNA, the second mRNA, and the third mRNA are formulated into the same LNP comprising: OF-02, cKK-E10, GL-HEPES-E3-E10-DS-3-E18-1, GL-HEPES-E3-E12-DS-4-E10, GL-HEPES-E3-E12-DS-3-E14, or IM-001 at a molar ratio of 40%; DMG-PEG2000 at a molar ratio of 1.5%; cholesterol at a molar ratio of 28.5%; and DOPE at a molar ratio of 30%.

In some embodiments, the composition comprises a first mRNA and a second mRNA, wherein: (i) the first mRNA encodes an hRSV F protein antigen comprising an amino acid sequence of SEQ ID NO: 1; and (ii) the second mRNA encodes an hMPV F protein antigen comprising an amino acid sequence of SEQ ID NO: 2, wherein the first mRNA and the second mRNA are formulated into the same LNP comprising: OF-02, cKK-E10, GL-HEPES-E3-E10-DS-3-E18-1, GL-HEPES-E3-E12-DS-4-E10, GL-HEPES-E3-E12-DS-3-E14, or IM-001. This composition may be administered with a separate composition comprising an hPIV3 F protein antigen. The hPIV3 F protein antigen may comprise an amino acid sequence of SEQ ID NO: 3.

In some embodiments, the composition comprises a first mRNA and a second mRNA, wherein: (i) the first mRNA encodes an hRSV F protein antigen comprising an amino acid sequence of SEQ ID NO: 1; and (ii) the second mRNA encodes an hMPV F protein antigen comprising an amino acid sequence of SEQ ID NO: 2, wherein the first mRNA and the second mRNA are formulated into a separate LNP comprising: OF-02, cKK-E10, GL-HEPES-E3-E10-DS-3-E18-1, GL-HEPES-E3-E12-DS-4-E10, GL-HEPES-E3-E12-DS-3-E14, or IM-001. This composition may be administered with a separate composition comprising an hPIV3 F protein antigen. The hPIV3 F protein antigen may comprise an amino acid sequence of SEQ ID NO: 3.

In some embodiments, the composition comprises a first mRNA and a second mRNA, wherein: (i) the first mRNA encodes an hRSV F protein antigen comprising an amino acid sequence of SEQ ID NO: 1; and (ii) the second mRNA encodes an hMPV F protein antigen comprising an amino acid sequence of SEQ ID NO: 2, wherein the first mRNA and the second mRNA are formulated into the same LNP comprising: OF-02, cKK-E10, GL-HEPES-E3-E10-DS-3-E18-1, GL-HEPES-E3-E12-DS-4-E10, GL-HEPES-E3-E12-DS-3-E14, or IM-001 at a molar ratio of 40%; DMG-PEG2000 at a molar ratio of 1.5%; cholesterol at a molar ratio of 28.5%; and DOPE at a molar ratio of 30%. This composition may be administered with a separate composition comprising an hPIV3 F protein antigen. The hPIV3 F protein antigen may comprise an amino acid sequence of SEQ ID NO: 3.

In some embodiments, the composition comprises a first mRNA and a second mRNA, wherein: (i) the first mRNA encodes an hRSV F protein antigen comprising an amino acid sequence of SEQ ID NO: 1; and (ii) the second mRNA encodes an hMPV F protein antigen comprising an amino acid sequence of SEQ ID NO: 2, wherein the first mRNA and the second mRNA are formulated into a separate LNP comprising: OF-02, cKK-E10, GL-HEPES-E3-E10-DS-3-E18-1, GL-HEPES-E3-E12-DS-4-E10, GL-HEPES-E3-E12-DS-3-E14, or IM-001 at a molar ratio of 40%; DMG-PEG2000 at a molar ratio of 1.5%; cholesterol at a molar ratio of 28.5%; and DOPE at a molar ratio of 30%. This composition may be administered with a separate composition comprising an hPIV3 F protein antigen. The hPIV3 F protein antigen may comprise an amino acid sequence of SEQ ID NO: 3.

In some embodiments, the composition comprises a first mRNA and a second mRNA, wherein: (i) the first mRNA encodes an hRSV F protein antigen comprising an amino acid sequence of SEQ ID NO: 1; and (ii) the second mRNA encodes an hMPV F protein antigen comprising an amino acid sequence of SEQ ID NO: 2, wherein the first mRNA and the second mRNA are formulated into the same LNP comprising GL-HEPES-E3-E12-DS-4-E10. This composition may be administered with a separate composition comprising an hPIV3 F protein antigen. The hPIV3 F protein antigen may comprise an amino acid sequence of SEQ ID NO: 3.

In some embodiments, the composition comprises a first mRNA and a second mRNA, wherein: (i) the first mRNA encodes an hRSV F protein antigen comprising an amino acid sequence of SEQ ID NO: 1; and (ii) the second mRNA encodes an hMPV F protein antigen comprising an amino acid sequence of SEQ ID NO: 2, wherein the first mRNA and the second mRNA are formulated into a separate LNP comprising GL-HEPES-E3-E12-DS-4-E10. This composition may be administered with a separate composition comprising an hPIV3 F protein antigen. The hPIV3 F protein antigen may comprise an amino acid sequence of SEQ ID NO: 3.

The compositions in any of the above embodiments may comprise hRSV F protein with an amino acid sequence having at least 90%, 91%, 92%, 93%, 94%, 95%, 96%, 97%, 98%, 99%, or 100% identity to SEQ ID NO: 1.

The compositions in any of the above embodiments may comprise an hMPV F protein with an amino acid sequence having at least 90%, 91%, 92%, 93%, 94%, 95%, 96%, 97%, 98%, 99%, or 100% identity to SEQ ID NO: 2.

The compositions in any of the above embodiments may comprise an hPIV3 F protein with an amino acid sequence having at least 90%, 91%, 92%, 93%, 94%, 95%, 96%, 97%, 98%, 99%, or 100% identity to SEQ ID NO: 3.

The compositions in any of the above embodiments may comprise a mRNA encoding an hRSV F protein antigen with a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 4.

The compositions in any of the above embodiments may comprise a mRNA encoding an hMPV F protein antigen with a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 5.

The compositions in any of the above embodiments may comprise a mRNA encoding an hPIV3 F protein antigen with a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 6.

The compositions in any of the above embodiments may comprise at least one F protein antigenic polypeptide that is a pre-fusion protein.

The compositions in any of the above embodiments may comprise at least one mRNA ORF that is codon-optimized.

The compositions in any of the above embodiments may comprise at least one mRNA comprising at least one 5′ untranslated region (5′ UTR), at least one 3′ untranslated region (3′ UTR), and at least one polyadenylation (poly(A)) sequence.

The compositions in any of the above embodiments may comprise at least one mRNA comprising a 5′ UTR with a nucleic acid sequence having at least 80% identity to SEQ ID NO: 7.

The compositions in any of the above embodiments may comprise at least one mRNA comprising a 3′ UTR with a nucleic acid sequence having at least 80% identity to SEQ ID NO: 8.

The compositions in any of the above embodiments may comprise at least one mRNA having at least one chemical modification.

The compositions in any of the above embodiments may comprise at least 20%, at least 30%, at least 40%, at least 50%, at least 60%, at least 70%, at least 80%, at least 85%, at least 90%, at least 95%, or 100% of the uracil nucleotides in at least one of the mRNAs are chemically modified.

The compositions in any of the above embodiments may comprise at least 30%, at least 40%, at least 50%, at least 60%, at least 70%, at least 80%, at least 85%, at least 90%, at least 95%, or 100% of the uracil nucleotides in at least one of the ORFs are chemically modified.

The compositions in any of the above embodiments may comprise chemical modifications selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

The compositions in any of the above embodiments may comprise chemical modifications selected from the group consisting of pseudouridine, N1-methylpseudouridine, 5-methylcytosine, 5-methoxyuridine, and a combination thereof.

The composition in any of the above embodiments may comprise the chemical modification N1-methylpseudouridine.

The compositions in any of the above embodiments may comprise mRNA encoding an hRSV F protein antigen, mRNA encoding an hMPV F protein antigen, and mRNA encoding an hPIV3 F protein antigen that are not covalently linked to one another.

The compositions in any of the above embodiments may comprise mRNA encoding an hRSV F protein antigen, mRNA encoding an hMPV F protein antigen, and/or mRNA encoding an hPIV3 F protein antigen that are covalently linked to one another.

The compositions in any of the above embodiments may comprise mRNA encoding an hRSV F protein antigen, mRNA encoding an hMPV F protein antigen, and mRNA encoding an hPIV3 F protein antigen each formulated into a separate lipid nanoparticle (LNP).

The compositions in any of the above embodiments may comprise mRNA encoding an hRSV F protein antigen, mRNA encoding an hMPV F protein antigen, and mRNA encoding an hPIV3 F protein antigen formulated into the same LNP.

The compositions in any of the above embodiments may comprise an LNP, wherein the LNP comprises at least one cationic lipid.

The compositions in any of the above embodiments may comprise an LNP, wherein the cationic lipid is biodegradable.

The compositions in any of the above embodiments may comprise an LNP, wherein the cationic lipid is not biodegradable.

The compositions in any of the above embodiments may comprise an LNP, wherein the cationic lipid is cleavable.

The compositions in any of the above embodiments may comprise an LNP, wherein the cationic lipid is not cleavable.

The compositions in any of the above embodiments may comprise an LNP, wherein the cationic lipid is selected from the group consisting of OF-02, cKK-E10, GL-HEPES-E3-E10-DS-3-E18-1, GL-HEPES-E3-E12-DS-4-E10, GL-HEPES-E3-E12-DS-3-E14, and IM-001.

The compositions in any of the above embodiments may comprise an LNP, wherein the cationic lipid is cKK-E10.

The compositions in any of the above embodiments may comprise an LNP, wherein the cationic lipid is GL-HEPES-E3-E12-DS-4-E10.

The compositions in any of the above embodiments may comprise an LNP, wherein the cationic lipid is IM-001.

The compositions in any of the above embodiments may comprise an LNP, wherein the LNP further comprises polyethylene glycol (PEG) conjugated (PEGylated) lipid, a cholesterol-based lipid, and a helper lipid.

The compositions in any of the above embodiments may comprise an LNP, wherein the LNP is comprised of: a cationic lipid at a molar ratio of 35% to 55%; a polyethylene glycol (PEG) conjugated (PEGylated) lipid at a molar ratio of 0.25% to 2.75%; a cholesterol-based lipid at a molar ratio of 20% to 45%; and a helper lipid at a molar ratio of 5% to 35%, wherein all of the molar ratios are relative to the total lipid content of the LNP.

The compositions in any of the above embodiments may comprise an LNP, wherein the LNP is comprised of: a cationic lipid at a molar ratio of 40%; a PEGylated lipid at a molar ratio of 1.5%; a cholesterol-based lipid at a molar ratio of 28.5%; and a helper lipid at a molar ratio of 30%, wherein all of the molar ratios are relative to the total lipid content of the LNP.

The compositions in any of the above embodiments may comprise an LNP, wherein the PEGylated lipid is dimyristoyl-PEG2000 (DMG-PEG2000) or 2-[(polyethylene glycol)-2000]-N,N-ditetradecylacetamide (ALC-0159).

The compositions in any of the above embodiments may comprise an LNP, wherein the cholesterol-based lipid is cholesterol.

The compositions in any of the above embodiments may comprise an LNP, wherein the helper lipid is 1,2-dioleoyl-SN-glycero-3-phosphoethanolamine (DOPE) or 1,2-distearoyl-sn-glycero-3-phosphocholine (DSPC).

The compositions in any of the above embodiments may comprise an LNP, wherein the LNP is comprised of: GL-HEPES-E3-E12-DS-4-E10 at a molar ratio of 40%; DMG-PEG2000 at a molar ratio of 1.5%; cholesterol at a molar ratio of 28.5%; and DOPE at a molar ratio of 30%, wherein all of the molar ratios are relative to the total lipid content of the LNP.

The compositions in any of the above embodiments may comprise an LNP, wherein the LNP is comprised of: cKK-E10 at a molar ratio of 40%; DMG-PEG2000 at a molar ratio of 1.5%; cholesterol at a molar ratio of 28.5%; and DOPE at a molar ratio of 30%, wherein all of the molar ratios are relative to the total lipid content of the LNP.

The compositions in any of the above embodiments may comprise an LNP, wherein the LNP is comprised of: IM-001 at a molar ratio of 40%; DMG-PEG2000 at a molar ratio of 1.5%; cholesterol at a molar ratio of 28.5%; and DOPE at a molar ratio of 30%, wherein all of the molar ratios are relative to the total lipid content of the LNP.

The compositions in any of the above embodiments may comprise an LNP, wherein the LNP has an average diameter of 30 nm to 200 nm.

The compositions in any of the above embodiments may comprise an LNP, wherein the LNP has an average diameter of 80 nm to 150 nm.

Section VII of the specification further describes LNPs that can be formulated with the combination respiratory mRNA compositions discussed above and is incorporated herein.

VI.A. Ratios of hRSV, hMPV, and hPIV3 mRNA

As discussed in the Examples, the compatibility of pre-fusion hRSV mRNA, pre-fusion hMPV mRNA, and pre-fusion hPIV3 mRNA was investigated in vitro and in vivo. Studies demonstrated that when pre-fusion hRSV mRNA, pre-fusion hMPV mRNA, and pre-fusion hPIV3 mRNA were co-formulated or co-administered at a 1:1:1 ratio, there was suppression of hMPV protein expression in vitro and of hMPV immunogenicity in mice relative to the monovalent hMPV immunization. It was determined that interference among components in the compositions may have occurred at the level of mRNA expression with no immunodominance component.

To determine if inhibition of hMPV immunogenicity could be overcome with dose adjustments by altering the ratio of the pre-fusion hRSV, hMPV, and hPIV3 mRNAs in co-formulation and co-administration, a variety of hRSV, hMPV, and hPIV3 ratios were explored. The co-formulation and co-administration studies both demonstrated that increasing hMPV mRNA doses overcomes interference without negatively effecting hRSV or hPIV3 immunogenicity, whereas decreasing hPIV3 mRNA doses did not overcome interference.

Accordingly, the ratio of hRSV mRNA, hMPV mRNA, and/or hPIV3 mRNA in combination vaccines is important for vaccine effectiveness.

The compositions in any of the above embodiments may comprise an hRSV:hMPV:hPIV3 mRNA ratio (w/w) of about 1:1:1, 1:1.1:1, 1:1.2:1, 1:1.3:1, 1:1.4:1, 1:1.5:1, 1:1.6:1, 1:1.7:1, 1:1.8:1, 1:1.9:1, 1:2:1, 1:2.1:1, 1:2.2:1, 1:2.3:1, 1:2.4:1, 1:2.5:1, 1:2.6:1, 1:2.7:1, 1:2.8:1, 1:2.9:1, 1:3:1, 1:3.1:1, 1:3.2:1, 1:3.3:1, 1:3.4:1, 1:3.5:1, 1:3.6:1, 1:3.7:1, 1:3.8:1, 1:3.9:1, 1:4:1, 1:4.1:1, 1:4.2:1, 1:4.3:1, 1:4.4:1, 1:4.5:1, 1:4.6:1, 1:4.7:1, 1:4.8:1, 1:4.9:1, 1:5:1, 1:5.1:1, 1:5.2:1, 1:5.3:1, 1:5.4:1, 1:5.5:1, 1:5.6:1, 1:5.7:1, 1:5.8:1, 1:5.9:1, 1:6:1, 1:6.1:1, 1:6.2:1, 1:6.3:1, 1:6.4:1, 1:6.5:1, 1:6.6:1, 1:6.7:1, 1:6.8:1, 1:6.9:1, 1:7:1, 1:7.1:1, 1:7.2:1, 1:7.3:1, 1:7.4:1, 1:7.5:1, 1:7.6:1, 1:7.7:1, 1:7.8:1, 1:7.9:1, 1:8:1, 1:8.1:1, 1:8.2:1, 1:8.3:1, 1:8.4:1, 1:8.5:1, 1:8.6:1, 1:8.7:1, 1:8.8:1, 1:8.9:1, 1:9:1, 1:9.1:1, 1:9.2:1, 1:9.3:1, 1:9.4:1, 1:9.5:1, 1:9.6:1, 1:9.7:1, 1:9.8:1, 1:9.9:1, 1:10:1, 1:1:0.9, 1:1:0.8, 1:1:0.7, 1:1:0.6, 1:1:0.5, 1:1:0.4, 1:1:0.3, 1:1:0.2, 1:1:0.1.

The compositions in any of the above embodiments may comprise an hRSV:hMPV:hPIV3 mRNA ratio (w/w) of 1:1:1, 1:1.1:1, 1:1.2:1, 1:1.3:1, 1:1.4:1, 1:1.5:1, 1:1.6:1, 1:1.7:1, 1:1.8:1, 1:1.9:1, 1:2:1, 1:2.1:1, 1:2.2:1, 1:2.3:1, 1:2.4:1, 1:2.5:1, 1:2.6:1, 1:2.7:1, 1:2.8:1, 1:2.9:1, 1:3:1, 1:3.1:1, 1:3.2:1, 1:3.3:1, 1:3.4:1, 1:3.5:1, 1:3.6:1, 1:3.7:1, 1:3.8:1, 1:3.9:1, 1:4:1, 1:4.1:1, 1:4.2:1, 1:4.3:1, 1:4.4:1, 1:4.5:1, 1:4.6:1, 1:4.7:1, 1:4.8:1, 1:4.9:1, 1:5:1, 1:5.1:1, 1:5.2:1, 1:5.3:1, 1:5.4:1, 1:5.5:1, 1:5.6:1, 1:5.7:1, 1:5.8:1, 1:5.9:1, 1:6:1, 1:6.1:1, 1:6.2:1, 1:6.3:1, 1:6.4:1, 1:6.5:1, 1:6.6:1, 1:6.7:1, 1:6.8:1, 1:6.9:1, 1:7:1, 1:7.1:1, 1:7.2:1, 1:7.3:1, 1:7.4:1, 1:7.5:1, 1:7.6:1, 1:7.7:1, 1:7.8:1, 1:7.9:1, 1:8:1, 1:8.1:1, 1:8.2:1, 1:8.3:1, 1:8.4:1, 1:8.5:1, 1:8.6:1, 1:8.7:1, 1:8.8:1, 1:8.9:1, 1:9:1, 1:9.1:1, 1:9.2:1, 1:9.3:1, 1:9.4:1, 1:9.5:1, 1:9.6:1, 1:9.7:1, 1:9.8:1, 1:9.9:1, 1:10:1, 1:1:0.9, 1:1:0.8, 1:1:0.7, 1:1:0.6, 1:1:0.5, 1:1:0.4, 1:1:0.3, 1:1:0.2, 1:1:0.1.

The compositions in any of the above embodiments may comprise an hRSV:hMPV mRNA ratio (w/w) of about 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9, 1:10.

The compositions in any of the above embodiments may comprise an hRSV:hMPV mRNA ratio (w/w) of 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9, 1:10.

The compositions in any of the above embodiments may comprise an hMPV:hPIV3 mRNA ratio (w/w) of about 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, 1:0.9, 1:0.8, 1:0.7, 1:0.6, 1:0.5, 1:0.4, 1:0.3, 1:0.2, 1:0.1.

The compositions in any of the above embodiments may comprise an hMPV:hPIV3 mRNA ratio (w/w) of 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, 1:0.9, 1:0.8, 1:0.7, 1:0.6, 1:0.5, 1:0.4, 1:0.3, 1:0.2, 1:0.1.

The compositions in any of the above embodiments may comprise an hRSV:hMPV:hPIV3 mRNA ratio (w/w) of about 1:1:1 to about 1:10:1.

The compositions in any of the above embodiments may comprise an hRSV:hMPV:hPIV3 mRNA ratio that is expressed in micrograms (pg).

The composition in any of the above embodiments wherein the hRSV mRNA, the hMPV mRNA, and the hPIV3 mRNA are present in a ratio (w/w) of about one microgram of the hRSV mRNA to about one microgram of the hMPV mRNA to about one microgram of the hPIV3 mRNA.

The composition in any of the above embodiments wherein the hRSV mRNA, the hMPV mRNA, and the hPIV3 mRNA are present in a ratio (w/w) of about one microgram of the hRSV mRNA to about two micrograms of the hMPV mRNA to about one microgram of the hPIV3 mRNA.

The composition in any of the above embodiments wherein the hRSV mRNA, the hMPV mRNA, and the hPIV3 mRNA are present in a ratio (w/w) of about 1 microgram of the hRSV mRNA to about 3 micrograms of the hMPV mRNA to about 1 microgram of the hPIV3 mRNA.

The composition in any of the above embodiments wherein the hRSV mRNA, the hMPV mRNA, and the hPIV3 mRNA are present in a ratio (w/w) of about one microgram of the hRSV mRNA to about five micrograms of the hMPV mRNA to about one microgram of the hPIV3 mRNA.

The composition in any of the above embodiments wherein the hRSV mRNA, the hMPV mRNA, and the hPIV3 mRNA are present in a ratio (w/w) of about 0.5 microgram of the hRSV mRNA to about 0.5 microgram of the hMPV mRNA to about 0.1 microgram of the hPIV3 mRNA.

The composition in any of the above embodiments wherein the hRSV mRNA, the hMPV mRNA, and the hPIV3 mRNA are present in a ratio (w/w) of about 0.5 microgram of the hRSV mRNA to about 0.5 microgram of the hMPV mRNA to about 0.5 microgram of the hPIV3 mRNA.

The composition in any of the above embodiments wherein the hRSV mRNA, the hMPV mRNA, and the hPIV3 mRNA are present in a ratio (w/w) of about 0.5 microgram of the hRSV mRNA to about 1.5 micrograms of the hMPV mRNA to about 0.5 microgram of the hPIV3 mRNA.

The composition in any of the above embodiments wherein the hRSV mRNA, the hMPV mRNA, and the hPIV3 mRNA are present in a ratio (w/w) of about 0.5 microgram of the hRSV mRNA to about 1 microgram of the hMPV mRNA to about 0.5 microgram of the hPIV3 mRNA.

The composition in any of the above embodiments wherein the hRSV mRNA, the hMPV mRNA, and the hPIV3 mRNA are present in a ratio (w/w) of about 0.5 microgram of the hRSV mRNA to about 2.5 micrograms of the hMPV mRNA to about 0.5 microgram of the hPIV3 mRNA.

The composition in any of the above embodiments wherein the hRSV mRNA, the hMPV mRNA, and the hPIV3 mRNA are present in a ratio (w/w) of 1 microgram of the hRSV mRNA to 1 microgram of the hMPV mRNA to 1 microgram of the hPIV3 mRNA.

The composition in any of the above embodiments wherein the hRSV mRNA, the hMPV mRNA, and the hPIV3 mRNA are present in a ratio (w/w) of 1 microgram of the hRSV mRNA to 2 micrograms of the hMPV mRNA to 1 microgram of the hPIV3 mRNA.

The composition in any of the above embodiments wherein the hRSV mRNA, the hMPV mRNA, and the hPIV3 mRNA are present in a ratio (w/w) of 1 microgram of the hRSV mRNA to 3 micrograms of the hMPV mRNA to 1 microgram of the hPIV3 mRNA.

The composition in any of the above embodiments wherein the hRSV mRNA, the hMPV mRNA, and the hPIV3 mRNA are present in a ratio (w/w) of 1 microgram of the hRSV mRNA to 5 micrograms of the hMPV mRNA to 1 microgram of the hPIV3 mRNA.

The composition in any of the above embodiments wherein the hRSV mRNA, the hMPV mRNA, and the hPIV3 mRNA are present in a ratio (w/w) of 0.5 microgram of the hRSV mRNA to 0.5 microgram of the hMPV mRNA to 0.1 microgram of the hPIV3 mRNA.

The composition in any of the above embodiments wherein the hRSV mRNA, the hMPV mRNA, and the hPIV3 mRNA are present in a ratio (w/w) of 0.5 microgram of the hRSV mRNA to 0.5 microgram of the hMPV mRNA to 0.5 microgram of the hPIV3 mRNA.

The composition in any of the above embodiments wherein the hRSV mRNA, the hMPV mRNA, and the hPIV3 mRNA are present in a ratio (w/w) of 0.5 microgram of the hRSV mRNA to 1.5 micrograms of the hMPV mRNA to 0.5 microgram of the hPIV3 mRNA.

The composition in any of the above embodiments wherein the hRSV mRNA, the hMPV mRNA, and the hPIV3 mRNA are present in a ratio (w/w) of 0.5 microgram of the hRSV mRNA to 1 microgram of the hMPV mRNA to 0.5 microgram of the hPIV3 mRNA.

The composition in any of the above embodiments wherein the hRSV mRNA, the hMPV mRNA, and the hPIV3 mRNA are present in a ratio (w/w) of 0.5 microgram of the hRSV mRNA to 2.5 micrograms of the hMPV mRNA to 0.5 microgram of the hPIV3 mRNA.

The compositions in any of the above embodiments may comprise an hRSV:hMPV mRNA ratio (w/w) of about 1:1 to about 1:10.

The compositions in any of the above embodiments may comprise an hRSV:hMPV mRNA ratio that is expressed in micrograms (pg).

The composition in any of the above embodiments wherein the hRSV mRNA and the hMPV mRNA are present in a ratio (w/w) of about 1 microgram of the hRSV mRNA to about 1 microgram of the hMPV mRNA.

The composition in any of the above embodiments wherein the hRSV mRNA and the hMPV mRNA are present in a ratio (w/w) of about 1 microgram of the hRSV mRNA to about 2 micrograms of the hMPV mRNA.

The composition in any of the above embodiments wherein the hRSV mRNA and the hMPV mRNA are present in a ratio (w/w) of about 1 microgram of the hRSV mRNA to about 3 micrograms of the hMPV mRNA.

The composition in any of the above embodiments wherein the hRSV mRNA and the hMPV mRNA are present in a ratio (w/w) of about 1 microgram of the hRSV mRNA to about 5 micrograms of the hMPV mRNA.

The composition in any of the above embodiments wherein the hRSV mRNA and the hMPV mRNA are present in a ratio (w/w) of about 0.5 microgram of the hRSV mRNA to about 0.5 microgram of the hMPV mRNA.

The composition in any of the above embodiments wherein the hRSV mRNA and the hMPV mRNA are present in a ratio (w/w) of about 0.5 microgram of the hRSV mRNA to about 1.5 microgram of the hMPV mRNA.

The composition in any of the above embodiments wherein the hRSV mRNA and the hMPV mRNA are present in a ratio (w/w) of about 0.5 microgram of the hRSV mRNA to about 1 microgram of the hMPV mRNA.

The composition in any of the above embodiments wherein the hRSV mRNA and the hMPV mRNA are present in a ratio (w/w) of about 0.5 microgram of the hRSV mRNA to about 2.5 micrograms of the hMPV mRNA.

The composition in any of the above embodiments wherein the hRSV mRNA and the hMPV mRNA are present in a ratio (w/w) of 1 microgram of the hRSV mRNA to 1 microgram of the hMPV mRNA.

The composition in any of the above embodiments wherein the hRSV mRNA and the hMPV mRNA are present in a ratio (w/w) of 1 microgram of the hRSV mRNA to 2 micrograms of the hMPV mRNA.

The composition in any of the above embodiments wherein the hRSV mRNA and the hMPV mRNA are present in a ratio (w/w) of 1 microgram of the hRSV mRNA to 3 micrograms of the hMPV mRNA.

The composition in any of the above embodiments wherein the hRSV mRNA and the hMPV mRNA are present in a ratio (w/w) of 1 microgram of the hRSV mRNA to 5 micrograms of the hMPV mRNA.

The composition in any of the above embodiments wherein the hRSV mRNA and the hMPV mRNA are present in a ratio (w/w) of 0.5 microgram of the hRSV mRNA to 0.5 microgram of the hMPV mRNA.

The composition in any of the above embodiments wherein the hRSV mRNA and the hMPV mRNA are present in a ratio (w/w) of 0.5 microgram of the hRSV mRNA to 1.5 microgram of the hMPV mRNA.

The composition in any of the above embodiments wherein the hRSV mRNA and the hMPV mRNA are present in a ratio (w/w) of 0.5 microgram of the hRSV mRNA to 1 microgram of the hMPV mRNA.

The composition in any of the above embodiments wherein the hRSV mRNA and the hMPV mRNA are present in a ratio (w/w) of 0.5 microgram of the hRSV mRNA to 2.5 microgram of the hMPV mRNA.

The compositions in any of the above embodiments may comprise an hRSV:hMPV:hPIV3 mRNA ratio (e.g., μg) of about 1:1:1, 1:1.1:1, 1:1.2:1, 1:1.3:1, 1:1.4:1, 1:1.5:1, 1:1.6:1, 1:1.7:1, 1:1.8:1, 1:1.9:1, 1:2:1, 1:2.1:1, 1:2.2:1, 1:2.3:1, 1:2.4:1, 1:2.5:1, 1:2.6:1, 1:2.7:1, 1:2.8:1, 1:2.9:1, 1:3:1, 1:3.1:1, 1:3.2:1, 1:3.3:1, 1:3.4:1, 1:3.5:1, 1:3.6:1, 1:3.7:1, 1:3.8:1, 1:3.9:1, 1:4:1, 1:4.1:1, 1:4.2:1, 1:4.3:1, 1:4.4:1, 1:4.5:1, 1:4.6:1, 1:4.7:1, 1:4.8:1, 1:4.9:1, 1:5:1, 1:5.1:1, 1:5.2:1, 1:5.3:1, 1:5.4:1, 1:5.5:1, 1:5.6:1, 1:5.7:1, 1:5.8:1, 1:5.9:1, 1:6:1, 1:6.1:1, 1:6.2:1, 1:6.3:1, 1:6.4:1, 1:6.5:1, 1:6.6:1, 1:6.7:1, 1:6.8:1, 1:6.9:1, 1:7:1, 1:7.1:1, 1:7.2:1, 1:7.3:1, 1:7.4:1, 1:7.5:1, 1:7.6:1, 1:7.7:1, 1:7.8:1, 1:7.9:1, 1:8:1, 1:8.1:1, 1:8.2:1, 1:8.3:1, 1:8.4:1, 1:8.5:1, 1:8.6:1, 1:8.7:1, 1:8.8:1, 1:8.9:1, 1:9:1, 1:9.1:1, 1:9.2:1, 1:9.3:1, 1:9.4:1, 1:9.5:1, 1:9.6:1, 1:9.7:1, 1:9.8:1, 1:9.9:1, 1:10:1, 1:1:0.9, 1:1:0.8, 1:1:0.7, 1:1:0.6, 1:1:0.5, 1:1:0.4, 1:1:0.3, 1:1:0.2, 1:1:0.1.

The compositions in any of the above embodiments may comprise an hRSV:hMPV:hPIV3 mRNA ratio (e.g., μg) of 1:1:1, 1:1.1:1, 1:1.2:1, 1:1.3:1, 1:1.4:1, 1:1.5:1, 1:1.6:1, 1:1.7:1, 1:1.8:1, 1:1.9:1, 1:2:1, 1:2.1:1, 1:2.2:1, 1:2.3:1, 1:2.4:1, 1:2.5:1, 1:2.6:1, 1:2.7:1, 1:2.8:1, 1:2.9:1, 1:3:1, 1:3.1:1, 1:3.2:1, 1:3.3:1, 1:3.4:1, 1:3.5:1, 1:3.6:1, 1:3.7:1, 1:3.8:1, 1:3.9:1, 1:4:1, 1:4.1:1, 1:4.2:1, 1:4.3:1, 1:4.4:1, 1:4.5:1, 1:4.6:1, 1:4.7:1, 1:4.8:1, 1:4.9:1, 1:5:1, 1:5.1:1, 1:5.2:1, 1:5.3:1, 1:5.4:1, 1:5.5:1, 1:5.6:1, 1:5.7:1, 1:5.8:1, 1:5.9:1, 1:6:1, 1:6.1:1, 1:6.2:1, 1:6.3:1, 1:6.4:1, 1:6.5:1, 1:6.6:1, 1:6.7:1, 1:6.8:1, 1:6.9:1, 1:7:1, 1:7.1:1, 1:7.2:1, 1:7.3:1, 1:7.4:1, 1:7.5:1, 1:7.6:1, 1:7.7:1, 1:7.8:1, 1:7.9:1, 1:8:1, 1:8.1:1, 1:8.2:1, 1:8.3:1, 1:8.4:1, 1:8.5:1, 1:8.6:1, 1:8.7:1, 1:8.8:1, 1:8.9:1, 1:9:1, 1:9.1:1, 1:9.2:1, 1:9.3:1, 1:9.4:1, 1:9.5:1, 1:9.6:1, 1:9.7:1, 1:9.8:1, 1:9.9:1, 1:10:1, 1:1:0.9, 1:1:0.8, 1:1:0.7, 1:1:0.6, 1:1:0.5, 1:1:0.4, 1:1:0.3, 1:1:0.2, 1:1:0.1.

The compositions in any of the above embodiments may comprise an hRSV:hMPV mRNA ratio (e.g., μg) of about 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9, 1:10.

The compositions in any of the above embodiments may comprise an hRSV:hMPV mRNA ratio (e.g., μg) of 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9, 1:10.

The compositions in any of the above embodiments may comprise an hMPV:hPIV3 mRNA ratio (e.g., μg) of about 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, 1:0.9, 1:0.8, 1:0.7, 1:0.6, 1:0.5, 1:0.4, 1:0.3, 1:0.2, 1:0.1.

The compositions in any of the above embodiments may comprise an hMPV:hPIV3 mRNA ratio (e.g., μg) of 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, 1:0.9, 1:0.8, 1:0.7, 1:0.6, 1:0.5, 1:0.4, 1:0.3, 1:0.2, 1:0.1.

The compositions in any of the above embodiments may comprise a ratio expressed, for example, in picograms (pg), nanograms (ng), micrograms (pg), milligrams (mg), etc.

The compositions in any of the above embodiments may comprise hMPV mRNA in an amount (e.g., μg) that is 1 time, or about 1 time, the amount of hRSV mRNA and/or hPIV3 mRNA.

The compositions in any of the above embodiments may comprise hMPV mRNA in an amount (e.g., μg) that is 1.1 times, or about 1.1 times, the amount of hRSV mRNA and/or hPIV3 mRNA.

The compositions in any of the above embodiments may comprise hMPV mRNA in an amount (e.g., μg) that is 1.2 times, or about 1.2 times, the amount of hRSV mRNA and/or hPIV3 mRNA.

The compositions in any of the above embodiments may comprise hMPV mRNA in an amount (e.g., μg) that is 1.3 times, or about 1.3 times, the amount of hRSV mRNA and/or hPIV3 mRNA.

The compositions in any of the above embodiments may comprise hMPV mRNA in an amount (e.g., μg) that is 1.4 times, or about 1.4 times, the amount of hRSV mRNA and/or hPIV3 mRNA.

The compositions in any of the above embodiments may comprise hMPV mRNA in an amount (e.g., μg) that is 1.5 times, or about 1.5 times, the amount of hRSV mRNA and/or hPIV3 mRNA.

The compositions in any of the above embodiments may comprise hMPV mRNA in an amount (e.g., μg) that is 1.6 times, or about 1.6 times, the amount of hRSV mRNA and/or hPIV3 mRNA.

The compositions in any of the above embodiments may comprise hMPV mRNA in an amount (e.g., μg) that is 1.7 times, or about 1.7 times, the amount of hRSV mRNA and/or hPIV3 mRNA.

The compositions in any of the above embodiments may comprise hMPV mRNA in an amount (e.g., μg) that is 1.8 times, or about 1.8 times, the amount of hRSV mRNA and/or hPIV3 mRNA.

The compositions in any of the above embodiments may comprise hMPV mRNA in an amount (e.g., μg) that is 1.9 times, or about 1.9 times, the amount of hRSV mRNA and/or hPIV3 mRNA.

The compositions in any of the above embodiments may comprise hMPV mRNA in an amount (e.g., μg) that is 2 times, or about 2 times, the amount of hRSV mRNA and/or hPIV3 mRNA.

The compositions in any of the above embodiments may comprise hMPV mRNA in an amount (e.g., μg) that is 2.1 times, or about 2.1 times, the amount of hRSV mRNA and/or hPIV3 mRNA.

The compositions in any of the above embodiments may comprise hMPV mRNA in an amount (e.g., μg) that is 2.2 times, or about 2.2 times, the amount of hRSV mRNA and/or hPIV3 mRNA.

The compositions in any of the above embodiments may comprise hMPV mRNA in an amount (e.g., μg) that is 2.3 times, or about 2.3 times, the amount of hRSV mRNA and/or hPIV3 mRNA.

The compositions in any of the above embodiments may comprise hMPV mRNA in an amount (e.g., μg) that is 2.4 times, or about 2.4 times, the amount of hRSV mRNA and/or hPIV3 mRNA.

The compositions in any of the above embodiments may comprise hMPV mRNA in an amount (e.g., μg) that is 2.5 times, or about 2.5 times, the amount of hRSV mRNA and/or hPIV3 mRNA.

The compositions in any of the above embodiments may comprise hMPV mRNA in an amount (e.g., μg) that is 2.6 times, or about 2.6 times, the amount of hRSV mRNA and/or hPIV3 mRNA.

The compositions in any of the above embodiments may comprise hMPV mRNA in an amount (e.g., μg) that is 2.7 times, or about 2.7 times, the amount of hRSV mRNA and/or hPIV3 mRNA.

The compositions in any of the above embodiments may comprise hMPV mRNA in an amount (e.g., μg) that is 2.8 times, or about 2.8 times, the amount of hRSV mRNA and/or hPIV3 mRNA.

The compositions in any of the above embodiments may comprise hMPV mRNA in an amount (e.g., μg) that is 2.9 times, or about 2.9 times, the amount of hRSV mRNA and/or hPIV3 mRNA.

The compositions in any of the above embodiments may comprise hMPV mRNA in an amount (e.g., μg) that is 3 times, or about 3 times, the amount of hRSV mRNA and/or hPIV3 mRNA.

The compositions in any of the above embodiments may comprise hMPV mRNA in an amount (e.g., μg) that is 3.1 times, or about 3.1 times, the amount of hRSV mRNA and/or hPIV3 mRNA.

The compositions in any of the above embodiments may comprise hMPV mRNA in an amount (e.g., μg) that is 3.2 times, or about 3.2 times, the amount of hRSV mRNA and/or hPIV3 mRNA.

The compositions in any of the above embodiments may comprise hMPV mRNA in an amount (e.g., μg) that is 3.3 times, or about 3.3 times, the amount of hRSV mRNA and/or hPIV3 mRNA.

The compositions in any of the above embodiments may comprise hMPV mRNA in an amount (e.g., μg) that is 3.4 times, or about 3.4 times, the amount of hRSV mRNA and/or hPIV3 mRNA.

The compositions in any of the above embodiments may comprise hMPV mRNA in an amount (e.g., μg) that is 3.5 times, or about 3.5 times, the amount of hRSV mRNA and/or hPIV3 mRNA.

The compositions in any of the above embodiments may comprise hMPV mRNA in an amount (e.g., μg) that is 3.6 times, or about 3.6 times, the amount of hRSV mRNA and/or hPIV3 mRNA.

The compositions in any of the above embodiments may comprise hMPV mRNA in an amount (e.g., μg) that is 3.7 times, or about 3.7 times, the amount of hRSV mRNA and/or hPIV3 mRNA.

The compositions in any of the above embodiments may comprise hMPV mRNA in an amount (e.g., μg) that is 3.8 times, or about 3.8 times, the amount of hRSV mRNA and/or hPIV3 mRNA.

The compositions in any of the above embodiments may comprise hMPV mRNA in an amount (e.g., μg) that is 3.9 times, or about 3.9 times, the amount of hRSV mRNA and/or hPIV3 mRNA.

The compositions in any of the above embodiments may comprise hMPV mRNA in an amount (e.g., μg) that is 4 times, or about 4 times, the amount of hRSV mRNA and/or hPIV3 mRNA.

The compositions in any of the above embodiments may comprise hMPV mRNA in an amount (e.g., μg) that is 4.1 times, or about 4.1 times, the amount of hRSV mRNA and/or hPIV3 mRNA.

The compositions in any of the above embodiments may comprise hMPV mRNA in an amount (e.g., μg) that is 4.2 times, or about 4.2 times, the amount of hRSV mRNA and/or hPIV3 mRNA.

The compositions in any of the above embodiments may comprise hMPV mRNA in an amount (e.g., μg) that is 4.3 times, or about 4.3 times, the amount of hRSV mRNA and/or hPIV3 mRNA.

The compositions in any of the above embodiments may comprise hMPV mRNA in an amount (e.g., μg) that is 4.4 times, or about 4.4 times, the amount of hRSV mRNA and/or hPIV3 mRNA.

The compositions in any of the above embodiments may comprise hMPV mRNA in an amount (e.g., μg) that is 4.5 times, or about 4.5 times, the amount of hRSV mRNA and/or hPIV3 mRNA.

The compositions in any of the above embodiments may comprise hMPV mRNA in an amount (e.g., μg) that is 4.6 times, or about 4.6 times, the amount of hRSV mRNA and/or hPIV3 mRNA.

The compositions in any of the above embodiments may comprise hMPV mRNA in an amount (e.g., μg) that is 4.7 times, or about 4.7 times, the amount of hRSV mRNA and/or hPIV3 mRNA.

The compositions in any of the above embodiments may comprise hMPV mRNA in an amount (e.g., μg) that is 4.8 times, or about 4.8 times, the amount of hRSV mRNA and/or hPIV3 mRNA.

The compositions in any of the above embodiments may comprise hMPV mRNA in an amount (e.g., μg) that is 4.9 times, or about 4.9 times, the amount of hRSV mRNA and/or hPIV3 mRNA.

The compositions in any of the above embodiments may comprise hMPV mRNA in an amount (e.g., μg) that is 5 times, or about 5 times, the amount of hRSV mRNA and/or hPIV3 mRNA.

The compositions in any of the above embodiments may comprise hMPV mRNA in an amount (e.g., μg) that is 5.1 times, or about 5.1 times, the amount of hRSV mRNA and/or hPIV3 mRNA.

The compositions in any of the above embodiments may comprise hMPV mRNA in an amount (e.g., μg) that is 5.2 times, or about 5.2 times, the amount of hRSV mRNA and/or hPIV3 mRNA.

The compositions in any of the above embodiments may comprise hMPV mRNA in an amount (e.g., μg) that is 5.3 times, or about 5.3 times, the amount of hRSV mRNA and/or hPIV3 mRNA.

The compositions in any of the above embodiments may comprise hMPV mRNA in an amount (e.g., μg) that is 5.4 times, or about 5.4 times, the amount of hRSV mRNA and/or hPIV3 mRNA.

The compositions in any of the above embodiments may comprise hMPV mRNA in an amount (e.g., μg) that is 5.5 times, or about 5.5 times, the amount of hRSV mRNA and/or hPIV3 mRNA.

The compositions in any of the above embodiments may comprise hMPV mRNA in an amount (e.g., μg) that is 5.6 times, or about 5.6 times, the amount of hRSV mRNA and/or hPIV3 mRNA.

The compositions in any of the above embodiments may comprise hMPV mRNA in an amount (e.g., μg) that is 5.7 times, or about 5.7 times, the amount of hRSV mRNA and/or hPIV3 mRNA.

The compositions in any of the above embodiments may comprise hMPV mRNA in an amount (e.g., μg) that is 5.8 times, or about 5.8 times, the amount of hRSV mRNA and/or hPIV3 mRNA.

The compositions in any of the above embodiments may comprise hMPV mRNA in an amount (e.g., μg) that is 5.9 times, or about 5.9 times, the amount of hRSV mRNA and/or hPIV3 mRNA.

The compositions in any of the above embodiments may comprise hMPV mRNA in an amount (e.g., μg) that is 6 times, or about 6 times, the amount of hRSV mRNA and/or hPIV3 mRNA.

The compositions in any of the above embodiments may comprise hMPV mRNA in an amount (e.g., μg) that is 6.1 times, or about 6.1 times, the amount of hRSV mRNA and/or hPIV3 mRNA.

The compositions in any of the above embodiments may comprise hMPV mRNA in an amount (e.g., μg) that is 6.2 times, or about 6.2 times, the amount of hRSV mRNA and/or hPIV3 mRNA.

The compositions in any of the above embodiments may comprise hMPV mRNA in an amount (e.g., μg) that is 6.3 times, or about 6.3 times, the amount of hRSV mRNA and/or hPIV3 mRNA.

The compositions in any of the above embodiments may comprise hMPV mRNA in an amount (e.g., μg) that is 6.4 times, or about 6.4 times, the amount of hRSV mRNA and/or hPIV3 mRNA.

The compositions in any of the above embodiments may comprise hMPV mRNA in an amount (e.g., μg) that is 6.5 times, or about 6.5 times, the amount of hRSV mRNA and/or hPIV3 mRNA.

The compositions in any of the above embodiments may comprise hMPV mRNA in an amount (e.g., μg) that is 6.6 times, or about 6.6 times, the amount of hRSV mRNA and/or hPIV3 mRNA.

The compositions in any of the above embodiments may comprise hMPV mRNA in an amount (e.g., μg) that is 6.7 times, or about 6.7 times, the amount of hRSV mRNA and/or hPIV3 mRNA.

The compositions in any of the above embodiments may comprise hMPV mRNA in an amount (e.g., μg) that is 6.8 times, or about 6.8 times, the amount of hRSV mRNA and/or hPIV3 mRNA.

The compositions in any of the above embodiments may comprise hMPV mRNA in an amount (e.g., μg) that is 6.9 times, or about 6.9 times, the amount of hRSV mRNA and/or hPIV3 mRNA.

The compositions in any of the above embodiments may comprise hMPV mRNA in an amount (e.g., μg) that is 7 times, or about 7 times, the amount of hRSV mRNA and/or hPIV3 mRNA.

The compositions in any of the above embodiments may comprise hMPV mRNA in an amount (e.g., μg) that is 7.1 times, or about 7.1 times, the amount of hRSV mRNA and/or hPIV3 mRNA.

The compositions in any of the above embodiments may comprise hMPV mRNA in an amount (e.g., μg) that is 7.2 times, or about 7.2 times, the amount of hRSV mRNA and/or hPIV3 mRNA.

The compositions in any of the above embodiments may comprise hMPV mRNA in an amount (e.g., μg) that is 7.3 times, or about 7.3 times, the amount of hRSV mRNA and/or hPIV3 mRNA.

The compositions in any of the above embodiments may comprise hMPV mRNA in an amount (e.g., μg) that is 7.4 times, or about 7.4 times, the amount of hRSV mRNA and/or hPIV3 mRNA.

The compositions in any of the above embodiments may comprise hMPV mRNA in an amount (e.g., μg) that is 7.5 times, or about 7.5 times, the amount of hRSV mRNA and/or hPIV3 mRNA.

The compositions in any of the above embodiments may comprise hMPV mRNA in an amount (e.g., μg) that is 7.6 times, or about 7.6 times, the amount of hRSV mRNA and/or hPIV3 mRNA.

The compositions in any of the above embodiments may comprise hMPV mRNA in an amount (e.g., μg) that is 7.7 times, or about 7.7 times, the amount of hRSV mRNA and/or hPIV3 mRNA.

The compositions in any of the above embodiments may comprise hMPV mRNA in an amount (e.g., μg) that is 7.8 times, or about 7.8 times, the amount of hRSV mRNA and/or hPIV3 mRNA.

The compositions in any of the above embodiments may comprise hMPV mRNA in an amount (e.g., μg) that is 7.9 times, or about 7.9 times, the amount of hRSV mRNA and/or hPIV3 mRNA.

The compositions in any of the above embodiments may comprise hMPV mRNA in an amount (e.g., μg) that is 8 times, or about 8 times, the amount of hRSV mRNA and/or hPIV3 mRNA.

The compositions in any of the above embodiments may comprise hMPV mRNA in an amount (e.g., μg) that is 8.1 times, or about 8.1 times, the amount of hRSV mRNA and/or hPIV3 mRNA.

The compositions in any of the above embodiments may comprise hMPV mRNA in an amount (e.g., μg) that is 8.2 times, or about 8.2 times, the amount of hRSV mRNA and/or hPIV3 mRNA.

The compositions in any of the above embodiments may comprise hMPV mRNA in an amount (e.g., μg) that is 8.3 times, or about 8.3 times, the amount of hRSV mRNA and/or hPIV3 mRNA.

The compositions in any of the above embodiments may comprise hMPV mRNA in an amount (e.g., μg) that is 8.4 times, or about 8.4 times, the amount of hRSV mRNA and/or hPIV3 mRNA.

The compositions in any of the above embodiments may comprise hMPV mRNA in an amount (e.g., μg) that is 8.5 times, or about 8.5 times, the amount of hRSV mRNA and/or hPIV3 mRNA.

The compositions in any of the above embodiments may comprise hMPV mRNA in an amount (e.g., μg) that is 8.6 times, or about 8.6 times, the amount of hRSV mRNA and/or hPIV3 mRNA.

The compositions in any of the above embodiments may comprise hMPV mRNA in an amount (e.g., μg) that is 8.7 times, or about 8.7 times, the amount of hRSV mRNA and/or hPIV3 mRNA.

The compositions in any of the above embodiments may comprise hMPV mRNA in an amount (e.g., μg) that is 8.8 times, or about 8.8 times, the amount of hRSV mRNA and/or hPIV3 mRNA.

The compositions in any of the above embodiments may comprise hMPV mRNA in an amount (e.g., μg) that is 8.9 times, or about 8.9 times, the amount of hRSV mRNA and/or hPIV3 mRNA.

The compositions in any of the above embodiments may comprise hMPV mRNA in an amount (e.g., μg) that is 9 times, or about 9 times, the amount of hRSV mRNA and/or hPIV3 mRNA.

The compositions in any of the above embodiments may comprise hMPV mRNA in an amount (e.g., μg) that is 9.1 times, or about 9.1 times, the amount of hRSV mRNA and/or hPIV3 mRNA.

The compositions in any of the above embodiments may comprise hMPV mRNA in an amount (e.g., μg) that is 9.2 times, or about 9.2 times, the amount of hRSV mRNA and/or hPIV3 mRNA.

The compositions in any of the above embodiments may comprise hMPV mRNA in an amount (e.g., μg) that is 9.3 times, or about 9.3 times, the amount of hRSV mRNA and/or hPIV3 mRNA.

The compositions in any of the above embodiments may comprise hMPV mRNA in an amount (e.g., μg) that is 9.4 times, or about 9.4 times, the amount of hRSV mRNA and/or hPIV3 mRNA.

The compositions in any of the above embodiments may comprise hMPV mRNA in an amount (e.g., μg) that is 9.5 times, or about 9.5 times, the amount of hRSV mRNA and/or hPIV3 mRNA.

The compositions in any of the above embodiments may comprise hMPV mRNA in an amount (e.g., μg) that is 9.6 times, or about 9.6 times, the amount of hRSV mRNA and/or hPIV3 mRNA.

The compositions in any of the above embodiments may comprise hMPV mRNA in an amount (e.g., μg) that is 9.7 times, or about 9.7 times, the amount of hRSV mRNA and/or hPIV3 mRNA.

The compositions in any of the above embodiments may comprise hMPV mRNA in an amount (e.g., μg) that is 9.8 times, or about 9.8 times, the amount of hRSV mRNA and/or hPIV3 mRNA.

The compositions in any of the above embodiments may comprise hMPV mRNA in an amount (e.g., μg) that is 9.9 times, or about 9.9 times, the amount of hRSV mRNA and/or hPIV3 mRNA.

The compositions in any of the above embodiments may comprise hMPV mRNA in an amount (e.g., μg) that is 10 times, or about 10 times, the amount of hRSV mRNA and/or hPIV3 mRNA.

The compositions in any of the above embodiments may comprise hPIV3 mRNA in an amount (e.g., μg) that is 0.9 times, or about 0.9 times, the amount of hRSV mRNA and/or hMPV mRNA.

The compositions in any of the above embodiments may comprise hPIV3 mRNA in an amount (e.g., μg) that is 0.8 times, or about 0.8 times, the amount of hRSV mRNA and/or hMPV mRNA.

The compositions in any of the above embodiments may comprise hPIV3 mRNA in an amount (e.g., μg) that is 0.7 times, or about 0.7 times, the amount of hRSV mRNA and/or hMPV mRNA.

The compositions in any of the above embodiments may comprise hPIV3 mRNA in an amount (e.g., μg) that is 0.6 times, or about 0.6 times, the amount of hRSV mRNA and/or hMPV mRNA.

The compositions in any of the above embodiments may comprise hPIV3 mRNA in an amount (e.g., μg) that is 0.5 times, or about 0.5 times, the amount of hRSV mRNA and/or hMPV mRNA.

The compositions in any of the above embodiments may comprise hPIV3 mRNA in an amount (e.g., μg) that is 0.4 times, or about 0.4 times, the amount of hRSV mRNA and/or hMPV mRNA.

The compositions in any of the above embodiments may comprise hPIV3 mRNA in an amount (e.g., μg) that is 0.3 times, or about 0.3 times, the amount of hRSV mRNA and/or hMPV mRNA.

The compositions in any of the above embodiments may comprise hPIV3 mRNA in an amount (e.g., μg) that is 0.2 times, or about 0.2 times, the amount of hRSV mRNA and/or hMPV mRNA.

The compositions in any of the above embodiments may comprise hPIV3 mRNA in an amount (e.g., μg) that is 0.1 times, or about 0.1 times, the amount of hRSV mRNA and/or hMPV mRNA.

The compositions in any of the above embodiments may comprise mRNA amounts expressed, for example, in picograms (pg), nanograms (ng), micrograms (pg), milligrams (mg), etc.

### VII. Lipid Nanoparticle (LNP)

The LNPs of the disclosure can comprise four categories of lipids: (i) an ionizable lipid (e.g., cationic lipid); (ii) a PEGylated lipid; (iii) a cholesterol-based lipid (e.g., cholesterol), and (iv) a helper lipid.

### VII.A. Cationic Lipid

An ionizable lipid facilitates mRNA encapsulation and may be a cationic lipid. A cationic lipid affords a positively charged environment at low pH to facilitate efficient encapsulation of the negatively charged mRNA drug substance. Exemplary cationic lipids are shown below in Table 1.

TABLE 1
Ionizable Lipids

| Name | Structure |
|---|---|
| OF-02 (ML7) | 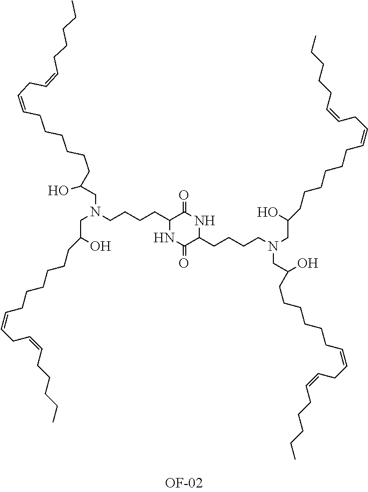 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC] |
| cKK-E10 | 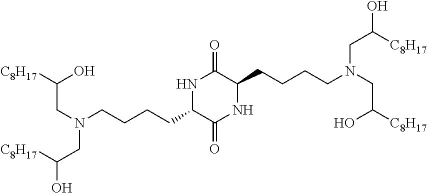 |
| GL-HEPES-E3- E10-DS-3-E18- 1((2-(4-(2-((3- (Bis((Z)-2- hydroxyoctadec- 9-en-1-yl)amino)- propyl)disulfane- yl)ethyl)piper- azin-1-yl)ethyl 4-(bis(2-hydroxy- decyl)amino)- butanoate) | 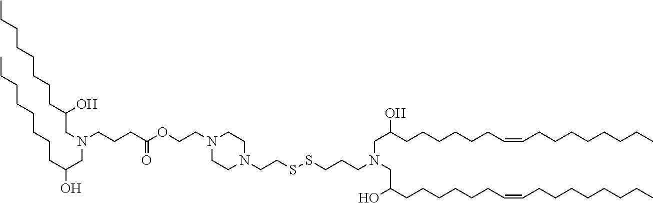 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC] |
| GL-HEPES-E3- E12-DS-4-E10 (2-(4-(2-((3-(bis- (2-hydroxydecyl)- amino)butyl)- disulfaneyl)ethyl)- piperazin-1-yl)- ethyl 4-(bis(2- hydroxydodecyl)- amino)butanoate) | 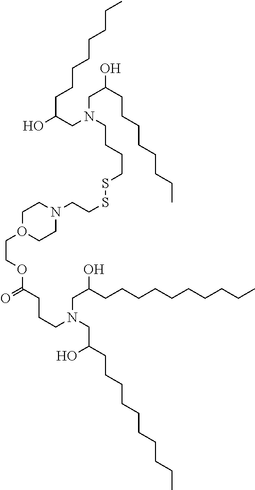 |
| GL-HEPES-E3- E12-DS-3-E14 (2-(4-(2-((3-(Bis- (2-hydroxytetra- decyl)amino)- propyl)disulfane- yl)ethyl)piper- azin-1-yl)ethyl 4-(bis(2-hydroxy- dodecyl)amino)- butanoate) | 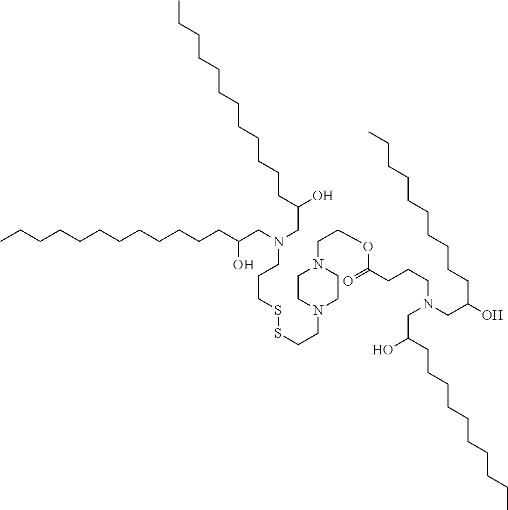 [SMILES: CCCCCCCCCCCCC(O)CN(CCCSSCCN1CCN(CCOC(=O)CCCN(CC(O)CCCCCCCCCC)CC(O)CCCCCCCCCC)CC1)CC(O)CCCCCCCCCCCC] |
| MC3 | 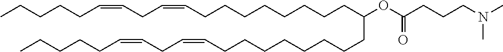 [SMILES: CCCCC/C=C\C/C=C\CCCCCCCCC(CCCCCCCC/C=C\C/C=C\CCCCC)OC(=O)CCCN(C)C] |
| SM-102 (9-hepta- decanyl 8-{(2- hydroxyethyl)[6- oxo-6-(undecyl- oxy)hexyl]amino}- octanoate) | 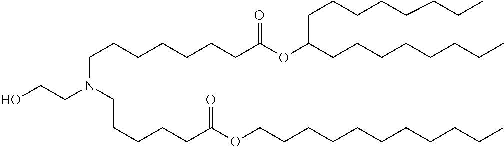 [SMILES: CCCCCCCCCCCOC(=O)CCCCCN(CCO)CCCCCCCC(=O)OC(CCCCCCCC)CCCCCCCC] |
| ALC-0315 [(4- hydroxybutyl)- azanediyl]- di(hexane-6,1- diyl)bis(2-hexyl- decanoate) | 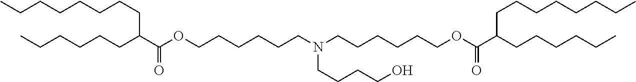 [SMILES: CCCCCCCCC(CCCCCC)C(=O)OCCCCCCN(CCCCO)CCCCCCOC(=O)C(CCCCCC)CCCCCCCC] |
| IM-001 | |

The cationic lipid may be selected from the group comprising [ckkE10]/[OF-02], [(6Z,9Z,28Z,31Z)-heptatriaconta-6,9,28,31-tetraen-19-yl] 4-(dimethylamino)butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl] (Z)-octadec-9-enoate (DODAP); 2,5-bis(3-aminopropylamino)-N-[2-[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl]N-[2-(dimethylamino)ethyl]carbamate (DC-Chol); tetrakis(8-methylnonyl) 3,3′,3″,3′″-(((methylazanediyl) bis(propane-3,1 diyl))bis (azanetriyl))tetrapropionate (306Oi10); decyl (2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-O16B); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate (FTT5); (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate) (OF-Deg-Lin); TT3; N¹,N³,N⁵-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl)(8-(nonyloxy)-8-oxooctyl)amino)octanoate (Lipid 5); IM-001 and combinations thereof.

In certain embodiments, the cationic lipid is biodegradable.

In various embodiments, the cationic lipid is not biodegradable.

In some embodiments, the cationic lipid is cleavable.

In certain embodiments, the cationic lipid is not cleavable.

Cationic lipids are described in further detail in Dong et al. (PNAS. 111(11):3955-60. 2014); Fenton et al. (Adv. Mater. 28:2939. 2016); U.S. Pat. Nos. 9,512,073; and 10,201,618, each of which is incorporated herein by reference.

### VII.B. PEGylated Lipid

The PEGylated lipid component provides control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al. FEBS Letters 268(1):235-7. 1990). These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C₆-C₂₀ (e.g., C₈, C₁₀, C₁₂, C₁₄, C₁₆, or C₁₈) length, such as a derivatized ceramide (e.g., N-octanoyl-sphingosine-1-[succinyl(methoxypolyethylene glycol)] (C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG), PEG-DAG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In certain embodiments, the PEG has a high molecular weight, e.g., 2000-2400 g/mol. In certain embodiments, the PEG is PEG2000 (or PEG-2K). In certain embodiments, the PEGylated lipid herein is DMG-PEG2000, DSPE-PEG2000, DLPE-PEG2000, DSG-PEG2000, C8 PEG2000, or ALC-0159 (2-[(polyethylene glycol)-2000]-N,N-ditetradecylacetamide). In certain embodiments, the PEGylated lipid herein is DMG-PEG2000.

### VII.C. Cholesterol-Based Lipid

The cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), I,4-bis(3-N-oleylamino-propyl)piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; International Pub. No. WO 2011/068810), sitosterol (22,23-dihydrostigmasterol), β-sitosterol, sitostanol, fucosterol, stigmasterol (stigmasta-5,22-dien-3-ol), ergosterol; desmosterol (3ß-hydroxy-5,24-cholestadiene); lanosterol (8,24-lanostadien-3b-ol); 7-dehydrocholesterol (Δ5,7-cholesterol); dihydrolanosterol (24,25-dihydrolanosterol); zymosterol (5α-cholesta-8,24-dien-3ß-ol); lathosterol (5a-cholest-7-en-3B-ol); diosgenin ((3β,25R)-spirost-5-en-3-ol); campesterol (campest-5-en-3ß-ol); campestanol (5a-campestan-3ß-ol); 24-methylene cholesterol (5,24(28)-cholestadien-24-methylen-3ß-ol); cholesteryl margarate (cholest-5-en-3ß-yl heptadecanoate); cholesteryl oleate; cholesteryl stearate and other modified forms of cholesterol. In some embodiments, the cholesterol-based lipid used in the LNPs is cholesterol.

### VII.D. Helper Lipid

A helper lipid enhances the structural stability of the LNP and helps the LNP in endosome escape. It improves uptake and release of the mRNA drug payload. In some embodiments, the helper lipid is a zwitterionic lipid, which has fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), DMPC, 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, sphingomyelins, ceramides, cerebrosides, gangliosides, 16-O-monomethyl PE, 16-O-dimethyl PE, 18-1-trans PE, I-stearoyl-2-oleoyl-phosphatidyethanolamine (SOPE), or a combination thereof. In certain embodiments, the helper lipid is DOPE. In certain embodiments, the helper lipid is DSPC.

In various embodiments, the present LNPs comprise (i) a cationic lipid selected from OF-02, cKK-E10, GL-HEPES-E3-E10-DS-3-E18-1, GL-HEPES-E3-E12-DS-4-E10, or GL-HEPES-E3-E12-DS-3-E14; (ii) DMG-PEG2000; (iii) cholesterol; and (iv) DOPE.

### VII.E. Molar Ratios of the Lipid Components

The molar ratios of the above components are important for the LNPs' effectiveness in delivering mRNA. The molar ratio of the cationic lipid, the PEGylated lipid, the cholesterol-based lipid, and the helper lipid is A:B:C:D, where A+B+C+D=100%. In some embodiments, the molar ratio of the cationic lipid in the LNPs relative to the total lipids (i.e., A) is 35-55%, such as 35-50% (e.g., 38-42% such as 40%, or 45-50%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-50% (e.g., 27-30% such as 28.5%, or 38-43%). In some embodiments, the molar ratio of the helper lipid relative to the total lipids (i.e., D) is 5-35% (e.g., 28-32% such as 30%, or 8-12%, such as 10%).

In some embodiments, the (PEGylated lipid+cholesterol) components have the same molar amount as the helper lipid. In some embodiments, the LNPs contain a molar ratio of the cationic lipid to the helper lipid that is more than 1.

In certain embodiments, the LNP of the disclosure comprises:
a cationic lipid at a molar ratio of 35% to 55% or 40% to 50% (e.g., a cationic lipid at a molar ratio of 35%, 36%, 37%, 38%, 39%, 40%, 41% 42%, 43%, 44%, 45%, 46%, 47%, 48%, 49%, 50%, 51%, 52%, 53%, 54%, or 55%);
a polyethylene glycol (PEG) conjugated (PEGylated) lipid at a molar ratio of 0.25% to 2.75% or 1.00% to 2.00% (e.g., a PEGylated lipid at a molar ratio of 0.25%, 0.50%, 0.75%, 1.00%, 1.25%, 1.50%, 1.75%, 2.00%, 2.25%, 2.50%, or 2.75%);
a cholesterol-based lipid at a molar ratio of 20% to 50%, 25% to 45%, or 28.5% to 43% (e.g., a cholesterol-based lipid at a molar ratio of 20%, 21%, 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, or 50%); and
a helper lipid at a molar ratio of 5% to 35%, 8% to 30%, or 10% to 30% (e.g., a helper lipid at a molar ratio of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%),
wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the PEGylated lipid is dimyristoyl-PEG2000 (DMG-PEG2000).

In various embodiments, the cholesterol-based lipid is cholesterol.

In some embodiments, the helper lipid is 1,2-dioleoyl-SN-glycero-3-phosphoethanolamine (DOPE).

In certain embodiments, the LNP comprises: OF-02 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: cKK-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E10-DS-3-E18-1 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-4-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-3-E14 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: SM-102 at a molar ratio of 35% to 55%; DMG-PEG2000 at a molar ratio of 0.25% to 2.75%; cholesterol at a molar ratio of 20% to 50%; and DSPC at a molar ratio of 5% to 35%.

In certain embodiments, the LNP comprises: ALC-0315 at a molar ratio of 35% to 55%; ALC-0159 at a molar ratio of 0.25% to 2.75%; cholesterol at a molar ratio of 20% to 50%; and DSPC at a molar ratio of 5% to 35%.

In certain embodiments, the LNP comprises: OF-02 at a molar ratio of 40%; DMG-PEG2000 at a molar ratio of 1.5%; cholesterol at a molar ratio of 28.5%; and DOPE at a molar ratio of 30%. This LNP formulation is designated “Lipid A” herein.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%. This LNP formulation is designated “Lipid B” herein.

In certain embodiments, the LNP comprises: GL-HEPES-E3-E10-DS-3-E18-1 at a molar ratio of 40%; DMG-PEG2000 at a molar ratio of 1.5%; cholesterol at a molar ratio of 28.5%; and DOPE at a molar ratio of 30%. This LNP formulation is designated “Lipid C” herein.

In certain embodiments, the LNP comprises: GL-HEPES-E3-E12-DS-4-E10 at a molar ratio of 40%; DMG-PEG2000 at a molar ratio of 1.5%; cholesterol at a molar ratio of 28.5%; and DOPE at a molar ratio of 30%. This LNP formulation is designated “Lipid D” herein.

In certain embodiments, the LNP comprises: GL-HEPES-E3-E12-DS-3-E14 at a molar ratio of 40%; DMG-PEG2000 at a molar ratio of 1.5%; cholesterol at a molar ratio of 28.5%; and DOPE at a molar ratio of 30%. This LNP formulation is designated “Lipid E” herein.

In certain embodiments, the LNP comprises: 9-heptadecanyl 8-{(2-hydroxyethyl)[6-oxo-6-(undecyloxy)hexyl]amino}octanoate (SM-102) at a molar ratio of 50%; 1,2-distearoyl-sn-glycero-3-phosphocholine (DSPC) at a molar ratio of 10%; cholesterol at a molar ratio of 38.5%; and 1,2-dimyristoyl-rac-glycero-3-methoxypolyethylene glycol-2000 (DMG-PEG2000) at a molar ratio of 1.5%.

In certain embodiments, the LNP comprises: (4-hydroxybutyl)azanediyl]di(hexane-6,1-diyl) bis(2-hexyldecanoate) (ALC-0315) at a molar ratio of 46.3%; 1,2-distearoyl-sn-glycero-3-phosphocholine (DSPC) at a molar ratio of 9.4%; cholesterol at a molar ratio of 42.7%; and 2-[(polyethylene glycol)-2000]-N,N-ditetradecylacetamide (ALC-0159) at a molar ratio of 1.6%.

In certain embodiments, the LNP comprises: (4-hydroxybutyl)azanediyl]di(hexane-6,1-diyl) bis(2-hexyldecanoate) (ALC-0315) at a molar ratio of 47.4%; 1,2-distearoyl-sn-glycero-3-phosphocholine (DSPC) at a molar ratio of 10%; cholesterol at a molar ratio of 40.9%; and 2-[(polyethylene glycol)-2000]-N,N-ditetradecylacetamide (ALC-0159) at a molar ratio of 1.7%.

In certain embodiments, the LNP comprises: IM-001 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: IM-001 at a molar ratio of 40%; DMG-PEG2000 at a molar ratio of 1.5%; cholesterol at a molar ratio of 28.5%; and DOPE at a molar ratio of 30%.

To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid.

### VII.F. Buffer and Other Components

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands, stabilizing reagents (e.g., preservatives and antioxidants), and/or other pharmaceutically acceptable excipients. Examples of such excipients are parabens, thimerosal, thiomersal, chlorobutanol, benzalkonium chloride, and chelators (e.g., EDTA).

The LNP compositions of the present disclosure can be provided as a frozen liquid form or a lyophilized form. A variety of cryoprotectants may be used, including, without limitation, sucrose, trehalose, glucose, mannitol, mannose, dextrose, and the like. The cryoprotectant may constitute 5-30% (w/v) of the LNP composition. In some embodiments, the LNP composition comprise trehalose, e.g., at 5-30% (e.g., 10%) (w/v). Once formulated with the cryoprotectant, the LNP compositions may be frozen (or lyophilized and cryopreserved) at −20° C. to −80° C.

The LNP compositions may be provided to a patient in an aqueous buffered solution—thawed if previously frozen, or if previously lyophilized, reconstituted in an aqueous buffered solution at bedside. The buffered solution can be isotonic and suitable, e.g., for intramuscular or intradermal injection. In some embodiments, the buffered solution is a phosphate-buffered saline (PBS).

### VIII. Processes for Making LNP Vaccines

The present LNPs can be prepared by various techniques. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication, or extrusion of the multilamellar vesicles. In addition, unilamellar vesicles can be formed by detergent removal techniques.

Various methods are described in Patent Application Pub. Nos. US 2011/0244026, US 2016/0038432, US 2018/0153822, US 2018/0125989, and US 2021/0046192 and can be used for making LNP vaccines. One exemplary process entails encapsulating mRNA by mixing it with a mixture of lipids, without first pre-forming the lipids into lipid nanoparticles, as described in Patent Application Pub. No. US 2016/0038432. Another exemplary process entails encapsulating mRNA by mixing pre-formed LNPs with mRNA, as described in Patent Application Pub. No. US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA, and the mixed solution comprising the LNP-encapsulated mRNA. In some embodiments, the process includes the step of heating one or both of the mRNA solution and the pre-formed LNP solution prior to the mixing step. In some embodiments, the process includes heating one or more of the solutions comprising the pre-formed LNPs, the solution comprising the mRNA, and the solution comprising the LNP-encapsulated mRNA during the mixing step. In some embodiments, the process includes the step of heating the LNP-encapsulated mRNA after the mixing step. In some embodiments, the temperature to which one or more of the solutions is heated is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature is about 65° C.

Various methods may be used to prepare an mRNA solution suitable for the present disclosure. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include, but are not limited to, gear pumps, peristaltic pumps, and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging from about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging from about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods are available for sizing of a population of lipid nanoparticles. In various embodiments, methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function. Average LNP diameter may be reduced by sonication of formed LNP. Intermittent sonication cycles may be alternated with quasi-elastic light scattering (QELS) assessment to guide efficient lipid nanoparticle synthesis.

In some embodiments, the majority of purified LNPs, i.e., greater than about 50%, 55%, 60%, 65%, 70%, 75%, 80%, 85%, 90%, 95%, 96%, 97%, 98%, or 99% of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80% or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In certain embodiments, the LNP has an average diameter of 30-200 nm.

In various embodiments, the LNP has an average diameter of 80-150 nm.

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm.

In some embodiments, greater than about 70%, 75%, 80%, 85%, 90%, 95%, 96%, 97%, 98%, or 99% of the LNPs in the present composition have a size ranging from about 40-90 nm (e.g., about 45-85 nm, about 50-80 nm, about 55-75 nm, or about 60-70 nm) or about 50-70 nm (e.g., about 55-65 nm) are suitable for pulmonary delivery via nebulization.

In some embodiments, the dispersity, or measure of heterogeneity in size of molecules (PDI), of LNPs in a pharmaceutical composition provided by the present disclosure is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08. The PDI may be measured by a Zetasizer machine as described above.

In some embodiments, greater than about 75%, 80%, 85%, 90%, 95%, 96%, 97%, 98%, or 99% of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of 50% to 99%; or greater than about 60%, 65%, 70%, 75%, 80%, 85%, 90%, 92%, 95%, 98%, or 99%. Typically, lipid nanoparticles for use herein have an encapsulation efficiency of at least 90% (e.g., at least 91%, 92%, 93%, 94%, or 95%).

In some embodiments, an LNP has a N/P ratio of 1 to 10. In some embodiments, a lipid nanoparticle has a N/P ratio above 1, about 1, about 2, about 3, about 4, about 5, about 6, about 7, or about 8. In certain embodiments, a typical LNP herein has an N/P ratio of 4.

In some embodiments, a pharmaceutical composition according to the present disclosure contains at least about 0.5 μg, 1 μg, 5 μg, 10 μg, 100 μg, 500 μg, or 1000 μg of encapsulated mRNA. In some embodiments, a pharmaceutical composition contains about 0.1 μg to 1000 μg, at least about 0.5 μg, at least about 0.8 μg, at least about 1 μg, at least about 5 μg, at least about 8 μg, at least about 10 μg, at least about 50 μg, at least about 100 μg, at least about 500 μg, or at least about 1000 μg of encapsulated mRNA.

In some embodiments, mRNA can be made by chemical synthesis or by in vitro transcription (IVT) of a DNA template. An exemplary process for making and purifying mRNA is described in Example 1. In this process, an IVT process, a cDNA template is used to produce an mRNA transcript and the DNA template is degraded by a DNase. The transcript is purified by depth filtration and tangential flow filtration (TFF). The purified transcript is further modified by adding a cap and a tail, and the modified RNA is purified again by depth filtration and TFF.

The mRNA is then prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol. The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5. The buffer may contain other components such as a salt (e.g., sodium, potassium, and/or calcium salts). In particular embodiments, the aqueous buffer has 1 mM citrate, 150 mM NaCl, pH 4.5.

An exemplary, nonlimiting process for making an mRNA-LNP composition involves mixing a buffered mRNA solution with a solution of lipids in ethanol in a controlled homogeneous manner, where the ratio of lipids:mRNA is maintained throughout the mixing process. In this illustrative example, the mRNA is presented in an aqueous buffer containing citric acid monohydrate, tri-sodium citrate dihydrate, and sodium chloride. The mRNA solution is added to the solution (1 mM citrate buffer, 150 mM NaCl, pH 4.5). The lipid mixture of four lipids (e.g., a cationic lipid, a PEGylated lipid, a cholesterol-based lipid, and a helper lipid) is dissolved in ethanol. The aqueous mRNA solution and the ethanol lipid solution are mixed at a volume ratio of 4:1 in a “T” mixer with a near “pulseless” pump system. The resultant mixture is then subjected for downstream purification and buffer exchange. The buffer exchange may be achieved using dialysis cassettes or a TFF system. TFF may be used to concentrate and buffer-exchange the resulting nascent LNP immediately after formation via the T-mix process. The diafiltration process is a continuous operation, keeping the volume constant by adding appropriate buffer at the same rate as the permeate flow.

### IX. Packaging and Use of the mRNA-LNP Vaccine

The mRNA-LNP vaccines can be formulated or packaged for parenteral (e.g., intramuscular, intradermal, or subcutaneous) administration or nasopharyngeal (e.g., intranasal) administration. In various embodiments, the mRNA-LNP vaccines may be formulated or packaged for pulmonary administration. In various embodiments, the mRNA-LNP vaccines may be formulated or packaged for intravenous administration. The vaccine compositions may be in the form of an extemporaneous formulation, where the LNP composition is lyophilized and reconstituted with a physiological buffer (e.g., PBS) just before use. The vaccine compositions also may be shipped and provided in the form of an aqueous solution or a frozen aqueous solution and can be directly administered to subjects without reconstitution (after thawing, if previously frozen).

Accordingly, the present disclosure provides an article of manufacture, such as a kit, that provides the mRNA-LNP vaccine in a single container or provides the mRNA-LNP vaccine in one container (e.g., a first container) and a physiological buffer for reconstitution in another container (e.g., a second container). The container(s) may contain a single-use dosage or multi-use dosage. The container(s) may be pre-treated glass vials or ampules. The article of manufacture may include instructions for use as well.

In certain embodiments, the mRNA-LNP vaccine is provided for use in intramuscular (IM) injection. The vaccine can be injected into a subject at, e.g., his/her deltoid muscle in the upper arm. In some embodiments, the vaccine is provided in a pre-filled syringe or injector (e.g., single-chambered or multi-chambered). In some embodiments, the vaccine is provided for use in inhalation and is provided in a pre-filled pump, aerosolizer, or inhaler.

In certain embodiments, nucleic acids, polypeptides, compositions of the invention are provided for use in skin injection, e.g. in the epidermis, the dermis or the hypodermis of the skin. In some embodiments, the nucleic acids, polypeptides, compositions are provided in a device suitable for skin injection, such as a needle (e.g. an epidermic, dermic or hypodermic needle), a needle free device, a microneedle device or a microprojection array device. Examples of microneedle or microprojection array devices suitable for the skin injection according to the invention are described in US20230270842A1, US20220339416A1, US20210085598A1, US20200246450A1, US20220143376A1, US20180264244A1, US20180263641A1, and US20110245776A1.

The mRNA-LNP vaccines can be administered to subjects in need thereof in a prophylactically effective amount, i.e., an amount that provides sufficient immune protection against a target pathogen for a sufficient amount of time (e.g., one year, two years, five years, ten years, or a lifetime). Sufficient immune protection may be, for example, prevention or alleviation of symptoms associated with infections by the pathogen. In some embodiments, multiple doses (e.g., two doses) of the vaccine are administered (e.g., injected) to subjects in need thereof to achieve the desired prophylactic effects. The doses (e.g., prime and booster doses) may be separated by an interval of at least, e.g., 2 weeks, 3 weeks, 4 weeks, one month, two months, three months, four months, five months, six months, one year, two years, five years, or ten years.

### X. Vectors

In one aspect, disclosed herein are vectors comprising the mRNA compositions disclosed herein. The RNA sequences encoding a protein of interest (e.g., mRNA encoding an RSV F protein, an hMPV F protein, or an hPIV3 F protein) can be cloned into a number of types of vectors. For example, the nucleic acids can be cloned into a vector including, but not limited to, a plasmid, a phagemid, a phage derivative, an animal virus, and a cosmid. Vectors of particular interest can include expression vectors, replication vectors, probe generation vectors, sequencing vectors, and vectors optimized for in vitro transcription.

In certain embodiments, the vector can be used to express mRNA in a host cell. In various embodiments, the vector can be used as a template for IVT. The construction of optimally translated IVT mRNA suitable for therapeutic use is disclosed in detail in Sahin, et al. (2014). Nat. Rev. Drug Discov. 13, 759-780; Weissman (2015). Expert Rev. Vaccines 14, 265-281.

In some embodiments, the vectors disclosed herein can comprise at least the following, from 5′ to 3′: an RNA polymerase promoter; a polynucleotide sequence encoding a 5′ UTR; a polynucleotide sequence encoding an ORF; a polynucleotide sequence encoding a 3′ UTR; and a polynucleotide sequence encoding at least one RNA aptamer. In some embodiments, the vectors disclosed herein may comprise a polynucleotide sequence encoding a poly(A) sequence and/or a polyadenylation signal.

A variety of RNA polymerase promoters are known. In some embodiments, the promoter can be a T7 RNA polymerase promoter. Other useful promoters can include, but are not limited to, T3 and SP6 RNA polymerase promoters. Consensus nucleotide sequences for T7, T3, and SP6 promoters are known.

Also disclosed herein are host cells (e.g., mammalian cells, e.g., human cells) comprising the vectors or RNA compositions disclosed herein.

Polynucleotides can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg, Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12(8):861-70, or the TranslT-RNA transfection Kit (Mirus, Madison, WI).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the mRNA sequence in the host cell a variety of assays may be performed.

### XI. Self-Replicating RNA and Trans-Replicating RNA

### Self-Replicating RNA:

In one aspect, disclosed herein are self-replicating RNAs encoding an RSV F protein, an hMPV F protein, and/or an hPIV3 protein.

Self-replicating RNA can be produced by using replication elements derived from, e.g., alphaviruses, and substituting the structural viral proteins with a nucleotide sequence encoding a protein of interest (e.g., RSV F protein, hMPV F protein, hPIV3 F protein). A self-replicating RNA is typically a positive-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen (i.e., an RSV F protein antigen, an hMPV F protein antigen, an hPIV3 F protein antigen), or may be transcribed to provide further transcripts with the same sense as the delivered RNA which are translated to provide in situ expression of the antigen. The overall result of this sequence of transcriptions is a large amplification in the number of the introduced replicon RNAs and so the encoded antigen becomes a major polypeptide product of the cells.

One suitable system for achieving self-replication in this manner is to use an alphavirus-based replicon. These replicons are positive stranded (positive sense-stranded) RNAs which lead to translation of a replicase (or replicase-transcriptase) after delivery to a cell. The replicase is translated as a polyprotein which auto-cleaves to provide a replication complex which creates genomic-strand copies of the positive-strand delivered RNA. These negative (−)-stranded transcripts can themselves be transcribed to give further copies of the positive-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, see the following reference: International Pub. No. WO 2005/113782, incorporated herein by reference.

In one embodiment, each self-replicating RNA described herein encodes (i) an RNA-dependent RNA polymerase which can transcribe RNA from the self-replicating RNA molecule and (ii) an RSV F protein antigen, an hMPV F protein antigen, or an hPIV3 F protein antigen. The polymerase can be an alphavirus replicase, e.g., comprising one or more of alphavirus proteins nsP1, nsP2, nsP3, and nsP4. Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins. Self-replicating RNA are described in further detail in International Pub. No. WO 2011/005799, incorporated herein by reference.

### Trans-Replicating RNA:

In one aspect, disclosed herein are trans-replicating RNAs encoding an RSV F protein, an hMPV F protein, and/or an hPIV3 protein.

Trans-replicating RNA possess similar elements as the self-replicating RNA described above. However, with trans replicating RNA, two separate RNA molecules are used. A first RNA molecule encodes for the RNA replicase described above (e.g., the alphavirus replicase) and a second RNA molecule encodes for the protein of interest (e.g., an RSV F protein antigen, an hMPV F protein antigen, an hPIV3 F protein antigen). The RNA replicase may replicate one or both of the first and second RNA molecule, thereby greatly increasing the copy number of RNA molecules encoding the protein of interest. Trans replicating RNA are described in further detail in International Pub. No. WO 2017/162265, incorporated herein by reference.

### XII. Pharmaceutical Compositions

RNA purified according to this disclosure can be useful as a component in pharmaceutical compositions, for example, for use as a vaccine. These compositions will typically include RNA and a pharmaceutically acceptable carrier. A pharmaceutical composition of the present disclosure can also include one or more additional components such as small molecule immunopotentiators (e.g., TLR agonists). A pharmaceutical composition of the present disclosure can also include a delivery system for the RNA, such as a liposome, an oil-in-water emulsion, or a microparticle. In some embodiments, the pharmaceutical composition comprises a lipid nanoparticle (LNP). In certain embodiments, the composition comprises an antigen-encoding nucleic acid molecule encapsulated within an LNP.

### XIII. Methods of Vaccination

The combination respiratory mRNA vaccine compositions disclosed herein may be administered to a subject to induce an immune response directed against the hRSV F protein, the hMPV F protein, and/or the hPIV3 protein wherein an anti-antigen antibody titer in the subject is increased following vaccination relative to an anti-antigen antibody titer in a subject that is not vaccinated with the compositions disclosed herein, or relative to an alternative vaccine against hRSV, hMPV, and/or hPIV3. An “anti-antigen antibody” is a serum antibody that binds specifically to the antigen.

In one aspect, the disclosure provides a method of eliciting an immune response to hRSV or a method of protecting a subject against hRSV infection comprising administering the compositions described herein to a subject. In another aspect, the disclosure provides a method of eliciting an immune response to hMPV or a method of protecting a subject against hMPV infection comprising administering the compositions described herein to a subject. In yet another aspect, the disclosure provides a method of eliciting an immune response to hPIV3, or a method of protecting a subject against hPIV3 infection comprising administering the compositions described herein to a subject.

The disclosure provides compositions for use in eliciting an immune response to hRSV or for protecting a subject against hRSV infection comprising administering the compositions described herein to a subject. The disclosure also provides compositions described herein for use in eliciting an immune response to hMPV or for protecting a subject against hMPV infection comprising administering the compositions described herein to a subject. The disclosure further provides compositions described herein for use in eliciting an immune response to hPIV3 or for protecting a subject against hPIV3 infection comprising administering the compositions described herein to a subject.

The disclosure provides compositions described herein for use in the manufacture of a medicament for eliciting an immune response to hRSV or protecting a subject against hRSV infection. The disclosure also provides compositions described herein for use in the manufacture of a medicament for eliciting an immune response to hMPV or protecting a subject against hMPV infection. The disclosure further provides compositions described herein for use in the manufacture of a medicament for eliciting an immune response to hPIV3 or protecting a subject against hPIV3 infection.

In certain embodiments, the compositions described herein increase the serum concentration of antibodies with binding specificity to site Ø of the RSV F protein. In certain embodiments, the compositions described herein increase the serum concentration of antibodies with binding specificity to site Ø of the hMPV F protein. In certain embodiments, the compositions described herein increase the serum concentration of antibodies with binding specificity to site Ø of the hPIV3 F protein.

In certain embodiments, the subject has about the same or higher serum concentration of neutralizing antibodies against RSV after administration of the compositions described herein, relative to a subject that is administered a single antigenic composition comprising an mRNA ORF encoding an hRSV F protein antigen of SEQ ID NO: 1. In certain embodiments, the subject has about the same or higher serum concentration of neutralizing antibodies against hMPV after administration of the compositions described herein, relative to a subject that is administered a single antigenic composition comprising an mRNA ORF encoding an hMPV F protein antigen of SEQ ID NO: 2. In certain embodiments, the subject has about the same or higher serum concentration of neutralizing antibodies against hPIV3 after administration of the compositions described herein, relative to a subject that is administered a single antigenic composition comprising an mRNA ORF encoding an hPIV3 F protein antigen of SEQ ID NO: 3.

In certain embodiments, the subject has a comparable serum concentration of neutralizing antibodies against hRSV after administration of the compositions described herein, relative to a subject that is administered a protein hRSV vaccine. In certain embodiments, the subject has a comparable serum concentration of neutralizing antibodies against hMPV after administration of the compositions described herein, relative to a subject that is administered a protein hMPV vaccine. In certain embodiments, the subject has a comparable serum concentration of neutralizing antibodies against hPIV3 after administration of the compositions described herein, relative to a subject that is administered a protein hPIV3 vaccine.

In certain embodiments, the compositions described herein increase the serum concentration of neutralizing antibodies in a subject with pre-existing hRSV immunity. In certain embodiments, the compositions described herein increases the serum concentration of neutralizing antibodies in a subject with pre-existing hMPV immunity. In certain embodiments, the compositions described herein increase the serum concentration of neutralizing antibodies in a subject with pre-existing hPIV3 immunity.

### EXAMPLES

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### Example 1: mRNAs Encoding hRSV F Protein, hMPV F Protein, and hPIV3 F Protein

RSV F, hMPV F, and hPIV3 F proteins were selected for testing as a combination mRNA-based vaccine. The F protein designated RSV FD1 corresponds to a WT RSV F protein. The F protein designated RSV FD3 corresponds to a pre-fusion RSV F protein. The F protein designated hMPV FD1 corresponds to a WT hMPV F protein. The F protein designated hMPV FD2 corresponds to a pre-fusion hMPV F protein. The F protein designated hPIV3 FD1 corresponds to a WT hPIV3 F protein. The F protein designated hPIV3 FD2 corresponds to a pre-fusion hPIV3 F protein. The amino acid sequences for each of the F proteins are recited below.

hRSV FD1:
(SEQ ID NO: 9)
MELLILKANAITTILTAVTFCFASGQNITEEFYQSTCSAVSKGYLSALRTGWYTSVITIELSNIKKNKCN
GTDAKVKLIKQELDKYKNAVTELQLLMQSTQATNNRARRELPRFMNYTLNNAKKTNVTLSKKRKRR
FLGFLLGVGSAIASGVAVSKVLHLEGEVNKIKSALLSTNKAVVSLSNGVSVLTSKVLDLKNYIDKQLL
PIVNKQSCSISNIETVIEFQQKNNRLLEITREFSVNAGVTTPVSTYMLTNSELLSLINDMPITNDQKKL
MSNNVQIVRQQSYSIMSIIKEEVLAYVVQLPLYGVIDTPCWKLHTSPLCTTNTKEGSNICLTRTDRG
WYCDNAGSVSFFPQAETCKVQSNRVFCDTMNSLTLPSEVNLCNVDIFNPKYDCKIMTSKTDVSSS
VITSLGAIVSCYGKTKCTASNKNRGIIKTFSNGCDYVSNKGVDTVSVGNTLYYVNKQEGKSLYVKGE
PIINFYDPLVFPSDEFDASISQVNEKINQSLAFIRKSDELLHNVNAGKSTTNIMITTIIIVIIVILLSL
IAVGLLLYCKARSTPVTLSKDQLSGINNIAFSN
hRSV FD3:
(SEQ ID NO: 1)
MELLILKANAITTILTAVTFCFASGQNITEEFYQSTCSAVSKGYLSALRTGWYTSVITIELSNIKENKCN
GTDAKVKLIKQELDKYKNAVTELQLLMGSGNVGLGGAIASGVAVSKVLHLEGEVNKIKSALLSTNKA
VVSLSNGVSVLTFKVLDLKNYIDKQLLPILNKQSCSISNPETVIEFQQKNNRLLEITREFSVNAGVTTP
VSTYMLTNSELLSLINDMPITNDQKKLMSNNVQIVRQQSYSIMSIIKEEVLAYVVQLPLYGVIDTPCW
KLHTSPLCTTNTKNGSNICLTRTDRGWYCDNAGNVSFFPQAETCKVQSNRVFCDTMNSRTLPSEV
NLCNVDIFNPKYDCKIMTSKTDVSSSVITSLGAIVSCYGKTKCTASNKNRGIIKTFSNGCDYVSNKGV
DTVSVGNTLYYVNKQEGKSLYVKGEPIINFYDPLVFPSDEFDASISQVNELINQSLAFINQSDELLHN
VNAGKSTTNIMITTIIIVIIVILLSLIAVGLLLYCKARSTPVTLSKDQLSGINNIAFSN
hMPV FD1:
(SEQ ID NO: 10)
MSWKVVIIFSLLITPQHGLKESYLEESCSTITEGYLSVLRTGWYTNVFTLEVGDVENLTCSDGPSLIK
TELDLTKSALRELKTVSADQLAREEQIENPRQSRFVLGAIALGVATAAAVTAGVAIAKTIRLESEVTAI
KNALKTTNEAVSTLGNGVRVLATAVRELKDFVSKNLTRAINKNKCDIDDLKMAVSFSQFNRRFLNVV
RQFSDNAGITPAISLDLMTDAELARAVSNMPTSAGQIKLMLENRAMVRRKGFGILIGVYGSSVIYMV
QLPIFGVIDTPCWIVKAAPSCSEKKGNYACLLREDQGWYCQNAGSTVYYPNEKDCETRGDHVFCD
TAAGINVAEQSKECNINISTTNYPCKVSTGRHPISMVALSPLGALVACYKGVSCSIGSNRVGIIKQLN
KGCSYITNQDADTVTIDNTVYQLSKVEGEQHVIKGRPVSSSFDPIKFPEDQFNVALDQVFENIENSQ
ALVDQSNRILSSAEKGNTGFIIVIILIAVLGSSMILVSIFIIIKKTKKPTGAPPELSGVTNNGFIPHN
hMPV FD2:
(SEQ ID NO: 2)
MSWKVVIIFSLLITPQHGLKESYLEESCSTITEGYLSVLRTGWYTVVFTLEVGDVENLTCSDGPSLIKT
ELDLTKSALRELKTVSADQLAREEQIENPRQSRFVLGAIALGVATAAAVTAGVAIAKTIRLESEVTAIK
NALKTTNEAVSTLGNGVRVLAFAVRELKDFVSKNLTRAINKNKCDIDDLKMAVSFSQFNRRFLNVVR
QFSDNAGITPAISLDLMTDAELARAVSNMPTSAGQIKLMLENRAMVRRKGFGILIGVYGSSVIYMVQ
LPIFGVIDTPCWIVKAAPSCSEKKGNYACLLREDQGWYCQNAGSTVYYPNEKDCETRGDHVFCDT
AAGINVAEQSKECNINISTTNYPCKVSTGRHPISMVALSPLGALVACYKGVSCSIGSNRVGIIKQLNK
GCSYITNQDADTVTIDNTVYQLSKVEGEQHVIKGRPVSSSFDPIKFPEDQFNVALDQVFENIENSQA
LVDQSNRILSSAEKGNTGFIIVIILIAVLGSSMILVSIFIIIKKTKKPTGAPPELSGVTNNGFIPHN
(Lower case amino acids denote a linker, foldon motif, linker,
HRV-3C cleavage site, linker, 8X-His-tag(SEQ ID NO:42),
and strep-tag Il region.)
hPIV3 FD1:
(SEQ ID NO: 11)
MLISILSIITTMIMASHCQIDITKLQHVGVLVNSPKGMKISQNFETRYLILSLIPKIEDSNSCGDQQIKQ
YKRLLDRLIIPLYDGLKLQKDVIVTNQESNENTDPRTERFFGGVIGTIALGVATSAQITAAVALVEAKQA
KSDIEKLKEAIRDTNKAVQSVQSSVGNLIVAIKSVQDYVNKEIVPSIARLGCEAAGLQLGIALTQHYSEL
TNIFGDNIGSLQEKGIKLQGIASLYRTNITEIFTTSTVDKYDIYDLLFTESIKVRVIDVDLNDYSITLQV
RLPLLTRLLNTQIYKVDSISYNIQNREWYIPLPSHIMTKGAFLGGADVKECIEAFSSYICPSDPGFVLNH
EMESCLSGNISQCPRTTVTSDIVPRYAFVNGGVVANCITTTCTCNGIGNRINQPPDQGVKIITHKEC
NTIGINGMLFNTNKEGTLAFYTPDDITLNNSVALDPIDISIELNKAKSDLEESKEWIRRSNQKLDSIGS
WHQSSTTIIIILIMMIILFIINITIITIAIKYYRIQKRNRVDQNDKPYVLTNK
hPIV3 FD2:
(SEQ ID NO: 3)
MLISILSIITTMIMASHCQIDITKLQHVGVLVNSPKGMKISQNFETRYLILSLIPKIEDSNSCGDQQIKQY
KRLLDRLIIPLYDGLKLQKDVIVTNQESNENTDPRTERFFGGVIGTIALGVATSAQITAAVALVEAKQA
KSDIEKLKEAIRDTNKAVQSVCSSVGNCIVAIKSVQDYVNKEIVPSIARLGCEAAGLQLGIALTQHYSE
LTNCFGDNIGSLQEKGIKLQCIASLYRTNITEIFTTSTVDKYDIYDLLFTESIKVRVIDVDLNDYSITLQ
VRLPLLTRLLNTQIYKVDSISYNIQNREWYIPLPSHIMTKGAFLGGADVKECIEAFSSYICPSDPGFVLN
HEMESCLSGNISQCPRTTVTSDIVPRYAFVNGGVVANCITTTCTCNGIGNRINQPPDQGVKIITHKE
CNTIGINGMLFNTNKEGTLAFYTPDDITLNNSVALDPIDISIELNKVKSDLEESKEWYRRSNQKLDSIG
SWHQSSTTIIIILIMMIILFIINITIITIAIKYYRIQKRNRVDQNDKPYVLTNK

The mRNA described herein comprise an open reading frame (ORF) encoding an F protein antigen, at least one 5′ untranslated region (5′ UTR), at least one 3′ untranslated region (3′ UTR), and at least one polyadenylation (poly(A)) sequence. The mRNA further comprises a 5′ cap with the following structure:

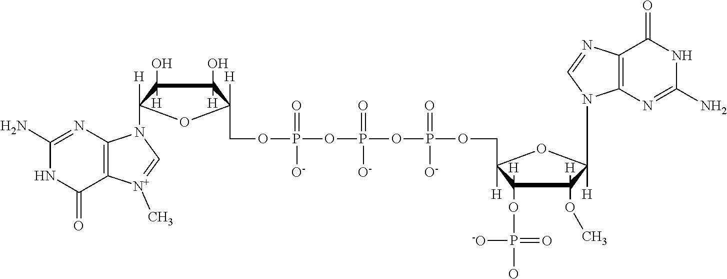

The nucleic acid sequences for each of the mRNA open reading frames (ORFs) encoding the hRSV FD3, hMPV FD2, and hPIV3 FD2 proteins are recited below.

hRSV FD3 mRNA ORF:
(SEQ ID NO: 4)
AUGGAACUGCUGAUCCUCAAAGCCAACGCAAUCACCACCAUUCUCACCGCUGUGACCUUCU
GCUUCGCAUCGGGGCAGAACAUCACUGAAGAGUUUUACCAGAGCACUUGCAGCGCGGUGU
CAAAGGGUUACCUUUCCGCACUGCGGACCGGAUGGUACACUUCCGUGAUCACCAUUGAGCU
CAGCAACAUCAAGGAAAACAAGUGCAAUGGCACCGACGCCAAGGUCAAGCUGAUCAAACAAG
AACUGGACAAGUACAAGAACGCCGUGACAGAAUUGCAGCUCCUGAUGGGAUCCGGAAACGU
CGGUCUGGGCGGAGCCAUCGCGAGUGGAGUGGCUGUGUCCAAGGUCUUGCACCUCGAGG
GAGAAGUGAACAAGAUCAAGUCCGCGCUGCUGUCAACGAACAAGGCCGUGGUGUCCCUGUC
UAACGGCGUCAGCGUGCUGACGUUCAAGGUCCUGGACCUGAAGAAUUACAUUGACAAGCAG
CUGCUGCCCAUCCUCAACAAGCAAUCCUGCUCCAUCUCCAACCCCGAAACCGUGAUCGAGU
UCCAGCAGAAGAACAACCGCCUGCUGGAAAUUACUCGCGAGUUCUCUGUGAAUGCCGGCGU
GACCACCCCUGUGUCCACCUACAUGCUGACCAACUCCGAGCUUCUCUCCCUUAUCAAUGAC
AUGCCUAUCACGAACGACCAGAAGAAGCUGAUGUCGAACAACGUGCAGAUUGUGCGGCAGC
AGUCAUACAGCAUCAUGUCGAUCAUCAAGGAAGAAGUGCUGGCGUACGUGGUGCAACUCCC
GCUGUACGGCGUCAUCGAUACCCCGUGCUGGAAGCUGCACACCUCGCCUUUGUGUACCAC
CAACACCAAGAACGGAUCCAACAUCUGCUUAACCCGGACUGAUCGGGGUUGGUACUGCGAC
AACGCCGGGAAUGUUUCGUUCUUCCCACAAGCCGAGACUUGUAAAGUGCAGUCAAACAGAG
UGUUCUGUGACACCAUGAACUCGAGAACCCUGCCCAGCGAAGUGAACCUGUGUAACGUCGA
CAUCUUUAACCCAAAAUACGAUUGCAAGAUUAUGACCAGCAAAACCGACGUGUCCUCCUCCG
UGAUAACAAGCCUGGGGGCGAUUGUGUCAUGCUACGGAAAGACUAAGUGCACCGCCUCGAA
CAAGAACCGCGGCAUCAUUAAGACUUUCUCGAAUGGUUGCGACUAUGUGUCCAACAAGGGC
GUGGAUACUGUGUCAGUCGGGAAUACUCUUUACUACGUGAACAAGCAGGAGGGGAAAAGCC
UCUACGUGAAGGGAGAGCCUAUUAUCAACUUUUACGAUCCGCUGGUGUUCCCGUCCGACGA
AUUCGACGCCAGCAUCAGCCAAGUCAACGAGCUGAUUAACCAGUCCCUCGCCUUCAUCAAC
CAAUCCGACGAGCUCCUGCAUAACGUGAACGCCGGAAAGUCCACCACCAACAUCAUGAUCA
CUACUAUUAUCAUCGUGAUCAUCGUCAUCCUGCUGAGCCUGAUUGCUGUGGGCCUGUUGC
UGUAUUGCAAAGCCAGGUCCACCCCGGUCACCCUGUCGAAGGAUCAGCUGUCCGGAAUCAA
CAACAUUGCCUUCUCCAACUAA
hMPV FD2 mRNA ORF:
(SEQ ID NO: 5)
AUGAGCUGGAAGGUUGUGAUUAUUUUCUCUCUGCUGAUUACUCCACAGCACGGCCUGAAG
GAGUCCUACCUGGAGGAGUCCUGUUCUACUAUCACUGAGGGGUAUCUCUCUGUGCUGCGG
ACAGGGUGGUAUACAGUGGUGUUCACCCUGGAGGUUGGCGAUGUGGAGAAUCUGACUUGC
AGCGAUGGCCCUUCUCUGAUCAAGACCGAGCUGGAUCUGACAAAAAGCGCCCUCAGAGAAC
UGAAAACCGUGUCCGCCGAUCAGCUGGCAAGGGAGGAGCAGAUCGAGAACCCACGGCAGA
GCAGGUUUGUGCUGGGCGCUAUCGCUCUGGGCGUGGCCACUGCAGCUGCUGUCACUGCA
GGGGUCGCAAUCGCUAAGACUAUCAGACUGGAAUCCGAGGUGACCGCCAUUAAGAAUGCCC
UGAAGACUACCAACGAGGCUGUGUCCACUCUGGGAAACGGAGUGAGGGUCCUGGCCUUCG
CAGUGAGGGAGCUGAAGGAUUUUGUGUCAAAGAACCUUACACGGGCCAUCAACAAGAAUAA
GUGCGAUAUCGAUGACCUGAAGAUGGCCGUGUCCUUCUCCCAGUUCAACCGGCGCUUUCU
GAAUGUGGUGCGCCAGUUUUCCGACAACGCUGGAAUCACCCCUGCUAUCAGCCUGGACCU
CAUGACCGACGCCGAACUCGCAAGGGCCGUUUCUAACAUGCCUACAUCCGCUGGACAGAUU
AAGCUGAUGCUGGAGAAUAGAGCAAUGGUGAGGAGAAAGGGAUUCGGCAUCCUGAUUGGC
GUGUACGGAUCUAGCGUGAUCUACAUGGUGCAGCUGCCGAUCUUCGGCGUGAUCGAUACU
CCUUGUUGGAUCGUCAAGGCCGCCCCUUCCUGCUCCGAGAAGAAGGGCAAUUACGCUUGU
CUGCUGCGGGAGGACCAGGGCUGGUAUUGCCAGAACGCCGGGUCUACAGUGUACUAUCCU
AACGAGAAGGAUUGCGAGACCAGAGGCGACCACGUUUUCUGUGAUACAGCCGCCGGAAUCA
AUGUCGCAGAGCAGUCUAAGGAGUGCAACAUCAAUAUCUCUACAACCAAUUACCCAUGUAAG
GUGAGCACUGGACGGCACCCUAUCAGUAUGGUGGCUCUGAGCCCACUGGGGGCACUGGUG
GCUUGCUACAAGGGGGUGAGCUGCAGUAUCGGCAGUAACAGAGUGGGCAUUAUCAAGCAG
CUGAACAAAGGGUGCUCUUAUAUUACAAACCAGGAUGCAGAUACUGUGACCAUCGACAACA
CUGUGUACCAGCUGUCCAAGGUGGAGGGGGAGCAGCAUGUGAUCAAAGGGAGACCCGUCU
CUUCUUCUUUCGAUCCCAUCAAGUUCCCUGAAGACCAGUUCAAUGUUGCCCUGGACCAGGU
UUUCGAGAACAUCGAAAAUAGCCAGGCCUUGGUCGAUCAAUCCAACAGGAUCCUGAGCAGC
GCAGAGAAAGGGAACACUGGCUUCAUCAUCGUGAUCAUUCUGAUCGCCGUGCUGGGGAGC
AGUAUGAUUCUGGUGUCCAUUUUCAUCAUCAUCAAGAAGACCAAGAAGCCUACAGGAGCAC
CCCCUGAGCUGAGCGGAGUGACCAACAACGGCUUUAUCCCUCACAACUAA
hPIV3 FD2 mRNA ORF:
(SEQ ID NO: 6)
AUGCUGAUUAGCAUUCUGUCCAUCAUCACAACCAUGAUUAUGGCAAGUCACUGUCAGAUCG
AUAUCACCAAGCUCCAGCACGUGGGCGUGCUGGUGAAUUCCCCAAAGGGGAUGAAGAUUUC
UCAGAACUUUGAGACUCGGUACCUGAUCCUGUCCCUGAUUCCUAAGAUUGAGGAUAGCAAC
UCCUGUGGCGACCAGCAGAUCAAACAGUACAAACGCCUGCUGGACAGACUGAUCAUCCCAC
UGUACGACGGCCUGAAGCUGCAGAAAGACGUCAUUGUUACAAACCAGGAAAGCAACGAGAA
UACCGACCCUAGGACUGAGCGGUUCUUCGGGGGCGUCAUCGGAACAAUCGCACUGGGCGU
UGCCACUAGCGCACAGAUCACCGCCGCCGUUGCACUGGUGGAGGCUAAGCAGGCAAAAUCU
GACAUCGAGAAGCUGAAAGAAGCUAUCCGCGAUACCAACAAGGCCGUGCAGUCAGUGUGCU
CCAGCGUCGGCAACUGCAUCGUUGCUAUUAAGUCCGUGCAGGACUACGUGAACAAGGAGAU
CGUGCCCAGUAUCGCAAGGCUGGGAUGUGAGGCCGCUGGACUGCAGUUGGGGAUCGCUCU
CACCCAGCACUACUCCGAGCUGACAAAUUGCUUCGGAGAUAACAUCGGAUCUCUGCAGGAG
AAGGGCAUCAAACUGCAGUGUAUUGCCUCCCUGUACAGAACAAAUAUCACAGAGAUUUUCA
CAACCAGCACUGUCGACAAAUAUGAUAUCUAUGACCUGCUGUUCACCGAGUCCAUUAAGGU
GCGGGUCAUCGACGUUGACCUGAACGAUUAUUCCAUCACCCUCCAGGUGAGACUGCCUCU
GCUGACACGGCUGCUGAACACUCAGAUCUACAAGGUCGACUCCAUCAGCUACAACAUCCAG
AACCGCGAGUGGUACAUCCCACUGCCUAGCCACAUCAUGACAAAGGGCGCUUUUCUGGGAG
GAGCUGAUGUGAAGGAAUGCAUUGAGGCCUUCAGCAGCUACAUCUGCCCUUCCGAUCCCG
GCUUCGUCCUGAACCACGAGAUGGAGAGCUGUCUGAGCGGGAACAUUAGCCAGUGCCCCA
GGACAACCGUCACCUCUGACAUUGUGCCAAGGUAUGCCUUCGUGAACGGCGGAGUGGUUG
CCAAUUGCAUCACCACUACAUGCACUUGCAAUGGGAUUGGCAACAGGAUCAACCAGCCACC
AGACCAGGGGGUGAAAAUCAUUACACACAAAGAGUGCAACACCAUCGGCAUCAACGGAAUG
CUGUUCAAUACCAACAAGGAGGGCACCCUGGCCUUCUACACACCCGAUGACAUCACCCUGA
ACAAUAGCGUGGCUCUGGAUCCCAUUGAUAUCAGCAUUGAGCUGAAUAAGGUCAAGUCUGA
UCUGGAGGAGAGCAAGGAGUGGUAUAGACGGUCUAAUCAGAAGCUCGACUCUAUCGGAUCC
UGGCACCAGUCCUCCACAACAAUCAUCAUUAUCCUGAUCAUGAUGAUCAUCCUGUUCAUCA
UCAACAUCACAAUUAUCACCAUUGCCAUCAAGUAUUACCGGAUUCAGAAGCGGAAUCGCGU
UGACCAGAACGACAAACCUUAUGUUCUGACCAACAAAUAA

The nucleic acid sequences for each of the DNA templates encoding the codon-optimized hRSV FD3, hMPV FD2, and hPIV3 FD2 mRNA are recited below.

hRSV FD3 DNA:
(SEQ ID NO: 12)
ATGGAACTGCTGATCCTCAAAGCCAACGCAATCACCACCATTCTCACCGCTGTGACCTTCTGC
TTCGCATCGGGGCAGAACATCACTGAAGAGTTTTACCAGAGCACTTGCAGCGCGGTGTCAAAG
GGTTACCTTTCCGCACTGCGGACCGGATGGTACACTTCCGTGATCACCATTGAGCTCAGCAAC
ATCAAGGAAAACAAGTGCAATGGCACCGACGCCAAGGTCAAGCTGATCAAACAAGAACTGGAC
AAGTACAAGAACGCCGTGACAGAATTGCAGCTCCTGATGGGATCCGGAAACGTCGGTCTGGG
CGGAGCCATCGCGAGTGGAGTGGCTGTGTCCAAGGTCTTGCACCTCGAGGGAGAAGTGAACA
AGATCAAGTCCGCGCTGCTGTCAACGAACAAGGCCGTGGTGTCCCTGTCTAACGGCGTCAGC
GTGCTGACGTTCAAGGTCCTGGACCTGAAGAATTACATTGACAAGCAGCTGCTGCCCATCCTC
AACAAGCAATCCTGCTCCATCTCCAACCCCGAAACCGTGATCGAGTTCCAGCAGAAGAACAAC
CGCCTGCTGGAAATTACTCGCGAGTTCTCTGTGAATGCCGGCGTGACCACCCCTGTGTCCACC
TACATGCTGACCAACTCCGAGCTTCTCTCCCTTATCAATGACATGCCTATCACGAACGACCAGA
AGAAGCTGATGTCGAACAACGTGCAGATTGTGCGGCAGCAGTCATACAGCATCATGTCGATCA
TCAAGGAAGAAGTGCTGGCGTACGTGGTGCAACTCCCGCTGTACGGCGTCATCGATACCCCG
TGCTGGAAGCTGCACACCTCGCCTTTGTGTACCACCAACACCAAGAACGGATCCAACATCTGC
TTAACCCGGACTGATCGGGGTTGGTACTGCGACAACGCCGGGAATGTTTCGTTCTTCCCACAA
GCCGAGACTTGTAAAGTGCAGTCAAACAGAGTGTTCTGTGACACCATGAACTCGAGAACCCTG
CCCAGCGAAGTGAACCTGTGTAACGTCGACATCTTTAACCCAAAATACGATTGCAAGATTATGA
CCAGCAAAACCGACGTGTCCTCCTCCGTGATAACAAGCCTGGGGGCGATTGTGTCATGCTAC
GGAAAGACTAAGTGCACCGCCTCGAACAAGAACCGCGGCATCATTAAGACTTTCTCGAATGGT
TGCGACTATGTGTCCAACAAGGGCGTGGATACTGTGTCAGTCGGGAATACTCTTTACTACGTG
AACAAGCAGGAGGGGAAAAGCCTCTACGTGAAGGGAGAGCCTATTATCAACTTTTACGATCCG
CTGGTGTTCCCGTCCGACGAATTCGACGCCAGCATCAGCCAAGTCAACGAGCTGATTAACCAG
TCCCTCGCCTTCATCAACCAATCCGACGAGCTCCTGCATAACGTGAACGCCGGAAAGTCCACC
ACCAACATCATGATCACTACTATTATCATCGTGATCATCGTCATCCTGCTGAGCCTGATTGCTG
TGGGCCTGTTGCTGTATTGCAAAGCCAGGTCCACCCCGGTCACCCTGTCGAAGGATCAGCTG
TCCGGAATCAACAACATTGCCTTCTCCAACTAA
hMPV FD2 DNA:
(SEQ ID NO: 13)
ATGAGCTGGAAGGTTGTGATTATTTTCTCTCTGCTGATTACTCCACAGCACGGCCTGAAGGAGT
CCTACCTGGAGGAGTCCTGTTCTACTATCACTGAGGGGTATCTCTCTGTGCTGCGGACAGGGT
GGTATACAGTGGTGTTCACCCTGGAGGTTGGCGATGTGGAGAATCTGACTTGCAGCGATGGC
CCTTCTCTGATCAAGACCGAGCTGGATCTGACAAAAAGCGCCCTCAGAGAACTGAAAACCGTG
TCCGCCGATCAGCTGGCAAGGGAGGAGCAGATCGAGAACCCACGGCAGAGCAGGTTTGTGCT
GGGCGCTATCGCTCTGGGCGTGGCCACTGCAGCTGCTGTCACTGCAGGGGTCGCAATCGCTA
AGACTATCAGACTGGAATCCGAGGTGACCGCCATTAAGAATGCCCTGAAGACTACCAACGAGG
CTGTGTCCACTCTGGGAAACGGAGTGAGGGTCCTGGCCTTCGCAGTGAGGGAGCTGAAGGAT
TTTGTGTCAAAGAACCTTACACGGGCCATCAACAAGAATAAGTGCGATATCGATGACCTGAAGA
TGGCCGTGTCCTTCTCCCAGTTCAACCGGCGCTTTCTGAATGTGGTGCGCCAGTTTTCCGACA
ACGCTGGAATCACCCCTGCTATCAGCCTGGACCTCATGACCGACGCCGAACTCGCAAGGGCC
GTTTCTAACATGCCTACATCCGCTGGACAGATTAAGCTGATGCTGGAGAATAGAGCAATGGTG
AGGAGAAAGGGATTCGGCATCCTGATTGGCGTGTACGGATCTAGCGTGATCTACATGGTGCA
GCTGCCGATCTTCGGCGTGATCGATACTCCTTGTTGGATCGTCAAGGCCGCCCCTTCCTGCTC
CGAGAAGAAGGGCAATTACGCTTGTCTGCTGCGGGAGGACCAGGGCTGGTATTGCCAGAACG
CCGGGTCTACAGTGTACTATCCTAACGAGAAGGATTGCGAGACCAGAGGCGACCACGTTTTCT
GTGATACAGCCGCCGGAATCAATGTCGCAGAGCAGTCTAAGGAGTGCAACATCAATATCTCTA
CAACCAATTACCCATGTAAGGTGAGCACTGGACGGCACCCTATCAGTATGGTGGCTCTGAGCC
CACTGGGGGCACTGGTGGCTTGCTACAAGGGGGTGAGCTGCAGTATCGGCAGTAACAGAGTG
GGCATTATCAAGCAGCTGAACAAAGGGTGCTCTTATATTACAAACCAGGATGCAGATACTGTGA
CCATCGACAACACTGTGTACCAGCTGTCCAAGGTGGAGGGGGAGCAGCATGTGATCAAAGGG
AGACCCGTCTCTTCTTCTTTCGATCCCATCAAGTTCCCTGAAGACCAGTTCAATGTTGCCCTGG
ACCAGGTTTTCGAGAACATCGAAAATAGCCAGGCCTTGGTCGATCAATCCAACAGGATCCTGA
GCAGCGCAGAGAAAGGGAACACTGGCTTCATCATCGTGATCATTCTGATCGCCGTGCTGGGG
AGCAGTATGATTCTGGTGTCCATTTTCATCATCATCAAGAAGACCAAGAAGCCTACAGGAGCAC
CCCCTGAGCTGAGCGGAGTGACCAACAACGGCTTTATCCCTCACAACTAA
hPIV3 FD2 DNA:
(SEQ ID NO: 14)
ATGCTGATTAGCATTCTGTCCATCATCACAACCATGATTATGGCAAGTCACTGTCAGATCGATA
TCACCAAGCTCCAGCACGTGGGCGTGCTGGTGAATTCCCCAAAGGGGATGAAGATTTCTCAGA
ACTTTGAGACTCGGTACCTGATCCTGTCCCTGATTCCTAAGATTGAGGATAGCAACTCCTGTGG
CGACCAGCAGATCAAACAGTACAAACGCCTGCTGGACAGACTGATCATCCCACTGTACGACGG
CCTGAAGCTGCAGAAAGACGTCATTGTTACAAACCAGGAAAGCAACGAGAATACCGACCCTAG
GACTGAGCGGTTCTTCGGGGGCGTCATCGGAACAATCGCACTGGGCGTTGCCACTAGCGCAC
AGATCACCGCCGCCGTTGCACTGGTGGAGGCTAAGCAGGCAAAATCTGACATCGAGAAGCTG
AAAGAAGCTATCCGCGATACCAACAAGGCCGTGCAGTCAGTGTGCTCCAGCGTCGGCAACTG
CATCGTTGCTATTAAGTCCGTGCAGGACTACGTGAACAAGGAGATCGTGCCCAGTATCGCAAG
GCTGGGATGTGAGGCCGCTGGACTGCAGTTGGGGATCGCTCTCACCCAGCACTACTCCGAGC
TGACAAATTGCTTCGGAGATAACATCGGATCTCTGCAGGAGAAGGGCATCAAACTGCAGTGTA
TTGCCTCCCTGTACAGAACAAATATCACAGAGATTTTCACAACCAGCACTGTCGACAAATATGA
TATCTATGACCTGCTGTTCACCGAGTCCATTAAGGTGCGGGTCATCGACGTTGACCTGAACGA
TTATTCCATCACCCTCCAGGTGAGACTGCCTCTGCTGACACGGCTGCTGAACACTCAGATCTA
CAAGGTCGACTCCATCAGCTACAACATCCAGAACCGCGAGTGGTACATCCCACTGCCTAGCCA
CATCATGACAAAGGGCGCTTTTCTGGGAGGAGCTGATGTGAAGGAATGCATTGAGGCCTTCAG
CAGCTACATCTGCCCTTCCGATCCCGGCTTCGTCCTGAACCACGAGATGGAGAGCTGTCTGAG
CGGGAACATTAGCCAGTGCCCCAGGACAACCGTCACCTCTGACATTGTGCCAAGGTATGCCTT
CGTGAACGGCGGAGTGGTTGCCAATTGCATCACCACTACATGCACTTGCAATGGGATTGGCAA
CAGGATCAACCAGCCACCAGACCAGGGGGTGAAAATCATTACACACAAAGAGTGCAACACCAT
CGGCATCAACGGAATGCTGTTCAATACCAACAAGGAGGGCACCCTGGCCTTCTACACACCCGA
TGACATCACCCTGAACAATAGCGTGGCTCTGGATCCCATTGATATCAGCATTGAGCTGAATAAG
GTCAAGTCTGATCTGGAGGAGAGCAAGGAGTGGTATAGACGGTCTAATCAGAAGCTCGACTCT
ATCGGATCCTGGCACCAGTCCTCCACAACAATCATCATTATCCTGATCATGATGATCATCCTGT
TCATCATCAACATCACAATTATCACCATTGCCATCAAGTATTACCGGATTCAGAAGCGGAATCG
CGTTGACCAGAACGACAAACCTTATGTTCTGACCAACAAATAA

The nucleic acid sequences for the 5′ UTR and 3′ UTR (with respect to hRSV F, hMPV F, and hPIV3 F) are recited below.

(SEQ ID NO: 8)
5′ UTR: GGACAGAUCGCCUGGAGACGCCAUCCACGCUGUUUUGACCUC
CAUAGAAGACACCGGGACCGAUCCAGCCUCCGCGGCCGGGAACGGUGCAU
UGGAACGCGGAUUCCCCGUGCCAAGAGUGACUCACCGUCCUUGACACG
(SEQ ID NO: 7)
3′ UTR: CGGGUGGCAUCCCUGUGACCCCUCCCCAGUGCCUCUCCUGGC
CCUGGAAGUUGCCACUCCAGUGCCCACCAGCCUUGUCCUAAUAAAAUUAA
GUUGCAUC

The nucleic acid sequences for each of the full-length mRNA encoding the hRSV FD3, hMPV FD2, and hPIV3 FD2 proteins are recited below.

hRSV FD3 mRNA:
(SEQ ID NO: 15)
GGACAGAUCGCCUGGAGACGCCAUCCACGCUGUUUUGACCUCCAUAGAAGACACCGGGACC
GAUCCAGCCUCCGCGGCCGGGAACGGUGCAUUGGAACGCGGAUUCCCCGUGCCAAGAGUG
ACUCACCGUCCUUGACACGAUGGAACUGCUGAUCCUCAAAGCCAACGCAAUCACCACCAUU
CUCACCGCUGUGACCUUCUGCUUCGCAUCGGGGCAGAACAUCACUGAAGAGUUUUACCAGA
GCACUUGCAGCGCGGUGUCAAAGGGUUACCUUUCCGCACUGCGGACCGGAUGGUACACUU
CCGUGAUCACCAUUGAGCUCAGCAACAUCAAGGAAAACAAGUGCAAUGGCACCGACGCCAA
GGUCAAGCUGAUCAAACAAGAACUGGACAAGUACAAGAACGCCGUGACAGAAUUGCAGCUC
CUGAUGGGAUCCGGAAACGUCGGUCUGGGCGGAGCCAUCGCGAGUGGAGUGGCUGUGUC
CAAGGUCUUGCACCUCGAGGGAGAAGUGAACAAGAUCAAGUCCGCGCUGCUGUCAACGAAC
AAGGCCGUGGUGUCCCUGUCUAACGGCGUCAGCGUGCUGACGUUCAAGGUCCUGGACCUG
AAGAAUUACAUUGACAAGCAGCUGCUGCCCAUCCUCAACAAGCAAUCCUGCUCCAUCUCCAA
CCCCGAAACCGUGAUCGAGUUCCAGCAGAAGAACAACCGCCUGCUGGAAAUUACUCGCGAG
UUCUCUGUGAAUGCCGGCGUGACCACCCCUGUGUCCACCUACAUGCUGACCAACUCCGAGC
UUCUCUCCCUUAUCAAUGACAUGCCUAUCACGAACGACCAGAAGAAGCUGAUGUCGAACAA
CGUGCAGAUUGUGCGGCAGCAGUCAUACAGCAUCAUGUCGAUCAUCAAGGAAGAAGUGCUG
GCGUACGUGGUGCAACUCCCGCUGUACGGCGUCAUCGAUACCCCGUGCUGGAAGCUGCAC
ACCUCGCCUUUGUGUACCACCAACACCAAGAACGGAUCCAACAUCUGCUUAACCCGGACUG
AUCGGGGUUGGUACUGCGACAACGCCGGGAAUGUUUCGUUCUUCCCACAAGCCGAGACUU
GUAAAGUGCAGUCAAACAGAGUGUUCUGUGACACCAUGAACUCGAGAACCCUGCCCAGCGA
AGUGAACCUGUGUAACGUCGACAUCUUUAACCCAAAAUACGAUUGCAAGAUUAUGACCAGCA
AAACCGACGUGUCCUCCUCCGUGAUAACAAGCCUGGGGGCGAUUGUGUCAUGCUACGGAAA
GACUAAGUGCACCGCCUCGAACAAGAACCGCGGCAUCAUUAAGACUUUCUCGAAUGGUUGC
GACUAUGUGUCCAACAAGGGCGUGGAUACUGUGUCAGUCGGGAAUACUCUUUACUACGUGA
ACAAGCAGGAGGGGAAAAGCCUCUACGUGAAGGGAGAGCCUAUUAUCAACUUUUACGAUCC
GCUGGUGUUCCCGUCCGACGAAUUCGACGCCAGCAUCAGCCAAGUCAACGAGCUGAUUAAC
CAGUCCCUCGCCUUCAUCAACCAAUCCGACGAGCUCCUGCAUAACGUGAACGCCGGAAAGU
CCACCACCAACAUCAUGAUCACUACUAUUAUCAUCGUGAUCAUCGUCAUCCUGCUGAGCCU
GAUUGCUGUGGGCCUGUUGCUGUAUUGCAAAGCCAGGUCCACCCCGGUCACCCUGUCGAA
GGAUCAGCUGUCCGGAAUCAACAACAUUGCCUUCUCCAACUAACGGGUGGCAUCCCUGUGA
CCCCUCCCCAGUGCCUCUCCUGGCCCUGGAAGUUGCCACUCCAGUGCCCACCAGCCUUGU
CCUAAUAAAAUUAAGUUGCAUC
hMPV FD2 mRNA:
(SEQ ID NO: 16)
GGACAGAUCGCCUGGAGACGCCAUCCACGCUGUUUUGACCUCCAUAGAAGACACCGGGACC
GAUCCAGCCUCCGCGGCCGGGAACGGUGCAUUGGAACGCGGAUUCCCCGUGCCAAGAGUG
ACUCACCGUCCUUGACACGAUGAGCUGGAAGGUUGUGAUUAUUUUCUCUCUGCUGAUUACU
CCACAGCACGGCCUGAAGGAGUCCUACCUGGAGGAGUCCUGUUCUACUAUCACUGAGGGG
UAUCUCUCUGUGCUGCGGACAGGGUGGUAUACAGUGGUGUUCACCCUGGAGGUUGGCGAU
GUGGAGAAUCUGACUUGCAGCGAUGGCCCUUCUCUGAUCAAGACCGAGCUGGAUCUGACAA
AAAGCGCCCUCAGAGAACUGAAAACCGUGUCCGCCGAUCAGCUGGCAAGGGAGGAGCAGAU
CGAGAACCCACGGCAGAGCAGGUUUGUGCUGGGCGCUAUCGCUCUGGGCGUGGCCACUGC
AGCUGCUGUCACUGCAGGGGUCGCAAUCGCUAAGACUAUCAGACUGGAAUCCGAGGUGAC
CGCCAUUAAGAAUGCCCUGAAGACUACCAACGAGGCUGUGUCCACUCUGGGAAACGGAGUG
AGGGUCCUGGCCUUCGCAGUGAGGGAGCUGAAGGAUUUUGUGUCAAAGAACCUUACACGG
GCCAUCAACAAGAAUAAGUGCGAUAUCGAUGACCUGAAGAUGGCCGUGUCCUUCUCCCAGU
UCAACCGGCGCUUUCUGAAUGUGGUGCGCCAGUUUUCCGACAACGCUGGAAUCACCCCUG
CUAUCAGCCUGGACCUCAUGACCGACGCCGAACUCGCAAGGGCCGUUUCUAACAUGCCUAC
AUCCGCUGGACAGAUUAAGCUGAUGCUGGAGAAUAGAGCAAUGGUGAGGAGAAAGGGAUUC
GGCAUCCUGAUUGGCGUGUACGGAUCUAGCGUGAUCUACAUGGUGCAGCUGCCGAUCUUC
GGCGUGAUCGAUACUCCUUGUUGGAUCGUCAAGGCCGCCCCUUCCUGCUCCGAGAAGAAG
GGCAAUUACGCUUGUCUGCUGCGGGAGGACCAGGGCUGGUAUUGCCAGAACGCCGGGUCU
ACAGUGUACUAUCCUAACGAGAAGGAUUGCGAGACCAGAGGCGACCACGUUUUCUGUGAUA
CAGCCGCCGGAAUCAAUGUCGCAGAGCAGUCUAAGGAGUGCAACAUCAAUAUCUCUACAAC
CAAUUACCCAUGUAAGGUGAGCACUGGACGGCACCCUAUCAGUAUGGUGGCUCUGAGCCCA
CUGGGGGCACUGGUGGCUUGCUACAAGGGGGUGAGCUGCAGUAUCGGCAGUAACAGAGUG
GGCAUUAUCAAGCAGCUGAACAAAGGGUGCUCUUAUAUUACAAACCAGGAUGCAGAUACUG
UGACCAUCGACAACACUGUGUACCAGCUGUCCAAGGUGGAGGGGGAGCAGCAUGUGAUCAA
AGGGAGACCCGUCUCUUCUUCUUUCGAUCCCAUCAAGUUCCCUGAAGACCAGUUCAAUGUU
GCCCUGGACCAGGUUUUCGAGAACAUCGAAAAUAGCCAGGCCUUGGUCGAUCAAUCCAACA
GGAUCCUGAGCAGCGCAGAGAAAGGGAACACUGGCUUCAUCAUCGUGAUCAUUCUGAUCGC
CGUGCUGGGGAGCAGUAUGAUUCUGGUGUCCAUUUUCAUCAUCAUCAAGAAGACCAAGAAG
CCUACAGGAGCACCCCCUGAGCUGAGCGGAGUGACCAACAACGGCUUUAUCCCUCACAACU
AACGGGUGGCAUCCCUGUGACCCCUCCCCAGUGCCUCUCCUGGCCCUGGAAGUUGCCACU
CCAGUGCCCACCAGCCUUGUCCUAAUAAAAUUAAGUUGCAUC
hPIV3 FD2 mRNA:
(SEQ ID NO: 17)
GGACAGAUCGCCUGGAGACGCCAUCCACGCUGUUUUGACCUCCAUAGAAGACACCGGGACC
GAUCCAGCCUCCGCGGCCGGGAACGGUGCAUUGGAACGCGGAUUCCCCGUGCCAAGAGUG
ACUCACCGUCCUUGACACGAUGCUGAUUAGCAUUCUGUCCAUCAUCACAACCAUGAUUAUG
GCAAGUCACUGUCAGAUCGAUAUCACCAAGCUCCAGCACGUGGGCGUGCUGGUGAAUUCCC
CAAAGGGGAUGAAGAUUUCUCAGAACUUUGAGACUCGGUACCUGAUCCUGUCCCUGAUUCC
UAAGAUUGAGGAUAGCAACUCCUGUGGCGACCAGCAGAUCAAACAGUACAAACGCCUGCUG
GACAGACUGAUCAUCCCACUGUACGACGGCCUGAAGCUGCAGAAAGACGUCAUUGUUACAA
ACCAGGAAAGCAACGAGAAUACCGACCCUAGGACUGAGCGGUUCUUCGGGGGCGUCAUCG
GAACAAUCGCACUGGGCGUUGCCACUAGCGCACAGAUCACCGCCGCCGUUGCACUGGUGG
AGGCUAAGCAGGCAAAAUCUGACAUCGAGAAGCUGAAAGAAGCUAUCCGCGAUACCAACAA
GGCCGUGCAGUCAGUGUGCUCCAGCGUCGGCAACUGCAUCGUUGCUAUUAAGUCCGUGCA
GGACUACGUGAACAAGGAGAUCGUGCCCAGUAUCGCAAGGCUGGGAUGUGAGGCCGCUGG
ACUGCAGUUGGGGAUCGCUCUCACCCAGCACUACUCCGAGCUGACAAAUUGCUUCGGAGAU
AACAUCGGAUCUCUGCAGGAGAAGGGCAUCAAACUGCAGUGUAUUGCCUCCCUGUACAGAA
CAAAUAUCACAGAGAUUUUCACAACCAGCACUGUCGACAAAUAUGAUAUCUAUGACCUGCUG
UUCACCGAGUCCAUUAAGGUGCGGGUCAUCGACGUUGACCUGAACGAUUAUUCCAUCACCC
UCCAGGUGAGACUGCCUCUGCUGACACGGCUGCUGAACACUCAGAUCUACAAGGUCGACUC
CAUCAGCUACAACAUCCAGAACCGCGAGUGGUACAUCCCACUGCCUAGCCACAUCAUGACA
AAGGGCGCUUUUCUGGGAGGAGCUGAUGUGAAGGAAUGCAUUGAGGCCUUCAGCAGCUAC
AUCUGCCCUUCCGAUCCCGGCUUCGUCCUGAACCACGAGAUGGAGAGCUGUCUGAGCGGG
AACAUUAGCCAGUGCCCCAGGACAACCGUCACCUCUGACAUUGUGCCAAGGUAUGCCUUCG
UGAACGGCGGAGUGGUUGCCAAUUGCAUCACCACUACAUGCACUUGCAAUGGGAUUGGCAA
CAGGAUCAACCAGCCACCAGACCAGGGGGUGAAAAUCAUUACACACAAAGAGUGCAACACCA
UCGGCAUCAACGGAAUGCUGUUCAAUACCAACAAGGAGGGCACCCUGGCCUUCUACACACC
CGAUGACAUCACCCUGAACAAUAGCGUGGCUCUGGAUCCCAUUGAUAUCAGCAUUGAGCUG
AAUAAGGUCAAGUCUGAUCUGGAGGAGAGCAAGGAGUGGUAUAGACGGUCUAAUCAGAAGC
UCGACUCUAUCGGAUCCUGGCACCAGUCCUCCACAACAAUCAUCAUUAUCCUGAUCAUGAU
GAUCAUCCUGUUCAUCAUCAACAUCACAAUUAUCACCAUUGCCAUCAAGUAUUACCGGAUUC
AGAAGCGGAAUCGCGUUGACCAGAACGACAAACCUUAUGUUCUGACCAACAAAUAACGGGU
GGCAUCCCUGUGACCCCUCCCCAGUGCCUCUCCUGGCCCUGGAAGUUGCCACUCCAGUGC
CCACCAGCCUUGUCCUAAUAAAAUUAAGUUGCAUC

### Example 2: hRSV, hMPV, and hPIV3 Pre-Fusion mRNA Compatibility Studies

Compatibility of the pre-fusion hRSV F, hPIV3 F, and hMPV F proteins recited above when co-formulated or co-administered in a 1:1:1 ratio was tested in vitro in human skeletal muscle (hSKM) cells and in vivo in mice. Full-length versions of the pre-fusion hRSV protein, the pre-fusion hPIV3 protein, and the pre-fusion hMPV protein were synthesized as mRNA and formulated as monovalent mRNA-LNP formulations and trivalent mRNA-LNP co-formulations in a 1:1:1 ratio. To test co-administration of the pre-fusion hRSV, hMPV, and hPIV3 proteins in vivo, the monovalent mRNA-LNP formulation of each immunogen was combined just before immunization in a 1:1:1 ratio to create an admix. mRNA constructs were formulated with LNP OF-02, unless otherwise stated.

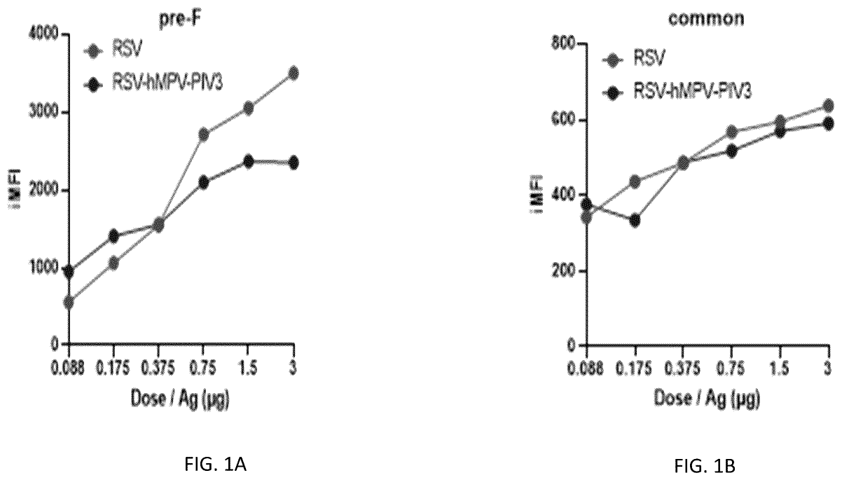

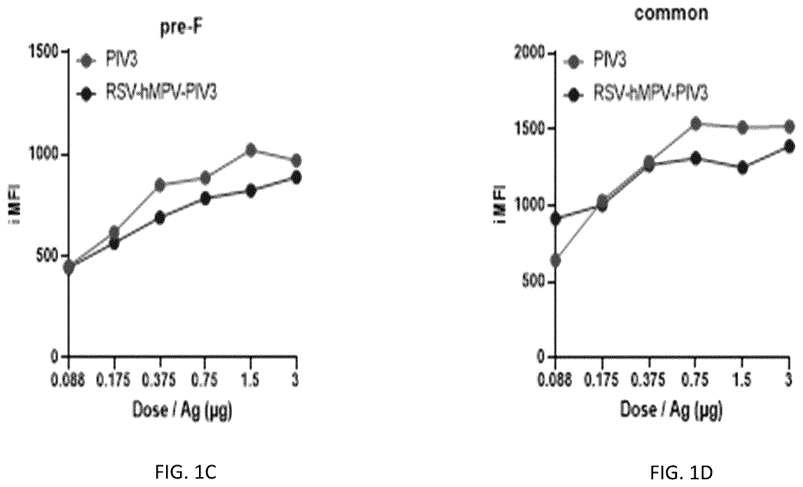

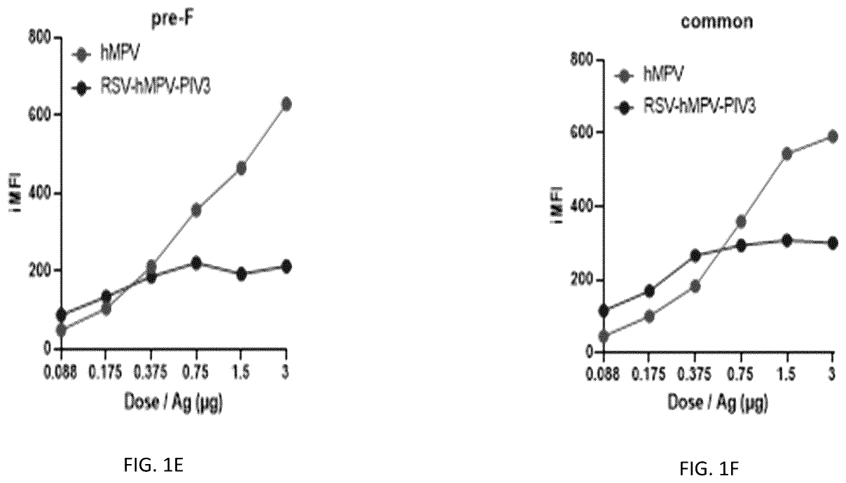

With respect to in vitro studies, hSKM cells were transfected with trivalent hRSV:hMPV:hPIV3 mRNA-LNP co-formulations and with monovalent mRNA-LNP formulations of either pre-fusion hRSV mRNA, pre-fusion hPIV3 mRNA, or pre-fusion hMPV mRNA at doses ranging from 0.088 μg to 3 μg. Interference between components of each formulation was determined using flow cytometry by assessing the expression levels of pre-fusion F specific epitopes and common region epitopes, which was computed as integrated Mean Fluorescent Intensity (iMFI). Transfection with monovalent mRNA-LNP formulations of pre-fusion hRSV F, hMPV F, and hPIV3 F resulted in a robust, dose-specific, expression with similar patterns for both pre-fusion F epitopes and common region epitopes, indicating that the pre-fusion F monoclonal antibody binding sites were well preserved in all three constructs following expression from mRNA. Transfection with the trivalent mRNA-LNP co-formulations produced patterns of expression similar to those following monovalent transfection for RSV and PIV3, as shown in FIG. 1A-FIG. 1D. However, at higher dose levels, the expression of hMPV F epitopes in cells transfected with the trivalent mRNA-LNP co-formulations was lower than the expression of hMPV F epitopes in cells after monovalent transfection, as shown in FIG. 1E. For example, at the 3 μg dose, the iMFI for the pre-fusion hMPV F epitope of the trivalent mRNA-LNP co-formulation was 3-fold lower than the monovalent mRNA-LNP formulation for hMPV. The iMFI for the hMPV common region epitope of the trivalent mRNA-LNP co-formulation was 2-fold lower than the monovalent mRNA-LNP formulation for hMPV, as shown in FIG. 1F.

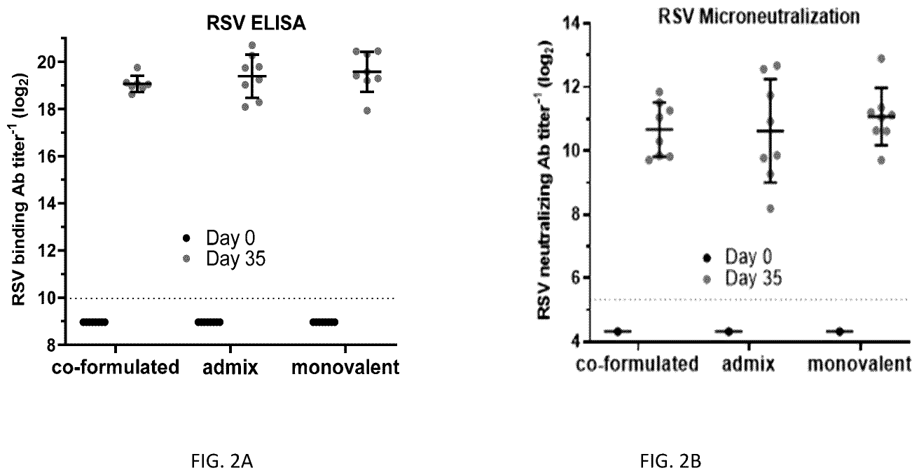

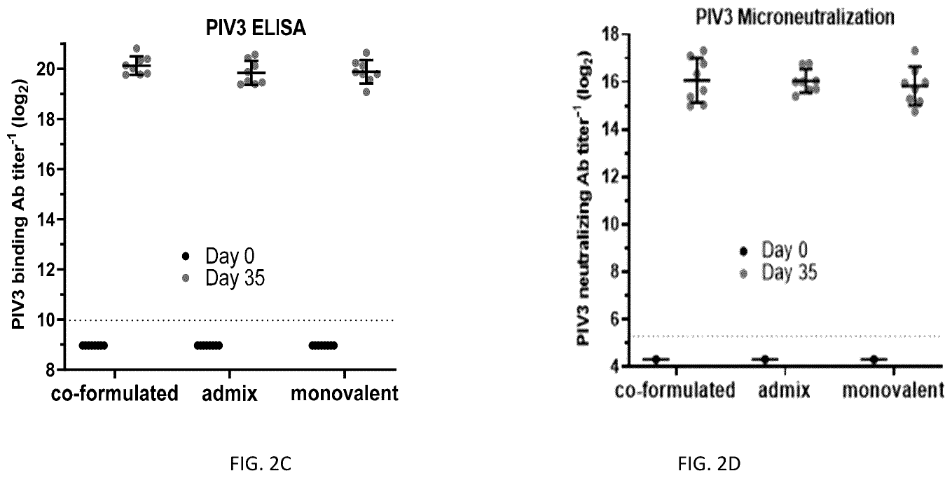

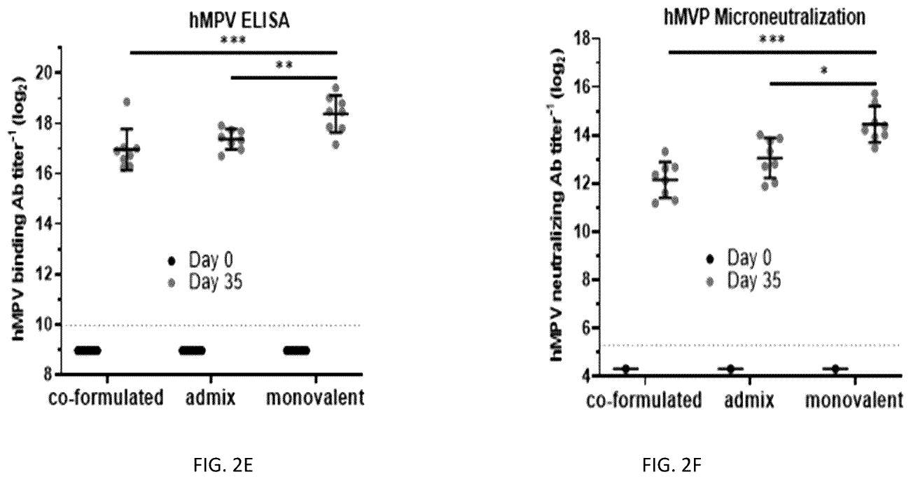

With respect to in vivo studies, interference was determined by comparing immunogenicity in mice vaccinated with the trivalent mRNA-LNP co-formulations (containing 0.5 μg of pre-fusion hRSV mRNA, 0.5 μg of pre-fusion hPIV3 mRNA, and 0.5 μg of pre-fusion hMPV mRNA per dose) or an admix of the three monovalent mRNA-LNP formulations (containing 0.5 μg/dose of pre-fusion hRSV mRNA, 0.5 μg/dose of pre-fusion hPIV3 mRNA, and 0.5 μg/dose of pre-fusion hMPV mRNA), with mice vaccinated with only monovalent mRNA-LNP formulations (containing 0.5 μg/dose of either pre-fusion hRSV mRNA, pre-fusion hPIV3 mRNA, or pre-fusion hMPV mRNA). Binding antibody titers were determined by ELISA using plates coated with pre-fusion hRSV, hPIV3, or hMPV. Neutralizing antibody titers were determined by microneutralization against hRSV, hPIV3, or hMPV expressing Green Fluorescent Protein (GFP) reporters. Similar levels of binding antibody titers and neutralizing antibody titers against RSV and hPIV3 were induced by all three immunization regimens (i.e., co-formulated, admix, and monovalent regimens), as shown in FIG. 2A-FIG. 2D. However, the hMPV binding antibody titers induced by the trivalent mRNA-LNP co-formulations were 2.7-fold lower (p<0.001), and the admix of the three monovalent mRNA-LNP formulations were 2.0-fold lower (p=0.0096), compared to those induced by monovalent hMPV immunization, as shown in FIG. 2E. Similarly, the neutralizing antibody titers induced by the trivalent mRNA-LNP co-formulations were 4.9-fold lower (p<0.001), and the admix of the three monovalent mRNA-LNP formulations were 2.6-fold lower (p=0.0107), compared to those induced by monovalent hMPV immunization, as shown in FIG. 2F.

Data from the above studies demonstrate that when pre-fusion hRSV mRNA, pre-fusion hMPV mRNA, and pre-fusion hPIV3 mRNA are co-formulated or co-administered at a 1:1:1 ratio, there is suppression of hMPV protein expression in vitro and of hMPV immunogenicity in mice relative to the monovalent hMPV immunization. No impact on hRSV and hPIV3 protein expression or immunogenicity was shown.

### Example 3: Trivalent Admix Studies and hMPV Immunogenicity

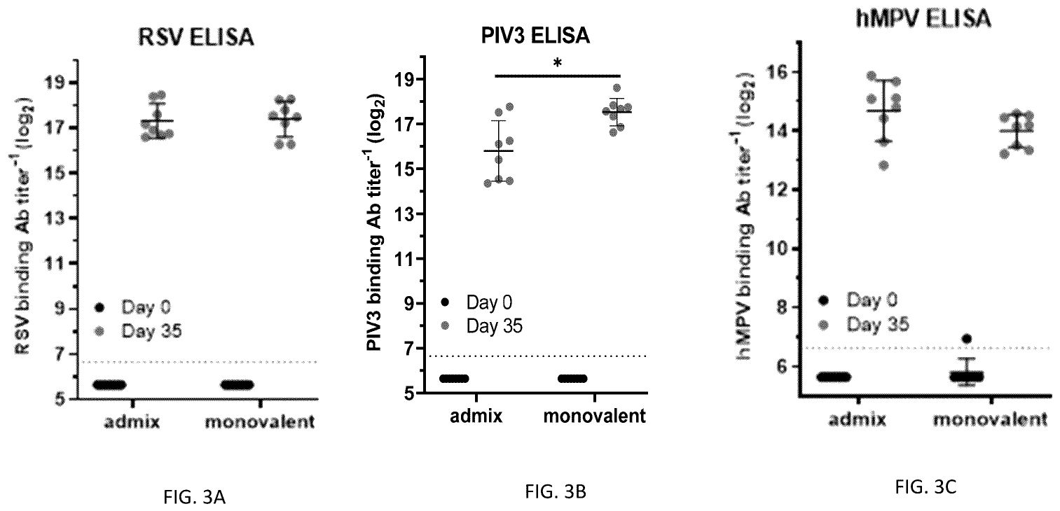

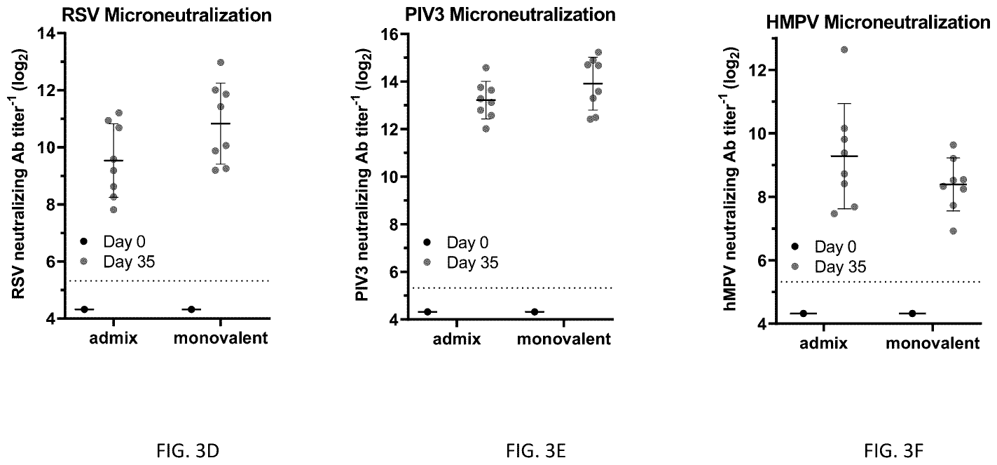

To determine if inhibition of hMPV immunogenicity stems primarily from decreased protein expression observed in vitro or if there was an additional immunodominance component, the compatibility of the three immunogens (i.e., hRSV, hMPV, and hPIV3) when administered as recombinant proteins were evaluated. Following immunization of mice with a 1:1:1 admix of adjuvanted recombinant proteins, the binding antibody titers induced against hRSV and hMPV did not vary significantly from those induced by immunization with monovalent protein controls, while those against hPIV3 were 3.3-fold lower (p=0.0118) than in the group receiving only hPIV3, as shown in FIG. 3A-FIG. 3C. However, there were no significant differences in the induction of neutralizing antibodies against any target between the protein admix immunized groups and the monovalent protein immunized groups, as shown in FIG. 3D-FIG. 3F. Data from this study demonstrates that interference likely occurs at the level of mRNA expression with no immunodominance component.

To determine if inhibition of hMPV immunogenicity could be overcome with dose adjustments by altering the ratio of the pre-fusion hRSV, hMPV, and hPIV3 mRNAs in the admix, two approaches were tested: (1) the hMPV dose was increased 2 or 5-fold relative to the hRSV and hPIV3 doses; and (2) the hPIV3 dose was decreased 5-fold relative to the hRSV and hMPV doses, as shown below in Table 1. mRNA constructs were formulated with LNP OF-02, unless otherwise stated.

TABLE 1
Doses/ratios of admix pre-fusion hRSV, hMPV,
and hPIV3 mRNA tested with LNP OF-02

| | | hRSV:hMPV:hPIV3 | |
|---|---|---|---|
| Group | Modality | ratio | Dose (μg) |
| 1 | mRNA | 1:1:1 | 0.5, 0.5, 0.5 |
| 2 | mRNA | 1:1:0.2 | 0.5, 0.5, 0.1 |
| 3 | mRNA | 1:2:1 | 0.5, 1, 0.5 |
| 4 | mRNA | 1:5:1 | 0.5, 2.5, 0.5 |
| 5 | mRNA | hMPV alone | 0.5 |
| | | 1x dose | (monovalent |
| | | | control for |
| | | | 1x dose of |
| | | | hMPV) |
| 6 | mRNA | hPIV3 alone | 0.1 |
| | | ⅕ dose | (monovalent |
| | | | control for |
| | | | ⅕ dose of |
| | | | hPIV3) |

The effect of hMPV dose increases in admix studies was investigated in groups of mice immunized with a 1:1:1, 1:2:1, or 1:5:1 admix of hRSV:hMPV:hPIV3 mRNA or monovalent hMPV mRNA (containing 0.5 μg/dose of pre-fusion hMPV mRNA). The antibody titers induced by these regimens were compared to those induced by a 1:1:1 admix of hRSV:hMPV:hPIV3 mRNA and, for hMPV only, to those induced by monovalent hMPV mRNA.

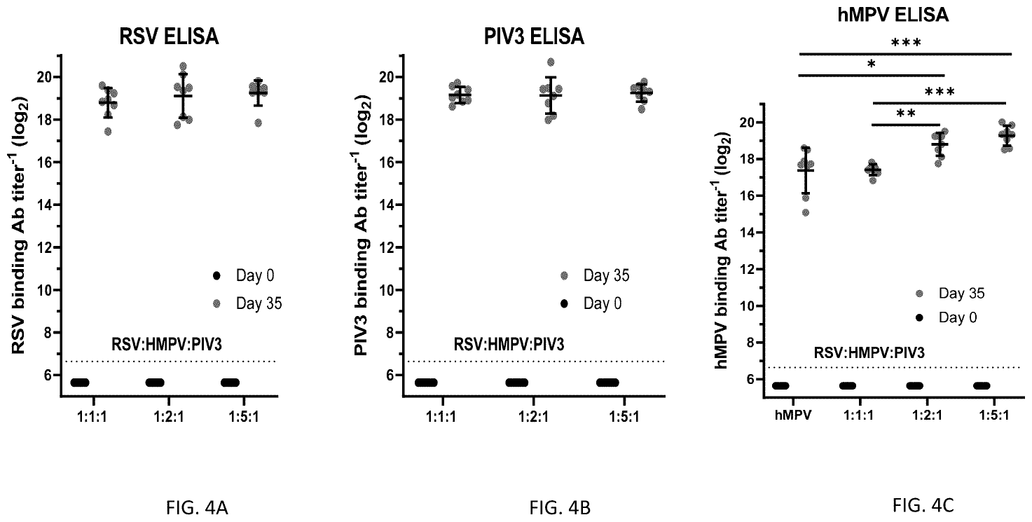

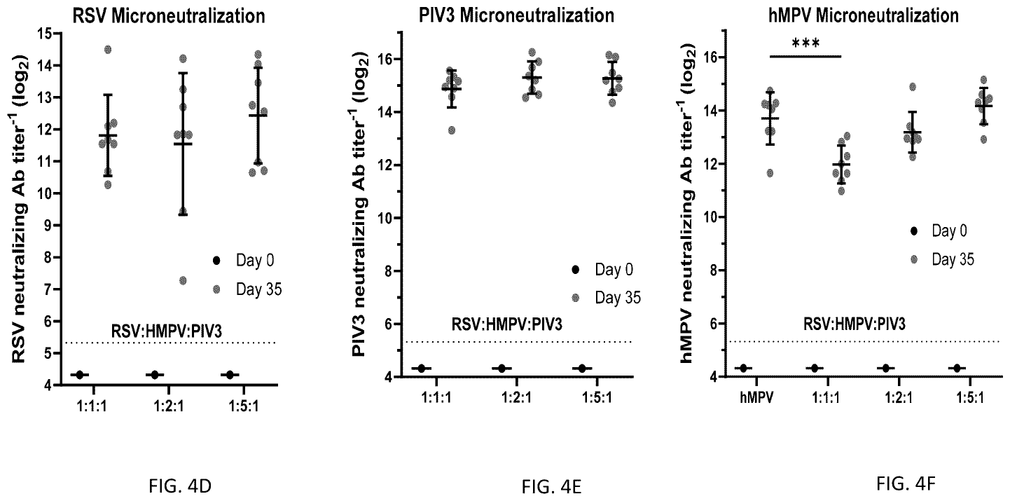

Increasing the hMPV dose 2 or 5-fold had an insignificant effect on immunogenicity against hRSV or hPIV3 as assessed by ELISA (FIG. 4A and FIG. 4B) and microneutralization (FIG. 4D and FIG. 4E). As discussed above, immunization with a 1:1:1 admix of hRSV:hMPV:hPIV3 mRNA resulted in significantly lower induction of hMPV neutralizing antibodies as compared to monovalent hMPV mRNA. A 2-fold increase in the hMPV dose (i.e., 1:2:1 admix of hRSV:hMPV:hPIV3 mRNA) increased neutralizing antibody titers to levels similar (1.4-fold lower, p=0.708) to those induced by monovalent hMPV mRNA (FIG. 4F). Increasing the hMPV dose 5-fold (i.e., 1:5:1 admix of hRSV:hMPV:hPIV3 mRNA) further enhanced immunogenicity, with binding antibody titers 3.7-fold higher (p<0.001) (FIG. 4C) and neutralizing antibody titers 1.4-fold higher (p=0.835) (FIG. 4F) compared to the 0.5 μg of monovalent hMPV. The data from this admix study demonstrates that a 5-fold increase in the hMPV dose overcomes interference without adversely impacting hRSV or hPIV3 immunogenicity.

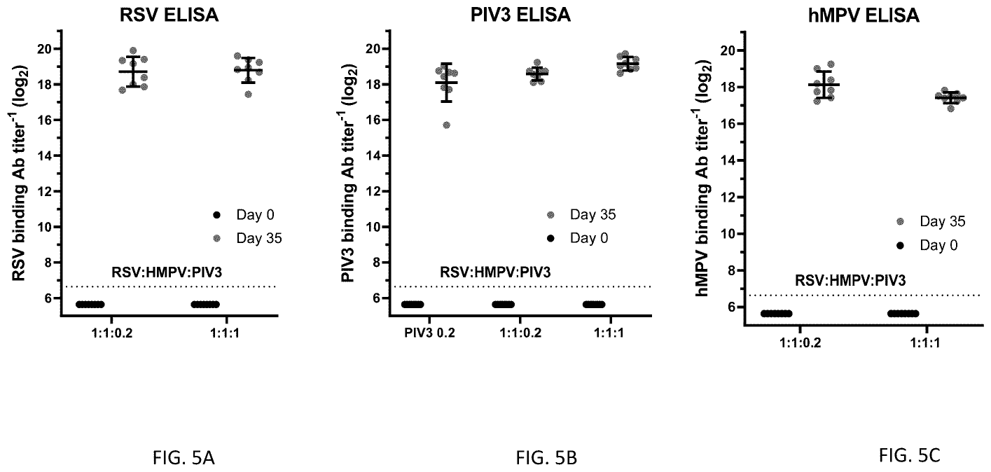

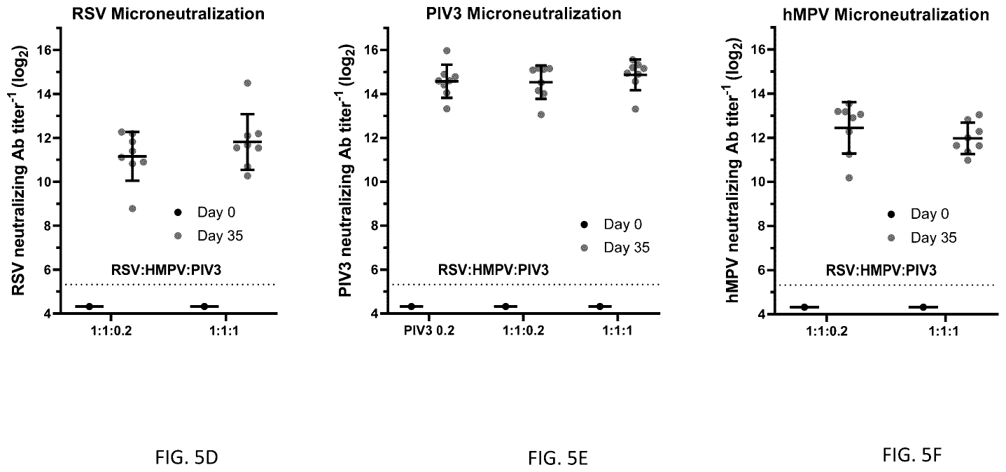

The effect of hPIV3 dose decreases in admix studies was investigated in groups of mice immunized with a 1:1:0.2 admix of hRSV:hMPV:hPIV3 mRNA or a monovalent hPIV3 mRNA (0.1 μg/dose of hPIV3 mRNA). The antibody titers induced by these regimens were compared to those induced by a 1:1:1 admix of hRSV:hMPV:hPIV3 mRNA and additionally, for hPIV3 only, to those induced by 0.1 μg of monovalent hPIV3 mRNA. Data from this admix study demonstrates that decreasing the hPIV3 dose 5-fold did not overcome interference and had no effect on immunogenicity against hRSV, hMPV, or hPIV3 as assessed by ELISA (FIG. 5A-FIG. 5C) and microneutralization (FIG. 5D-FIG. 5F).

Data from the above admix studies demonstrate that: (1) interference appears to be due to mRNA expression; (2) no significant interference was observed when hRSV, hMPV, and hPIV3 recombinant proteins were co-administered; (3) increasing hMPV mRNA dose overcomes interference without negatively effecting hRSV or hPIV3 immunogenicity; and (3) decreasing hPIV3 mRNA dose not overcome interference.

### Example 4: Trivalent Co-Formulation Studies and hMPV Immunogenicity

The effect of hMPV dose increases in co-formulation studies was also investigated using the same ratios of hRSV, hMPV, and hPIV3 in the admix studies discussed above. The doses are shown below in Table 2. mRNA constructs were formulated with LNP OF-02, unless otherwise stated.

TABLE 2
Doses/ratios of co-formulated pre-fusion hRSV,
hMPV, and hPIV3 mRNA tested with LNP OF-02

| | | hRSV:hMPV:hPIV3 | |
|---|---|---|---|
| Group | Modality | ratio | Dose (μg) |
| 1 | mRNA | 1:1:1 | 0.5, 0.5, 0.5 |
| 2 | mRNA | 1:2:1 | 0.5, 1, 0.5 |
| 3 | mRNA | 1:5:1 | 0.5, 2.5, 0.5 |
| 4 | mRNA | RSV alone | 0.5 |
| 5 | mRNA | hMPV alone | 0.5 |
| 6 | mRNA | hPIV3 alone | 0.5 |

To evaluate the effect of hMPV dose increases in co-formulation studies, groups of mice were immunized with a 1:2:1 or 1:5:1 co-formulation of hRSV:hMPV:hPIV3 mRNA, or with monovalent mRNAs (containing 0.5 μg/dose of either pre-fusion hRSV mRNA, pre-fusion hPIV3 mRNA, or pre-fusion hMPV mRNA). The antibody titers induced by the co-formulated hRSV:hMPV:hPIV3 mRNA were compared to those induced by a 1:1:1 co-formulation of hRSV:hMPV:hPIV3 mRNA and those induced by the monovalent controls.

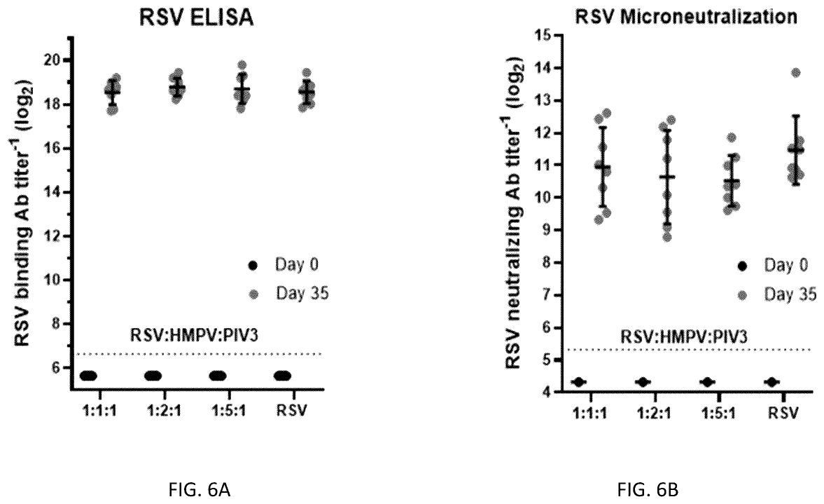

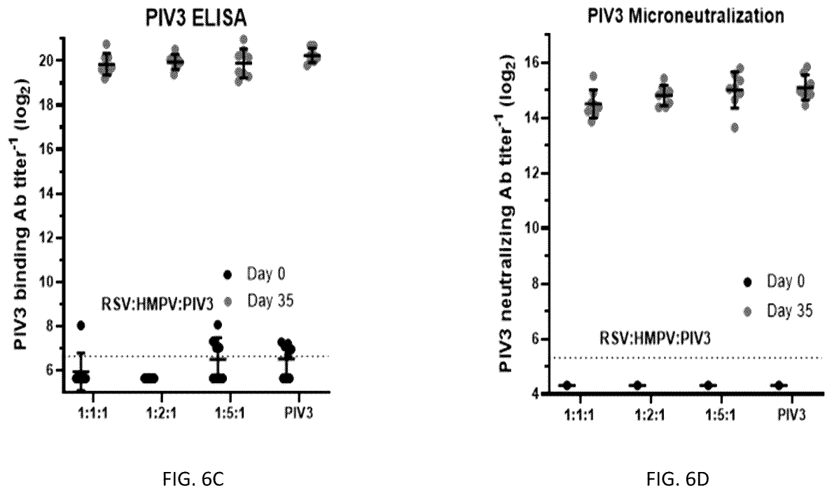

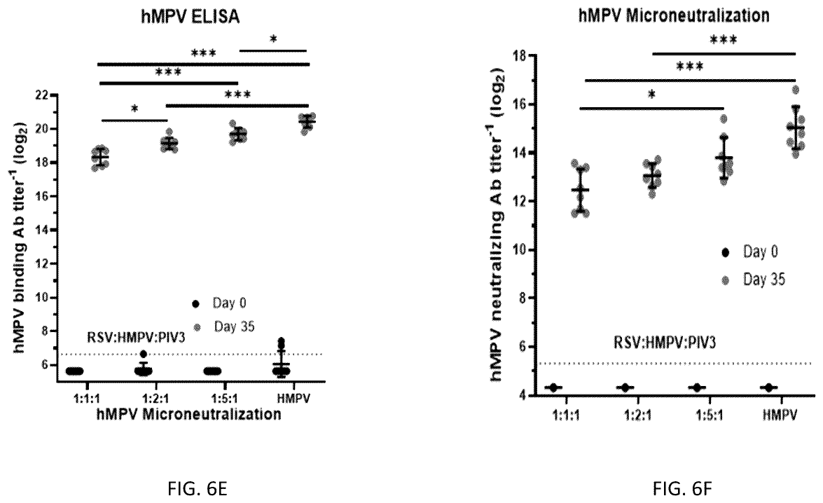

Immunization of the 1:1:1 co-formulation induced similar hRSV binding antibody titers and neutralizing antibody titers compared to immunization with monovalent RSV. Neither the 2-fold nor 5-fold increases in the hMVP dose significantly affected the RSV antibody titers (FIG. 6A and FIG. 6B). Similar results were observed with hPIV3 antibody titers, with no appreciable differences between any groups (FIG. 6C and FIG. 6D). This data demonstrates that interference for hRSV mRNA or hPIV3 mRNA in co-formulations was not observed and there was no significant difference in induction of hRSV and hPIV3 binding antibody titers or neutralizing antibody titers.

With respect to hMPV, immunization of the 1:1:1 co-formulation induced a 4.3-fold lower (p <0.001) binding antibody titer (FIG. 6E) and a 6-fold lower (p<0.001) neutralizing antibody titer (FIG. 6F) compared to immunization with monovalent hMPV. A 2-fold increase in the hMPV dose (i.e., 1:2:1 co-formulation of hRSV:hMPV:hPIV3 mRNA) increased the binding antibody titers and neutralizing antibody titers 1.75-fold (p=0.0159) and 1.52-fold (p=0.8), respectively, relative to the 1:1:1 co-formulation. However, both remained significantly lower than the monovalent control (2.44-fold (p<0.001) and 3.94-fold (p<0.001), respectively). Increasing the hMPV dose 5-fold (i.e., 1:5:1 co-formulation of hRSV:hMPV:hPIV3 mRNA) increased the binding antibody titers and neutralizing antibody titers 2.57-fold (p<0.001) and 2.53-fold (p=0.047), respectively, relative to the 1:1:1 co-formulation immunization. The difference in binding antibody titers relative to monovalent hMPV was very slight (1.66-fold, p=0.0389), while the difference in neutralizing antibody titers relative to monovalent hMPV was not statistically different (2.37-fold, p=0.079).

To further evaluate for presence of interference and the effect of hMPV dose increases, co-formulation studies were formulated with LNP cKK-E10. The doses used are shown below in Table 3.

TABLE 3
Doses/ratios of co-formulated pre-fusion hRSV,
hMPV, and hPIV3 mRNA tested with LNP cKK-E10

| | | hRSV:hMPV:hPIV3 | |
|---|---|---|---|
| Group | Modality | ratio | Dose (μg) |
| 1 | mRNA | 1:2:1 | 0.5, 1, 0.5 |
| 2 | mRNA | 1:5:1 | 0.5, 2.5, 0.5 |
| 3 | mRNA | RSV alone | 0.5 |
| 4 | mRNA | hMPV alone | 0.5 |
| 5 | mRNA | hPIV3 alone | 0.5 |

To evaluate the effect of hMPV dose increases in co-formulation studies with LNP cKK-E10, groups of mice were immunized with a 1:2:1 or 1:5:1 co-formulation of hRSV:hMPV:hPIV3 mRNA, or with monovalent mRNAs (containing 0.5 μg/dose of either pre-fusion hRSV mRNA, pre-fusion hPIV3 mRNA, or pre-fusion hMPV mRNA). The antibody titers induced by the co-formulated hRSV:hMPV:hPIV3 mRNA were then compared to those induced by the monovalent controls.

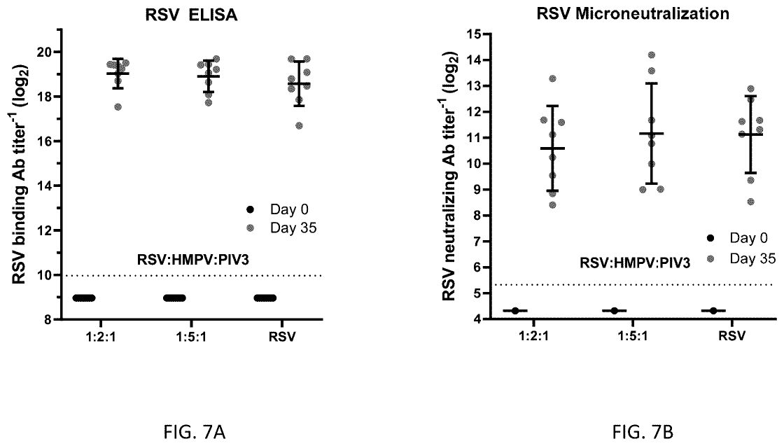

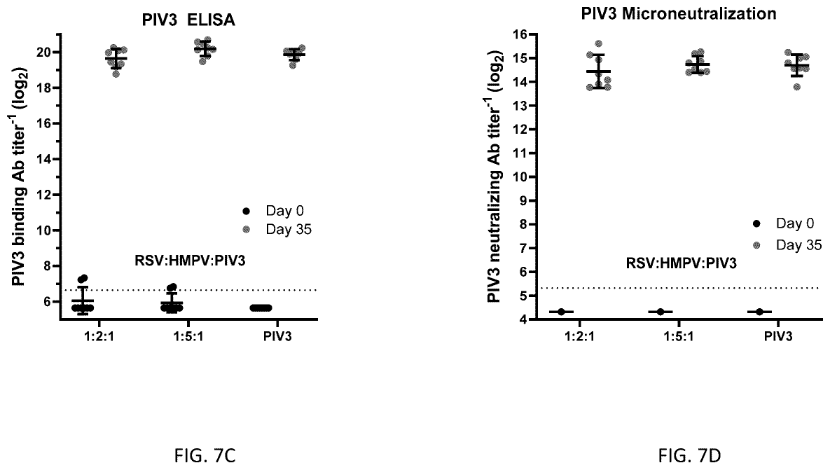

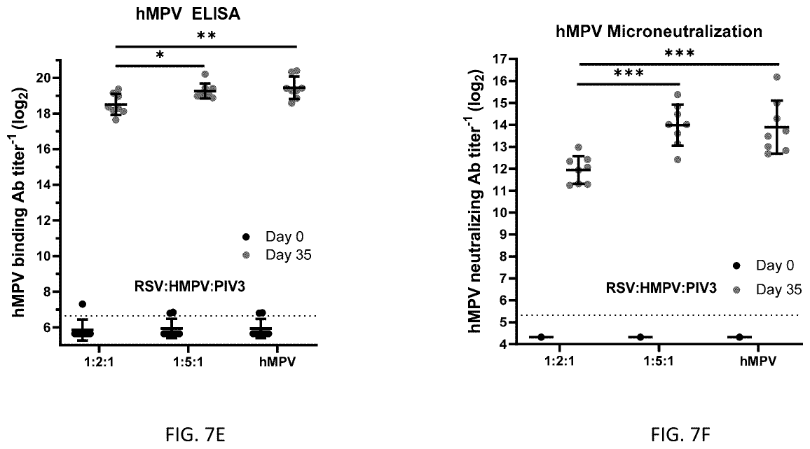

Immunization with the 1:2:1 and 1:5:1 co-formulations induced similar hRSV binding antibody titers and neutralizing antibody titers compared to immunization with monovalent RSV (FIG. 7A and FIG. 7B). Similar results were observed with hPIV3 antibody titers, with no appreciable differences between any groups (FIG. 7C and FIG. 7D). This data demonstrates that interference for hRSV mRNA or hPIV3 mRNA in co-formulations with LNP cKK-E10 was not observed and there was no significant difference in induction of hRSV and hPIV3 binding antibody titers or neutralizing antibody titers.

With respect to hMPV, immunization of the 1:2:1 co-formulation induced a 1.9-fold lower (p=0.0027) binding antibody titer (FIG. 7E) and a 3.9-fold lower (p<0.001) neutralizing antibody titer (FIG. 7F) compared to immunization with monovalent hMPV. Increasing the hMPV dose 5-fold (i.e., 1:5:1 co-formulation of hRSV:hMPV:hPIV3 mRNA) increased the binding antibody titers and neutralizing antibody titers to levels similar to those induced by monovalent hMPV.

Data from co-formulation studies demonstrate that a 5-fold increase in the hMPV dose overcomes interference without adversely impacting hRSV or hPIV3 immunogenicity. Further, mRNA constructs formulated with LNP OF-02 and LNP cKK-E10 exhibit similar patterns of interference with respect to the 1:5:1 co-formulation.

### Example 5: Trivalent Co-Formulation Studies with Various LNPs and Impact on Immunogenicity

Immunogenicity was evaluated for trivalent co-formulations of pre-fusion hRSV, hMPV, and hPIV3 that were formulated with LNP OF-02, LNP cKK-E10, or LNP GL-HEPES-E3-E12-DS-4-E10. The doses/ratios of pre-fusion hRSV, hMPV, and hPIV3 are shown below in Table 4.

TABLE 4
Doses/ratios of co-formulated pre-fusion hRSV,
hMPV, and hPIV3 mRNA tested with LNP OF-02,
LNP cKK-E10, or LNP GL-HEPES-E3-E12-DS-4-E10

| | | hRSV:hMPV:hPIV3 | | |
|---|---|---|---|---|
| Group | Modality | ratio | LNP | Dose (μg) |
| 1 | mRNA | 1:1:1 | OF-02 | 0.5, 0.5, 0.5 |
| | | (Trehalose | | |
| | | bridging group) | | |
| 2 | mRNA | 1:1:1 | OF-02 | 0.5, 0.5, 0.5 |
| 3 | mRNA | 1:3:1 | OF-02 | 0.5, 1.5, 0.5 |
| 4 | mRNA | RSV alone | OF-02 | 0.5 |
| 5 | mRNA | hMPV alone | OF-02 | 0.5 |
| 6 | mRNA | hPIV3 alone | OF-02 | 0.5 |
| 7 | mRNA | 1:1:1 | cKK-E10 | 0.5, 0.5, 0.5 |
| 8 | mRNA | 1:3:1 | cKK-E10 | 0.5, 1.5, 0.5 |
| 9 | mRNA | RSV alone | cKK-E10 | 0.5 |
| 10 | mRNA | hMPV alone | cKK-E10 | 0.5 |
| 11 | mRNA | hPIV3 alone | cKK-E10 | 0.5 |
| 12 | mRNA | 1:1:1 | GL-HEPES- | 0.5, 0.5, 0.5 |
| | | | E3-E12-DS- | |
| | | | 4-E10 | |
| 13 | mRNA | 1:3:1 | GL-HEPES- | 0.5, 1.5, 0.5 |
| | | | E3-E12-DS- | |
| | | | 4-E10 | |
| 14 | mRNA | RSV alone | GL-HEPES- | 0.5 |
| | | | E3-E12-DS- | |
| | | | 4-E10 | |
| 15 | mRNA | hMPV alone | GL-HEPES- | 0.5 |
| | | | E3-E12-DS- | |
| | | | 4-E10 | |
| 16 | mRNA | hPIV3 alone | GL-HEPES- | 0.5 |
| | | | E3-E12-DS- | |
| | | | 4-E10 | |

To evaluate the effect on immunogenicity in co-formulations of pre-fusion hRSV, hMPV, and hPIV3 formulated with LNP OF-02, LNP cKK-E10, or LNP GL-HEPES-E3-E12-DS-4-E10, groups of mice were immunized with a 1:1:1 or 1:3:1 co-formulation of hRSV:hMPV:hPIV3 mRNA, or with monovalent mRNAs (containing 0.5 μg/dose of either pre-fusion hRSV mRNA, pre-fusion hPIV3 mRNA, or pre-fusion hMPV mRNA). The mice in Groups 1-6 (Table 4) were administered said mRNA constructs formulated with LNP OF-02. The mice in Groups 7-11 were administered said mRNA constructs formulated with LNP cKK-E10, and the mice in Groups 12-16 were administered said mRNA constructs formulated with LNP GL-HEPES-E3-E12-DS-4-E10. Each mRNA construct was diluted in PBS except for Group 1 (i.e., the trehalose bridging group), in which the mRNA construct was diluted in trehalose. The antibody titers induced by the co-formulated hRSV:hMPV:hPIV3 mRNA constructs tested with each of the three different LNPs were compared to those induced by the monovalent controls.

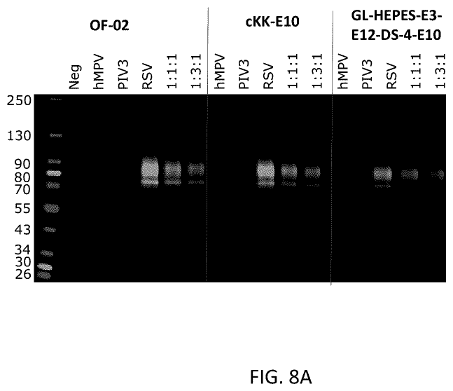

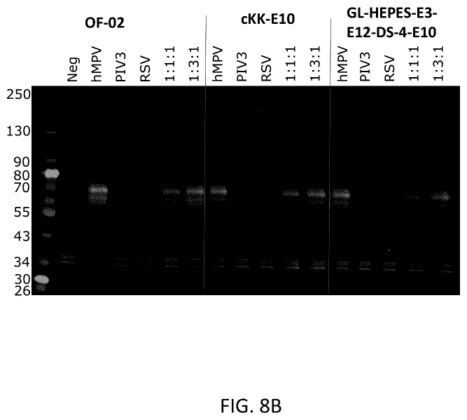

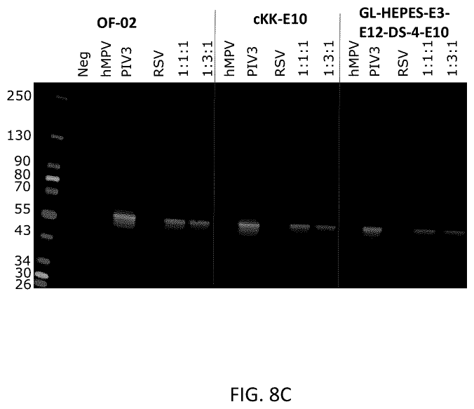

Western blots were performed on lysates from cells transfected with the 1:1:1 and 1:3:1 co-formulation of hRSV:hMPV:hPIV3 mRNA and the monovalent hRSV, hMPV, and hPIV3 mRNAs formulated with LNP OF-02, LNP cKK-E10, or LNP GL-HEPES-E3-E12-DS-4-E10 to detect hRSV, hMPV, and hPIV3 protein expression in each of the formulations (FIG. 8A-8C). All formulations containing hRSV were positive for high levels of protein expression (FIG. 8A). All formulations containing hMPV were positive for high levels of protein expression (FIG. 8B). All formulations containing hPIV3 were positive for high levels of protein expression (FIG. 8C).

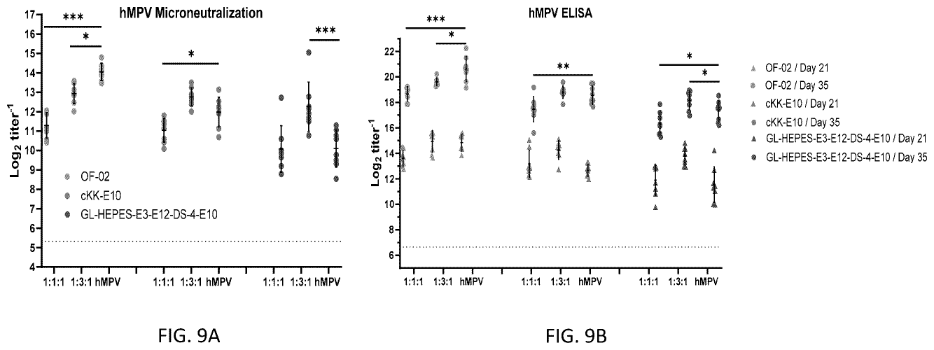

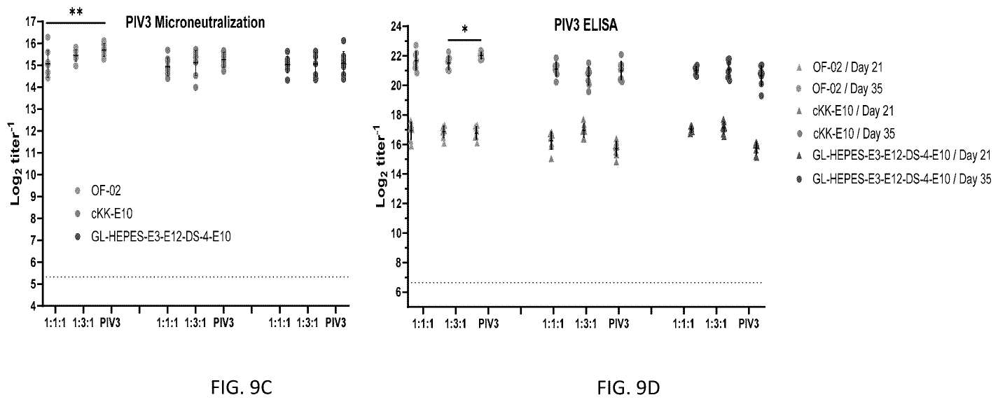

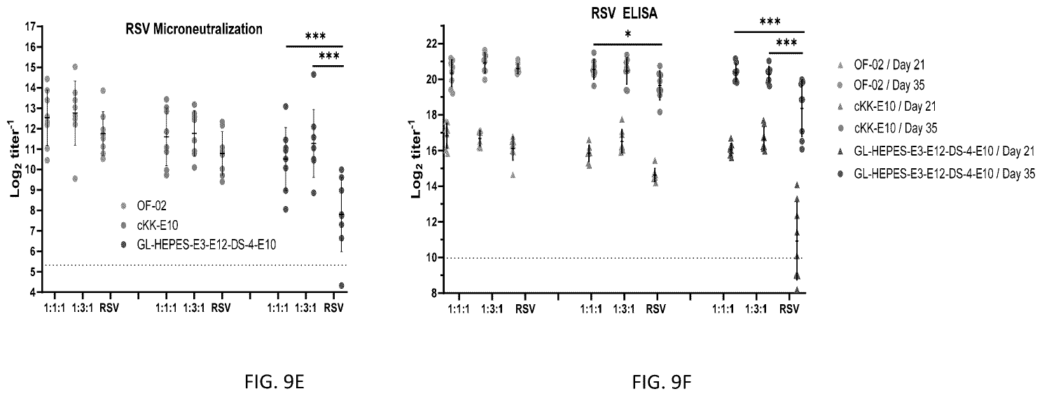

With respect to hMPV, immunization of the 1:1:1 co-formulation with LNP OF-02 induced 3.9-fold lower (p<00.1) binding antibody titer and a 6.8-fold lower (p<0.001) neutralizing antibody titer compared to immunization with monovalent hMPV (FIGS. 9A and 9B). A 3-fold increase in the hMPV dose (i.e., 1:3:1 co-formulation of hRSV:hMPV:hPIV3 mRNA) tested with LNP OF-02 increased the binding antibody titers and neutralizing antibody titers but both remained lower than those induced by the monovalent hMPV control (2-fold lower (p=0.0145) and 2.2-fold lower (p=0.0128) respectively). Immunization of the 1:1:1 co-formulation with LNP cKK-E10 induced a 2.1-fold lower (p<0.008) binding antibody titer and a 1.9-fold lower (p<0.0245) neutralizing antibody titer compared to immunization with monovalent hMPV (FIGS. 9A and 9B). A 3-fold increase in the hMPV dose (i.e., 1:3:1 co-formulation of hRSV:hMPV:hPIV3 mRNA) tested with LNP cKK-E10 increased the binding antibody titers and neutralizing antibody titers to levels similar to those induced by the monovalent hMPV control. Immunization of the 1:1:1 co-formulation with LNP GL-HEPES-E3-E12-DS-4-E10 induced 2-fold lower (p=0.0147) hMPV binding antibody titers and similar neutralizing antibody titers compared to immunization with monovalent hMPV (FIGS. 9A and 9B). Thus, no interference on induction of functional anti-hMPV antibodies was observed. Further, a 3-fold increase in the hMPV dose (i.e., 1:3:1 co-formulation of hRSV:hMPV:hPIV3 mRNA) tested with LNP GL-HEPES-E3-E12-DS-4-E10 induced a 1.8-fold higher (p<0.0395) binding antibody titer and a 4.5-fold higher (p<0.001) neutralizing antibody titer compared to immunization with monovalent hMPV (FIGS. 9A and 9B). Moreover, the data indicates that monovalent hMPV formulated with LNP OF-02 was more immunogenic than monovalent hMPV formulated with cKK-E10 or GL-HEPES-E3-E12-DS-4-E10.

With respect to hPIV3, immunization with the 1:1:1 and 1:3:1 co-formulations with either LNP cKK-E10 or LNP GL-HEPES-E3-E12-DS-4-E10 induced similar hPIV3 binding antibody titers and neutralizing antibody titers compared to immunization with monovalent hPIV3 (FIGS. 9C and 9D). No interference on hPIV3 immunogenicity in co-formulations with LNP cKK-E10 and LNP GL-HEPES-E3-E12-DS-4-E10 was observed. Further, there was no significant difference in induction of hPIV3 binding antibody titers or neutralizing antibody titers with either LNP. Immunization with 1:1:1 and 1:3:1 co-formulations with LNP OF-02 induced similar hPIV3 binding antibody titers and neutralizing antibody titers compared to immunization with monovalent hPIV3.

With respect to RSV, monovalent RSV formulated with LNP cKK-E10 induced a 1.9-fold lower (p<0.0316) binding antibody titer and a 1.8-fold lower (difference not statistically significant) neutralizing antibody titer compared to the 1:1:1 co-formulation with LNP cKK-E10 (FIGS. 9E and 9F). Further, monovalent RSV formulated with LNP GL-HEPES-E3-E12-DS-4-E10 was less immunogenic than expected. Interference was not observed with respect to hRSV in 1:1:1 and 1:3:1 co-formulations with LNP OF-02, LNP cKK-E10, or LNP GL-HEPES-E3-E12-DS-4-E10.

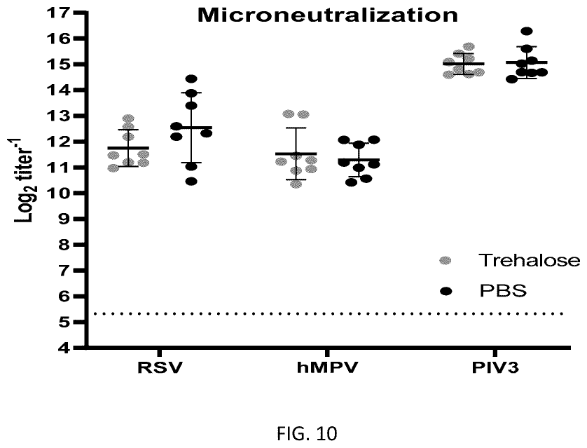

Further, to determine if PBS had any impact on immunogenicity, neutralizing antibody titers for hRSV, hMPV, and hPIV3 mRNA constructs diluted with PBS were compared to neutralizing antibody titers for hRSV, hMPV, and hPIV3 mRNA constructs diluted with trehalose. The results indicated that mRNA constructs diluted with PBS induced similar hRSV, hMPV, and hPIV3 neutralizing antibody titers compared to mRNA constructs diluted with trehalose (FIG. 10). Thus, mRNA constructs diluted in PBS had no impact on immunogenicity compared to mRNA constructs diluted in trehalose.

Data from the above trivalent co-formulation study with LNP OF-02, LNP cKK-E10, and LNP GL-HEPES-E3-E12-DS-4-E10 demonstrate that: (1) different LNPs have varying effects on mRNA expression, which may result in interference; (2) no interference on hMPV immunogenicity was observed in co-formulations with LNP GL-HEPES-E3-E12-DS-4-E1; (3) a slight interference on hMPV immunogenicity was observed in the 1:1:1 co-formulation with LNP cKK-E10, but no interference was observed in the 1:3:1 co-formulation with LNP cKK-E1; (4) no interference on hRSV or hPIV3 immunogenicity was observed in co-formulations with LNP OF-02, LNP cKK-E10, or LNP GL-HEPES-E3-E12-DS-4-E10; and (5) switching from trehalose to PBS as a diluent did not affect immunogenicity.

### Example 6: Bivalent Co-Formulation Studies with Various LNPs and Impact Immunogenicity

Interference on hRSV and hMPV immunogenicity was evaluated for bivalent 1:1 co-formulations of pre-fusion hRSV and hMPV formulated with LNP IM-1, LNP GL-HEPES-E3-E12-DS-4-E10, or LNP OF-02. Further, hRSV and hMPV immunogenicity for the bivalent 1:1 co-formulation tested with IM-001 was compared to the bivalent 1:1 co-formulations tested with GL-HEPES-E3-E12-DS-4-E10 and OF-02. The doses/ratios of pre-fusion hRSV and hMPV are shown below in Table 5.

TABLE 5
Doses/ratios of co-formulated pre-fusion hRSV and hMPV tested
with LNP IM-001, LNP GL-HEPES-E3-E12-DS-4-E10, or OF-02

| | | hRSV:hMPV | | |
|---|---|---|---|---|
| Group | Modality | ratio | LNP | Dose (μg) |
| 1 | mRNA | 1:1 | OF-02 | 0.5, 0.5 |
| 2 | mRNA | 1:1 | GL-HEPES-E3- | 0.5, 0.5 |
| | | | E12-DS-4-E10 | |
| 3 | mRNA | 1:1 | IM-001 | 0.5, 0.5 |
| 4 | mRNA | RSV | OF-02 | 0.5 |
| 5 | mRNA | RSV | GL-HEPES-E3- | 0.5 |
| | | | E12-DS-4-E10 | |
| 6 | mRNA | RSV | IM-001 | 0.5 |
| 7 | mRNA | hMPV | OF-02 | 0.5 |
| 8 | mRNA | hMPV | GL-HEPES-E3- | 0.5 |
| | | | E12-DS-4-E10 | |
| 9 | mRNA | hMPV | IM-001 | 0.5 |

To evaluate hRSV and hMPV interference and immunogenicity in co-formulations of pre-fusion hRSV and hMPV formulated with LNP IM-001, LNP GL-HEPES-E3-E12-DS-4-E10, or LNP OF-02, groups of mice were immunized with a 1:1 co-formulation of hRSV:hMPV mRNA, or with monovalent mRNAs (containing 0.5 μg/dose of either pre-fusion hRSV mRNA or pre-fusion hPIV3 mRNA). The mice in Groups 1-3 (Table 5) were administered a 1:1 co-formulation of hRSV:hMPV mRNA with LNP IM-001, LNP GL-HEPES-E3-E12-DS-4-E10, or LNP OF-02. The mice in Groups 4-6 were administered a monovalent hRSV mRNA with LNP IM-001, LNP GL-HEPES-E3-E12-DS-4-E10, or LNP OF-02, and the mice in Groups 7-9 were administered a monovalent hMPV mRNA with LNP IM-001, LNP GL-HEPES-E3-E12-DS-4-E10, or LNP OF-02. The antibody titers induced by the co-formulated hRSV:hMPV mRNA constructs tested with each of the three different LNPs were compared to each other and those induced by the monovalent controls.

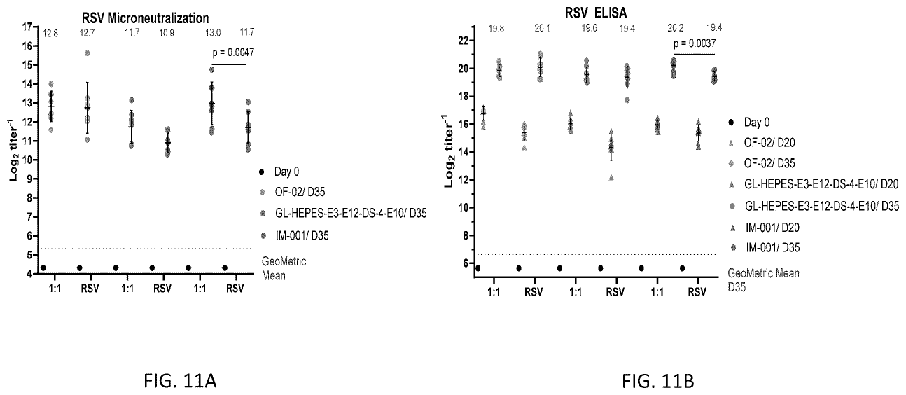

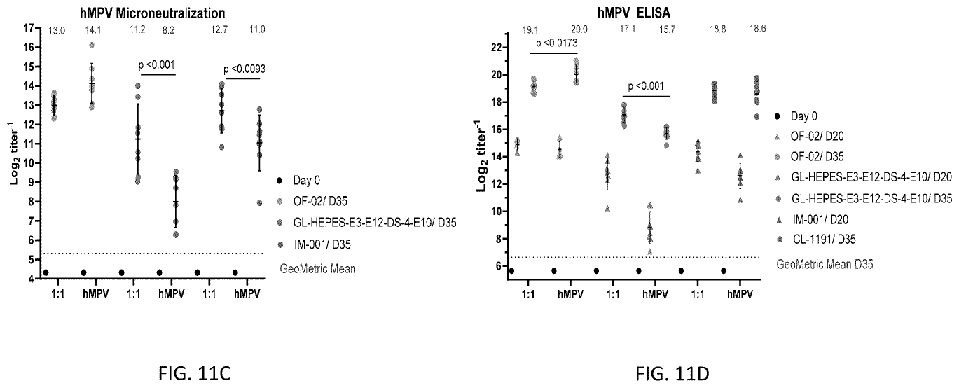

Interference on hRSV immunogenicity was not observed in any of the bivalent 1:1 co-formulations of pre-fusion hRSV and hMPV formulated with LNP IM-001, LNP GL-HEPES-E3-E12-DS-4-E10, or LNP OF-02 (FIGS. 11A and 11B). Further, interference on hMPV immunogenicity was not observed in the bivalent 1:1 co-formulations of pre-fusion hRSV and hMPV formulated with LNP IM-001 and LNP GL-HEPES-E3-E12-DS-4-E10 (FIGS. 11C and 11D). However, the bivalent 1:1 co-formulation tested with OF-02 induced a 1.9-fold lower (p<0.0173) binding antibody titer and a 2.2-fold lower (difference not statistically significant) neutralizing antibody titer compared to immunization with monovalent hMPV (FIGS. 11C and 11D).

With respect to hRSV immunogenicity, the bivalent 1:1 co-formulation tested with LNP IM-001 was more immunogenic and induced a 2.4-fold higher RSV neutralizing antibody titer compared to the bivalent 1:1 co-formulation tested with LNP GL-HEPES-E3-E12-DS-4-E10 (FIG. 11A). Further, the bivalent 1:1 co-formulation tested with LNP IM-001 induced similar hRSV binding antibody titers and neutralizing antibody titers compared to immunization with the bivalent 1:1 co-formulation tested with LNP OF-02 (FIGS. 11A and 11B). Further the bivalent 1:1 co-formulation tested with LNP IM-001 was more immunogenic than monovalent hRSV inducing 2.4-fold higher (p=0.0047) neutralizing and 1.7-fold higher (p=0.0037) binding antibody titers.

With respect to hMPV immunogenicity, the bivalent 1:1 co-formulation tested with LNP IM-001 was more immunogenic and induced a 2.8-fold higher (p=0.0211) hMPV neutralizing antibody titer compared to the bivalent 1:1 co-formulation tested with LNP GL-HEPES-E3-E12-DS-4-E10. (FIG. 11C). Further, the bivalent 1:1 co-formulation tested with LNP IM-001 induced similar hMPV binding antibody and neutralizing antibody titers compared to immunization with the bivalent 1:1 co-formulation tested with LNP OF-02 (FIGS. 11C and 11D). Further the bivalent 1:1 co-formulation tested with LNP IM-001 was more immunogenic than monovalent hMPV inducing 3.2-fold higher (p<0.0093) neutralizing antibody titers, while the bivalent 1:1 co-formulation tested with LNP GL-HEPES-E3-E12-DS-4-E10 induced neutralizing antibody titers 9.5-fold higher (p<0.001) than monovalent hMPV tested with LNP GL-HEPES-E3-E12-DS-4-E10.

Data from the above bivalent co-formulation studies with LNP IM-001, LNP GL-HEPES-E3-E12-DS-4-E10, and LNP OF-02 demonstrate that: (1) no interference on hRSV or hMPV immunogenicity was observed in bivalent co-formulations tested with either LNP IM-001 or LNP GL-HEPES-E3-E12-DS-4-E10; (2) bivalent co-formulations tested with IM-001 were more immunogenic than those tested with LNP GL-HEPES-E3-E12-DS-4-E10; and (3) antibody titers in the 1:1 co-formulations were generally higher compared to the antibody titers in the monovalent controls.

### Example 7: Compatibility of RSV and hMPV Bivalent Co-Formulation Administered with hPIV3 Admix

Interference on hRSV, hMPV, and hPIV3 immunogenicity was evaluated when bivalent co-formulations of pre-fusion hRSV and hMPV were administered with an hPIV3 admix. The doses of pre-fusion hRSV, hMPV, and hPIV3 are shown below in Table 6.

TABLE 6
Doses of bivalent co-formulated pre-fusion
hRSV and hMPV, and pre-fusion hPIV3

| Group | Modality | Construct | Dose (μg) |
|---|---|---|---|
| 1 | mRNA | RSV/hMPV | 0.5, 0.5 |
| 2 | mRNA | RSV/hMPV + hPIV3 | 0.5, 0.5, 0.5 |
| 3 | mRNA | hPIV3 | 0.5 |

To evaluate the effect of interference on hRSV, hMPV, and hPIV3 immunogenicity in bivalent co-formulations of pre-fusion hRSV and hMPV administered with an hPIV3 admix, groups of mice were immunized with a bivalent 1:1 co-formulation of hRSV:hMPV, a bivalent 1:1 co-formulation of hRSV:hMPV administered with hPIV3 (containing 0.5 μg/dose of pre-fusion hPIV3 mRNA), or monovalent hPIV3 (containing 0.5 μg/dose of pre-fusion hPIV3 mRNA) as the control. Each mRNA construct was formulated with LNP GL-HEPES-E3-E12-DS-4-E10 and diluted in PBS. The antibody titers induced by the bivalent 1:1 co-formulation of hRSV:hMPV administered with hPIV3 was compared to antibody titers induced by the bivalent 1:1 co-formulation of hRSV:hMPV and the antibody titers induced by the monovalent hPIV3 control.

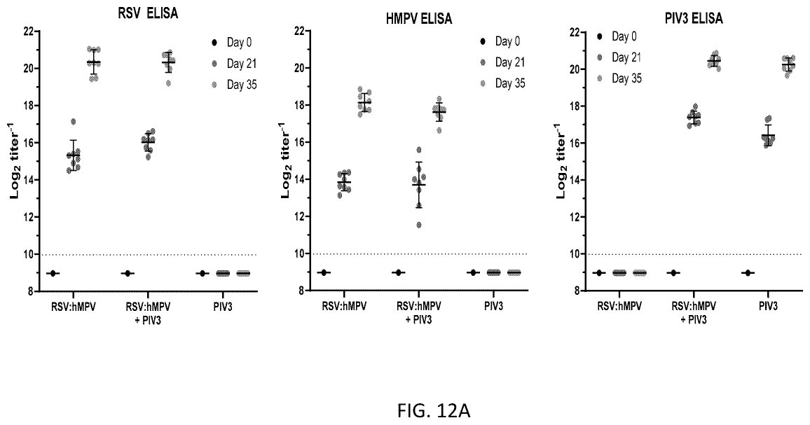

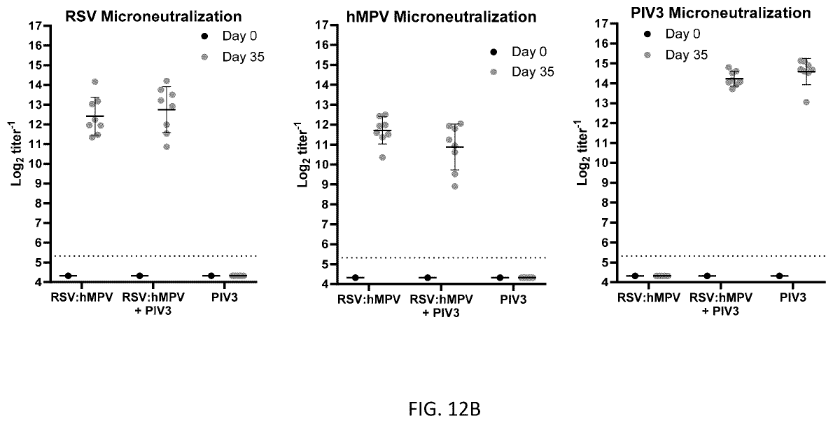

Immunization with the bivalent 1:1 co-formulation of hRSV:hMPV together with the hPIV3 admix induced similar hRSV, hMPV, and hPIV3 binding antibody titers and neutralizing antibody titers compared to immunization with the bivalent 1:1 co-formulation of hRSV:hMPV alone and the monovalent hPIV3 control alone (FIG. 12A and FIG. 12B). No statistically significant differences were observed for hRSV, hMPV, and hPIV3 binding or neutralizing antibody titers. This data demonstrates that there was no interference on hRSV, hMPV, or hPIV3 immunogenicity when bivalent co-formulations of hRSV:hMPV were administered with an hPIV3 admix with LNP GL-HEPES-E3-E12-DS-4-E10.

1. A composition comprising at least two messenger RNAs (mRNAs), wherein the at least two mRNAs comprise an open reading frame (ORF) encoding a recombinant F protein antigenic polypeptide selected from the group consisting of:
(i) a first mRNA encoding a human respiratory syncytial virus (hRSV) F protein antigen comprising an amino acid sequence of SEQ ID NO: 1;
(ii) a second mRNA encoding a human metapneumovirus (hMPV) F protein antigen comprising an amino acid sequence of SEQ ID NO: 2; and
(iii) a third mRNA encoding a human parainfluenza virus 3 (hPIV3) F protein antigen comprising an amino acid sequence of SEQ ID NO: 3.

2. The composition of claim 1, comprising:
the first mRNA encoding the amino acid sequence of SEQ ID NO: 1; and
the second mRNA encoding the amino acid sequence of SEQ ID NO: 2,
optionally wherein the first mRNA encodes the amino acid sequence of SEQ ID NO: 1;
and the third mRNA encodes the amino acid sequence of SEQ ID NO: 3,
optionally wherein the second mRNA encodes the amino acid sequence of SEQ ID NO: 2;
and the third mRNA encodes the amino acid sequence of SEQ ID NO: 3,
optionally wherein the first mRNA encodes the amino acid sequence of SEQ ID NO: 1;
the second mRNA encodes the amino acid sequence of SEQ ID NO: 2; and the third mRNA encodes the amino acid sequence of SEQ ID NO: 3.

3-6. (canceled)

7. The composition of claim 1, wherein the hRSV F protein comprises an amino acid sequence having at least 90% identity to SEQ ID NO: 1,
optionally wherein the hMPV F protein comprises an amino acid sequence having at least 90% identity to SEQ ID NO: 2,
optionally wherein the hPIV3 F protein comprises an amino acid sequence having at least 90% identity to SEQ ID NO: 3,
optionally wherein the first mRNA comprises a nucleic acid sequence having at least 80% identity to SEQ ID NO: 4,
optionally wherein the second mRNA comprises a nucleic acid sequence having at least 80% identity to SEQ ID NO: 5,
optionally wherein the third mRNA comprises a nucleic acid sequence having at least 80% identity to SEQ ID NO: 6.

8-12. (canceled)

13. The composition of claim 1, wherein at least one of the recombinant F protein antigenic polypeptides is a pre-fusion protein,
optionally wherein at least one of the mRNAs comprises a codon-optimized ORF.

14-17. (canceled)

18. The composition of claim 1, wherein at least one of the mRNAs comprises at least one chemical modification,
optionally wherein at least 20%, at least 30%, at least 40%, at least 50%, at least 60%, at least 70%, at least 80%, at least 85%, at least 90%, at least 95%, or 100% of the uracil nucleotides in at least one of the mRNAs are chemically modified,
optionally wherein at least 20%, at least 30%, at least 40%, at least 50%, at least 60%, at least 70%, at least 80%, at least 85%, at least 90%, at least 95%, or 100% of the uracil nucleotides in at least one of the ORFs are chemically modified,
optionally wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine,
optionally wherein the chemical modification is selected from the group consisting of pseudouridine, N1-methylpseudouridine, 5-methylcytosine, 5-methoxyuridine, and a combination thereof.

19-23. (canceled)

24. The composition of claim 1, wherein the first mRNA, the second mRNA, and the third mRNA are present in a ratio (w/w) of about 1:1:1;
optionally wherein the first mRNA, the second mRNA, and the third mRNA are present in a ratio (w/w) of about 1:2:1,
optionally wherein the first mRNA, the second mRNA, and the third mRNA are present in a ratio (w/w) of about 1:3:1,
optionally wherein the first mRNA, the second mRNA, and the third mRNA are present in a ratio (w/w) of about 1:5:1,
optionally wherein the first mRNA, the second mRNA, and the third mRNA are present in a ratio (w/w) of about 1:1:1 to about 1:10:1,
optionally wherein the first mRNA, the second mRNA, and the third mRNA are present in a ratio (w/w) of about 1:1:0.1 to about 1:1:1,
optionally wherein the ratio is expressed in micrograms (μg),
optionally wherein the first mRNA, the second mRNA, and the third mRNA are present in a ratio (w/w) of about one microgram of the first mRNA to about one microgram of the second mRNA to about one microgram of the third mRNA,
optionally wherein the first mRNA, the second mRNA, and the third mRNA are present in a ratio (w/w) of about one microgram of the first mRNA to about one microgram of the second mRNA to about one microgram of the third mRNA,
optionally wherein the first mRNA, the second mRNA, and the third mRNA are present in a ratio (w/w) of about one microgram of the first mRNA to about two micrograms of the second mRNA to about one microgram of the third mRNA,
optionally wherein the first mRNA, the second mRNA, and the third mRNA are present in a ratio (w/w) of about one microgram of the first mRNA to about three micrograms of the second mRNA to about one microgram of the third mRNA,
optionally wherein the first mRNA, the second mRNA, and the third mRNA are present in a ratio (w/w) of about one microgram of the first mRNA to about five micrograms of the second mRNA to about one microgram of the third mRNA,
optionally wherein the first mRNA, the second mRNA, and the third mRNA are present in a ratio (w/w) of about 0.5 microgram of the first mRNA to about 0.5 micrograms of the second mRNA to about 0.5 microgram of the third mRNA,
optionally wherein the first mRNA, the second mRNA, and the third mRNA are present in a ratio (w/w) of about 0.5 microgram of the first mRNA to about 1.5 micrograms of the second mRNA to about 0.5 microgram of the third mRNA,
optionally wherein the first mRNA, the second mRNA, and the third mRNA are present in a ratio (w/w) of about 0.5 microgram of the first mRNA to about 0.5 microgram of the second mRNA to about 0.1 microgram of the third mRNA,
optionally wherein the first mRNA, the second mRNA, and the third mRNA are present in a ratio (w/w) of about 0.5 microgram of the first mRNA to about 1 microgram of the second mRNA to about 0.5 microgram of the third mRNA,
optionally wherein the first mRNA, the second mRNA, and the third mRNA are present in a ratio (w/w) of about 0.5 microgram of the first mRNA to about 2.5 micrograms of the second mRNA to about 0.5 microgram of the third mRNA.

25-39. (canceled)

40. The composition of claim 1, wherein the first mRNA, the second mRNA, and the third mRNA are not covalently linked to one another, or wherein one or more of the first mRNA, the second mRNA, and the third mRNA are covalently linked to one another.

41. (canceled)

42. The composition of claim 1, wherein the first mRNA, the second mRNA, and the third mRNA are each formulated into a separate nanoparticle (LNP), or wherein the first mRNA, the second mRNA, and the third mRNA are formulated into the same LNP,
optionally wherein the LNP comprises at least one cationic lipid,
optionally wherein the cationic lipid is biodegradable or not biodegradable, optionally wherein the cationic lipid is cleavable or not cleavable.

43-48. (canceled)

49. The composition of claim 42, wherein the cationic lipid is selected from the group consisting of OF-02, cKK-E10, GL-HEPES-E3-E10-DS-3-E18-1, GL-HEPES-E3-E12-DS-4-E10, GL-HEPES-E3-E12-DS-3-E14, and IM-001, optionally wherein the LNP further comprises a polyethylene glycol (PEG) conjugated (PEGylated) lipid, a cholesterol-based lipid, and a helper lipid.

50-53. (canceled)

54. The composition of claim 49, wherein the LNP comprises:
a cationic lipid at a molar ratio of 35% to 55%;
a polyethylene glycol (PEG) conjugated (PEGylated) lipid at a molar ratio of 0.25% to 2.75%;
a cholesterol-based lipid at a molar ratio of 20% to 45%; and
a helper lipid at a molar ratio of 5% to 35%,
wherein all of the molar ratios are relative to the total lipid content of the LNP,
optionally wherein the LNP comprises:
a cationic lipid at a molar ratio of 40%;
a PEGylated lipid at a molar ratio of 1.5%;
a cholesterol-based lipid at a molar ratio of 28.5%; and
a helper lipid at a molar ratio of 30%;
optionally wherein the PEGylated lipid is dimyristoyl-PEG2000 (DMG-PEG2000) or 2-[(polyethylene glycol)-2000]-N,N-ditetradecylacetamide (ALC-0159),
optionally wherein the cholesterol-based lipid is cholesterol,
optionally wherein the helper lipid is 1,2-dioleoyl-SN-glycero-3-phosphoethanolamine (DOPE) or 1,2-distearoyl-sn-glycero-3-phosphocholine (DSPC),
optionally wherein the LNP comprises:
GL-HEPES-E3-E12-DS-4-E10 at a molar ratio of 40%;
DMG-PEG2000 at a molar ratio of 1.5%;
cholesterol at a molar ratio of 28.5%; and
DOPE at a molar ratio of 30%,
optionally wherein the LNP comprises:
cKK-E10 at a molar ratio of 40%;
DMG-PEG2000 at a molar ratio of 1.5%;
cholesterol at a molar ratio of 28.5%; and
DOPE at a molar ratio of 30%,
optionally wherein the LNP comprises:
IM-001 at a molar ratio of 40%;
DMG-PEG2000 at a molar ratio of 1.5%;
cholesterol at a molar ratio of 28.5%; and
DOPE at a molar ratio of 30%,
optionally wherein the LNP has an average diameter of 30 nm to 200 nm,
optionally wherein the LNP has an average diameter of 80 nm to 150 nm.

55-64. (canceled)

65. A composition comprising at least two messenger RNAs (mRNAs), wherein each of the at least two mRNAs comprises an open reading frame (ORF) encoding a recombinant F protein antigenic polypeptide, wherein at least one of the mRNAs comprises the following structural elements:
A)
(i) a 5′ cap with the following structure: 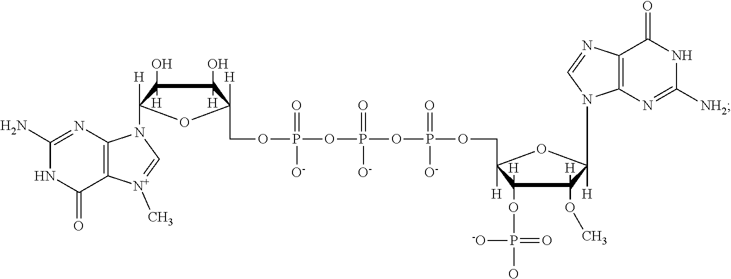
(ii) a 5′ untranslated region (5′ UTR) having the nucleic acid sequence of SEQ ID NO: 7;
(iii) a protein coding region having the nucleic acid sequence of SEQ ID NO: 4, 5, or 6;
(iv) a 3′ untranslated region (3′ UTR) having the nucleic acid sequence of SEQ ID NO: 8; and
(v) a poly(A) tail;
wherein the mRNA is formulated in a lipid nanoparticle (LNP) comprising:
GL-HEPES-E3-E12-DS-4-E10 at a molar ratio of 40%;
DMG-PEG2000 at a molar ratio of 1.5%;
cholesterol at a molar ratio of 28.5%; and
DOPE at a molar ratio of 30%;_or
(B)
(i) a 5′ cap with the following structure: 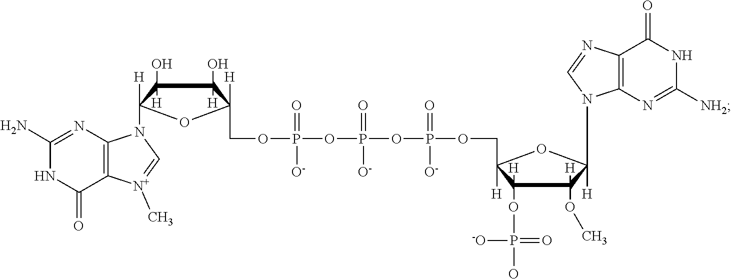
(ii) a 5′ untranslated region (5′ UTR) having the nucleic acid sequence of SEQ ID NO: 7;
(iii) a protein coding region having the nucleic acid sequence of SEQ ID NO: 4, 5, or 6:
(iv) a 3′ untranslated region (3′ UTR) having the nucleic acid sequence of SEQ ID NO: 8; and
(v) a poly(A) tail:
wherein the mRNA is formulated in a lipid nanoparticle (LNP) comprising:
cKK-E10 at a molar ratio of 40%;
DMG-PEG2000 at a molar ratio of 1.5%;
cholesterol at a molar ratio of 28.5%; and
DOPE at a molar ratio of 30%; or
(C)
(i) a 5′ cap with the following structure: 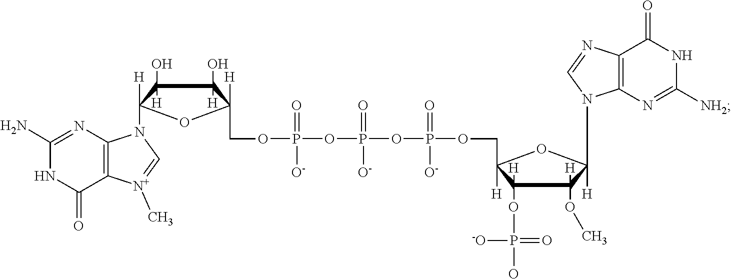
(ii) a 5′ untranslated region (5′ UTR) having the nucleic acid sequence of SEQ ID NO: 7;
(iii) a protein coding region having the nucleic acid sequence of SEQ ID NO: 4, 5, or 6;
(iv) a 3′ untranslated region (3′ UTR) having the nucleic acid sequence of SEQ ID NO: 8; and
(v) a poly(A) tail:
wherein the mRNA is formulated in a lipid nanoparticle (LNP) comprising:
IM-001 at a molar ratio of 40%;
DMG-PEG2000 at a molar ratio of 1.5%;
cholesterol at a molar ratio of 28.5%; and
DOPE at a molar ratio of 30%; or
(D)
(i) a 5′ cap with the following structure: 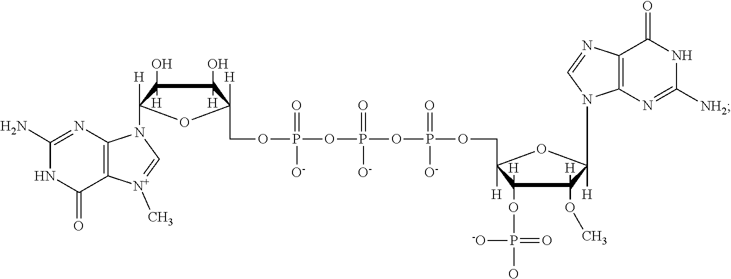
(ii) a 5′ untranslated region (5′ UTR) having the nucleic acid sequence of SEQ ID NO: 7;
(iii) a protein coding region having the nucleic acid sequence of SEQ ID NO: 4, 5, or 6;
(iv) a 3′ untranslated region (3′ UTR) having the nucleic acid sequence of SEQ ID NO: 8; and
(v) a poly(A) tail;
wherein the mRNA is formulated in a lipid nanoparticle (LNP) comprising GL-HEPES-E3-E12-DS-4-E10, IM-001, OF-02, or cKK-E10 at a molar ratio of 35% to 55%.

66-68. (canceled)

69. A method of eliciting an immune response to hRSV or protecting a subject against hRSV infection, eliciting an immune response to hMPV or protecting a subject against hMPV infection, and/or eliciting an immune response to hPIV3 or protecting a subject against hPIV3 infection comprising administering the composition of claim 1 to a subject,
optionally wherein the subject has about the same or higher serum concentration of neutralizing antibodies against hRSV after administration of the composition, relative to a subject that is administered a single antigenic composition comprising an mRNA ORF encoding an hRSV F protein antigen of SEQ ID NO: 1,
optionally wherein the subject has about the same or higher serum concentration of neutralizing antibodies against hMPV after administration of the composition, relative to a subject that is administered a single antigenic composition comprising an mRNA ORF encoding an hMPV F protein antigen of SEQ ID NO: 2,
optionally wherein the subject has about the same or higher serum concentration of neutralizing antibodies against hPIV3 after administration of the composition, relative to a subject that is administered a single antigenic composition comprising an mRNA ORF encoding an hPIV3 F protein antigen of SEQ ID NO: 3,
optionally wherein the subject has a comparable serum concentration of neutralizing antibodies against hRSV, hMPV, and/or hPIV3 after administration of the composition, relative to a subject that is administered a protein hRSV vaccine, a protein hMPV vaccine, and/or a protein hPIV3 vaccine,
optionally wherein the composition increases the serum concentration of neutralizing antibodies in a subject with pre-existing hRSV immunity, pre-existing hMPV immunity, or pre-existing hPIV3 immunity.

70-86. (canceled)

87. A composition comprising three messenger RNAs (mRNAs), wherein:
A)
(i) a first mRNA encodes a human respiratory syncytial virus (hRSV) F protein antigen comprising an amino acid sequence of SEQ ID NO: 1;
(ii) a second mRNA encodes a human metapneumovirus (hMPV) F protein antigen comprising an amino acid sequence of SEQ ID NO: 2; and
(iii) a third mRNA encodes a human parainfluenza virus 3 (hPIV3) F protein antigen comprising an amino acid sequence of SEQ ID NO: 3;
wherein the first mRNA, the second mRNA, and the third mRNA are formulated into the same lipid nanoparticle (LNP) comprising:
OF-02, cKK-E10, GL-HEPES-E3-E10-DS-3-E18-1, GL-HEPES-E3-E12-DS-4-E10, GL-HEPES-E3-E12-DS-3-E14, or IM-001 at a molar ratio of 40%;
DMG-PEG2000 at a molar ratio of 1.5%;
cholesterol at a molar ratio of 28.5%; and
DOPE at a molar ratio of 30%;_or
(B)
(i) a first mRNA encodes a human respiratory syncytial virus (hRSV) F protein antigen comprising an amino acid sequence of SEQ ID NO: 1:
(ii) a second mRNA encodes a human metapneumovirus (hMPV) F protein antigen comprising an amino acid sequence of SEQ ID NO: 2; and
(iii) a third mRNA encodes a human parainfluenza virus 3 (hPIV3) F protein antigen comprising an amino acid sequence of SEQ ID NO: 3.

88-103. (canceled)

104. A method of eliciting an immune response to hRSV or protecting a subject against hRSV infection, eliciting an immune response to hMPV or protecting a subject against hMPV infection, and/or eliciting an immune response to hPIV3 or protecting a subject against hPIV3 infection comprising administering the composition of claim 87 to a subject,
optionally wherein the subject has about the same or higher serum concentration of neutralizing antibodies against hRSV after administration of the composition, relative to a subject that is administered a single antigenic composition comprising an mRNA ORF encoding an hRSV F protein antigen of SEQ ID NO: 1,
optionally wherein the subject has about the same or higher serum concentration of neutralizing antibodies against hMPV after administration of the composition, relative to a subject that is administered a single antigenic composition comprising an mRNA ORF encoding an hMPV F protein antigen of SEQ ID NO: 2,
optionally wherein the subject has about the same or higher serum concentration of neutralizing antibodies against hPIV3 after administration of the composition, relative to a subject that is administered a single antigenic composition comprising an mRNA ORF encoding an hPIV3 F protein antigen of SEQ ID NO: 3,
optionally wherein the subject has a comparable serum concentration of neutralizing antibodies against hRSV, hMPV, and/or hPIV3 after administration of the composition, relative to a subject that is administered a protein hRSV vaccine, a protein hMPV vaccine, and/or a protein hPIV3 vaccine,
optionally wherein the composition increases the serum concentration of neutralizing antibodies in a subject with pre-existing hRSV immunity, pre-existing hMPV immunity, or pre-existing hPIV3 immunity.

105-138. (canceled)

139. A method of eliciting an immune response to hRSV or protecting a subject against hRSV infection, eliciting an immune response to hMPV or protecting a subject against hMPV infection, and/or eliciting an immune response to hPIV3 or protecting a subject against hPIV3 infection comprising administering the composition of claim 122 to a subject,
optionally wherein the subject has about the same or higher serum concentration of neutralizing antibodies against hRSV after administration of the composition, relative to a subject that is administered a single antigenic composition comprising an mRNA ORF encoding an hRSV F protein antigen of SEQ ID NO: 1,
optionally wherein the subject has about the same or higher serum concentration of neutralizing antibodies against hMPV after administration of the composition, relative to a subject that is administered a single antigenic composition comprising an mRNA ORF encoding an hMPV F protein antigen of SEQ ID NO: 2,
optionally wherein the subject has about the same or higher serum concentration of neutralizing antibodies against hPIV3 after administration of the composition, relative to a subject that is administered a single antigenic composition comprising an mRNA ORF encoding an hPIV3 F protein antigen of SEQ ID NO: 3,
optionally wherein the subject has a comparable serum concentration of neutralizing antibodies against hRSV, hMPV, and/or hPIV3 after administration of the composition, relative to a subject that is administered a protein hRSV vaccine, a protein hMPV vaccine, and/or a protein hPIV3 vaccine,
optionally wherein the composition increases the serum concentration of neutralizing antibodies in a subject with pre-existing hRSV immunity, pre-existing hMPV immunity, or pre-existing hPIV3 immunity.

140-156. (canceled)

157. A composition comprising a first messenger RNA (mRNA) and a second mRNA, wherein:
(i) the first mRNA encodes a human respiratory syncytial virus (hRSV) F protein antigen comprising an amino acid sequence of SEQ ID NO: 1; and
(ii) the second mRNA encodes a human metapneumovirus (hMPV) F protein antigen comprising an amino acid sequence of SEQ ID NO: 2.

158-193. (canceled)

194. A method of eliciting an immune response to hRSV or protecting a subject against hRSV infection, and/or eliciting an immune response to hMPV or protecting a subject against hMPV infection comprising administering the composition of claim 87 to a subject,
optionally wherein the subject has about the same or higher serum concentration of neutralizing antibodies against hRSV after administration of the composition, relative to a subject that is administered a single antigenic composition comprising an mRNA ORF encoding an hRSV F protein antigen of SEQ ID NO: 1,
optionally wherein the subject has about the same or higher serum concentration of neutralizing antibodies against hMPV after administration of the composition, relative to a subject that is administered a single antigenic composition comprising an mRNA ORF encoding an hMPV F protein antigen of SEQ ID NO: 2,
optionally wherein the subject has a comparable serum concentration of neutralizing antibodies against hRSV and/or hMPV after administration of the composition, relative to a subject that is administered a protein hRSV vaccine and/or a protein hMPV vaccine,
optionally wherein the composition increases the serum concentration of neutralizing antibodies in a subject with pre-existing hRSV immunity or pre-existing hMPV immunity.

195-205. (canceled)
